(19) United States
(12) Patent Application Publication
(10) Pub. No.: US 2025/0084436 A1
(43) Pub. Date: Mar. 13, 2025

(54) ADENO-ASSOCIATED VIRUS VECTORS

(71) Applicant: University of Pittsburgh - Of the Commonwealth System of Higher Education, Pittsburgh, PA (US)

(72) Inventors: Leah Caroline THOMAS BYRNE, Pittsburgh, PA (US); Molly E. JOHNSON, Pittsburgh, PA (US); William Richard STAUFFER, Pittsburgh, PA (US); Bilge Esin OZTURK, Pittsburgh, PA (US)

(21) Appl. No.: 18/851,427

(22) PCT Filed: Mar. 30, 2023
(86) PCT No.: PCT/US2023/016867

§ 371 Date: Sep. 26, 2024

Related U.S. Application Data
(60) Provisional application No. 63/325,562, filed on Mar. 30, 2022.
(60) Provisional application No. 63/325,540, filed on Mar. 30, 2022.
(60) Provisional application No. 63/325,542, filed on Mar. 30, 2022.
(60) Provisional application No. 63/325,543, filed on Mar. 30, 2022.
(60) Provisional application No. 63/325,544, filed on Mar. 30, 2022.
(60) Provisional application No. 63/325,548, filed on Mar. 30, 2022.
(60) Provisional application No. 63/325,550, filed on Mar. 30, 2022.
(60) Provisional application No. 63/325,551, filed on Mar. 30, 2022.
(60) Provisional application No. 63/325,553, filed on Mar. 30, 2022.
(60) Provisional application No. 63/325,555, filed on Mar. 30, 2022.
(60) Provisional application No. 63/325,557, filed on Mar. 30, 2022.
(60) Provisional application No. 63/325,558, filed on Mar. 30, 2022.
(60) Provisional application No. 63/325,559, filed on Mar. 30, 2022.

(51) Int. Cl. C12N 15/86; A61K 48/00; C07K 14/005; C12N 5/079; C12N 15/11

(52) U.S. Cl. CPC C12N 15/86; A61K 48/005; A61K 48/0075; C07K 14/005; C12N 5/0621; C12N 15/11; C12N 2310/141; C12N 2510/00; C12N 2750/14122; C12N 2750/14143; C12N 2750/14145

(57) ABSTRACT

This document relates to AAV vectors (e.g., AAV2 vectors). For example, AAV vectors (e.g., AAV2 vectors) containing an AAV capsid polypeptide that includes an amino acid sequence set forth in any one or more of Tables 1A-1L (or a variant thereof) or according to Formula A based on such a set forth sequence, such AAV capsid polypeptides, nucleic acid molecules encoding such vectors, nucleic acid molecules encoding such AAV capsid polypeptides, host cells containing and/or expressing such nucleic acid molecules, and methods and materials for making or using such vectors and/or AAV capsid polypeptides are provided.

### CROSS-REFERENCE TO RELATED APPLICATIONS

This application claims the benefit of U.S. Patent Application Ser. No. 63/325,562, filed on Mar. 30, 2022, of U.S. Patent Application Ser. No. 63/325,540, filed on Mar. 30, 2022, of U.S. Patent Application Ser. No. 63/325,542, filed on Mar. 30, 2022, of U.S. Patent Application Ser. No. 63/325,543, filed on Mar. 30, 2022, of U.S. Patent Application Ser. No. 63/325,544, filed on Mar. 30, 2022, of U.S. Patent Application Ser. No. 63/325,548, filed on Mar. 30, 2022, of U.S. Patent Application Ser. No. 63/325,550, filed on Mar. 30, 2022, of U.S. Patent Application Ser. No. 63/325,551, filed on Mar. 30, 2022, of U.S. Patent Application Ser. No. 63/325,553, filed on Mar. 30, 2022, of U.S. Patent Application Ser. No. 63/325,555, filed on Mar. 30, 2022, of U.S. Patent Application Ser. No. 63/325,557, filed on Mar. 30, 2022, of U.S. Patent Application Ser. No. 63/325,558, filed on Mar. 30, 2022, and of U.S. Patent Application Ser. No. 63/325,559, filed on Mar. 30, 2022. The disclosures of the prior applications are considered part of (and are incorporated by reference in) the disclosure of this application.

### STATEMENT REGARDING FEDERAL FUNDING

This invention was made with government support under MH120094 awarded by the National Institutes of Health. The government has certain rights in the invention.

### TECHNICAL FIELD

This document relates to adeno-associated virus (AAV) vectors. For example, this document provides methods and materials for making and using AAV vectors (e.g., AAV2 vectors) having (a) the ability to deliver nucleic acid to foveal cones, (b) the ability to deliver nucleic acid to retinal cells and drive high expression levels of nucleic acid within retinal cells, (c) the ability to deliver nucleic acid to retinal cells across retinal regions (e.g., across at least two retinal regions), (d) the ability to deliver nucleic acid to retinal cells of the parafovea region of the eye, (e) the ability to deliver nucleic acid to two or more different retinal cell types within an eye, (f) the ability to deliver nucleic acid to retinal pigment epithelial (RPE) cells, (g) an increased efficiency to deliver nucleic acid to photoreceptor cells of the retina, (h) an increased efficiency to deliver nucleic acid to retinal ganglion cells of the retina, (i) an increased efficiency to deliver nucleic acid to bipolar cells of the retina, (j) an increased efficiency to deliver nucleic acid to ON-retinal ganglion cells, (k) an increased efficiency to deliver nucleic acid to OFF-retinal ganglion cells, and/or (1) increased packaging efficiency and the ability to deliver nucleic acid to cells (e.g., retinal cells).

### BACKGROUND

Viral vectors, such as AAV vectors, are efficient vehicles for in vivo nucleic acid delivery, and their use in the clinic is expanding. Improved AAV vectors and AAV production techniques for making effective AAV vector preparations should further expand the use of AAV vectors in the laboratory and clinic.

### SUMMARY

This document provides AAV vectors (e.g., AAV2 vectors). For example, this document provides AAV vectors (e.g., AAV2 vectors) containing a capsid polypeptide that includes an amino acid sequence set forth in Table 1A (or a variant thereof) or according to Formula A based on such a set forth sequence. The AAV vectors (e.g., AAV2 vectors) described herein containing a capsid polypeptide that includes an amino acid sequence set forth in Table 1A (or a variant thereof) or according to Formula A based on such a set forth sequence can have the ability to infect foveal cones in vivo and deliver exogenous nucleic acid to the infected foveal cones such that the infected foveal cones express the exogenous nucleic acid. This document also provides methods and materials for making and using AAV vectors (e.g., AAV2 vectors) having the ability to deliver nucleic acid to foveal cones.

As described herein, an AAV vector (e.g., an AAV2 vector) provided herein having an AAV capsid polypeptide that includes an amino acid sequence set forth in Table 1A (or a variant thereof) or according to Formula A based on such a set forth sequence can have the ability to infect foveal cones in vivo and deliver exogenous nucleic acid to the infected foveal cones such that the infected foveal cones express the exogenous nucleic acid. In some cases, an AAV vector (e.g., an AAV2 vector) provided herein can have the ability to infect and drive mRNA expression of an exogenous nucleic acid in cone cells present in the fovea of a mammal (e.g., a human or a non-human primate). In some cases, an AAV vector (e.g., an AAV2 vector) provided herein can have the ability to drive a level of mRNA expression of an exogenous nucleic acid in foveal cones of a mammal (e.g., a human or a non-human primate) that is greater than (e.g., at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO:1 (e.g., a wild-type AAV2 vector) in foveal cones of a control mammal (e.g., a control human or a control non-human primate). In some cases, an AAV vector (e.g., an AAV2 vector) provided herein having an AAV capsid polypeptide that includes an amino acid sequence set forth in Table 1A (or a variant thereof) or according to Formula A based on such a set forth sequence can be used in place of the 7m8 AAV2 vector (Dalkara et al., Sci. Transl. Med., 5(189):189ra76 (2013) and Bennett et al., J. Struct. Biol., 209(2):107433 (2020)) or in place of the K912 AAV2 vector (Öztürk et al., eLife, 10:e64175 (2021)) to deliver nucleic acid to foveal cones.

In another aspect, this document provides AAV vectors (e.g., AAV2 vectors) containing a capsid polypeptide that includes an amino acid sequence set forth in Table 1B (or a variant thereof) or according to Formula A based on such a set forth sequence. The AAV vectors (e.g., AAV2 vectors) described herein containing a capsid polypeptide that includes an amino acid sequence set forth in Table 1B (or a variant thereof) or according to Formula A based on such a set forth sequence can have the ability to infect retinal cells (e.g., retinal ganglion cells, photoreceptor cells, and bipolar cells) in vivo and deliver exogenous nucleic acid to the infected retinal cells such that the infected retinal cells express the exogenous nucleic acid at high levels.

This document also provides methods and materials for making and using AAV vectors (e.g., AAV2 vectors) having the ability to deliver nucleic acid to retinal cells and drive high expression levels of nucleic acid within retinal cells.

As described herein, an AAV vector (e.g., an AAV2 vector) provided herein having an AAV capsid polypeptide that includes an amino acid sequence set forth in Table 1B (or a variant thereof) or according to Formula A based on such a set forth sequence can have the ability to infect retinal cells (e.g., retinal ganglion cells, photoreceptor cells, and bipolar cells) in vivo and deliver exogenous nucleic acid to the infected retinal cells such that the infected retinal cells express the exogenous nucleic acid at high levels. In some cases, an AAV vector (e.g., an AAV2 vector) provided herein can have the ability to infect and drive mRNA expression of an exogenous nucleic acid in at least 2 percent (e.g., (e.g., at least 2.5 percent, at least 5 percent, at least 7.5 percent, at least 10 percent, or at least 25 percent) of retinal cells (e.g., retinal ganglion cells, photoreceptor cells, and bipolar cells) in an eye of a mammal (e.g., a human or a non-human primate). In some cases, an AAV vector (e.g., an AAV2 vector) provided herein can have the ability to drive a level of mRNA expression of an exogenous nucleic acid in retinal cells (e.g., retinal ganglion cells, photoreceptor cells, and bipolar cells) of a mammal (e.g., a human or a non-human primate) that is greater than (e.g., at least 2 percent greater than, at least 2.5 percent greater than, at least 5 percent greater than, at least 7.5 percent greater than, at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO:1 (e.g., a wild-type AAV2 vector) in retinal cells of a control mammal (e.g., a control human or a control non-human primate). In some cases, an AAV vector (e.g., an AAV2 vector) provided herein having an AAV capsid polypeptide that includes an amino acid sequence set forth in Table 1B (or a variant thereof) or according to Formula A based on such a set forth sequence can be used in place of the 7m8 AAV2 vector (Dalkara et al., Sci. Transl. Med., 5(189):189ra76 (2013) and Bennett et al., J. Struct. Biol., 209(2):107433 (2020)) or in place of the K912 AAV2 vector (Öztürk et al., eLife, 10:e64175 (2021)) to deliver nucleic acid to retinal cells (e.g., retinal ganglion cells, photoreceptor cells, and bipolar cells).

In another aspect, this document provides AAV vectors (e.g., AAV2 vectors) containing a capsid polypeptide that includes an amino acid sequence set forth in Table 1C (or a variant thereof) or according to Formula A based on such a set forth sequence. The AAV vectors (e.g., AAV2 vectors) described herein containing a capsid polypeptide that includes an amino acid sequence set forth in Table 1C (or a variant thereof) or according to Formula A based on such a set forth sequence can have the ability to infect retinal cells (e.g., retinal ganglion cells, photoreceptor cells, and bipolar cells) across retinal regions (e.g., across at least two retinal regions) in vivo and deliver exogenous nucleic acid to the infected retinal cells such that the infected retinal cells express the exogenous nucleic acid. This document also provides methods and materials for making and using AAV vectors (e.g., AAV2 vectors) having the ability to deliver nucleic acid to retinal cells across retinal regions and drive expression of delivered nucleic acid within the retinal cells. For example, the AAV vectors (e.g., AAV2 vectors) described herein can deliver nucleic acid to at least about 2 percent (e.g., at least about 2.5 percent, at least about 5 percent, at least about 7.5 percent, at least about 10 percent, or at least about 25 percent) of the retinal cells in the fovea region, at least about 2 percent (e.g., at least about 2.5 percent, at least about 5 percent, at least about 7.5 percent, at least about 10 percent, or at least about 25 percent) of the retinal cells in the parafovea region, at least about 2 percent (e.g., at least about 2.5 percent, at least about 5 percent, at least about 7.5 percent, at least about 10 percent, or at least about 25 percent) of the retinal cells in the vascular arcade region, and/or at least about 2 percent (e.g., at least about 2.5 percent, at least about 5 percent, at least about 7.5 percent, at least about 10 percent, or at least about 25 percent) of the retinal cells in the periphery region.

As described herein, an AAV vector (e.g., an AAV2 vector) provided herein having an AAV capsid polypeptide that includes an amino acid sequence set forth in Table 1C (or a variant thereof) or according to Formula A based on such a set forth sequence can have the ability to infect retinal cells (e.g., retinal ganglion cells, photoreceptor cells, and bipolar cells) across retinal regions (e.g., across at least two retinal regions) in vivo and deliver exogenous nucleic acid to the infected retinal cells such that the infected retinal cells express the exogenous nucleic acid. In some cases, an AAV vector (e.g., an AAV2 vector) provided herein can have the ability to infect and drive mRNA expression of an exogenous nucleic acid in at least about 2 percent (e.g., at least about 2.5 percent, at least about 5 percent, at least about 7.5 percent, at least about percent, or at least about 25 percent) of the retinal cells in the fovea region, at least about 2 percent (e.g., at least about 2.5 percent, at least about 5 percent, at least about 7.5 percent, at least about 10 percent, or at least about 25 percent) of the retinal cells in the parafovea region, at least about 2 percent (e.g., at least about 2.5 percent, at least about 5 percent, at least about 7.5 percent, at least about 10 percent, or at least about 25 percent) of the retinal cells in the vascular arcade region, and/or at least about 2 percent (e.g., at least about 2.5 percent, at least about 5 percent, at least about 7.5 percent, at least about 10 percent, or at least about 25 percent) of the retinal cells in the periphery region of an eye of a mammal (e.g., a human or a non-human primate). In some cases, an AAV vector (e.g., an AAV2 vector) provided herein can have the ability to drive a level of mRNA expression of an exogenous nucleic acid in retinal cells of the fovea region, the parafovea region, the vascular arcade region, and/or the periphery region of an eye of a mammal (e.g., a human or a non-human primate) that is greater than the level of mRNA expression of an exogenous nucleic acid driven by a comparable AAV vector having an AAV capsid polypeptide that consists of the amino acid sequence of SEQ ID NO:1 (e.g., a wild-type AAV2 vector) in retinal cells of those regions in a control mammal (e.g., a control human or a control non-human primate). In some cases, an AAV vector (e.g., an AAV2 vector) provided herein having an AAV capsid polypeptide that includes an amino acid sequence set forth in Table 1C (or a variant thereof) or according to Formula A based on such a set forth sequence can be used in place of the 7m8 AAV2 vector (Dalkara et al., Sci. Transl. Med., 5(189):189ra76 (2013) and Bennett et al., J. Struct. Biol., 209(2):107433 (2020)) or in place of the K912 AAV2 vector (Öztürk et al., eLife, 10:e64175 (2021)) to deliver nucleic acid to retinal cells (e.g., retinal ganglion cells, photoreceptor cells, and bipolar cells).

In another aspect, this document provides AAV vectors (e.g., AAV2 vectors) containing a capsid polypeptide that includes an amino acid sequence set forth in Table 1D (or a variant thereof) or according to Formula A based on such a set forth sequence. The AAV vectors (e.g., AAV2 vectors) described herein containing a capsid polypeptide that includes an amino acid sequence set forth in Table 1D (or a variant thereof) or according to Formula A based on such a set forth sequence can have the ability to infect retinal cells of the parafovea region of the eye in vivo and deliver exogenous nucleic acid to the infected retinal cells of the parafovea region such that the infected retinal cells express the exogenous nucleic acid. This document also provides methods and materials for making and using AAV vectors (e.g., AAV2 vectors) having the ability to deliver nucleic acid to retinal cells of the parafovea region of the eye.

As described herein, an AAV vector (e.g., an AAV2 vector) provided herein having an AAV capsid polypeptide that includes an amino acid sequence set forth in Table 1D (or a variant thereof) or according to Formula A based on such a set forth sequence can have the ability to infect retinal cells of the parafovea region of the eye in vivo and deliver exogenous nucleic acid to the infected retinal cells such that the infected retinal cells express the exogenous nucleic acid. In some cases, an AAV vector (e.g., an AAV2 vector) provided herein can have the ability to infect and drive mRNA expression of an exogenous nucleic acid in at least about 2 percent (e.g., at least about 2.5 percent, at least about 5 percent, at least about 7.5 percent, at least about 10 percent, or at least about 25 percent) of the retinal cells present in the parafovea region of an eye of a mammal (e.g., a human or a non-human primate). In some cases, an AAV vector (e.g., an AAV2 vector) provided herein can have the ability to drive a level of mRNA expression of an exogenous nucleic acid in retinal cells of the parafovea region of the eye of a mammal (e.g., a human or a non-human primate) that is greater than (e.g., at least 10 percent greater than, at least percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO:1 (e.g., a wild-type AAV2 vector) in retinal cells of the parafovea region of the eye of a control mammal (e.g., a control human or a control non-human primate). In some cases, an AAV vector (e.g., an AAV2 vector) provided herein having an AAV capsid polypeptide that includes an amino acid sequence set forth in Table 1D (or a variant thereof) or according to Formula A based on such a set forth sequence can be used in place of the 7m8 AAV2 vector (Dalkara et al., Sci. Transl. Med., 5(189):189ra76 (2013) and Bennett et al., J. Struct. Biol., 209(2):107433 (2020)) or in place of the K912 AAV2 vector (Öztürk et al., eLife, 10:e64175 (2021)) to deliver nucleic acid to retinal cells (e.g., retinal ganglion cells, photoreceptor cells, and bipolar cells) of the parafovea region of the eye.

In another aspect, this document provides AAV vectors (e.g., AAV2 vectors) containing a capsid polypeptide that includes an amino acid sequence set forth in Table 1E (or a variant thereof) or according to Formula A based on such a set forth sequence. The AAV vectors (e.g., AAV2 vectors) described herein containing a capsid polypeptide that includes an amino acid sequence set forth in Table 1E (or a variant thereof) or according to Formula A based on such a set forth sequence can have the ability to infect two or more (e.g., two or more, three or more, four or more, five or more, six or more, or seven or more) different retinal cell types within an eye in vivo and deliver exogenous nucleic acid to the infected retinal cells such that the infected retinal cells express the exogenous nucleic acid. This document also provides methods and materials for making and using AAV vectors (e.g., AAV2 vectors) having the ability to deliver nucleic acid to two or more (e.g., two or more, three or more, four or more, five or more, six or more, or seven or more) different retinal cell types within an eye and drive expression of delivered nucleic acid within those retinal cells. For example, the AAV vectors (e.g., AAV2 vectors) described herein can deliver nucleic acid to two, three, four, five, six, or seven of the following retinal cell types of an eye: retinal ganglion cells, amacrine cells, horizontal cells, bipolar cells, Muller glia cells, photoreceptor cells, and retinal pigment epithelial (RPE) cells. In some cases, an AAV vector (e.g., an AAV2 vector) described herein can deliver nucleic acid to at least some (e.g., at least 2 percent, at least 2.5 percent, at least 5 percent, at least 10 percent, or at least 25 percent) of the retinal ganglion cells, amacrine cells, horizontal cells, bipolar cells, Muller glia cells, photoreceptor cells, and RPE cells of an eye of a mammal (e.g., a human or a non-human primate) following an intravitreal administration.

As described herein, an AAV vector (e.g., an AAV2 vector) provided herein having an AAV capsid polypeptide that includes an amino acid sequence set forth in Table 1E (or a variant thereof) or according to Formula A based on such a set forth sequence can have the ability to infect two or more (e.g., two or more, three or more, four or more, five or more, six or more, or seven or more) different retinal cell types within an eye in vivo and deliver exogenous nucleic acid to the infected retinal cells such that the infected retinal cells express the exogenous nucleic acid. In some cases, an AAV vector (e.g., an AAV2 vector) provided herein can have the ability to infect and drive mRNA expression of an exogenous nucleic acid in at least about 2 percent (e.g., at least about 2.5 percent, at least about 5 percent, at least about 7.5 percent, at least about percent, or at least about 25 percent) of the retinal ganglion cells, at least about 2 percent (e.g., at least about 2.5 percent, at least about 5 percent, at least about 7.5 percent, at least about percent, or at least about 25 percent) of the amacrine cells, at least about 2 percent (e.g., at least about 2.5 percent, at least about 5 percent, at least about 7.5 percent, at least about 10 percent, or at least about 25 percent) of the horizontal cells, at least about 2 percent (e.g., at least about 2.5 percent, at least about 5 percent, at least about 7.5 percent, at least about 10 percent, or at least about 25 percent) of the bipolar cells, at least about 2 percent (e.g., at least about 2.5 percent, at least about 5 percent, at least about 7.5 percent, at least about 10 percent, or at least about 25 percent) of the Muller glia cells, at least about 2 percent (e.g., at least about 2.5 percent, at least about 5 percent, at least about 7.5 percent, at least about 10 percent, or at least about 25 percent) of the photoreceptor cells, and/or at least about 2 percent (e.g., at least about 2.5 percent, at least about 5 percent, at least about 7.5 percent, at least about 10 percent, or at least about 25 percent) of the RPE cells of an eye of a mammal (e.g., a human or a non-human primate) following, for example, an intravitreal administration. In some cases, an AAV vector (e.g., an AAV2 vector) provided herein can have the ability to drive a level of mRNA expression of an exogenous nucleic acid in retinal ganglion cells, amacrine cells, horizontal cells, bipolar cells, Muller glia cells, photoreceptor cells, and/or RPE cells of an eye of a mammal (e.g., a human or a non-human primate) that is greater than (e.g., at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO:1 (e.g., a wild-type AAV2 vector) in those retinal cells in a control mammal (e.g., a control human or a control non-human primate). In some cases, an AAV vector (e.g., an AAV2 vector) provided herein having an AAV capsid polypeptide that includes an amino acid sequence set forth in Table 1E (or a variant thereof) or according to Formula A based on such a set forth sequence can be used in place of the 7m8 AAV2 vector (Dalkara et al., Sci. Transl. Med., 5(189):189ra76 (2013) and Bennett et al., J. Struct. Biol., 209(2):107433 (2020)) or in place of the K912 AAV2 vector (Öztürk et al., eLife, 10:e64175 (2021)) to deliver nucleic acid to two or more (e.g., two or more, three or more, four or more, five or more, six or more, or seven or more) different retinal cell types within an eye of a mammal (e.g., a human or a non-human primate).

In another aspect, this document provides AAV vectors (e.g., AAV2 vectors) containing a capsid polypeptide that includes an amino acid sequence set forth in Table 1F (or a variant thereof) or according to Formula A based on such a set forth sequence. The AAV vectors (e.g., AAV2 vectors) described herein containing a capsid polypeptide that includes an amino acid sequence set forth in Table 1F (or a variant thereof) or according to Formula A based on such a set forth sequence can have the ability to infect RPE cells in vivo and deliver exogenous nucleic acid to the infected RPE cells such that the infected RPE cells express the exogenous nucleic acid. This document also provides methods and materials for making and using AAV vectors (e.g., AAV2 vectors) having the ability to deliver nucleic acid to RPE cells and drive expression of delivered nucleic acid within the RPE cells. For example, the AAV vectors (e.g., AAV2 vectors) described herein can deliver nucleic acid to at least about 2 percent (e.g., at least about 2.5 percent, at least about 5 percent, at least about 7.5 percent, at least about 10 percent, or at least about 25 percent) of the RPE cells of an eye of a mammal after, for example, an intravitreal administration.

As described herein, an AAV vector (e.g., an AAV2 vector) provided herein having an AAV capsid polypeptide that includes an amino acid sequence set forth in Table 1F (or a variant thereof) or according to Formula A based on such a set forth sequence can have the ability to infect RPE cells in vivo and deliver exogenous nucleic acid to the infected RPE cells such that the infected RPE cells express the exogenous nucleic acid. In some cases, an AAV vector (e.g., an AAV2 vector) provided herein can have the ability to infect and drive mRNA expression of an exogenous nucleic acid in at least about 2 percent (e.g., at least about 2.5 percent, at least about 5 percent, at least about 7.5 percent, at least about 10 percent, or at least about 25 percent) of the RPE cells of an eye of a mammal (e.g., a human or a non-human primate). In some cases, an AAV vector (e.g., an AAV2 vector) provided herein can have the ability to drive a level of mRNA expression of an exogenous nucleic acid in RPE cells of an eye of a mammal (e.g., a human or a non-human primate) that is greater than (e.g., at least 10 percent greater than, at least percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO:1 (e.g., a wild type AAV2 vector) in RPE cells in a control mammal (e.g., a control human or a control non-human primate). In some cases, an AAV vector (e.g., an AAV2 vector) provided herein having an AAV capsid polypeptide that includes an amino acid sequence set forth in Table 1F (or a variant thereof) or according to Formula A based on such a set forth sequence can be used in place of the 7m8 AAV2 vector (Dalkara et al., Sci. Transl. Med., 5(189):189ra76 (2013) and Bennett et al., J. Struct. Biol., 209(2):107433 (2020)) or in place of the K912 AAV2 vector (Öztürk et al., eLife, 10:e64175 (2021)) to deliver nucleic acid to RPE cells.

In another aspect, this document provides AAV vectors (e.g., AAV2 vectors) containing a capsid polypeptide that includes an amino acid sequence set forth in Table 1G (or a variant thereof) or according to Formula A based on such a set forth sequence. The AAV vectors (e.g., AAV2 vectors) described herein containing a capsid polypeptide that includes an amino acid sequence set forth in Table 1G (or a variant thereof) or according to Formula A based on such a set forth sequence can have the ability to infect photoreceptor cells of the retina in vivo and deliver exogenous nucleic acid to the infected photoreceptor cells such that the infected photoreceptor cells express the exogenous nucleic acid. This document also provides methods and materials for making and using AAV vectors (e.g., AAV2 vectors) having the ability to deliver nucleic acid to photoreceptor cells of the retina.

As described herein, an AAV vector (e.g., an AAV2 vector) provided herein having an AAV capsid polypeptide that includes an amino acid sequence set forth in Table 1G (or a variant thereof) or according to Formula A based on such a set forth sequence can have the ability to infect photoreceptor cells of the retina in vivo and deliver exogenous nucleic acid to the infected photoreceptor cells such that the infected photoreceptor cells express the exogenous nucleic acid. In some cases, an AAV vector (e.g., an AAV2 vector) provided herein can have the ability to infect and drive mRNA expression of an exogenous nucleic acid in at least about 2 percent (e.g., at least about 2.5 percent, at least about 5 percent, at least about 7.5 percent, at least about 10 percent, or at least about 25 percent) of photoreceptor cells of an eye of a mammal (e.g., a human or a non-human primate). In some cases, an AAV vector (e.g., an AAV2 vector) provided herein can have the ability to drive a level of mRNA expression of an exogenous nucleic acid in photoreceptor cells of the retina of a mammal (e.g., a human or a non-human primate) that is greater than (e.g., at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO: 1 (e.g., a wild-type AAV2 vector) in photoreceptor cells of the retina of a control mammal (e.g., a control human or a control non-human primate). In some cases, an AAV vector (e.g., an AAV2 vector) provided herein having an AAV capsid polypeptide that includes an amino acid sequence set forth in Table 1G (or a variant thereof) or according to Formula A based on such a set forth sequence can be used in place of the 7m8 AAV2 vector (Dalkara et al., Sci. Transl. Med., 5(189):189ra76 (2013) and Bennett et al., J. Struct. Biol., 209(2):107433 (2020)) or in place of the K912 AAV2 vector (Öztürk et al., eLife, 10:e64175 (2021)) to deliver nucleic acid to photoreceptor cells of the retina.

In another aspect, this document provides AAV vectors (e.g., AAV2 vectors) containing a capsid polypeptide that includes an amino acid sequence set forth in Table 1H (or a variant thereof) or according to Formula A based on such a set forth sequence. The AAV vectors (e.g., AAV2 vectors) described herein containing a capsid polypeptide that includes an amino acid sequence set forth in Table 1H (or a variant thereof) or according to Formula A based on such a set forth sequence can have the ability to infect retinal ganglion cells in vivo and deliver exogenous nucleic acid to the infected retinal ganglion cells such that the infected retinal ganglion cells express the exogenous nucleic acid. This document also provides methods and materials for making and using AAV vectors (e.g., AAV2 vectors) having the ability to deliver nucleic acid to retinal ganglion cells.

As described herein, an AAV vector (e.g., an AAV2 vector) provided herein having an AAV capsid polypeptide that includes an amino acid sequence set forth in Table 1H (or a variant thereof) or according to Formula A based on such a set forth sequence can have the ability to infect retinal ganglion cells in vivo and deliver exogenous nucleic acid to the infected retinal ganglion cells such that the infected retinal ganglion cells express the exogenous nucleic acid. In some cases, an AAV vector (e.g., an AAV2 vector) provided herein can have the ability to infect and drive mRNA expression of an exogenous nucleic acid in at least about 2 percent (e.g., at least about 2.5 percent, at least about 5 percent, at least about 7.5 percent, at least about 10 percent, or at least about 25 percent) of retinal ganglion cells of an eye of a mammal (e.g., a human or a non-human primate). In some cases, an AAV vector (e.g., an AAV2 vector) provided herein can have the ability to drive a level of mRNA expression of an exogenous nucleic acid in retinal ganglion cells of a mammal (e.g., a human or a non-human primate) that is greater than (e.g., at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO:1 (e.g., a wild-type AAV2 vector) in retinal ganglion cells of an eye of a control mammal (e.g., a control human or a control non-human primate). In some cases, an AAV vector (e.g., an AAV2 vector) provided herein having an AAV capsid polypeptide that includes an amino acid sequence set forth in Table 1H (or a variant thereof) or according to Formula A based on such a set forth sequence can be used in place of the 7m8 AAV2 vector (Dalkara et al., Sci. Transl. Med., 5(189):189ra76 (2013) and Bennett et al., J Struct. Biol., 209(2):107433 (2020)) or in place of the K912 AAV2 vector (Öztürk et al., eLife, 10:e64175 (2021)) to deliver nucleic acid to photoreceptor cells of the retina.

In another aspect, this document provides AAV vectors (e.g., AAV2 vectors) containing a capsid polypeptide that includes an amino acid sequence set forth in Table 1I (or a variant thereof) or according to Formula A based on such a set forth sequence. The AAV vectors (e.g., AAV2 vectors) described herein containing a capsid polypeptide that includes an amino acid sequence set forth in Table 1I (or a variant thereof) or according to Formula A based on such a set forth sequence can have the ability to infect bipolar cells of the retina in vivo and deliver exogenous nucleic acid to the infected bipolar cells such that the infected retinal bipolar cells express the exogenous nucleic acid. This document also provides methods and materials for making and using AAV vectors (e.g., AAV2 vectors) having the ability to deliver nucleic acid to bipolar cells of the retina.

As described herein, an AAV vector (e.g., an AAV2 vector) provided herein having an AAV capsid polypeptide that includes an amino acid sequence set forth in Table 1I (or a variant thereof) or according to Formula A based on such a set forth sequence can have the ability to infect bipolar cells of the retina in vivo and deliver exogenous nucleic acid to the infected bipolar cells such that the infected bipolar cells express the exogenous nucleic acid. In some cases, an AAV vector (e.g., an AAV2 vector) provided herein can have the ability to infect and drive mRNA expression of an exogenous nucleic acid in at least about 2 percent (e.g., at least about 2.5 percent, at least about 5 percent, at least about 7.5 percent, at least about 10 percent, or at least about 25 percent) of bipolar cells of the retina of a mammal (e.g., a human or a non-human primate). In some cases, an AAV vector (e.g., an AAV2 vector) provided herein can have the ability to drive a level of mRNA expression of an exogenous nucleic acid in bipolar cells of the retina of a mammal (e.g., a human or a non-human primate) that is greater than (e.g., at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO:1 (e.g., a wild-type AAV2 vector) in bipolar cells of the retina of a control mammal (e.g., a control human or a control non-human primate). In some cases, an AAV vector (e.g., an AAV2 vector) provided herein having an AAV capsid polypeptide that includes an amino acid sequence set forth in Table 1I (or a variant thereof) or according to Formula A based on such a set forth sequence can be used in place of the 7m8 AAV2 vector (Dalkara et al., Sci. Transl. Med., 5(189):189ra76 (2013) and Bennett et al., J. Struct. Biol., 209(2):107433 (2020)) or in place of the K912 AAV2 vector (Öztürk et al., eLife, 10:e64175 (2021)) to deliver nucleic acid to photoreceptor cells of the retina.

In another aspect, this document provides AAV vectors (e.g., AAV2 vectors) containing a capsid polypeptide that includes an amino acid sequence set forth in Table 1J (or a variant thereof) or according to Formula A based on such a set forth sequence. The AAV vectors (e.g., AAV2 vectors) described herein containing a capsid polypeptide that includes an amino acid sequence set forth in Table 1J (or a variant thereof) or according to Formula A based on such a set forth sequence can have the ability to infect ON-retinal ganglion cells in vivo and deliver exogenous nucleic acid to the infected ON-retinal ganglion cells such that the infected ON-retinal ganglion cells express the exogenous nucleic acid. This document also provides methods and materials for making and using AAV vectors (e.g., AAV2 vectors) having the ability to deliver nucleic acid to ON-retinal ganglion cells.

As described herein, an AAV vector (e.g., an AAV2 vector) provided herein having an AAV capsid polypeptide that includes an amino acid sequence set forth in Table 1J (or a variant thereof) or according to Formula A based on such a set forth sequence can have the ability to infect ON-retinal ganglion cells in vivo and deliver exogenous nucleic acid to the infected ON-retinal ganglion cells such that the infected ON-retinal ganglion cells express the exogenous nucleic acid. In some cases, an AAV vector (e.g., an AAV2 vector) provided herein can have the ability to infect and drive mRNA expression of an exogenous nucleic acid in at least about 2 percent (e.g., at least about 2.5 percent, at least about 5 percent, at least about 7.5 percent, at least about 10 percent, or at least about 25 percent) of ON-retinal ganglion cells of a mammal (e.g., a human or a non-human primate). In some cases, an AAV vector (e.g., an AAV2 vector) provided herein can have the ability to drive a level of mRNA expression of an exogenous nucleic acid in ON-retinal ganglion cells of a mammal (e.g., a human or a non-human primate) that is greater than (e.g., at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO:1 (e.g., a wild-type AAV2 vector) in ON-retinal ganglion cells of a control mammal (e.g., a control human or a control non-human primate). In some cases, an AAV vector (e.g., an AAV2 vector) provided herein having an AAV capsid polypeptide that includes an amino acid sequence set forth in Table 1J (or a variant thereof) or according to Formula A based on such a set forth sequence can be used in place of the 7m8 AAV2 vector (Dalkara et al., Sci. Transl. Med., 5(189):189ra76 (2013) and Bennett et al., J. Struct. Biol., 209(2):107433 (2020)) or in place of the K912 AAV2 vector (Öztürk et al., eLife, 10:e64175 (2021)) to deliver nucleic acid to photoreceptor cells of the retina.

In another aspect, this document provides AAV vectors (e.g., AAV2 vectors) containing a capsid polypeptide that includes an amino acid sequence set forth in Table 1K (or a variant thereof) or according to Formula A based on such a set forth sequence. The AAV vectors (e.g., AAV2 vectors) described herein containing a capsid polypeptide that includes an amino acid sequence set forth in Table 1K (or a variant thereof) or according to Formula A based on such a set forth sequence can have the ability to infect OFF-retinal ganglion cells in vivo and deliver exogenous nucleic acid to the infected OFF-retinal ganglion cells such that the infected OFF-retinal ganglion cells express the exogenous nucleic acid. This document also provides methods and materials for making and using AAV vectors (e.g., AAV2 vectors) having the ability to deliver nucleic acid to OFF-retinal ganglion cells.

As described herein, an AAV vector (e.g., an AAV2 vector) provided herein having an AAV capsid polypeptide that includes an amino acid sequence set forth in Table 1K (or a variant thereof) or according to Formula A based on such a set forth sequence can have the ability to infect OFF-retinal ganglion cells in vivo and deliver exogenous nucleic acid to the infected OFF-retinal ganglion cells such that the infected OFF-retinal ganglion cells express the exogenous nucleic acid. In some cases, an AAV vector (e.g., an AAV2 vector) provided herein can have the ability to infect and drive mRNA expression of an exogenous nucleic acid in at least about 2 percent (e.g., at least about 2.5 percent, at least about 5 percent, at least about 7.5 percent, at least about 10 percent, or at least about 25 percent) of OFF-retinal ganglion cells of a mammal (e.g., a human or a non-human primate). In some cases, an AAV vector (e.g., an AAV2 vector) provided herein can have the ability to drive a level of mRNA expression of an exogenous nucleic acid in OFF-retinal ganglion cells of a mammal (e.g., a human or a non-human primate) that is greater than (e.g., at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO:1 (e.g., a wild-type AAV2 vector) in OFF-retinal ganglion cells of a control mammal (e.g., a control human or a control non-human primate). In some cases, an AAV vector (e.g., an AAV2 vector) provided herein having an AAV capsid polypeptide that includes an amino acid sequence set forth in Table 1K (or a variant thereof) or according to Formula A based on such a set forth sequence can be used in place of the 7m8 AAV2 vector (Dalkara et al., Sci. Transl. Med., 5(189):189ra76 (2013) and Bennett et al., J Struct. Biol., 209(2):107433 (2020)) or in place of the K912 AAV2 vector (Öztürk et al., eLife, 10:e64175 (2021)) to deliver nucleic acid to photoreceptor cells of the retina.

In another aspect, this document provides AAV vectors (e.g., AAV2 vectors) containing a capsid polypeptide that includes an amino acid sequence set forth in Table 1L (or a variant thereof) or according to Formula A based on such a set forth sequence. The AAV vectors (e.g., AAV2 vectors) described herein containing a capsid polypeptide that includes an amino acid sequence set forth in Table 1L (or a variant thereof) or according to Formula A based on such a set forth sequence can have increased packaging efficiency, the ability to infect cells (e.g., retinal cells) in vivo or in vitro, and the ability to deliver exogenous nucleic acid to the infected cells such that the infected cells express the exogenous nucleic acid. This document also provides methods and materials for making and using AAV vectors (e.g., AAV2 vectors) having increased packaging efficiency, the ability to deliver nucleic acid to cells (e.g., retinal cells) in vivo or in vitro, and the ability to drive expression of delivered nucleic acid within the cells. For example, the AAV vectors (e.g., AAV2 vectors) described herein can have a packaging efficiency greater than that of a comparable AAV vector having an AAV capsid polypeptide that consists of the amino acid sequence of SEQ ID NO:1 (e.g., a wild type AAV2 vector).

As described herein, an AAV vector (e.g., an AAV2 vector) provided herein having an AAV capsid polypeptide that includes an amino acid sequence set forth in Table 1L (or a variant thereof) or according to Formula A based on such a set forth sequence can have increased packaging efficiency (e.g., package efficiency greater than that of a comparable AAV vector having an AAV capsid polypeptide that consists of the amino acid sequence of SEQ ID NO:1 (e.g., a wild type AAV2 vector)), the ability to infect cells (e.g., retinal cells) in vivo or in vitro, and the ability to drive exogenous nucleic acid to the infected cells such that the infected cells express the exogenous nucleic acid. In some cases, an AAV vector (e.g., an AAV2 vector) provided herein can have a packaging efficiency that is greater than (e.g., at least 10 percent greater than, at least 25 percent greater than, at least 50 percent greater than, at least 75 percent greater than, or at least 100 percent greater than) the packaging efficiency of a comparable AAV vector having an AAV capsid polypeptide that consists of the amino acid sequence of SEQ ID NO:1 (e.g., a wild-type AAV2 vector). In some cases, an AAV vector (e.g., an AAV2 vector) provided herein having an AAV capsid polypeptide that includes an amino acid sequence set forth in Table 1L (or a variant thereof) or according to Formula A based on such a set forth sequence can have a packaging efficiency greater than that of the 7m8 AAV2 vector (Dalkara et al., Sci. Transl. Med., 5(189):189ra76 (2013) and Bennett et al., J Struct. Biol., 209(2):107433 (2020)) or the K912 AAV2 vector (Öztürk et al., eLife, 10:e64175 (2021)).

In general, one aspect of this document features an adeno-associated virus (AAV) vector comprising an AAV capsid polypeptide, wherein the capsid polypeptide comprises the amino acid sequence of any one of SEQ ID NOs:A2-A19. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:A2-A19 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:A2-A18 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:A2-A19. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of SEQ ID NO:A19. The vector can be an AAV2 vector. The vector can infect greater than 50 percent of foveal cones when a titer of at least 1×10⁷ of the vector is administered intravitreally to an eye of a human (or a non-human primate) (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in foveal cones than the level of expression from a comparable AAV vector comprising a capsid polypeptide consisting of the amino acid sequence set forth in SEQ ID NO:1.

In another aspect, this document features an AAV capsid polypeptide comprising the amino acid sequence of any one of SEQ ID NOs:A2-A19. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:A2-A19 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:A2-A18 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:A2-A19. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of SEQ ID NO:A19. An AAV vector comprising the polypeptide can infect greater than 50 percent of foveal cones when a titer of at least 1×10⁷ of the vector is administered intravitreally to an eye of a human (or a non-human primate) (or a non-human primate). An AAV vector comprising the polypeptide can express more nucleic acid in foveal cones than the level of expression from a comparable AAV vector comprising a capsid polypeptide consisting of the amino acid sequence set forth in SEQ ID NO:1.

In another aspect, this document features a nucleic acid molecule encoding a vector. The nucleic acid molecule can be DNA. The vector can be an adeno-associated virus (AAV) vector comprising an AAV capsid polypeptide, wherein the capsid polypeptide comprises the amino acid sequence of any one of SEQ ID NOs:A2-A19. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:A2-A19 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:A2-A18 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:A2-A19. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of SEQ ID NO:A19. The vector can be an AAV2 vector. The vector can infect greater than 50 percent of foveal cones when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in foveal cones than the level of expression from a comparable AAV vector comprising a capsid polypeptide consisting of the amino acid sequence set forth in SEQ ID NO:1.

In another aspect, this document features a nucleic acid molecule encoding a polypeptide. The nucleic acid molecule can be DNA. The polypeptide can be an AAV capsid polypeptide comprising the amino acid sequence of any one of SEQ ID NOs:A2-A19. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:A2-A19 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:A2-A18 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:A2-A19. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of SEQ ID NO:A19. An AAV vector comprising the polypeptide can infect greater than 50 percent of foveal cones when a titer of at least 1×10⁷ of the vector is administered intravitreally to an eye of a human (or a non-human primate). An AAV vector comprising the polypeptide can express more nucleic acid in foveal cones than the level of expression from a comparable AAV vector comprising a capsid polypeptide consisting of the amino acid sequence set forth in SEQ ID NO:1.

In another aspect, this document features a host cell comprising a nucleic acid molecule of either of the two preceding paragraphs. The host cell can express the vector. The host cell can express the polypeptide.

In another aspect, this document features a host cell comprising a vector. The host cell can be a retinal cell. The vector can be an adeno-associated virus (AAV) vector comprising an AAV capsid polypeptide, wherein the capsid polypeptide comprises the amino acid sequence of any one of SEQ ID NOs:A2-A19. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acid sequence of any one of SEQ ID NOs:A2-A19 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:A2-A18 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:A2-A19. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of SEQ ID NO:A19. The vector can be an AAV2 vector. The vector can infect greater than 50 percent of foveal cones when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in foveal cones than the level of expression from a comparable AAV vector comprising a capsid polypeptide consisting of the amino acid sequence set forth in SEQ ID NO:1.

In another aspect, this document features a host cell comprising a polypeptide. The host cell can be a retinal cell. The polypeptide can be an AAV capsid polypeptide comprising the amino acid sequence of any one of SEQ ID NOs:A2-A19. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:A2-A19 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:A2-A18 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:A2-A19. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of SEQ ID NO:A19. An AAV vector comprising the polypeptide can infect greater than 50 percent of foveal cones when a titer of at least 1×10⁷ of the vector is administered intravitreally to an eye of a human (or a non-human primate). An AAV vector comprising the polypeptide can express more nucleic acid in foveal cones than the level of expression from a comparable AAV vector comprising a capsid polypeptide consisting of the amino acid sequence set forth in SEQ ID NO:1.

In another aspect, this document features a composition comprising an adeno-associated virus (AAV) vector comprising an AAV capsid polypeptide, wherein the capsid polypeptide comprises the amino acid sequence of any one of SEQ ID NOs:A2-A19. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:A2-A19 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:A2-A18 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:A2-A19. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of SEQ ID NO:A19. The vector can be an AAV2 vector. The vector can infect greater than 50 percent of foveal cones when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in foveal cones than the level of expression from a comparable AAV vector comprising a capsid polypeptide consisting of the amino acid sequence set forth in SEQ ID NO:1. The composition can comprise from about 1×10⁷ to about 1×10¹⁴ of the vector. The composition can comprise phosphate buffered saline, Hank's Balanced Salt Solution, or Pluronic F68.

In another aspect, this document features a method for delivering an exogenous nucleic acid sequence to a foveal cone within a mammal. The method comprises (or consists essentially of, or consists of) contacting the foveal cone with an AAV vector comprising an AAV capsid polypeptide and the exogenous nucleic acid sequence, wherein the capsid polypeptide comprises the amino acid sequence of any one of SEQ ID NOs:A2-A19, wherein the AAV vector infects the foveal cone, thereby delivering the exogenous nucleic acid sequence to the foveal cone. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:A2-A19 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:A2-A18 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:A2-A19. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of SEQ ID NO:A19. The mammal can be a human (or a non-human primate). The vector can be an AAV2 vector. The vector can infect greater than 50 percent of foveal cones when a titer of at least 1×10⁷ of the vector is administered intravitreally to an eye of a human (or a non-human primate). The exogenous nucleic acid sequence can encode an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more of the exogenous nucleic acid sequence in the foveal cone than the level of expression in a foveal cone from a comparable AAV vector comprising a capsid polypeptide consisting of the amino acid sequence set forth in SEQ ID NO:1. The method can comprise intravitreally administering a composition comprising the vector to the mammal, thereby contacting the foveal cone with the vector. The composition can comprise from about 1×10⁷ to about 1×10¹⁴ of the vector.

In another aspect, this document features a method for treating a retinal condition. The method comprises (or consists essentially of, or consists of) contacting foveal cones of a mammal having the retinal condition with AAV vectors comprising an AAV capsid polypeptide and an exogenous nucleic acid sequence, wherein the capsid polypeptide comprises the amino acid sequence of any one of SEQ ID NOs:A2-A19, wherein the AAV vectors infect the foveal cones and drive expression of the exogenous nucleic acid sequence within the foveal cones, thereby treating the retinal condition. The mammal can be a human (or a non-human primate). The retinal condition can be selected from the group consisting of cone dystrophy, cone/rod dystrophy, retinitis pigmentosa, macular degeneration, achromatopsia, blue cone monochromcy, and color blindness. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:A2-A19 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:A2-A18 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO: 1 are replaced with the amino acid sequence of any one of SEQ ID NOs:A2-A19. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of SEQ ID NO:A19. The vectors can be AAV2 vectors. The vectors can infect greater than 50 percent of foveal cones when a titer of at least 1×10⁷ of the vectors is administered intravitreally to an eye of the mammal. The exogenous nucleic acid sequence can encode an RNA. The RNA can be an siRNA or a microRNA. The exogenous nucleic acid can encode a polypeptide. The polypeptide can be an ABCA4 polypeptide, a CRB1 polypeptide, an NPHP5 polypeptide, and an NR2E3 polypeptide.

The vectors can express more of the exogenous nucleic acid sequence in the foveal cones than the level of expression in foveal cones from a comparable AAV vector comprising a capsid polypeptide consisting of the amino acid sequence set forth in SEQ ID NO:1. The method can comprise intravitreally administering a composition comprising the vectors to the mammal, thereby contacting the foveal cones with the vectors. The composition can comprise from about 1×10⁷ to about 1×10¹⁴ of the vectors.

In another aspect, this document features an adeno-associated virus (AAV) vector comprising an AAV capsid polypeptide, wherein the capsid polypeptide comprises the amino acid sequence of any one of SEQ ID NOs:B2-B317. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B317 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B298 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:B2-B317. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:B299-B317. The vector can be an AAV2 vector. The vector can infect greater than 2.5 percent of retinal cells when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal cells than the level of expression from a comparable AAV vector comprising a capsid polypeptide consisting of the amino acid sequence set forth in SEQ ID NO:1.

In another aspect, this document features an AAV capsid polypeptide comprising the amino acid sequence of any one of SEQ ID NOs:B2-B317. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B317 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B298 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:B2-B317. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:B299-B317. An AAV vector comprising the polypeptide can infect greater than 2.5 percent of retinal cells when a titer of at least 1×10⁷ of the vector is administered intravitreally to an eye of a human (or a non-human primate). An AAV vector comprising the polypeptide can express more nucleic acid in retinal cells than the level of expression from a comparable AAV vector comprising a capsid polypeptide consisting of the amino acid sequence set forth in SEQ ID NO:1.

In another aspect, this document features a nucleic acid molecule encoding an AAV vector comprising an AAV capsid polypeptide, wherein the capsid polypeptide comprises the amino acid sequence of any one of SEQ ID NOs:B2-B317. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B317 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B298 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:B2-B317. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:B299-B317. The vector can be an AAV2 vector. The vector can infect greater than 2.5 percent of retinal cells when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal cells than the level of expression from a comparable AAV vector comprising a capsid polypeptide consisting of the amino acid sequence set forth in SEQ ID NO:1. The nucleic acid molecule can be DNA.

In another aspect, this document features a nucleic acid molecule encoding an AAV capsid polypeptide comprising the amino acid sequence of any one of SEQ ID NOs:B2-B317. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B317 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B298 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:B2-B317. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:B299-B317. An AAV vector comprising the polypeptide can infect greater than 2.5 percent of retinal cells when a titer of at least 1×10⁷ of the vector is administered intravitreally to an eye of a human (or a non-human primate). An AAV vector comprising the polypeptide can express more nucleic acid in retinal cells than the level of expression from a comparable AAV vector comprising a capsid polypeptide consisting of the amino acid sequence set forth in SEQ ID NO:1. The nucleic acid molecule can be DNA.

In another aspect, this document features a host cell comprising a nucleic acid molecule of either of the two preceding paragraphs. The host cell can express the vector. The host cell can express the polypeptide.

In another aspect, this document features a host cell comprising an AAV vector comprising an AAV capsid polypeptide, wherein the capsid polypeptide comprises the amino acid sequence of any one of SEQ ID NOs:B2-B317. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B317 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B298 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:B2-B317. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:B299-B317. The vector can be an AAV2 vector. The vector can infect greater than 2.5 percent of retinal cells when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal cells than the level of expression from a comparable AAV vector comprising a capsid polypeptide consisting of the amino acid sequence set forth in SEQ ID NO:1. The host cell can be a retinal cell.

In another aspect, this document features a host cell comprising an AAV capsid polypeptide comprising the amino acid sequence of any one of SEQ ID NOs:B2-B317. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B317 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B298 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:B2-B317. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:B299-B317. An AAV vector comprising the polypeptide can infect greater than 2.5 percent of retinal cells when a titer of at least 1×10⁷ of the vector is administered intravitreally to an eye of a human (or a non-human primate). An AAV vector comprising the polypeptide can express more nucleic acid in retinal cells than the level of expression from a comparable AAV vector comprising a capsid polypeptide consisting of the amino acid sequence set forth in SEQ ID NO:1. The host cell can be a retinal cell.

In another aspect, this document features a composition comprising an AAV vector comprising an AAV capsid polypeptide, wherein the capsid polypeptide comprises the amino acid sequence of any one of SEQ ID NOs:B2-B317. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B317 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B298 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:B2-B317. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:B299-B317. The vector can be an AAV2 vector. The vector can infect greater than 2.5 percent of retinal cells when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal cells than the level of expression from a comparable AAV vector comprising a capsid polypeptide consisting of the amino acid sequence set forth in SEQ ID NO:1. The composition can comprise from about 1×10⁷ to about 1×10¹⁴ of the vector. The composition can comprise phosphate buffered saline, Hank's Balanced Salt Solution, or Pluronic F68.

In another aspect, this document features a method for delivering an exogenous nucleic acid sequence to a retinal cell within a mammal. The method comprises (or consists essentially of, or consists of) contacting the retinal cell with an AAV vector comprising an AAV capsid polypeptide and the exogenous nucleic acid sequence, wherein the capsid polypeptide comprises the amino acid sequence of any one of SEQ ID NOs:B2-B317, wherein the AAV vector infects the retinal cell, thereby delivering the exogenous nucleic acid sequence to the retinal cell. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B317 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B298 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:B2-B317. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:B299-B317. The mammal can be a human (or a non-human primate). The vector can be an AAV2 vector. The vector can infect greater than 2.5 percent of retinal cells when a titer of at least 1×10⁷ of the vector is administered intravitreally to an eye of a human (or a non-human primate). The exogenous nucleic acid sequence can encode an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more of the exogenous nucleic acid sequence in the retinal cell than the level of expression in a retinal cell from a comparable AAV vector comprising a capsid polypeptide consisting of the amino acid sequence set forth in SEQ ID NO:1. The method can comprise intravitreally administering a composition comprising the vector to the mammal, thereby contacting the retinal cell with the vector. The composition can comprise from about 1×10⁷ to about 1×10¹⁴ of the vector.

In another aspect, this document features a method for treating a retinal condition. The method comprises (or consists essentially of, or consists of) contacting retinal cells of a mammal having the retinal condition with AAV vectors comprising an AAV capsid polypeptide and an exogenous nucleic acid sequence, wherein the capsid polypeptide comprises the amino acid sequence of any one of SEQ ID NOs:B2-B317, wherein the AAV vectors infect the retinal cells and drive expression of the exogenous nucleic acid sequence within the retinal cells, thereby treating the retinal condition. The mammal can be a human (or a non-human primate). The retinal condition can be selected from the group consisting of LCA, OCA1, retinitis pigmentosa, rod/cone dystrophy, cone dystrophy, Stargardt Disease, Usher syndrome, XLRP, and XLRS. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B317 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:B2-B298 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO: 1 are replaced with the amino acid sequence of any one of SEQ ID NOs:B2-B317. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:B299-B317. The vectors can be AAV2 vectors. The vectors can infect greater than 2.5 percent of retinal cells when a titer of at least 1×10⁷ of the vectors is administered intravitreally to an eye of the mammal. The exogenous nucleic acid sequence can encode an RNA. The RNA can be an siRNA or a microRNA. The exogenous nucleic acid can encode a polypeptide. The polypeptide can be an ABCA4 polypeptide, a CRB1 polypeptide, an NPHP5 polypeptide, and an NR2E3 polypeptide. The vectors can express more of the exogenous nucleic acid sequence in the retinal cells than the level of expression in retinal cells from a comparable AAV vector comprising a capsid polypeptide consisting of the amino acid sequence set forth in SEQ ID NO:1. The method can comprise intravitreally administering a composition comprising the vectors to the mammal, thereby contacting the retinal cells with the vectors. The composition can comprise from about 1×10⁷ to about 1×10¹⁴ of the vectors.

In another aspect, this document features an adeno-associated virus (AAV) vector comprising an AAV capsid polypeptide, wherein the capsid polypeptide comprises the amino acid sequence of any one of SEQ ID NOs:C2-C45. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:C2-C45 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:C2-C43 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:C2-C45. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:C44-C45. The vector can be an AAV2 vector. The vector can infect greater than 2 percent of retinal cells within two or more retinal regions when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal cells in at least two retinal regions than the level of expression from a comparable AAV vector comprising a capsid polypeptide consisting of the amino acid sequence set forth in SEQ ID NO:1, wherein the at least two retinal regions are selected from the group consisting of a fovea region, a parafovea region, a vascular arcade region, and a periphery region.

In another aspect, this document features an AAV capsid polypeptide comprising the amino acid sequence of any one of SEQ ID NOs:C2-C45. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:C2-C45 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:C2-C43 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:C2-C45. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:C44-C45. An AAV vector comprising the polypeptide can infect greater than 2 percent of retinal cells within two or more retinal regions when a titer of at least 1×10⁷ of the vector is administered intravitreally to an eye of a human (or a non-human primate). An AAV vector comprising the polypeptide can express more nucleic acid in retinal cells in at least two retinal regions than the level of expression from a comparable AAV vector comprising a capsid polypeptide consisting of the amino acid sequence set forth in SEQ ID NO:1, wherein the at least two retinal regions are selected from the group consisting of a fovea region, a parafovea region, a vascular arcade region, and a periphery region.

In another aspect, this document features a nucleic acid molecule encoding an AAV vector comprising an AAV capsid polypeptide, wherein the capsid polypeptide comprises the amino acid sequence of any one of SEQ ID NOs:C2-C45. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:C2-C45 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:C2-C43 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:C2-C45. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:C44-C45. The vector can be an AAV2 vector. The vector can infect greater than 2 percent of retinal cells within two or more retinal regions when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal cells in at least two retinal regions than the level of expression from a comparable AAV vector comprising a capsid polypeptide consisting of the amino acid sequence set forth in SEQ ID NO:1, wherein the at least two retinal regions are selected from the group consisting of a fovea region, a parafovea region, a vascular arcade region, and a periphery region. The nucleic acid molecule can be DNA.

In another aspect, this document features a nucleic acid molecule encoding an AAV capsid polypeptide comprising the amino acid sequence of any one of SEQ ID NOs:C2-C45.

The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:C2-C45 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:C2-C43 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO: 1 are replaced with the amino acid sequence of any one of SEQ ID NOs:C2-C45. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:C44-C45. An AAV vector comprising the polypeptide can infect greater than 2 percent of retinal cells within two or more retinal regions when a titer of at least 1×10⁷ of the vector is administered intravitreally to an eye of a human (or a non-human primate). An AAV vector comprising the polypeptide can express more nucleic acid in retinal cells in at least two retinal regions than the level of expression from a comparable AAV vector comprising a capsid polypeptide consisting of the amino acid sequence set forth in SEQ ID NO:1, wherein the at least two retinal regions are selected from the group consisting of a fovea region, a parafovea region, a vascular arcade region, and a periphery region. The nucleic acid molecule can be DNA.

In another aspect, this document features a host cell comprising a nucleic acid molecule of either of the two preceding paragraphs. The host cell can express the vector. The host cell can express the polypeptide.

In another aspect, this document features a host cell comprising an AAV vector comprising an AAV capsid polypeptide, wherein the capsid polypeptide comprises the amino acid sequence of any one of SEQ ID NOs:C2-C45. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:C2-C45 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:C2-C43 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:C2-C45. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:C44-C45. The vector can be an AAV2 vector. The vector can infect greater than 2 percent of retinal cells within two or more retinal regions when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal cells in at least two retinal regions than the level of expression from a comparable AAV vector comprising a capsid polypeptide consisting of the amino acid sequence set forth in SEQ ID NO:1, wherein the at least two retinal regions are selected from the group consisting of a fovea region, a parafovea region, a vascular arcade region, and a periphery region. The host cell can be a retinal cell.

In another aspect, this document features a host cell comprising an AAV capsid polypeptide comprising the amino acid sequence of any one of SEQ ID NOs:C2-C45. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:C2-C45 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:C2-C43 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:C2-C45. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:C44-C45. An AAV vector comprising the polypeptide can infect greater than 2 percent of retinal cells within two or more retinal regions when a titer of at least 1×10⁷ of the vector is administered intravitreally to an eye of a human (or a non-human primate). An AAV vector comprising the polypeptide can express more nucleic acid in retinal cells in at least two retinal regions than the level of expression from a comparable AAV vector comprising a capsid polypeptide consisting of the amino acid sequence set forth in SEQ ID NO:1, wherein the at least two retinal regions are selected from the group consisting of a fovea region, a parafovea region, a vascular arcade region, and a periphery region. The host cell can be a retinal cell.

In another aspect, this document features a composition comprising an AAV vector comprising an AAV capsid polypeptide, wherein the capsid polypeptide comprises the amino acid sequence of any one of SEQ ID NOs:C2-C45. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:C2-C45 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:C2-C43 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:C2-C45. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:C44-C45. The vector can be an AAV2 vector. The vector can infect greater than 2 percent of retinal cells within two or more retinal regions when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal cells in at least two retinal regions than the level of expression from a comparable AAV vector comprising a capsid polypeptide consisting of the amino acid sequence set forth in SEQ ID NO:1, wherein the at least two retinal regions are selected from the group consisting of a fovea region, a parafovea region, a vascular arcade region, and a periphery region. The composition can comprise from about 1×10⁷ to about 1×10¹⁴ of the vector. The composition can comprise phosphate buffered saline, Hank's Balanced Salt Solution, or Pluronic F68.

In another aspect, this document features a method for delivering an exogenous nucleic acid sequence to retinal cells within at least two different retinal regions of an eye of a mammal. The method comprises (or consists essentially of, or consists of) contacting the retinal cells with an AAV vector comprising an AAV capsid polypeptide and the exogenous nucleic acid sequence, wherein the capsid polypeptide comprises the amino acid sequence of any one of SEQ ID NOs:C2-C45, wherein the AAV vector infects retinal cells within the at least two different retinal regions, thereby delivering the exogenous nucleic acid sequence to the retinal cells, wherein the at least two retinal regions are selected from the group consisting of a fovea region, a parafovea region, a vascular arcade region, and a periphery region. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:C2-C45 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:C2-C43 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:C2-C45. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:C44-C45. The mammal can be a human (or a non-human primate). The vector can be an AAV2 vector. The vector can infect greater than 2 percent of retinal cells within the at least two retinal regions when a titer of at least 1×10⁷ of the vector is administered intravitreally to an eye of a human (or a non-human primate). The exogenous nucleic acid sequence can encode an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more of the exogenous nucleic acid sequence in the retinal cells of the at least two retinal regions than the level of expression in a retinal cell from a comparable AAV vector comprising a capsid polypeptide consisting of the amino acid sequence set forth in SEQ ID NO:1. The method can comprise intravitreally administering a composition comprising the vector to the mammal, thereby contacting the retinal cells with the vector. The composition can comprise from about 1×10⁷ to about 1×10¹⁴ of the vector.

In another aspect, this document features a method for treating a retinal condition. The method comprises (or consists essentially of, or consists of) contacting retinal cells of at least two retinal regions of an eye of a mammal having the retinal condition with AAV vectors comprising an AAV capsid polypeptide and an exogenous nucleic acid sequence, wherein the capsid polypeptide comprises the amino acid sequence of any one of SEQ ID NOs:C2-C45, wherein the AAV vectors infect the retinal cells of the at least two retinal regions and drive expression of the exogenous nucleic acid sequence within the retinal cells of the at least two retinal regions, thereby treating the retinal condition. The mammal can be a human (or a non-human primate). The retinal condition can be selected from the group consisting of LCA, OCA1, retinitis pigmentosa, rod/cone dystrophy, cone dystrophy, Stargardt Disease, Usher syndrome, XLRP, and XLRS. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:C2-C45 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acid sequence of any one of SEQ ID NOs:C2-C43 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:C2-C45. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:C44-C45. The vectors can be AAV2 vectors. The vectors can infect greater than 2 percent of retinal cells in the at least two retinal regions when a titer of at least 1×10¹⁴ of the vectors is administered intravitreally to an eye of the mammal. The exogenous nucleic acid sequence can encode an RNA. The RNA can be an siRNA or a microRNA. The exogenous nucleic acid can encode a polypeptide. The polypeptide can be an ABCA4 polypeptide, a CRB1 polypeptide, an NPHP5 polypeptide, and an NR2E3 polypeptide. The vectors can express more of the exogenous nucleic acid sequence in the retinal cells of the at least two retinal regions than the level of expression in retinal cells of the at least two retinal regions from a comparable AAV vector comprising a capsid polypeptide consisting of the amino acid sequence set forth in SEQ ID NO:1. The method can comprise intravitreally administering a composition comprising the vectors to the mammal, thereby contacting the retinal cells of the at least two retinal regions with the vectors. The composition can comprise from about 1×10⁷ to about 1×10¹⁴ of the vectors. The at least two retinal regions can be selected from the group consisting of a fovea region, a parafovea region, a vascular arcade region, and a periphery region.

In another aspect, this document features an adeno-associated virus (AAV) vector comprising an AAV capsid polypeptide, wherein the capsid polypeptide comprises the amino acid sequence of any one of SEQ ID NOs:D2-D78. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D78 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D76 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:D2-D78. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:D77-D78. The vector can be an AAV2 vector. The vector can infect greater than 2 percent of retinal cells of a parafovea region of an eye when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal cells of a parafovea region of an eye than the level of expression from a comparable AAV vector comprising a capsid polypeptide consisting of the amino acid sequence set forth in SEQ ID NO:1.

In another aspect, this document features an AAV capsid polypeptide comprising the amino acid sequence of any one of SEQ ID NOs:D2-D78. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D78 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D76 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:D2-D78. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:D77-D78. An AAV vector comprising the polypeptide can infect greater than 2 percent of retinal cells of a parafovea region of an eye when a titer of at least 1×10⁷ of the vector is administered intravitreally to an eye of a human (or a non-human primate). An AAV vector comprising the polypeptide can express more nucleic acid in retinal cells of a parafovea region of an eye than the level of expression from a comparable AAV vector comprising a capsid polypeptide consisting of the amino acid sequence set forth in SEQ ID NO:1.

In another aspect, this document features a nucleic acid molecule encoding an AAV vector comprising an AAV capsid polypeptide, wherein the capsid polypeptide comprises the amino acid sequence of any one of SEQ ID NOs:D2-D78. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D78 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D76 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:D2-D78. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:D77-D78. The vector can be an AAV2 vector. The vector can infect greater than 2 percent of retinal cells of a parafovea region of an eye when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal cells of a parafovea region of an eye than the level of expression from a comparable AAV vector comprising a capsid polypeptide consisting of the amino acid sequence set forth in SEQ ID NO:1. The nucleic acid molecule can be DNA.

In another aspect, this document features a nucleic acid molecule encoding an AAV capsid polypeptide comprising the amino acid sequence of any one of SEQ ID NOs:D2-D78. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D78 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D76 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:D2-D78. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:D77-D78. An AAV vector comprising the polypeptide can infect greater than 2 percent of retinal cells of a parafovea region of an eye when a titer of at least 1×10⁷ of the vector is administered intravitreally to an eye of a human (or a non-human primate). An AAV vector comprising the polypeptide can express more nucleic acid in retinal cells of a parafovea region of an eye than the level of expression from a comparable AAV vector comprising a capsid polypeptide consisting of the amino acid sequence set forth in SEQ ID NO:1. The nucleic acid molecule can be DNA.

In another aspect, this document features a host cell comprising a nucleic acid molecule of either of the two preceding paragraphs. The host cell can express the vector. The host cell can express the polypeptide.

In another aspect, this document features a host cell comprising an AAV vector comprising an AAV capsid polypeptide, wherein the capsid polypeptide comprises the amino acid sequence of any one of SEQ ID NOs:D2-D78. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D78 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D76 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:D2-D78. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:D77-D78. The vector can be an AAV2 vector. The vector can infect greater than 2 percent of retinal cells of a parafovea region of an eye when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal cells of a parafovea region of an eye than the level of expression from a comparable AAV vector comprising a capsid polypeptide consisting of the amino acid sequence set forth in SEQ ID NO:1. The host cell can be a retinal cell.

In another aspect, this document features a host cell comprising an AAV capsid polypeptide comprising the amino acid sequence of any one of SEQ ID NOs:D2-D78. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D78 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D76 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:D2-D78. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:D77-D78. An AAV vector comprising the polypeptide can infect greater than 2 percent of retinal cells of a parafovea region of an eye when a titer of at least 1×10⁷ of the vector is administered intravitreally to an eye of a human (or a non-human primate). An AAV vector comprising the polypeptide can express more nucleic acid in retinal cells of a parafovea region of an eye than the level of expression from a comparable AAV vector comprising a capsid polypeptide consisting of the amino acid sequence set forth in SEQ ID NO:1. The host cell can be a retinal cell.

In another aspect, this document features a composition comprising an AAV vector comprising an AAV capsid polypeptide, wherein the capsid polypeptide comprises the amino acid sequence of any one of SEQ ID NOs:D2-D78. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D78 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D76 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:D2-D78. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:D77-D78. The vector can be an AAV2 vector. The vector can infect greater than 2 percent of retinal cells of a parafovea region of an eye when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal cells of a parafovea region of an eye than the level of expression from a comparable AAV vector comprising a capsid polypeptide consisting of the amino acid sequence set forth in SEQ ID NO:1. The composition can comprise from about 1×10⁷ to about 1×10¹⁴ of the vector. The composition can comprise phosphate buffered saline, Hank's Balanced Salt Solution, or Pluronic F68.

In another aspect, this document features a method for delivering an exogenous nucleic acid sequence to retinal cells of a parafovea region of an eye within a mammal. The method comprises (or consists essentially of, or consists of) contacting the retinal cells with an AAV vector comprising an AAV capsid polypeptide and the exogenous nucleic acid sequence, wherein the capsid polypeptide comprises the amino acid sequence of any one of SEQ ID NOs:D2-D78, wherein the AAV vector infects the retinal cells, thereby delivering the exogenous nucleic acid sequence to the retinal cells. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D78 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D76 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:D2-D78. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:D77-D78. The mammal can be a human (or a non-human primate). The vector can be an AAV2 vector. The vector can infect greater than 2 percent of retinal cells of the parafovea region of the eye when a titer of at least 1×10⁷ of the vector is administered intravitreally. The exogenous nucleic acid sequence can encode an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more of the exogenous nucleic acid sequence in the retinal cells than the level of expression in retinal cells of a parafovea region of an eye from a comparable AAV vector comprising a capsid polypeptide consisting of the amino acid sequence set forth in SEQ ID NO:1. The method can comprise intravitreally administering a composition comprising the vector to the mammal, thereby contacting the retinal cells of the parafovea region with the vector. The composition can comprise from about 1×10⁷ to about 1×10¹⁴ of the vector.

In another aspect, this document features a method for treating a retinal condition. The method comprises (or consists essentially of, or consists of) contacting retinal cells of a parafovea region of an eye of a mammal having the retinal condition with AAV vectors comprising an AAV capsid polypeptide and an exogenous nucleic acid sequence, wherein the capsid polypeptide comprises the amino acid sequence of any one of SEQ ID NOs:D2-D78, wherein the AAV vectors infect the retinal cells and drive expression of the exogenous nucleic acid sequence within the retinal cells, thereby treating the retinal condition. The mammal can be a human (or a non-human primate). The retinal condition can be selected from the group consisting of cone dystrophy, cone/rod dystrophy, retinitis pigmentosa, macular degeneration, achromatopsia, blue cone monochromcy, and color blindness. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D78 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:D2-D76 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:D2-D78. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:D77-D78. The vectors can be AAV2 vectors. The vectors can infect greater than 2 percent of retinal cells of the parafovea region when a titer of at least 1×10⁷ of the vectors is administered intravitreally. The exogenous nucleic acid sequence can encode an RNA. The RNA can be an siRNA or a microRNA. The exogenous nucleic acid can encode a polypeptide. The polypeptide can be an ABCA4 polypeptide, a CRB1 polypeptide, an NPHP5 polypeptide, and an NR2E3 polypeptide. The vectors can express more of the exogenous nucleic acid sequence in the retinal cells of the parafovea region than the level of expression in retinal cells of a parafovea region from a comparable AAV vector comprising a capsid polypeptide consisting of the amino acid sequence set forth in SEQ ID NO:1. The method can comprise intravitreally administering a composition comprising the vectors to the mammal, thereby contacting the retinal cells of the parafovea region with the vectors. The composition can comprise from about 1×10⁷ to about 1×10¹⁴ of the vectors.

In another aspect, this document features an adeno-associated virus (AAV) vector comprising an AAV capsid polypeptide, wherein the capsid polypeptide comprises the amino acid sequence of any one of SEQ ID NOs:E2-E83. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E83 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E75 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:E2-E83. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:E76-E83. The vector can be an AAV2 vector. The vector can infect at least three different retinal cell types when a titer of at least 1×10⁷ of the vector is administered intravitreally to an eye of a human (or a non-human primate), wherein the at least three different retinal cell types are selected from the group consisting of retinal ganglion cells, amacrine cells, horizontal cells, bipolar cells, Muller glia cells, photoreceptor cells, and RPE cells. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in at least three different retinal cell types than the level of expression from a comparable AAV vector comprising a capsid polypeptide consisting of the amino acid sequence set forth in SEQ ID NO:1, wherein the at least three different retinal cell types are selected from the group consisting of retinal ganglion cells, amacrine cells, horizontal cells, bipolar cells, Muller glia cells, photoreceptor cells, and RPE cells.

In another aspect, this document features an AAV capsid polypeptide comprising the amino acid sequence of any one of SEQ ID NOs:E2-E83. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E83 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E75 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:E2-E83. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:E76-E83. An AAV vector comprising the polypeptide can infect at least three different retinal cell types when a titer of at least 1×10⁷ of the vector is administered intravitreally to an eye of a human (or a non-human primate), wherein the at least three different retinal cell types are selected from the group consisting of retinal ganglion cells, amacrine cells, horizontal cells, bipolar cells, Muller glia cells, photoreceptor cells, and RPE cells. An AAV vector comprising the polypeptide can express more nucleic acid in at least three different retinal cell types than the level of expression from a comparable AAV vector comprising a capsid polypeptide consisting of the amino acid sequence set forth in SEQ ID NO:1, wherein the at least three different retinal cell types are selected from the group consisting of retinal ganglion cells, amacrine cells, horizontal cells, bipolar cells, Muller glia cells, photoreceptor cells, and RPE cells.

In another aspect, this document features a nucleic acid molecule encoding an AAV vector comprising an AAV capsid polypeptide, wherein the capsid polypeptide comprises the amino acid sequence of any one of SEQ ID NOs:E2-E83. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E83 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E75 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:E2-E83. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:E76-E83. The vector can be an AAV2 vector. The vector can infect at least three different retinal cell types when a titer of at least 1×10⁷ of the vector is administered intravitreally to an eye of a human (or a non-human primate), wherein the at least three different retinal cell types are selected from the group consisting of retinal ganglion cells, amacrine cells, horizontal cells, bipolar cells, Muller glia cells, photoreceptor cells, and RPE cells. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in at least three different retinal cell types than the level of expression from a comparable AAV vector comprising a capsid polypeptide consisting of the amino acid sequence set forth in SEQ ID NO:1, wherein the at least three different retinal cell types are selected from the group consisting of retinal ganglion cells, amacrine cells, horizontal cells, bipolar cells, Muller glia cells, photoreceptor cells, and RPE cells. The nucleic acid molecule can be DNA.

In another aspect, this document features a nucleic acid molecule encoding an AAV capsid polypeptide comprising the amino acid sequence of any one of SEQ ID NOs:E2-E83. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E83 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E75 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:E2-E83. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:E76-E83. An AAV vector comprising the polypeptide can infect at least three different retinal cell types when a titer of at least 1×10⁷ of the vector is administered intravitreally to an eye of a human (or a non-human primate), wherein the at least three different retinal cell types are selected from the group consisting of retinal ganglion cells, amacrine cells, horizontal cells, bipolar cells, Muller glia cells, photoreceptor cells, and RPE cells. An AAV vector comprising the polypeptide can express more nucleic acid in at least three different retinal cell types than the level of expression from a comparable AAV vector comprising a capsid polypeptide consisting of the amino acid sequence set forth in SEQ ID NO:1, wherein the at least three different retinal cell types are selected from the group consisting of retinal ganglion cells, amacrine cells, horizontal cells, bipolar cells, Muller glia cells, photoreceptor cells, and RPE cells. The nucleic acid molecule can be DNA.

In another aspect, this document features a host cell comprising a nucleic acid molecule of either of the two preceding paragraphs. The host cell can express the vector. The host cell can express the polypeptide.

In another aspect, this document features a host cell comprising an AAV vector comprising an AAV capsid polypeptide, wherein the capsid polypeptide comprises the amino acid sequence of any one of SEQ ID NOs:E2-E83. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E83 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E75 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:E2-E83. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:E76-E83. The vector can be an AAV2 vector. The vector can infect at least three different retinal cell types when a titer of at least 1×10⁷ of the vector is administered intravitreally to an eye of a human (or a non-human primate), wherein the at least three different retinal cell types are selected from the group consisting of retinal ganglion cells, amacrine cells, horizontal cells, bipolar cells, Muller glia cells, photoreceptor cells, and RPE cells. The vector can comprise an exogenous nucleic acid encoding an RNA or a polypeptide.

The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in at least three different retinal cell types than the level of expression from a comparable AAV vector comprising a capsid polypeptide consisting of the amino acid sequence set forth in SEQ ID NO:1, wherein the at least three different retinal cell types are selected from the group consisting of retinal ganglion cells, amacrine cells, horizontal cells, bipolar cells, Muller glia cells, photoreceptor cells, and RPE cells. The host cell can be a retinal cell.

In another aspect, this document features a host cell comprising an AAV capsid polypeptide comprising the amino acid sequence of any one of SEQ ID NOs:E2-E83. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E83 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E75 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:E2-E83. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:E76-E83. An AAV vector comprising the polypeptide can infect at least three different retinal cell types when a titer of at least 1×10⁷ of the vector is administered intravitreally to an eye of a human (or a non-human primate), wherein the at least three different retinal cell types are selected from the group consisting of retinal ganglion cells, amacrine cells, horizontal cells, bipolar cells, Muller glia cells, photoreceptor cells, and RPE cells. An AAV vector comprising the polypeptide can express more nucleic acid in at least three different retinal cell types than the level of expression from a comparable AAV vector comprising a capsid polypeptide consisting of the amino acid sequence set forth in SEQ ID NO:1, wherein the at least three different retinal cell types are selected from the group consisting of retinal ganglion cells, amacrine cells, horizontal cells, bipolar cells, Muller glia cells, photoreceptor cells, and RPE cells. The host cell can be a retinal cell.

In another aspect, this document features a composition comprising an AAV vector comprising an AAV capsid polypeptide, wherein the capsid polypeptide comprises the amino acid sequence of any one of SEQ ID NOs:E2-E83. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E83 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E75 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:E2-E83. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:E76-E83. The vector can be an AAV2 vector. The vector can infect at least three different retinal cell types when a titer of at least 1×10⁷ of the vector is administered intravitreally to an eye of a human (or a non-human primate), wherein the at least three different retinal cell types are selected from the group consisting of retinal ganglion cells, amacrine cells, horizontal cells, bipolar cells, Muller glia cells, photoreceptor cells, and RPE cells. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in at least three different retinal cell types than the level of expression from a comparable AAV vector comprising a capsid polypeptide consisting of the amino acid sequence set forth in SEQ ID NO:1, wherein the at least three different retinal cell types are selected from the group consisting of retinal ganglion cells, amacrine cells, horizontal cells, bipolar cells, Muller glia cells, photoreceptor cells, and RPE cells. The composition can comprise from about 1×10⁷ to about 1×10¹⁴ of the vector. The composition can comprise phosphate buffered saline, Hank's Balanced Salt Solution, or Pluronic F68.

In another aspect, this document features a method for delivering an exogenous nucleic acid sequence to at least three different retinal cell types of an eye of a mammal. The method comprises (or consists essentially of, or consists of) contacting the at least three different retinal cell types with an AAV vector comprising an AAV capsid polypeptide and the exogenous nucleic acid sequence, wherein the capsid polypeptide comprises the amino acid sequence of any one of SEQ ID NOs:E2-E83, wherein the AAV vector infects the at least three different retinal cell types, thereby delivering the exogenous nucleic acid sequence to the at least three different retinal cell types, wherein the at least three different retinal cell types are selected from the group consisting of retinal ganglion cells, amacrine cells, horizontal cells, bipolar cells, Muller glia cells, photoreceptor cells, and RPE cells. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E83 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E75 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:E2-E83. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:E76-E83. The mammal can be a human (or a non-human primate). The vector can be an AAV2 vector. The vector can infect greater than 2 percent of the at least three different retinal cell types within an eye when a titer of at least 1×10⁷ of the vector is administered intravitreally to the eye. The exogenous nucleic acid sequence can encode an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more of the exogenous nucleic acid sequence in the at least three different retinal cell types than the level of expression in the at least three different retinal cell types from a comparable AAV vector comprising a capsid polypeptide consisting of the amino acid sequence set forth in SEQ ID NO:1. The method can comprise intravitreally administering a composition comprising the vector to the mammal, thereby contacting the at least three different retinal cell types with the vector. The composition can comprise from about 1×10⁷ to about 1×10¹⁴ of the vector.

In another aspect, this document features a method for treating a retinal condition. The method comprises (or consists essentially of, or consists of) contacting at least three different retinal cell types of an eye of a mammal having the retinal condition with AAV vectors comprising an AAV capsid polypeptide and an exogenous nucleic acid sequence, wherein the capsid polypeptide comprises the amino acid sequence of any one of SEQ ID NOs:E2-E83, wherein the AAV vectors infect the at least three different retinal cell types and drive expression of the exogenous nucleic acid sequence within the at least three different retinal cell types, thereby treating the retinal condition. The mammal can be a human (or a non-human primate). The retinal condition can be selected from the group consisting of cone dystrophy, cone/rod dystrophy, retinitis pigmentosa, macular degeneration, achromatopsia, blue cone monochromcy, and color blindness. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E83 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:E2-E75 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:E2-E83. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:E76-E83. The vectors can be AAV2 vectors. The vectors can infect greater than 2 percent of the at least three different retinal cell types within the eye when a titer of at least 1×10⁷ of the vectors is administered intravitreally. The exogenous nucleic acid sequence can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vectors can express more of the exogenous nucleic acid sequence in the at least three different retinal cell types than the level of expression in the at least three different retinal cell types from a comparable AAV vector comprising a capsid polypeptide consisting of the amino acid sequence set forth in SEQ ID NO:1. The method can comprise intravitreally administering a composition comprising the vectors to the mammal, thereby contacting the at least three different retinal cell types with the vectors. The composition can comprise from about 1×10⁷ to about 1×10¹⁴ of the vector. The at least three different retinal cell types can be selected from the group consisting of retinal ganglion cells, amacrine cells, horizontal cells, bipolar cells, Muller glia cells, photoreceptor cells, and RPE cells.

In another aspect, this document features an adeno-associated virus (AAV) vector comprising an AAV capsid polypeptide, wherein the capsid polypeptide comprises the amino acid sequence of any one of SEQ ID NOs:F2-F10. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F5 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F6-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:F2-F10. The vector can be an AAV2 vector. The vector can infect greater than 2 percent of RPE cells when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in RPE cells than the level of expression from a comparable AAV vector comprising a capsid polypeptide consisting of the amino acid sequence set forth in SEQ ID NO:1.

In another aspect, this document features an AAV capsid polypeptide comprising the amino acid sequence of any one of SEQ ID NOs:F2-F10. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F5 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F6-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:F2-F10. An AAV vector comprising the polypeptide can infect greater than 2 percent of RPE cells when a titer of at least 1×10⁷ of the vector is administered intravitreally to an eye of a human (or a non-human primate). An AAV vector comprising the polypeptide can express more nucleic acid in RPE cells than the level of expression from a comparable AAV vector comprising a capsid polypeptide consisting of the amino acid sequence set forth in SEQ ID NO:1.

In another aspect, this document features a nucleic acid molecule encoding an AAV vector comprising an AAV capsid polypeptide, wherein the capsid polypeptide comprises the amino acid sequence of any one of SEQ ID NOs:F2-F10. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F5 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acid sequence of any one of SEQ ID NOs:F6-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:F2-F10. The vector can be an AAV2 vector. The vector can infect greater than 2 percent of RPE cells when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an N1R2E3 polypeptide. The vector can express more nucleic acid in RPE cells than the level of expression from a comparable AAV vector comprising a capsid polypeptide consisting of the amino acid sequence set forth in SEQ ID NO:1. The nucleic acid molecule can be DNA.

In another aspect, this document features a nucleic acid molecule encoding an AAV capsid polypeptide comprising the amino acid sequence of any one of SEQ ID NOs:F2-F10. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F5 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F6-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:F2-F10. An AAV vector comprising the polypeptide can infect greater than 2 percent of RPE cells when a titer of at least 1×10⁷ of the vector is administered intravitreally to an eye of a human (or a non-human primate). An AAV vector comprising the polypeptide can express more nucleic acid in RPE cells than the level of expression from a comparable AAV vector comprising a capsid polypeptide consisting of the amino acid sequence set forth in SEQ ID NO:1. The nucleic acid molecule can be DNA.

In another aspect, this document features a host cell comprising a nucleic acid molecule of either of the two preceding paragraphs. The host cell can express the vector. The host cell can express the polypeptide.

In another aspect, this document features a host cell comprising an AAV vector comprising an AAV capsid polypeptide, wherein the capsid polypeptide comprises the amino acid sequence of any one of SEQ ID NOs:F2-F10. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F5 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F6-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:F2-F10. The vector can be an AAV2 vector. The vector can infect greater than 2 percent of RPE cells when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in RPE cells than the level of expression from a comparable AAV vector comprising a capsid polypeptide consisting of the amino acid sequence set forth in SEQ ID NO:1. The host cell can be a retinal cell.

In another aspect, this document features a host cell comprising an AAV capsid polypeptide comprising the amino acid sequence of any one of SEQ ID NOs:F2-F10. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F5 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F6-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:F2-F10. An AAV vector comprising the polypeptide can infect greater than 2 percent of RPE cells when a titer of at least 1×10⁷ of the vector is administered intravitreally to an eye of a human (or a non-human primate). An AAV vector comprising the polypeptide can express more nucleic acid in RPE cells than the level of expression from a comparable AAV vector comprising a capsid polypeptide consisting of the amino acid sequence set forth in SEQ ID NO:1. The host cell can be a retinal cell.

In another aspect, this document features a composition comprising an AAV vector comprising an AAV capsid polypeptide, wherein the capsid polypeptide comprises the amino acid sequence of any one of SEQ ID NOs:F2-F10. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F5 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F6-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:F2-F10. The vector can be an AAV2 vector. The vector can infect greater than 2 percent of RPE cells when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in RPE cells than the level of expression from a comparable AAV vector comprising a capsid polypeptide consisting of the amino acid sequence set forth in SEQ ID NO:1. The composition can comprise from about 1×10⁷ to about 1×10¹⁴ of the vector. The composition can comprise phosphate buffered saline, Hank's Balanced Salt Solution, or Pluronic F68.

In another aspect, this document features a method for delivering an exogenous nucleic acid sequence to RPE cells of an eye of a mammal. The method comprises (or consists essentially of, or consists of) contacting the RPE cells with an AAV vector comprising an AAV capsid polypeptide and the exogenous nucleic acid sequence, wherein the capsid polypeptide comprises the amino acid sequence of any one of SEQ ID NOs:F2-F10, wherein the AAV vector infects RPE cells, thereby delivering the exogenous nucleic acid sequence to the RPE cells. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F5 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F6-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:F2-F10. The mammal can be a human (or a non-human primate). The vector can be an AAV2 vector. The vector can infect greater than 2 percent of RPE cells of an eye when a titer of at least 1×10⁷ of the vector is administered intravitreally to the eye. The exogenous nucleic acid sequence can encode an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more of the exogenous nucleic acid sequence in the RPE cells than the level of expression in a RPE cell from a comparable AAV vector comprising a capsid polypeptide consisting of the amino acid sequence set forth in SEQ ID NO:1. The method can comprise intravitreally administering a composition comprising the vector to the mammal, thereby contacting the RPE cells with the vector. The composition can comprise from about 1×10⁷ to about 1×10¹⁴ of the vector.

In another aspect, this document features a method for treating a retinal condition. The method comprises (or consists essentially of, or consists of) contacting RPE cells of an eye of a mammal having the retinal condition with AAV vectors comprising an AAV capsid polypeptide and an exogenous nucleic acid sequence, wherein the capsid polypeptide comprises the amino acid sequence of any one of SEQ ID NOs:F2-F10, wherein the AAV vectors infect the RPE cells and drive expression of the exogenous nucleic acid sequence within the RPE cells, thereby treating the retinal condition. The mammal can be a human (or a non-human primate). The retinal condition can be selected from the group consisting of cone dystrophy, cone/rod dystrophy, retinitis pigmentosa, macular degeneration, achromatopsia, blue cone monochromcy, and color blindness. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F2-F5 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:F6-F10 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:F2-F10. The vectors can be AAV2 vectors. The vectors can infect greater than 2 percent of RPE cells when a titer of at least 1×10⁷ of the vectors is administered intravitreally to an eye of the mammal. The exogenous nucleic acid sequence can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vectors can express more of the exogenous nucleic acid sequence in the RPE cells than the level of expression in RPE cells from a comparable AAV vector comprising a capsid polypeptide consisting of the amino acid sequence set forth in SEQ ID NO:1. The method can comprise intravitreally administering a composition comprising the vectors to the mammal, thereby contacting the RPE cells with the vectors. The composition can comprise from about 1×10⁷ to about 1×10¹⁴ of the vector.

In another aspect, this document features an adeno-associated virus (AAV) vector comprising an AAV capsid polypeptide, wherein the capsid polypeptide comprises the amino acid sequence of any one of SEQ ID NOs:G2-G77. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:G2-G77 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:G2-G70 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:G2-G77. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:G71-G77. The vector can be an AAV2 vector. The vector can infect greater than 2 percent of photoreceptor cells of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in photoreceptor cells than the level of expression from a comparable AAV vector comprising a capsid polypeptide consisting of the amino acid sequence set forth in SEQ ID NO:1.

In another aspect, this document features an AAV capsid polypeptide comprising the amino acid sequence of any one of SEQ ID NOs:G2-G77. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:G2-G77 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:G2-G70 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:G2-G77. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:G71-G77. An AAV vector comprising the polypeptide can infect greater than 2 percent of photoreceptor cells of an eye when a titer of at least 1×10¹ of the vector is administered intravitreally to the eye. An AAV vector comprising the polypeptide can express more nucleic acid in photoreceptor cells than the level of expression from a comparable AAV vector comprising a capsid polypeptide consisting of the amino acid sequence set forth in SEQ ID NO:1.

In another aspect, this document features a nucleic acid molecule encoding an AAV vector comprising an AAV capsid polypeptide, wherein the capsid polypeptide comprises the amino acid sequence of any one of SEQ ID NOs:G2-G77. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:G2-G77 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:G2-G70 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:G2-G77. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:G71-G77. The vector can be an AAV2 vector. The vector can infect greater than 2 percent of photoreceptor cells of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in photoreceptor cells than the level of expression from a comparable AAV vector comprising a capsid polypeptide consisting of the amino acid sequence set forth in SEQ ID NO:1. The nucleic acid molecule can be DNA.

In another aspect, this document features a nucleic acid molecule encoding an AAV capsid polypeptide comprising the amino acid sequence of any one of SEQ ID NOs:G2-G77. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:G2-G77 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:G2-G70 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:G2-G77. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:G71-G77. An AAV vector comprising the polypeptide can infect greater than 2 percent of photoreceptor cells of an eye when a titer of at least 1×10⁷ of the vector is administered intravitreally to the eye. An AAV vector comprising the polypeptide can express more nucleic acid in photoreceptor cells than the level of expression from a comparable AAV vector comprising a capsid polypeptide consisting of the amino acid sequence set forth in SEQ ID NO:1. The nucleic acid molecule can be DNA.

In another aspect, this document features a host cell comprising a nucleic acid molecule of either of the two preceding paragraphs. The host cell can express the vector. The host cell can express the polypeptide.

In another aspect, this document features a host cell comprising an AAV vector comprising an AAV capsid polypeptide, wherein the capsid polypeptide comprises the amino acid sequence of any one of SEQ ID NOs:G2-G77. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:G2-G77 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:G2-G70 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:G2-G77. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:G71-G77. The vector can be an AAV2 vector. The vector can infect greater than 2 percent of photoreceptor cells of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in photoreceptor cells than the level of expression from a comparable AAV vector comprising a capsid polypeptide consisting of the amino acid sequence set forth in SEQ ID NO:1. The host cell can be a retinal cell.

In another aspect, this document features a host cell comprising an AAV capsid polypeptide comprising the amino acid sequence of any one of SEQ ID NOs:G2-G77. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:G2-G77 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:G2-G70 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:G2-G77. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:G71-G77. An AAV vector comprising the polypeptide can infect greater than 2 percent of photoreceptor cells of an eye when a titer of at least 1×10⁷ of the vector is administered intravitreally to the eye. An AAV vector comprising the polypeptide can express more nucleic acid in photoreceptor cells than the level of expression from a comparable AAV vector comprising a capsid polypeptide consisting of the amino acid sequence set forth in SEQ ID NO:1. The host cell can be a retinal cell.

In another aspect, this document features a composition comprising an AAV vector comprising an AAV capsid polypeptide, wherein the capsid polypeptide comprises the amino acid sequence of any one of SEQ ID NOs:G2-G77. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:G2-G77 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acid sequence of any one of SEQ ID NOs:G2-G70 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:G2-G77. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:G71-G77. The vector can be an AAV2 vector. The vector can infect greater than 2 percent of photoreceptor cells of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in photoreceptor cells than the level of expression from a comparable AAV vector comprising a capsid polypeptide consisting of the amino acid sequence set forth in SEQ ID NO:1. The composition can comprise from about 1×10⁷ to about 1×10¹⁴ of the vector. The composition can comprise phosphate buffered saline, Hank's Balanced Salt Solution, or Pluronic F68.

In another aspect, this document features a method for delivering an exogenous nucleic acid sequence to a photoreceptor cell within a mammal. The method comprises (or consists essentially of, or consists of) contacting the photoreceptor cell with an AAV vector comprising an AAV capsid polypeptide and the exogenous nucleic acid sequence, wherein the capsid polypeptide comprises the amino acid sequence of any one of SEQ ID NOs:G2-G77, wherein the AAV vector infects the photoreceptor cell, thereby delivering the exogenous nucleic acid sequence to the photoreceptor cell. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:G2-G77 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:G2-G70 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:G2-G77. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:G71-G77. The mammal can be a human (or a non-human primate). The vector can be an AAV2 vector. The vector can infect greater than 2 percent of photoreceptor cells of an eye when a titer of at least 1×10⁷ of the vector is administered intravitreally to the eye. The exogenous nucleic acid sequence can encode an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more of the exogenous nucleic acid sequence in the photoreceptor cell than the level of expression in a photoreceptor cell from a comparable AAV vector comprising a capsid polypeptide consisting of the amino acid sequence set forth in SEQ ID NO:1. The method can comprise intravitreally administering a composition comprising the vector to the mammal, thereby contacting the photoreceptor cell with the vector. The composition can comprise from about 1×10⁷ to about 1×10¹⁴ of the vector.

In another aspect, this document features a method for treating a retinal condition. The method comprises (or consists essentially of, or consists of) contacting photoreceptor cells of a mammal having the retinal condition with AAV vectors comprising an AAV capsid polypeptide and an exogenous nucleic acid sequence, wherein the capsid polypeptide comprises the amino acid sequence of any one of SEQ ID NOs:G2-G77, wherein the AAV vectors infect the photoreceptor cells and drive expression of the exogenous nucleic acid sequence within the photoreceptor cells, thereby treating the retinal condition. The mammal can be a human (or a non-human primate). The retinal condition can be selected from the group consisting of cone dystrophy, cone/rod dystrophy, retinitis pigmentosa, macular degeneration, achromatopsia, blue cone monochromcy, and color blindness. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:G2-G77 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:G2-G70 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:G2-G77. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:G71-G77. The vectors can be AAV2 vectors. The vectors can infect greater than 2 percent of photoreceptor cells when a titer of at least 1×10⁷ of the vectors is administered intravitreally to an eye of the mammal. The exogenous nucleic acid sequence can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vectors can express more of the exogenous nucleic acid sequence in the photoreceptor cells than the level of expression in photoreceptor cells from a comparable AAV vector comprising a capsid polypeptide consisting of the amino acid sequence set forth in SEQ ID NO:1. The method can comprise intravitreally administering a composition comprising the vectors to the mammal, thereby contacting the photoreceptor cells with the vectors. The composition can comprise from about 1×10⁷ to about 1×10¹⁴ of the vector.

In another aspect, this document features an adeno-associated virus (AAV) vector comprising an AAV capsid polypeptide, wherein the capsid polypeptide comprises the amino acid sequence of any one of SEQ ID NOs:H2-H258. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:H2-H258 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:H2-H243 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:H2-H258. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:H244-H258. The vector can be an AAV2 vector. The vector can infect greater than 2 percent of retinal ganglion cells of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal ganglion cells than the level of expression from a comparable AAV vector comprising a capsid polypeptide consisting of the amino acid sequence set forth in SEQ ID NO:1.

In another aspect, this document features an AAV capsid polypeptide comprising the amino acid sequence of any one of SEQ ID NOs:H2-H258. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:H2-H258 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:H2-H243 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:H2-H258. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:H244-H258. An AAV vector comprising the polypeptide can infect greater than 2 percent of retinal ganglion cells of an eye when a titer of at least 1×10⁷ of the vector is administered intravitreally to the eye. An AAV vector comprising the polypeptide can express more nucleic acid in retinal ganglion cells than the level of expression from a comparable AAV vector comprising a capsid polypeptide consisting of the amino acid sequence set forth in SEQ ID NO:1.

In another aspect, this document features a nucleic acid molecule encoding an AAV vector comprising an AAV capsid polypeptide, wherein the capsid polypeptide comprises the amino acid sequence of any one of SEQ ID NOs:H2-H258. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:H2-H258 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:H2-H243 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:H2-H258. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:H244-H258. The vector can be an AAV2 vector. The vector can infect greater than 2 percent of retinal ganglion cells of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal ganglion cells than the level of expression from a comparable AAV vector comprising a capsid polypeptide consisting of the amino acid sequence set forth in SEQ ID NO:1. The nucleic acid molecule can be DNA.

In another aspect, this document features a nucleic acid molecule encoding an AAV capsid polypeptide comprising the amino acid sequence of any one of SEQ ID NOs:H2-H258. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:H2-H258 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:H2-H243 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:H2-H258. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:H244-H258. An AAV vector comprising the polypeptide can infect greater than 2 percent of retinal ganglion cells of an eye when a titer of at least 1×10⁷ of the vector is administered intravitreally to the eye. An AAV vector comprising the polypeptide can express more nucleic acid in retinal ganglion cells than the level of expression from a comparable AAV vector comprising a capsid polypeptide consisting of the amino acid sequence set forth in SEQ ID NO:1. The nucleic acid molecule can be DNA.

In another aspect, this document features a host cell comprising a nucleic acid molecule of either of the two preceding paragraphs. The host cell can express the vector. The host cell can express the polypeptide.

In another aspect, this document features a host cell comprising an AAV vector comprising an AAV capsid polypeptide, wherein the capsid polypeptide comprises the amino acid sequence of any one of SEQ ID NOs:H2-H258. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:H2-H258 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:H2-H243 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:H2-H258. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:H244-H258. The vector can be an AAV2 vector. The vector can infect greater than 2 percent of retinal ganglion cells of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal ganglion cells than the level of expression from a comparable AAV vector comprising a capsid polypeptide consisting of the amino acid sequence set forth in SEQ ID NO:1. The host cell can be a retinal cell.

In another aspect, this document features a host cell comprising an AAV capsid polypeptide comprising the amino acid sequence of any one of SEQ ID NOs:H2-H258. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:H2-H258 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:H2-H243 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:H2-H258. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:H244-H258. An AAV vector comprising the polypeptide can infect greater than 2 percent of retinal ganglion cells of an eye when a titer of at least 1×10⁷ of the vector is administered intravitreally to the eye. An AAV vector comprising the polypeptide can express more nucleic acid in retinal ganglion cells than the level of expression from a comparable AAV vector comprising a capsid polypeptide consisting of the amino acid sequence set forth in SEQ ID NO:1. The host cell can be a retinal cell.

In another aspect, this document features a composition comprising an AAV vector comprising an AAV capsid polypeptide, wherein the capsid polypeptide comprises the amino acid sequence of any one of SEQ ID NOs:H2-H258. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:H2-H258 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acid sequence of any one of SEQ ID NOs:H2-H243 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:H2-H258. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:H244-H258. The vector can be an AAV2 vector. The vector can infect greater than 2 percent of retinal ganglion cells of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal ganglion cells than the level of expression from a comparable AAV vector comprising a capsid polypeptide consisting of the amino acid sequence set forth in SEQ ID NO:1. The composition can comprise from about 1×10⁷ to about 1×10¹⁴ of the vector. The composition can comprise phosphate buffered saline, Hank's Balanced Salt Solution, or Pluronic F68.

In another aspect, this document features a method for delivering an exogenous nucleic acid sequence to a retinal ganglion cell within a mammal. The method comprises (or consists essentially of, or consists of) contacting the retinal ganglion cell with an AAV vector comprising an AAV capsid polypeptide and the exogenous nucleic acid sequence, wherein the capsid polypeptide comprises the amino acid sequence of any one of SEQ ID NOs:H2-H258, wherein the AAV vector infects the photoreceptor cell, thereby delivering the exogenous nucleic acid sequence to the photoreceptor cell. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:H2-H258 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:H2-H243 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:H2-H258. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:H244-H258. The mammal can be a human (or a non-human primate). The vector can be an AAV2 vector. The vector can infect greater than 2 percent of retinal ganglion cells of an eye when a titer of at least 1×10⁷ of the vector is administered intravitreally to the eye. The exogenous nucleic acid sequence can encode an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more of the exogenous nucleic acid sequence in the retinal ganglion cell than the level of expression in a retinal ganglion cell from a comparable AAV vector comprising a capsid polypeptide consisting of the amino acid sequence set forth in SEQ ID NO:1. The method can comprise intravitreally administering a composition comprising the vector to the mammal, thereby contacting the retinal ganglion cell with the vector. The composition can comprise from about 1×10⁷ to about 1×10¹⁴ of the vector.

In another aspect, this document features a method for treating a retinal condition. The method comprises (or consists essentially of, or consists of) contacting retinal ganglion cells of a mammal having the retinal condition with AAV vectors comprising an AAV capsid polypeptide and an exogenous nucleic acid sequence, wherein the capsid polypeptide comprises the amino acid sequence of any one of SEQ ID NOs:H2-H258, wherein the AAV vectors infect the retinal ganglion cells and drive expression of the exogenous nucleic acid sequence within the retinal ganglion cells, thereby treating the retinal condition. The mammal can be a human (or a non-human primate). The retinal condition can be selected from the group consisting of cone dystrophy, cone/rod dystrophy, retinitis pigmentosa, macular degeneration, achromatopsia, blue cone monochromcy, and color blindness. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:H2-H258 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:H2-H243 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:H2-H258. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:H244-H258. The vectors can be AAV2 vectors. The vectors can infect greater than 2 percent of retinal ganglion cells when a titer of at least 1×10¹⁴ of the vectors is administered intravitreally to an eye of the mammal. The exogenous nucleic acid sequence can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vectors can express more of the exogenous nucleic acid sequence in the retinal ganglion cells than the level of expression in retinal ganglion cells from a comparable AAV vector comprising a capsid polypeptide consisting of the amino acid sequence set forth in SEQ ID NO:1. The method can comprise intravitreally administering a composition comprising the vectors to the mammal, thereby contacting the retinal ganglion cells with the vectors. The composition can comprise from about 1×10⁷ to about 1×10¹⁴ of the vector.

In another aspect, this document features an adeno-associated virus (AAV) vector comprising an AAV capsid polypeptide, wherein the capsid polypeptide comprises the amino acid sequence of any one of SEQ ID NOs:I2-I74. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:I2-I74 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:I2-I67 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:I2-I74. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:I68-I74. The vector can be an AAV2 vector. The vector can infect greater than 2 percent of bipolar cells of the retina of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in bipolar cells of the retina than the level of expression from a comparable AAV vector comprising a capsid polypeptide consisting of the amino acid sequence set forth in SEQ ID NO:1.

In another aspect, this document features an AAV capsid polypeptide comprising the amino acid sequence of any one of SEQ ID NOs:I2-I74. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:I2-174 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:I2-167 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:I2-I74. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:I68-I74. An AAV vector comprising the polypeptide can infect greater than 2 percent of bipolar cells of the retina of an eye when a titer of at least 1×10⁷ of the vector is administered intravitreally to the eye. An AAV vector comprising the polypeptide can express more nucleic acid in bipolar cells of the retina than the level of expression from a comparable AAV vector comprising a capsid polypeptide consisting of the amino acid sequence set forth in SEQ ID NO:1.

In another aspect, this document features a nucleic acid molecule encoding an AAV vector comprising an AAV capsid polypeptide, wherein the capsid polypeptide comprises the amino acid sequence of any one of SEQ ID NOs:I2-I74. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:I2-I74 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:I2-I67 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:I2-I74. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:I68-I74. The vector can be an AAV2 vector. The vector can infect greater than 2 percent of bipolar cells of the retina of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in bipolar cells of the retina than the level of expression from a comparable AAV vector comprising a capsid polypeptide consisting of the amino acid sequence set forth in SEQ ID NO:1. The nucleic acid molecule can be DNA.

In another aspect, this document features a nucleic acid molecule encoding an AAV capsid polypeptide comprising the amino acid sequence of any one of SEQ ID NOs:I2-I74. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:I2-I74 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:I2-167 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:I2-174. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:I68-I74. An AAV vector comprising the polypeptide can infect greater than 2 percent of bipolar cells of the retina of an eye when a titer of at least 1×10⁷ of the vector is administered intravitreally to the eye. An AAV vector comprising the polypeptide can express more nucleic acid in bipolar cells of the retina than the level of expression from a comparable AAV vector comprising a capsid polypeptide consisting of the amino acid sequence set forth in SEQ ID NO:1. The nucleic acid molecule can be DNA.

In another aspect, this document features a host cell comprising a nucleic acid molecule of either of the two preceding paragraphs. The host cell can express the vector. The host cell can express the polypeptide.

In another aspect, this document features a host cell comprising an AAV vector comprising an AAV capsid polypeptide, wherein the capsid polypeptide comprises the amino acid sequence of any one of SEQ ID NOs:I2-I74. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:I2-I74 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:I2-167 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:I2-I74. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:I68-I74. The vector can be an AAV2 vector. The vector can infect greater than 2 percent of bipolar cells of the retina of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in bipolar cells of the retina than the level of expression from a comparable AAV vector comprising a capsid polypeptide consisting of the amino acid sequence set forth in SEQ ID NO:1. The host cell can be a retinal cell.

In another aspect, this document features a host cell comprising an AAV capsid polypeptide comprising the amino acid sequence of any one of SEQ ID NOs:I2-I74. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:I2-174 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:I2-167 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:I2-174. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:I68-I74. An AAV vector comprising the polypeptide can infect greater than 2 percent of bipolar cells of the retina of an eye when a titer of at least 1×10⁷ of the vector is administered intravitreally to the eye. An AAV vector comprising the polypeptide can express more nucleic acid in bipolar cells of the retina than the level of expression from a comparable AAV vector comprising a capsid polypeptide consisting of the amino acid sequence set forth in SEQ ID NO:1. The host cell can be a retinal cell.

In another aspect, this document features a composition comprising an AAV vector comprising an AAV capsid polypeptide, wherein the capsid polypeptide comprises the amino acid sequence of any one of SEQ ID NOs:I2-174. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in 10.5 SEQ ID NO:1) except that the amino acid sequence of any one of SEQ ID NOs:I2-I74 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acid sequence of any one of SEQ ID NOs:I2-167 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:I2-I74. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:I68-I74. The vector can be an AAV2 vector. The vector can infect greater than 2 percent of bipolar cells of the retina of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in bipolar cells of the retina than the level of expression from a comparable AAV vector comprising a capsid polypeptide consisting of the amino acid sequence set forth in SEQ ID NO:1. The composition can comprise from about 1×10⁷ to about 1×10¹⁴ of the vector. The composition can comprise phosphate buffered saline, Hank's Balanced Salt Solution, or Pluronic F68.

In another aspect, this document features a method for delivering an exogenous nucleic acid sequence to a bipolar cell of the retina within a mammal. The method comprises (or consists essentially of, or consists of) contacting the bipolar cell with an AAV vector comprising an AAV capsid polypeptide and the exogenous nucleic acid sequence, wherein the capsid polypeptide comprises the amino acid sequence of any one of SEQ ID NOs:I2-I74, wherein the AAV vector infects the bipolar cell, thereby delivering the exogenous nucleic acid sequence to the bipolar cell. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:I2-I74 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:I2-I67 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO: 1 are replaced with the amino acid sequence of any one of SEQ ID NOs:I2-I74. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:I68-I74. The mammal can be a human (or a non-human primate). The vector can be an AAV2 vector. The vector can infect greater than 2 percent of bipolar cells of the retina of an eye when a titer of at least 1×10⁷ of the vector is administered intravitreally to the eye. The exogenous nucleic acid sequence can encode an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more of the exogenous nucleic acid sequence in the bipolar cell than the level of expression in a bipolar cell of the retina from a comparable AAV vector comprising a capsid polypeptide consisting of the amino acid sequence set forth in SEQ ID NO:1. The method can comprise intravitreally administering a composition comprising the vector to the mammal, thereby contacting the bipolar cell with the vector. The composition can comprise from about 1×10⁷ to about 1×10¹⁴ of the vector.

In another aspect, this document features a method for treating a retinal condition. The method comprises (or consists essentially of, or consists of) contacting bipolar cells of the retina of a mammal having the retinal condition with AAV vectors comprising an AAV capsid polypeptide and an exogenous nucleic acid sequence, wherein the capsid polypeptide comprises the amino acid sequence of any one of SEQ ID NOs:I2-174, wherein the AAV vectors infect the bipolar cells and drive expression of the exogenous nucleic acid sequence within the bipolar cells, thereby treating the retinal condition. The mammal can be a human (or a non-human primate). The retinal condition can be selected from the group consisting of cone dystrophy, cone/rod dystrophy, retinitis pigmentosa, macular degeneration, achromatopsia, blue cone monochromcy, and color blindness. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:I2-174 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:I2-I67 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:I2-174. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:I68-174. The vectors can be AAV2 vectors. The vectors can infect greater than 2 percent of bipolar cells of the retina when a titer of at least 1×10⁷ of the vectors is administered intravitreally to an eye of the mammal. The exogenous nucleic acid sequence can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vectors can express more of the exogenous nucleic acid sequence in the bipolar cells than the level of expression in bipolar cells of the retina from a comparable AAV vector comprising a capsid polypeptide consisting of the amino acid sequence set forth in SEQ ID NO:1. The method can comprise intravitreally administering a composition comprising the vectors to the mammal, thereby contacting the bipolar cells with the vectors. The composition can comprise from about 1×10⁷ to about 1×10¹⁴ of the vector.

In another aspect, this document features an adeno-associated virus (AAV) vector comprising an AAV capsid polypeptide, wherein the capsid polypeptide comprises the amino acid sequence of any one of SEQ ID NOs:J2-J64. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J64 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J61 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:J2-J64. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:J62-J64. The vector can be an AAV2 vector. The vector can infect greater than 2 percent of ON-retinal ganglion cells of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in ON-retinal ganglion cells than the level of expression from a comparable AAV vector comprising a capsid polypeptide consisting of the amino acid sequence set forth in SEQ ID NO:1.

In another aspect, this document features an AAV capsid polypeptide comprising the amino acid sequence of any one of SEQ ID NOs:J2-J64. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J64 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J61 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:J2-J64. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:J62-J64. An AAV vector comprising the polypeptide can infect greater than 2 percent of ON-retinal ganglion cells of an eye when a titer of at least 1×10⁷ of the vector is administered intravitreally to the eye. An AAV vector comprising the polypeptide can express more nucleic acid in ON-retinal ganglion cells than the level of expression from a comparable AAV vector comprising a capsid polypeptide consisting of the amino acid sequence set forth in SEQ ID NO:1.

In another aspect, this document features a nucleic acid molecule encoding an AAV vector comprising an AAV capsid polypeptide, wherein the capsid polypeptide comprises the amino acid sequence of any one of SEQ ID NOs:J2-J64. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J64 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J61 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:J2-J64. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:J62-J64. The vector can be an AAV2 vector. The vector can infect greater than 2 percent of ON-retinal ganglion cells of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in ON-retinal ganglion cells than the level of expression from a comparable AAV vector comprising a capsid polypeptide consisting of the amino acid sequence set forth in SEQ ID NO:1. The nucleic acid molecule can be DNA.

In another aspect, this document features a nucleic acid molecule encoding an AAV capsid polypeptide comprising the amino acid sequence of any one of SEQ ID NOs:J2-J64. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J64 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J61 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:J2-J64. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:J62-J64. An AAV vector comprising the polypeptide can infect greater than 2 percent of ON-retinal ganglion cells of an eye when a titer of at least 1×10⁷ of the vector is administered intravitreally to the eye. An AAV vector comprising the polypeptide can express more nucleic acid in ON-retinal ganglion cells than the level of expression from a comparable AAV vector comprising a capsid polypeptide consisting of the amino acid sequence set forth in SEQ ID NO:1. The nucleic acid molecule can be DNA.

In another aspect, this document features a host cell comprising a nucleic acid molecule of either of the two preceding paragraphs. The host cell can express the vector. The host cell can express the polypeptide.

In another aspect, this document features a host cell comprising an AAV vector comprising an AAV capsid polypeptide, wherein the capsid polypeptide comprises the amino acid sequence of any one of SEQ ID NOs:J2-J64. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J64 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J61 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:J2-J64. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:J62-J64. The vector can be an AAV2 vector. The vector can infect greater than 2 percent of ON-retinal ganglion cells of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in ON-retinal ganglion cells than the level of expression from a comparable AAV vector comprising a capsid polypeptide consisting of the amino acid sequence set forth in SEQ ID NO:1. The host cell can be a retinal cell.

In another aspect, this document features a host cell comprising an AAV capsid polypeptide comprising the amino acid sequence of any one of SEQ ID NOs:J2-J64. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J64 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J61 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:J2-J64. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:J62-J64. An AAV vector comprising the polypeptide can infect greater than 2 percent of ON-retinal ganglion cells of an eye when a titer of at least 1×10⁷ of the vector is administered intravitreally to the eye. An AAV vector comprising the polypeptide can express more nucleic acid in ON-retinal ganglion cells than the level of expression from a comparable AAV vector comprising a capsid polypeptide consisting of the amino acid sequence set forth in SEQ ID NO:1. The host cell can be a retinal cell.

In another aspect, this document features a composition comprising an AAV vector comprising an AAV capsid polypeptide, wherein the capsid polypeptide comprises the amino acid sequence of any one of SEQ ID NOs:J2-J64. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J64 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J61 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:J2-J64. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:J62-J64. The vector can be an AAV2 vector. The vector can infect greater than 2 percent of ON-retinal ganglion cells of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in ON-retinal ganglion cells than the level of expression from a comparable AAV vector comprising a capsid polypeptide consisting of the amino acid sequence set forth in SEQ ID NO:1. The composition can comprise from about 1×10⁷ to about 1×10¹⁴ of the vector. The composition can comprise phosphate buffered saline, Hank's Balanced Salt Solution, or Pluronic F68.

In another aspect, this document features a method for delivering an exogenous nucleic acid sequence to an ON-retinal ganglion cell within a mammal. The method comprises (or consists essentially of, or consists of) contacting the ON-retinal ganglion cell with an AAV vector comprising an AAV capsid polypeptide and the exogenous nucleic acid sequence, wherein the capsid polypeptide comprises the amino acid sequence of any one of SEQ ID NOs:J2-J64, wherein the AAV vector infects the ON-retinal ganglion cell, thereby delivering the exogenous nucleic acid sequence to the ON-retinal ganglion cell. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J64 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J61 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:J2-J64. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:J62-J64. The mammal can be a human (or a non-human primate). The vector can be an AAV2 vector. The vector can infect greater than 2 percent of ON-retinal ganglion cells of an eye when a titer of at least 1×10⁷ of the vector is administered intravitreally to the eye. The exogenous nucleic acid sequence can encode an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more of the exogenous nucleic acid sequence in the ON-retinal ganglion cell than the level of expression in an ON-retinal ganglion cell from a comparable AAV vector comprising a capsid polypeptide consisting of the amino acid sequence set forth in SEQ ID NO:1. The method can comprise intravitreally administering a composition comprising the vector to the mammal, thereby contacting the ON-retinal ganglion cell with the vector. The composition can comprise from about 1×10⁷ to about 1×10¹⁴ of the vector.

In another aspect, this document features a method for treating a retinal condition. The method comprises (or consists essentially of, or consists of) contacting ON-retinal ganglion cells of a mammal having the retinal condition with AAV vectors comprising an AAV capsid polypeptide and an exogenous nucleic acid sequence, wherein the capsid polypeptide comprises the amino acid sequence of any one of SEQ ID NOs:J2-J64, wherein the AAV vectors infect the ON-retinal ganglion cells and drive expression of the exogenous nucleic acid sequence within the ON-retinal ganglion cells, thereby treating the retinal condition. The mammal can be a human (or a non-human primate). The retinal condition can be selected from the group consisting of cone dystrophy, cone/rod dystrophy, retinitis pigmentosa, macular degeneration, achromatopsia, blue cone monochromcy, and color blindness. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J64 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:J2-J61 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:J2-J64. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:J62-J64. The vectors can be AAV2 vectors. The vectors can infect greater than 2 percent of ON-retinal ganglion cells when a titer of at least 1×10⁷ of the vectors is administered intravitreally to an eye of the mammal. The exogenous nucleic acid sequence can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vectors can express more of the exogenous nucleic acid sequence in the ON-retinal ganglion cells than the level of expression in ON-retinal ganglion cells from a comparable AAV vector comprising a capsid polypeptide consisting of the amino acid sequence set forth in SEQ ID NO:1. The method can comprise intravitreally administering a composition comprising the vectors to the mammal, thereby contacting the ON-retinal ganglion cells with the vectors. The composition can comprise from about 1×10⁷ to about 1×10¹⁴ of the vector.

In another aspect, this document features an adeno-associated virus (AAV) vector comprising an AAV capsid polypeptide, wherein the capsid polypeptide comprises the amino acid sequence of any one of SEQ ID NOs:K2-K63. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K63 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K60 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:K2-K63. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:K61-K63. The vector can be an AAV2 vector. The vector can infect greater than 2 percent of OFF-retinal ganglion cells of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in OFF-retinal ganglion cells than the level of expression from a comparable AAV vector comprising a capsid polypeptide consisting of the amino acid sequence set forth in SEQ ID NO:1.

In another aspect, this document features an AAV capsid polypeptide comprising the amino acid sequence of any one of SEQ ID NOs:K2-K63. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K63 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K60 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:K2-K63. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:K61-K63. An AAV vector comprising the polypeptide can infect greater than 2 percent of OFF-retinal ganglion cells of an eye when a titer of at least 1×10⁷ of the vector is administered intravitreally to the eye. An AAV vector comprising the polypeptide can express more nucleic acid in OFF-retinal ganglion cells than the level of expression from a comparable AAV vector comprising a capsid polypeptide consisting of the amino acid sequence set forth in SEQ ID NO:1.

In another aspect, this document features a nucleic acid molecule encoding an AAV vector comprising an AAV capsid polypeptide, wherein the capsid polypeptide comprises the amino acid sequence of any one of SEQ ID NOs:K2-K63. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K63 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K60 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:K2-K63. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO: 1 are replaced with the amino acid sequence of any one of SEQ ID NOs:K61-K63. The vector can be an AAV2 vector. The vector can infect greater than 2 percent of OFF-retinal ganglion cells of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in OFF-retinal ganglion cells than the level of expression from a comparable AAV vector comprising a capsid polypeptide consisting of the amino acid sequence set forth in SEQ ID NO:1. The nucleic acid molecule can be DNA.

In another aspect, this document features a nucleic acid molecule encoding an AAV capsid polypeptide comprising the amino acid sequence of any one of SEQ ID NOs:K2-K63.

The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K63 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K60 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:K2-K63. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:K61-K63. An AAV vector comprising the polypeptide can infect greater than 2 percent of OFF-retinal ganglion cells of an eye when a titer of at least 1×10⁷ of the vector is administered intravitreally to the eye. An AAV vector comprising the polypeptide can express more nucleic acid in OFF-retinal ganglion cells than the level of expression from a comparable AAV vector comprising a capsid polypeptide consisting of the amino acid sequence set forth in SEQ ID NO:1. The nucleic acid molecule can be DNA.

In another aspect, this document features a host cell comprising a nucleic acid molecule of either of the two preceding paragraphs. The host cell can express the vector. The host cell can express the polypeptide.

In another aspect, this document features a host cell comprising an AAV vector comprising an AAV capsid polypeptide, wherein the capsid polypeptide comprises the amino acid sequence of any one of SEQ ID NOs:K2-K63. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K63 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K60 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:K2-K63. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:K61-K63. The vector can be an AAV2 vector. The vector can infect greater than 2 percent of OFF-retinal ganglion cells of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in OFF-retinal ganglion cells than the level of expression from a comparable AAV vector comprising a capsid polypeptide consisting of the amino acid sequence set forth in SEQ ID NO:1. The host cell can be a retinal cell.

In another aspect, this document features a host cell comprising an AAV capsid polypeptide comprising the amino acid sequence of any one of SEQ ID NOs:K2-K63. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K63 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K60 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:K2-K63. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:K61-K63. An AAV vector comprising the polypeptide can infect greater than 2 percent of OFF-retinal ganglion cells of an eye when a titer of at least 1×10⁷ of the vector is administered intravitreally to the eye. An AAV vector comprising the polypeptide can express more nucleic acid in OFF-retinal ganglion cells than the level of expression from a comparable AAV vector comprising a capsid polypeptide consisting of the amino acid sequence set forth in SEQ ID NO:1. The host cell can be a retinal cell.

In another aspect, this document features a composition comprising an AAV vector comprising an AAV capsid polypeptide, wherein the capsid polypeptide comprises the amino acid sequence of any one of SEQ ID NOs:K2-K63. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K63 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K60 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:K2-K63. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:K61-K63. The vector can be an AAV2 vector. The vector can infect greater than 2 percent of OFF-retinal ganglion cells of an eye when a titer of at least 1×10⁷ of the vector is administered intravitreally to the eye. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in OFF-retinal ganglion cells than the level of expression from a comparable AAV vector comprising a capsid polypeptide consisting of the amino acid sequence set forth in SEQ ID NO:1. The composition can comprise from about 1×10⁷ to about 1×10¹⁴ of the vector. The composition can comprise phosphate buffered saline, Hank's Balanced Salt Solution, or Pluronic F68.

In another aspect, this document features a method for delivering an exogenous nucleic acid sequence to an OFF-retinal ganglion cell within a mammal. The method comprises (or consists essentially of, or consists of) contacting the OFF-retinal ganglion cell with an AAV vector comprising an AAV capsid polypeptide and the exogenous nucleic acid sequence, wherein the capsid polypeptide comprises the amino acid sequence of any one of SEQ ID NOs:K2-K63, wherein the AAV vector infects the OFF-retinal ganglion cell, thereby delivering the exogenous nucleic acid sequence to the OFF-retinal ganglion cell. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K63 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K60 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:K2-K63. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:K61-K63. The mammal can be a human (or a non-human primate). The vector can be an AAV2 vector. The vector can infect greater than 2 percent of OFF-retinal ganglion cells of an eye when a titer of at least 1×10⁷ of the vector is administered intravitreally to the eye. The exogenous nucleic acid sequence can encode an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more of the exogenous nucleic acid sequence in the OFF-retinal ganglion cell than the level of expression in an OFF-retinal ganglion cell from a comparable AAV vector comprising a capsid polypeptide consisting of the amino acid sequence set forth in SEQ ID NO:1. The method can comprise intravitreally administering a composition comprising the vector to the mammal, thereby contacting the OFF-retinal ganglion cell with the vector. The composition can comprise from about 1×10⁷ to about 1×10¹⁴ of the vector.

In another aspect, this document features a method for treating a retinal condition. The method comprises (or consists essentially of, or consists of) contacting OFF-retinal ganglion cells of a mammal having the retinal condition with AAV vectors comprising an AAV capsid polypeptide and an exogenous nucleic acid sequence, wherein the capsid polypeptide comprises the amino acid sequence of any one of SEQ ID NOs:K2-K63, wherein the AAV vectors infect the OFF-retinal ganglion cells and drive expression of the exogenous nucleic acid sequence within the OFF-retinal ganglion cells, thereby treating the retinal condition. The mammal can be a human (or a non-human primate). The retinal condition can be selected from the group consisting of cone dystrophy, cone/rod dystrophy, retinitis pigmentosa, macular degeneration, achromatopsia, blue cone monochromcy, and color blindness. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K63 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:K2-K60 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:K2-K63. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:K61-K63. The vectors can be AAV2 vectors. The vectors can infect greater than 2 percent of OFF-retinal ganglion cells when a titer of at least 1×10⁷ of the vectors is administered intravitreally to an eye of the mammal. The exogenous nucleic acid sequence can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vectors can express more of the exogenous nucleic acid sequence in the OFF-retinal ganglion cells than the level of expression in OFF-retinal ganglion cells from a comparable AAV vector comprising a capsid polypeptide consisting of the amino acid sequence set forth in SEQ ID NO:1. The method can comprise intravitreally administering a composition comprising the vectors to the mammal, thereby contacting the OFF-retinal ganglion cells with the vectors. The composition can comprise from about 1×10⁷ to about 1×10¹⁴ of the vector.

In another aspect, this document features an adeno-associated virus (AAV) vector comprising an AAV capsid polypeptide, wherein the capsid polypeptide comprises the amino acid sequence of any one of SEQ ID NOs:L2-L27. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:L2-L27 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:L2-L19 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:L2-L27. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:L20-L27. The vector can be an AAV2 vector. The vector can have a packaging efficiency greater than that of a comparable AAV vector comprising a capsid polypeptide consisting of the amino acid sequence set forth in SEQ ID NO:1. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can have a packaging efficiency at least 10 percent greater than that of a comparable AAV vector comprising a capsid polypeptide consisting of the amino acid sequence set forth in SEQ ID NO:1.

In another aspect, this document features an AAV capsid polypeptide comprising the amino acid sequence of any one of SEQ ID NOs:L2-L27. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:L2-L27 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:L2-L19 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:L2-L27. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 are replaced with the amino acid sequence of any one of SEQ ID NOs:L20-L27. The vector can have a packaging efficiency greater than that of a comparable AAV vector comprising a capsid polypeptide consisting of the amino acid sequence set forth in SEQ ID NO:1. The vector can have a packaging efficiency at least 10 percent greater than that of a comparable AAV vector comprising a capsid polypeptide consisting of the amino acid sequence set forth in SEQ ID NO:1.

In another aspect, this document features a nucleic acid molecule encoding an AAV vector comprising an AAV capsid polypeptide, wherein the capsid polypeptide comprises the amino acid sequence of any one of SEQ ID NOs:L2-L27. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:L2-L27 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:L2-L19 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:L2-L27. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:L20-L27. The vector can be an AAV2 vector. The vector can have a packaging efficiency greater than that of a comparable AAV vector comprising a capsid polypeptide consisting of the amino acid sequence set forth in SEQ ID NO:1. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can have a packaging efficiency at least 10 percent greater than that of a comparable AAV vector comprising a capsid polypeptide consisting of the amino acid sequence set forth in SEQ ID NO:1. The nucleic acid molecule can be DNA.

In another aspect, this document features a nucleic acid molecule encoding an AAV capsid polypeptide comprising the amino acid sequence of any one of SEQ ID NOs:L2-L27. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:L2-L27 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:L2-L19 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:L2-L27. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:L20-L27. The vector can have a packaging efficiency greater than that of a comparable AAV vector comprising a capsid polypeptide consisting of the amino acid sequence set forth in SEQ ID NO:1. The vector can have a packaging efficiency at least 10 percent greater than that of a comparable AAV vector comprising a capsid polypeptide consisting of the amino acid sequence set forth in SEQ ID NO:1. The nucleic acid molecule can be DNA.

In another aspect, this document features a host cell comprising a nucleic acid molecule of either of the two preceding paragraphs. The host cell can express the vector. The host cell can express the polypeptide.

In another aspect, this document features a host cell comprising an AAV vector comprising an AAV capsid polypeptide, wherein the capsid polypeptide comprises the amino acid sequence of any one of SEQ ID NOs:L2-L27. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:L2-L27 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:L2-L19 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:L2-L27. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:L20-L27. The vector can be an AAV2 vector. The vector can have a packaging efficiency greater than that of a comparable AAV vector comprising a capsid polypeptide consisting of the amino acid sequence set forth in SEQ ID NO:1. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can have a packaging efficiency at least 10 percent greater than that of a comparable AAV vector comprising a capsid polypeptide consisting of the amino acid sequence set forth in SEQ ID NO:1. The host cell can be a retinal cell.

In another aspect, this document features a host cell comprising an AAV capsid polypeptide comprising the amino acid sequence of any one of SEQ ID NOs:L2-L27. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:L2-L27 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:L2-L19 is located between amino acid positions 587 and 588 of SEQ ID NO:1 (or the appropriate amino acid positions of the alternative sequence). The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:L2-L27. The polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:L20-L27. The vector can have a packaging efficiency greater than that of a comparable AAV vector comprising a capsid polypeptide consisting of the amino acid sequence set forth in SEQ ID NO:1. The vector can have a packaging efficiency at least 10 percent greater than that of a comparable AAV vector comprising a capsid polypeptide consisting of the amino acid sequence set forth in SEQ ID NO: 1. The host cell can be a retinal cell.

In another aspect, this document features a composition comprising an AAV vector comprising an AAV capsid polypeptide, wherein the capsid polypeptide comprises the amino acid sequence of any one of SEQ ID NOs:L2-L27. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:L2-L27 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) except that the amino acid sequence of any one of SEQ ID NOs:L2-L19 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:L2-L27. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:L20-L27. The vector can be an AAV2 vector. The vector can have a packaging efficiency greater than that of a comparable AAV vector comprising a capsid polypeptide consisting of the amino acid sequence set forth in SEQ ID NO:1. The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can have a packaging efficiency at least 10 percent greater than that of a comparable AAV vector comprising a capsid polypeptide consisting of the amino acid sequence set forth in SEQ ID NO:1. The composition can comprise from about 1×10¹⁴ to about 1×10¹⁴ of the vector. The composition can comprise phosphate buffered saline, Hank's Balanced Salt Solution, or Pluronic F68.

In another aspect, this document features a method for delivering an exogenous nucleic acid sequence to a cell within a mammal. The method comprises (or consists essentially of, or consists of) contacting the cell with an AAV vector comprising an AAV capsid polypeptide and the exogenous nucleic acid sequence, wherein the capsid polypeptide comprises the amino acid sequence of any one of SEQ ID NOs:L2-L27, wherein the AAV vector infects the cell, thereby delivering the exogenous nucleic acid sequence to the cell. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:L2-L27 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:L2-L19 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:L2-L27. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:L20-L27. The mammal can be a human (or a non-human primate). The vector can be an AAV2 vector. The vector can have a packaging efficiency greater than that of a comparable AAV vector comprising a capsid polypeptide consisting of the amino acid sequence set forth in SEQ ID NO:1. The exogenous nucleic acid sequence can encode an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can have a packaging efficiency at least 10 percent greater than that of a comparable AAV vector comprising a capsid polypeptide consisting of the amino acid sequence set forth in SEQ ID NO: 1. The method can comprise intravitreally administering a composition comprising the vector to the mammal, thereby contacting the cell with the vector. The composition can comprise from about 1×10⁷ to about 1×10¹⁴ of the vector.

In another aspect, this document features a method for treating a retinal condition. The method comprises (or consists essentially of, or consists of) contacting retinal cells of a mammal having the retinal condition with AAV vectors comprising an AAV capsid polypeptide and an exogenous nucleic acid sequence, wherein the capsid polypeptide comprises the amino acid sequence of any one of SEQ ID NOs:L2-L27, wherein the AAV vectors infect the retinal cells and drive expression of the exogenous nucleic acid sequence within the retinal cells, thereby treating the retinal condition. The mammal can be a human (or a non-human primate). The retinal condition can be selected from the group consisting of cone dystrophy, cone/rod dystrophy, retinitis pigmentosa, macular degeneration, achromatopsia, blue cone monochromcy, and color blindness. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:L2-L27 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs:L2-L19 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO: 1 are replaced with the amino acid sequence of any one of SEQ ID NOs:L2-L27. The capsid polypeptide can comprise the amino acid sequence of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO:1 are replaced with the amino acid sequence of any one of SEQ ID NOs:L20-L27. The vectors can be AAV2 vectors. The vector can have a packaging efficiency greater than that of a comparable AAV vector comprising a capsid polypeptide consisting of the amino acid sequence set forth in SEQ ID NO:1. The exogenous nucleic acid sequence can encode an RNA.

The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can have a packaging efficiency at least 10 percent greater than that of a comparable AAV vector comprising a capsid polypeptide consisting of the amino acid sequence set forth in SEQ ID NO:1. The method can comprise intravitreally administering a composition comprising the vectors to the mammal, thereby contacting the retinal cells with the vectors. The composition can comprise from about 1×10⁷ to about 1×10¹⁴ of the vector.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and methods are described below. All publications, patent applications, patents, and other references mentioned herein are incorporated by reference in their entirety. In case of conflict, the present specification, including definitions will control. In addition, the materials, methods, and examples are illustrative only and not intended to be limiting.

The details of one or more embodiments of the invention are set forth in the accompanying description below. Other features, objects, and advantages of the invention will be apparent from the description and from the claims.

### DESCRIPTION OF DRAWINGS

FIG. 1 is a listing of SEQ ID NOs:2207-2208.

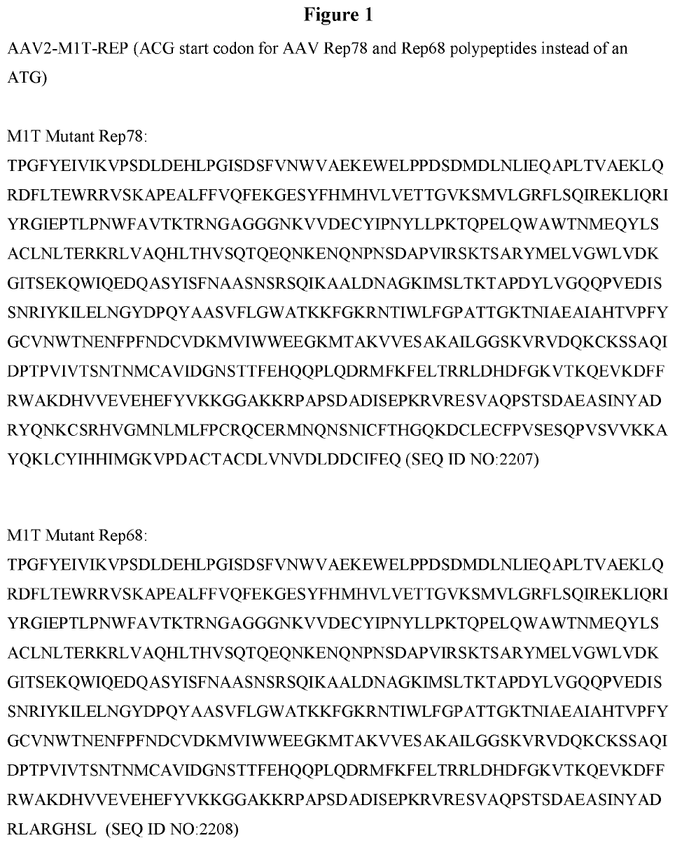

FIG. 2 a diagram of AAV vectors that include a wild type AAV2 Rep polypeptide and an indicated AAV2 capsid polypeptide engineered to include an insert sequence (e.g., any one of SEQ ID NOs:2-1103 or a sequence of Formula A) located between positions 587 and 588 (using SEQ ID NO:1 numbering), according to some embodiments.

FIG. 3 is a diagram of AAV vectors that include a mutant AAV2 Rep polypeptide (AAV2-M1T-REP) and an indicated AAV2 capsid polypeptide engineered to include an insert sequence (e.g., any one of SEQ ID NOs:2-1103 or a sequence of Formula A) located between positions 587 and 588 (using SEQ ID NO:1 numbering), according to some embodiments.

FIG. 4 is a diagram of AAV vectors that include a wild type AAV2 Rep polypeptide and an indicated AAV2 capsid polypeptide engineered to include an insert sequence (e.g., any one of SEQ ID NOs:2-1103 or a sequence of Formula A) as a replacement of amino acid residues at positions 585 to 590 (using SEQ ID NO:1 numbering), according to some embodiments.

FIG. 5 is a diagram of AAV vectors that include a mutant AAV2 Rep polypeptide (AAV2-M1T-REP) and an indicated AAV2 capsid polypeptide engineered to include an insert sequence (e.g., any one of SEQ ID NOs:2-1103 or a sequence of Formula A) as a replacement of amino acid residues at positions 585 to 590 (using SEQ ID NO:1 numbering), according to some embodiments.

### DETAILED DESCRIPTION

This document provides AAV vectors (e.g., AAV2 vectors). For example, this document provides AAV vectors (e.g., AAV2 vectors) containing a capsid polypeptide that includes an amino acid sequence set forth in any one or more of Tables 1A-1L (or a variant thereof) or according to Formula A based on such a set forth sequence. Any appropriate AAV vector can be designed to include a capsid polypeptide described herein (e.g., a capsid polypeptide that includes an amino acid sequence set forth in any one or more of Tables 1A-1L (or a variant thereof) or according to Formula A based on such a set forth sequence). For example, AAV2, AAV8, and AAV9 can be designed to include a capsid polypeptide that includes an amino acid sequence set forth in any one or more of Tables 1A-1L (or a variant thereof) or according to Formula A based on such a set forth sequence. In some cases, an AAV2 having an ACG start codon for the AAV Rep polypeptides (e.g., AAV2 Rep78 and Rep68 polypeptides; see, e.g., SEQ ID NOs:2207-2208) instead of an ATG start codon (e.g., an AAV2-M1T-REP) can be designed to include a capsid polypeptide that includes an amino acid sequence set forth in Table 1 (or a variant thereof) or Formula A.

Any appropriate AAV capsid polypeptide can be designed to include an amino acid sequence set forth in any one or more of Tables 1A-1L (or a variant thereof) or according to Formula A based on such a set forth sequence. For example, AAV2, AAV6, AAV8, and AAV9 capsid polypeptides can be designed to include an amino acid sequence set forth in any one or more of Tables 1A-1L (or a variant thereof) or according to Formula A based on such a set forth sequence. In some cases, an AAV2 capsid polypeptide can be designed to include an amino acid sequence set forth in any one or more of Tables 1A-1L (or a variant thereof) or according to Formula A based on such a set forth sequence. In some cases, an AAV2 capsid polypeptide having the following amino acid sequence can be designed to include an amino acid sequence set forth in any one or more of Tables 1A-1L (or a variant thereof) or according to Formula A based on such a set forth sequence: MAADGYLPDWLEDTLSEGIRQWWKLKPG PPPPKPAERHKDDSRGLVLPGYKYLGPFNGLDKGEPVNEADAAALEHDKAYDRQLDSG DNPYLKYNHADAEFQERLKEDTSFGGNLGRAVFQAKKRVLEPLGLVEEPVKTAPGKKR PVEHSPVEPDSSSGTGKAGQQPARKRLNFGQTGDADSVPDPQPLGQPPAAPSGLGTNTM ATGSGAPMADNNEGADGVGNSSGNWHCDSTWMGDRVITTSTRTWALPTYNNHLYKQI SSQSGASNDNHYFGYSTPWGYFDFNRFHCHFSPRDWQRLINNNWGFRPKRLNFKLFNIQ VKEVTQNDGTTTIANNLTSTVQVFTDSEYQLPYVLGSAHQGCLPPFPADVFMVPQYGYL TLNNGSQAVGRSSFYCLEYFPSQMLRTGNNFTFSYTFEDVPFHSSYAHSQSLDRLMNPLI DQYLYYLSRTNTPSGT TTQSRLQFSQAGASDIRDQSRNWLPGPCYRQQRVSKTSADNNN SEYSWTGATKYHLNGRDSLVNPGPAMASHKDDEEKFFPQSGVLIFGKQGSEKTNVDIEK VMITDEEEIRTTNPVATEQYGSVSTNLQRGNRQAATADVNTQGVLPGMVWQDRDVYL QGPIWAKIPHTDGHFHPSPLMGGFGLKHPPPQILIKNTPVPANPSTTFSAAKFASFITQYST GQVSVEIEWELQKENSKRWNPEIQYTSNYNKSVNVDFTVDTNGVYSEPRPIGTRYLTRN L (SEQ ID NO:1). The two bold amino acid residues are at positions 587 and 588, and the underlined amino acids are at positions 585 to 590.

In some cases, an AAV capsid polypeptide (e.g., an AAV2 capsid polypeptide) having the following amino acid sequence can be designed to include an amino acid sequence set forth in Table 1 (or a variant thereof) or Formula A: MAADGYLPDWLEDTLSEGIRQWWKLKPG PPPPKPAERHKDDSRGLVLPGYKYLGPFNGLDKGEPVNX₁ADAAALEHDKAYDRQLDS GDNPYLKYNHADAEFQERLKEDTSFGGNLGRAVFQAKKRVLEPLGLVEEPVKTAPGKK RPVEHSPVEPDSSSGTGKAGQQPARKRLNFGQTGDADSVPDPQPLGQPPAAPSGLGTNT MATGSGAPMADNNEGADGVGNSSGNWHCDSTWMGDRVITTSTRTWALPTYNNHLYK QISSQ SGASNDNHYFGYSTPWGYFDFNRFHCHFSPRDWQRLINNNWGFRPKRLNFKLFN IQVKEVTQNDGTTTIANNLTSTVQVFTDSEYQLPYVLGSAHQGCLPPFPADVFMVPQYG YLTLNNGSQAVGRSSFYCLEYFPSQMLRTGNNFTFSYTFEDVPFHSSYAHSQSLDRLMN PLIDQYLYYLSRTNTPSGTTTQSRLQFSQAGASDIRDQSRNWLPGPCYRQQRVSKTSAD NNNSEYSWTGATKYHLNGRDSLVNPGP AMASHKDDEEKFFPQSGVLIFGKQGSEKTNV DIEKVMITDEEEIRTTNPVATEQYGSVSTNLQRGNRQAATADVNTQGVLPGMVWQDRD VYLQGPIWAKIPHTDGHFHPSPLMGGFGLKHPPPQILIKNTPVPANPST TFSAAKFASFIT QYSTGQVSVEIEWELQKENSKRWNPEIQYTSNYNKSX₂NVDFTVDTNGVYSEPRPIGTR YLTRNL, wherein X₁ is E or A, and wherein X₂ is V or I (SEQ ID NO:2206).

In some cases, an AAV capsid polypeptide (e.g., an AAV2 capsid polypeptide) that is at least 95%, at least 96%, at least 97%, at least 98%, or at least 99% identical to the amino acid sequence set forth in SEQ ID NO:1 can be designed to include an amino acid sequence set forth in Table 1 (or a variant thereof) or Formula A.

In some cases, certain AAV2 sequences contemplated herein can include modifications or mutations of SEQ ID NO:1 such as a V708I substitution.

When designing an AAV capsid polypeptide (e.g., an AAV2 capsid polypeptide) to include an amino acid sequence set forth in any one or more of Tables 1A-1L (or a variant thereof) or according to Formula A based on such a set forth sequence, that included amino acid sequence can be located at any appropriate location along the AAV capsid polypeptide (e.g., the AAV2 capsid polypeptide). For example, an amino acid sequence set forth in any one or more of Tables 1A-1L (or a variant thereof) or according to Formula A based on such a set forth sequence such as any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-174, SEQ ID NOs:J2-J64, SEQ ID NOs:K2-K63, and SEQ ID NOs:L2-L27 can be located between the naturally-occurring amino acid residues at positions 587 and 588 of an AAV capsid polypeptide (e.g., an AAV2 capsid polypeptide), can be located between the naturally-occurring amino acid residues at positions 452 and 453 of an AAV capsid polypeptide (e.g., an AAV2 capsid polypeptide), or can be located between the naturally-occurring amino acid residues at positions 453 and 454 of an AAV capsid polypeptide (e.g., an AAV2 capsid polypeptide).

TABLE 1A
Amino acid sequences that can be inserted
into an AAV capsid polypeptide.

| | | | Nucleic Acid | | | |
|---|---|---|---|---|---|---|
| | | | Sequence en- | | | |
| Amino | SEQ | SEQ | coding the | SEQ | SEQ | |
| Acid | ID | ID | Amino Acid | ID | ID | Activity |
| Sequence | NO: | NO:** | Sequence | NO: | NO:** | Level* |
| TGYSSTNG | A2 | 2 | ACCGGCTACAGCAG | A20 | 1104 | +++ |
| | | | CACCAACGGC | | | |
| LADLSDHHA | A3 | 3 | CTAGCAGACCTGAG | A21 | 1105 | +++ |
| | | | CGACCACCACGCT | | | |
| SGTEATRG | A4 | 4 | AGCGGCACCGAGGC | A22 | 1106 | +++ |
| | | | CACCCGGGGC | | | |
| TGQDKPTG | A5 | 5 | ACCGGCCAGGACAA | A23 | 1107 | +++ |
| | | | GCCCACCGGC | | | |
| SSMDPRHG | A6 | 6 | AGCAGCATGGACCC | A24 | 1108 | +++ |
| | | | CCGGCACGGC | | | |
| LANLKENEA | A7 | 7 | CTAGCAAACCTGAA | A25 | 1109 | +++ |
| | | | GGAGAACGAGGCT | | | |
| LAIKMIAYA | A8 | 8 | CTAGCAATCAAGAT | A26 | 1110 | ++ |
| | | | GATCGCCTACGCT | | | |
| TGEGPTKG | A9 | 9 | ACCGGCGAGGGCCC | A27 | 1111 | ++ |
| | | | CACCAAGGGC | | | |
| SGSVGYVG | A10 | 10 | AGCGGCAGCGTGGG | A28 | 1112 | ++ |
| | | | CTACGTGGGC | | | |
| LAQVQVEGA | All | 11 | CTAGCACAGGTGCA | A29 | 1113 | ++ |
| | | | GGTGGAGGGCGCT | | | |
| SGFDATKG | A12 | 12 | AGCGGCTTCGACGC | A30 | 1114 | ++ |
| | | | CACCAAGGGC | | | |
| TTDLNHKG | A13 | 13 | ACCACCGACCTGAA | A31 | 1115 | + |
| | | | CCACAAGGGC | | | |
| SSRAYQET | A14 | 14 | AGCAGCCGGGCCTA | A32 | 1116 | + |
| | | | CCAGGAGACC | | | |
| LAKVASMNA | A15 | 15 | CTAGCAAAGGTGGC | A32 | 1117 | + |
| | | | CAGCATGAACGCT | | | |
| TSQEKQNS | A16 | 16 | ACCAGCCAGGAGAA | A34 | 1118 | + |
| | | | GCAGAACAGC | | | |
| LAAHQMASA | A17 | 17 | CTAGCAGCCCACCA | A35 | 1119 | + |
| | | | GATGGCCAGCGCT | | | |
| LAKGQAQNA | A18 | 18 | CTAGCAAAGGGCCA | A36 | 1120 | + |
| | | | GGCCCAGAACGCT | | | |
| RTGRNT | A19 | 19 | CGGACCGGCCGGAA | A37 | 1121 | ++ |
| | | | CACC | | | |

*AAV2 vectors designed to include SEQ ID NO: 1 with the indicated sequence inclusion. The 8-mers and 9-mers were inserted between amino acid residues 587 and 588 of SEQ ID NO: 1, and the 6-mers were used as replacements for amino acid residues 585 to 590 of SEQ ID NO: 1.
+++ means that the modified AAV2 vectors had a performance that placed them within the top 1/3 of the rescued AAV vectors;
++ means the AAV2 vectors had a performance that placed them within the middle 1/3 of the rescued AAV vectors; and
+ means the AAV2 vectors had a performance that placed them within the bottom 1/3 of the rescued AAV vectors.
**SEQ ID NOs used for Sequence Listing.

TABLE 1B
Amino acid sequences that can be inserted into an AAV capsid
polypeptide.

| Amino | SEQ | | SEQ | Nucleic Acid Sequence | SEQ | | SEQ | |
|---|---|---|---|---|---|---|---|---|
| Acid | ID | | ID | encoding the Amino | ID | | ID | Activity |
| Sequence | NO: | | NO:** | Acid Sequence | NO: | | NO:** | Level* |
| LATTTMGTA | B | 2 | 20 | CTAGCAACCACCACCATGGGCACCGCT | B | 318 | 1122 | +++ |
| LARRKSNEA | B | 3 | 21 | CTAGCACGGCGGAAGAGCAACGAGGCT | B | 319 | 1123 | +++ |
| LAHGGLREA | B | 4 | 22 | CTAGCACACGGCGGCCTGCGGGAGGCT | B | 320 | 1124 | +++ |
| LAMFVWVPA | B | 5 | 23 | CTAGCAATGTTCGTGTGGGTGCCCGCT | B | 321 | 1125 | +++ |
| LATDRTKTA | B | 6 | 24 | CTAGCAACCGACCGGACCAAGACCGCT | B | 322 | 1126 | +++ |
| LAFNSCSYA | B | 7 | 25 | CTAGCATTCAACAGCTGCAGCTACGCT | B | 323 | 1127 | +++ |
| LAYAEQSKA | B | 8 | 26 | CTAGCATACGCCGAGCAGAGCAAGGCT | B | 324 | 1128 | +++ |
| LAWRIMKHA | B | 9 | 27 | CTAGCATGGCGGATCATGAAGCACGCT | B | 325 | 1129 | +++ |
| LAVDNIWAA | B | 10 | 28 | CTAGCAGTGGACAACATCTGGGCCGCT | B | 326 | 1130 | +++ |
| LAGQAMMQA | B | 11 | 29 | CTAGCAGGCCAGGCCATGATGCAGGCT | B | 327 | 1131 | +++ |
| LAELALIKA | B | 12 | 30 | CTAGCAGAGCTGGCCCTGATCAAGGCT | B | 328 | 1132 | +++ |
| LAMTKITDA | B | 13 | 31 | CTAGCAATGACCAAGATCACCGACGCT | B | 329 | 1133 | +++ |
| LALENIGQA | B | 14 | 32 | CTAGCACTGGAGAACATCGGCCAGGCT | B | 330 | 1134 | +++ |
| LAFTHGTNA | B | 15 | 33 | CTAGCATTCACCCACGGCACCAACGCT | B | 331 | 1135 | +++ |
| LAKINAQPA | B | 16 | 34 | CTAGCAAAGATCAACGCCCAGCCCGCT | B | 332 | 1136 | +++ |
| LADLSDHHA | B | 17 | 35 | CTAGCAGACCTGAGCGACCACCACGCT | B | 333 | 1137 | +++ |
| LADPLYAVA | B | 18 | 36 | CTAGCAGACCCCCTGTACGCCGTGGCT | B | 334 | 1138 | +++ |
| LAYVALFYA | B | 19 | 37 | CTAGCATACGTGGCCCTGTTCTACGCT | B | 335 | 1139 | +++ |
| LAGGEYETA | B | 20 | 38 | CTAGCAGGCGGCGAGTACGAGACCGCT | B | 336 | 1140 | +++ |
| LAALVDANA | B | 21 | 39 | CTAGCAGCCCTGGTGGACGCCAACGCT | B | 337 | 1141 | +++ |
| LADPTETPA | B | 22 | 40 | CTAGCAGACCCCACCGAGACCCCCGCT | B | 338 | 1142 | +++ |
| LAHRAPDSA | B | 23 | 41 | CTAGCACACCGGGCCCCCGACAGCGCT | B | 339 | 1143 | +++ |
| LAEGQEDSA | B | 24 | 42 | CTAGCAGAGGGCCAGGAGGACAGCGCT | B | 340 | 1144 | +++ |
| LAEEEKYGA | B | 25 | 43 | CTAGCAGAGGAGGAGAAGTACGGCGCT | B | 341 | 1145 | +++ |
| LALNDNVGA | B | 26 | 44 | CTAGCACTGAACGACAACGTGGGCGCT | B | 342 | 1146 | +++ |
| LAPDAKDWA | B | 27 | 45 | CTAGCACCCGACGCCAAGGACTGGGCT | B | 343 | 1147 | +++ |
| LAPHIAKDA | B | 28 | 46 | CTAGCACCCCACATCGCCAAGGACGCT | B | 344 | 1148 | +++ |
| LADPGWKYA | B | 29 | 47 | CTAGCAGACCCCGGCTGGAAGTACGCT | B | 345 | 1149 | +++ |
| LASRTAVAA | B | 30 | 48 | CTAGCAAGCCGGACCGCCGTGGCCGCT | B | 346 | 1150 | +++ |
| LAFCIKHCA | B | 31 | 49 | CTAGCATTCTGCATCAAGCACTGCGCT | B | 347 | 1151 | +++ |
| LAEPDHTLA | B | 32 | 50 | CTAGCAGAGCCCGACCACACCTTGGCT | B | 348 | 1152 | +++ |
| LAPMRGVYA | B | 33 | 51 | CTAGCACCCATGCGGGGCGTGTACGCT | B | 349 | 1153 | +++ |
| LAAVTKDSA | B | 34 | 52 | CTAGCAGCCGTGACCAAGGACAGCGCT | B | 350 | 1154 | +++ |
| LAITQSDIA | B | 35 | 53 | CTAGCAATCACCCAGAGCGACATCGCT | B | 351 | 1155 | +++ |
| LAPNQGTPA | B | 36 | 54 | CTAGCACCCAACCAGGGCACCCCCGCT | B | 352 | 1156 | +++ |
| LAVMGNMLA | B | 37 | 55 | CTAGCAGTGATGGGCAACATGCTGGCT | B | 353 | 1157 | +++ |
| LASNMERIA | B | 38 | 56 | CTAGCAAGCAACATGGAGCGGATCGCT | B | 354 | 1158 | +++ |
| LAHWQEEWA | B | 39 | 57 | CTAGCACACTGGCAGGAGGAGTGGGCT | B | 355 | 1159 | +++ |
| LADDMIFVA | B | 40 | 58 | CTAGCAGACGACATGATCTTCGTGGCT | B | 356 | 1160 | +++ |
| LAHEPMFNA | B | 41 | 59 | CTAGCACACGAGCCCATGTTCAACGCT | B | 357 | 1161 | +++ |
| LATVYKSPA | B | 42 | 60 | CTAGCAACCGTGTACAAGAGCCCCGCT | B | 358 | 1162 | +++ |
| LADNLNTPA | B | 43 | 61 | CTAGCAGACAACCTGAACACCCCCGCT | B | 359 | 1163 | +++ |
| LAYVIENDA | B | 44 | 62 | CTAGCATACGTGATCGAGAACGACGCT | B | 360 | 1164 | +++ |
| LALEVGDHA | B | 45 | 63 | CTAGCACTGGAGGTGGGCGACCACGCT | B | 361 | 1165 | +++ |
| LAHEGRQAA | B | 46 | 64 | CTAGCACACGAGGGCCGGCAGGCCGCT | B | 362 | 1166 | +++ |
| LASTTMNIA | B | 47 | 65 | CTAGCAAGCACCACCATGAACATCGCT | B | 363 | 1167 | +++ |
| LAEFHIPTA | B | 48 | 66 | CTAGCAGAGTTCCACATCCCCACCGCT | B | 364 | 1168 | +++ |
| LAEFRVGQA | B | 49 | 67 | CTAGCAGAGTTCCGGGTGGGCCAGGCT | B | 365 | 1169 | +++ |
| LADAHSVTA | B | 50 | 68 | CTAGCAGACGCCCACAGCGTGACCGCT | B | 366 | 1170 | +++ |
| LAKPPPKDA | B | 51 | 69 | CTAGCAAAGCCCCCCCCCAAGGACGCT | B | 367 | 1171 | +++ |
| LAIGSDTSA | B | 52 | 70 | CTAGCAATCGGCAGCGACACCAGCGCT | B | 368 | 1172 | +++ |
| LAFQQQRCA | B | 53 | 71 | CTAGCATTCCAGCAGCAGCGGTGCGCT | B | 369 | 1173 | +++ |
| LAAVHLMGA | B | 54 | 72 | CTAGCAGCCGTGCACCTGATGGGCGCT | B | 370 | 1174 | +++ |
| LACTYTYAA | B | 55 | 73 | CTAGCATGCACCTACACCTACGCCGCT | B | 371 | 1175 | +++ |
| LAVRNEINA | B | 56 | 74 | CTAGCAGTGCGGAACGAGATCAACGCT | B | 372 | 1176 | +++ |
| LADSSKWIA | B | 57 | 75 | CTAGCAGACAGCAGCAAGTGGATCGCT | B | 373 | 1177 | +++ |
| LADYRNDKA | B | 58 | 76 | CTAGCAGACTACCGGAACGACAAGGCT | B | 374 | 1178 | +++ |
| LANANTRPA | B | 59 | 77 | CTAGCAAACGCCAACACCCGGCCCGCT | B | 375 | 1179 | +++ |
| LAPQNLDEA | B | 60 | 78 | CTAGCACCCCAGAACCTGGACGAGGCT | B | 376 | 1180 | +++ |
| LAHPGTTFA | B | 61 | 79 | CTAGCACACCCCGGCACCACCTTCGCT | B | 377 | 1181 | +++ |
| LAYDAGGHA | B | 62 | 80 | CTAGCATACGACGCCGGCGGCCACGCT | B | 378 | 1182 | +++ |
| LAKALGMEA | B | 63 | 81 | CTAGCAAAGGCCCTGGGCATGGAGGCT | B | 379 | 1183 | +++ |
| LAGENHISA | B | 64 | 82 | CTAGCAGGCGAGAACCACATCAGCGCT | B | 380 | 1184 | +++ |
| LARDQLKLA | B | 65 | 83 | CTAGCACGGGACCAGCTGAAGCTGGCT | B | 381 | 1185 | +++ |
| LACPVAEWA | B | 66 | 84 | CTAGCATGCCCCGTGGCCGAGTGGGCT | B | 382 | 1186 | +++ |
| LAQVKQGPA | B | 67 | 85 | CTAGCACAGGTGAAGCAGGGCCCCGCT | B | 383 | 1187 | +++ |
| LALPEGRFA | B | 68 | 86 | CTAGCACTGCCCGAGGGCCGGTTCGCT | B | 384 | 1188 | +++ |
| LAMNEPVTA | B | 69 | 87 | CTAGCAATGAACGAGCCCGTGACCGCT | B | 385 | 1189 | +++ |
| LAAVTEYVA | B | 70 | 88 | CTAGCAGCCGTGACCGAGTACGTGGCT | B | 386 | 1190 | +++ |
| LADGGTRPA | B | 71 | 89 | CTAGCAGACGGCGGCACCCGGCCCGCT | B | 387 | 1191 | +++ |
| LAPPGNPLA | B | 72 | 90 | CTAGCACCCCCCGGCAACCCCCTGGCT | B | 388 | 1192 | +++ |
| LANIVQQDA | B | 73 | 91 | CTAGCAAACATCGTGCAGCAGGACGCT | B | 389 | 1193 | +++ |
| LAVSRHGEA | B | 74 | 92 | CTAGCAGTGAGCCGGCACGGCGAGGCT | B | 390 | 1194 | +++ |
| LANEAQGKA | B | 75 | 93 | CTAGCAAACGAGGCCCAGGGCAAGGCT | B | 391 | 1195 | +++ |
| LAPMGQNMA | B | 76 | 94 | CTAGCACCCATGGGCCAGAACATGGCT | B | 392 | 1196 | +++ |
| LAAYHDSAA | B | 77 | 95 | CTAGCAGCCTACCACGACAGCGCCGCT | B | 393 | 1197 | +++ |
| LAGIQQHVA | B | 78 | 96 | CTAGCAGGCATCCAGCAGCACGTGGCT | B | 394 | 1198 | +++ |
| LASVSTIQA | B | 79 | 97 | CTAGCAAGCGTGAGCACCATCCAGGCT | B | 395 | 1199 | +++ |
| LAAVGMKMA | B | 80 | 98 | CTAGCAGCCGTGGGCATGAAGATGGCT | B | 396 | 1200 | +++ |
| LAQVTEMRA | B | 81 | 99 | CTAGCACAGGTGACCGAGATGCGGGCT | B | 397 | 1201 | +++ |
| LADTHTTYA | B | 82 | 100 | CTAGCAGACACCCACACCACCTACGCT | B | 398 | 1202 | +++ |
| LAIGERTTA | B | 83 | 101 | CTAGCAATCGGCGAGCGGACCACCGCT | B | 399 | 1203 | +++ |
| LANKNTAIA | B | 84 | 102 | CTAGCAAACAAGAACACCGCCATCGCT | B | 400 | 1204 | +++ |
| LANLKENEA | B | 85 | 103 | CTAGCAAACCTGAAGGAGAACGAGGCT | B | 401 | 1205 | +++ |
| LAEYHNNDA | B | 86 | 104 | CTAGCAGAGTACCACAACAACGACGCT | B | 402 | 1206 | +++ |
| LAFNGSTSA | B | 87 | 105 | CTAGCATTCAACGGCAGCACCAGCGCT | B | 403 | 1207 | +++ |
| LAGFMEYYA | B | 88 | 106 | CTAGCAGGCTTCATGGAGTACTACGCT | B | 404 | 1208 | +++ |
| LAMGHNYVA | B | 89 | 107 | CTAGCAATGGGCCACAACTACGTGGCT | B | 405 | 1209 | +++ |
| LAGEFTGKA | B | 90 | 108 | CTAGCAGGCGAGTTCACCGGCAAGGCT | B | 406 | 1210 | ++ |
| LAKGFKTEA | B | 91 | 109 | CTAGCAAAGGGCTTCAAGACCGAGGCT | B | 407 | 1211 | ++ |
| LAWNKCPYA | B | 92 | 110 | CTAGCATGGAACAAGTGCCCCTACGCT | B | 408 | 1212 | ++ |
| LAQEQNAAA | B | 93 | 111 | CTAGCACAGGAGCAGAACGCCGCCGCT | B | 409 | 1213 | ++ |
| LAKDGMIGA | B | 94 | 112 | CTAGCAAAGGACGGCATGATCGGCGCT | B | 410 | 1214 | ++ |
| LAPTTIGGA | B | 95 | 113 | CTAGCACCCACCACCATCGGCGGCGCT | B | 411 | 1215 | ++ |
| LAVKGLVNA | B | 96 | 114 | CTAGCAGTGAAGGGCCTGGTGAACGCT | B | 412 | 1216 | ++ |
| LALNSGKMA | B | 97 | 115 | CTAGCACTGAACAGCGGCAAGATGGCT | B | 413 | 1217 | ++ |
| LALYQCDTA | B | 98 | 116 | CTAGCACTGTACCAGTGCGACACCGCT | B | 414 | 1218 | ++ |
| LAPQKQPVA | B | 99 | 117 | CTAGCACCCCAGAAGCAGCCCGTGGCT | B | 415 | 1219 | ++ |
| LAEHKNMHA | B | 100 | 118 | CTAGCAGAGCACAAGAACATGCACGCT | B | 416 | 1220 | ++ |
| LAEFDGDGA | B | 101 | 119 | CTAGCAGAGTTCGACGGCGACGGCGCT | B | 417 | 1221 | ++ |
| LASRLNSPA | B | 102 | 120 | CTAGCAAGCCGGCTGAACAGCCCCGCT | B | 418 | 1222 | ++ |
| LAPCYEPPA | B | 103 | 121 | CTAGCACCCTGCTACGAGCCCCCCGCT | B | 419 | 1223 | ++ |
| LALKLRTEA | B | 104 | 122 | CTAGCACTGAAGCTGCGGACCGAGGCT | B | 420 | 1224 | ++ |
| LAAGQAQSA | B | 105 | 123 | CTAGCAGCCGGCCAGGCCCAGAGCGCT | B | 421 | 1225 | ++ |
| LATPNVNAA | B | 106 | 124 | CTAGCAACCCCCAACGTGAACGCCGCT | B | 422 | 1226 | ++ |
| LARAITNEA | B | 107 | 125 | CTAGCACGGGCCATCACCAACGAGGCT | B | 423 | 1227 | ++ |
| LAEVTYTGA | B | 108 | 126 | CTAGCAGAGGTGACCTACACCGGCGCT | B | 424 | 1228 | ++ |
| LAPSGDRHA | B | 109 | 127 | CTAGCACCCAGCGGCGACCGGCACGCT | B | 425 | 1229 | ++ |
| LAYITEKSA | B | 110 | 128 | CTAGCATACATCACCGAGAAGAGCGCT | B | 426 | 1230 | ++ |
| LAKDHHVIA | B | 111 | 129 | CTAGCAAAGGACCACCACGTGATCGCT | B | 427 | 1231 | ++ |
| LASADHQPA | B | 112 | 130 | CTAGCAAGCGCCGACCACCAGCCCGCT | B | 428 | 1232 | ++ |
| LATMHNFEA | B | 113 | 131 | CTAGCAACCATGCACAACTTCGAGGCT | B | 429 | 1233 | ++ |
| LARGVAPIA | B | 114 | 132 | CTAGCACGGGGCGTGGCCCCCATCGCT | B | 430 | 1234 | ++ |
| LAWKCKGTA | B | 115 | 133 | CTAGCATGGAAGTGCAAGGGCACCGCT | B | 431 | 1235 | ++ |
| LAKGILMEA | B | 116 | 134 | CTAGCAAAGGGCATCCTGATGGAGGCT | B | 432 | 1236 | ++ |
| LANIVSHDA | B | 117 | 135 | CTAGCAAACATCGTGAGCCACGACGCT | B | 433 | 1237 | ++ |
| LAEFVHKGA | B | 118 | 136 | CTAGCAGAGTTCGTGCACAAGGGCGCT | B | 434 | 1238 | ++ |
| LASAVMYWA | B | 119 | 137 | CTAGCAAGCGCCGTGATGTACTGGGCT | B | 435 | 1239 | ++ |
| LAQKKLLEA | B | 120 | 138 | CTAGCACAGAAGAAGCTGCTGGAGGCT | B | 436 | 1240 | ++ |
| LAGERETVA | B | 121 | 139 | CTAGCAGGCGAGCGGGAGACCGTGGCT | B | 437 | 1241 | ++ |
| LAFLRGPEA | B | 122 | 140 | CTAGCATTCCTGCGGGGCCCCGAGGCT | B | 438 | 1242 | ++ |
| LAAVGVGYA | B | 123 | 141 | CTAGCAGCCGTGGGCGTGGGCTACGCT | B | 439 | 1243 | ++ |
| LADLGYPNA | B | 124 | 142 | CTAGCAGACCTGGGCTACCCCAACGCT | B | 440 | 1244 | ++ |
| LAAKPKDGA | B | 125 | 143 | CTAGCAGCCAAGCCCAAGGACGGCGCT | B | 441 | 1245 | ++ |
| LADKNRLQA | B | 126 | 144 | CTAGCAGACAAGAACCGGCTGCAGGCT | B | 442 | 1246 | ++ |
| LADMTRFHA | B | 127 | 145 | CTAGCAGACATGACCCGGTTCCACGCT | B | 443 | 1247 | ++ |
| LARSSTLNA | B | 128 | 146 | CTAGCACGGAGCAGCACCCTGAACGCT | B | 444 | 1248 | ++ |
| LACYPPTCA | B | 129 | 147 | CTAGCATGCTACCCCCCCACCTGCGCT | B | 445 | 1249 | ++ |
| LAKVSKGDA | B | 130 | 148 | CTAGCAAAGGTGAGCAAGGGCGACGCT | B | 446 | 1250 | ++ |
| LAGGKQALA | B | 131 | 149 | CTAGCAGGCGGCAAGCAGGCCCTGGCT | B | 447 | 1251 | ++ |
| LADIVGESA | B | 132 | 150 | CTAGCAGACATCGTGGGCGAGAGCGCT | B | 448 | 1252 | ++ |
| LADFPSEKA | B | 133 | 151 | CTAGCAGACTTCCCCAGCGAGAAGGCT | B | 449 | 1253 | ++ |
| LAHIQEPKA | B | 134 | 152 | CTAGCACACATCCAGGAGCCCAAGGCT | B | 450 | 1254 | ++ |
| LAAANHDNA | B | 135 | 153 | CTAGCAGCCGCCAACCACGACAACGCT | B | 451 | 1255 | ++ |
| LAALNSQTA | B | 136 | 154 | CTAGCAGCCCTGAACAGCCAGACCGCT | B | 452 | 1256 | ++ |
| LAEPFPVAA | B | 137 | 155 | CTAGCAGAGCCCTTCCCCGTGGCCGCT | B | 453 | 1257 | ++ |
| LAELDFKWA | B | 138 | 156 | CTAGCAGAGCTGGACTTCAAGTGGGCT | B | 454 | 1258 | ++ |
| LAKFMWIHA | B | 139 | 157 | CTAGCAAAGTTCATGTGGATCCACGCT | B | 455 | 1259 | ++ |
| LAQVQVEGA | B | 140 | 158 | CTAGCACAGGTGCAGGTGGAGGGCGCT | B | 456 | 1260 | ++ |
| LADILVGQA | B | 141 | 159 | CTAGCAGACATCCTGGTGGGCCAGGCT | B | 457 | 1261 | ++ |
| LAQWFLWRA | B | 142 | 160 | CTAGCACAGTGGTTCCTGTGGCGGGCT | B | 458 | 1262 | ++ |
| LALHQERQA | B | 143 | 161 | CTAGCACTGCACCAGGAGCGGCAGGCT | B | 459 | 1263 | ++ |
| LACSSTWYA | B | 144 | 162 | CTAGCATGCAGCAGCACCTGGTACGCT | B | 460 | 1264 | ++ |
| LAGSMAKLA | B | 145 | 163 | CTAGCAGGCAGCATGGCCAAGCTGGCT | B | 461 | 1265 | ++ |
| LAPNTPKIA | B | 146 | 164 | CTAGCACCCAACACCCCCAAGATCGCT | B | 462 | 1266 | ++ |
| LALDMWEPA | B | 147 | 165 | CTAGCACTGGACATGTGGGAGCCCGCT | B | 463 | 1267 | ++ |
| LAGHAACFA | B | 148 | 166 | CTAGCAGGCCACGCCGCCTGCTTCGCT | B | 464 | 1268 | ++ |
| LAVRTGSMA | B | 149 | 167 | CTAGCAGTGCGGACCGGCAGCATGGCT | B | 465 | 1269 | ++ |
| LAGVVKTDA | B | 150 | 168 | CTAGCAGGCGTGGTGAAGACCGACGCT | B | 466 | 1270 | ++ |
| LAKFAAKDA | B | 151 | 169 | CTAGCAAAGTTCGCCGCCAAGGACGCT | B | 467 | 1271 | ++ |
| LAVNRVDMA | B | 152 | 170 | CTAGCAGTGAACCGGGTGGACATGGCT | B | 468 | 1272 | ++ |
| LANNAEPSA | B | 153 | 171 | CTAGCAAACAACGCCGAGCCCAGCGCT | B | 469 | 1273 | ++ |
| LAAKMYNGA | B | 154 | 172 | CTAGCAGCCAAGATGTACAACGGCGCT | B | 470 | 1274 | ++ |
| LAEADKWYA | B | 155 | 173 | CTAGCAGAGGCCGACAAGTGGTACGCT | B | 471 | 1275 | ++ |
| LARGTLMTA | B | 156 | 174 | CTAGCACGGGGCACCCTGATGACCGCT | B | 472 | 1276 | ++ |
| LAKDKGLIA | B | 157 | 175 | CTAGCAAAGGACAAGGGCCTGATCGCT | B | 473 | 1277 | ++ |
| LAQGNNTGA | B | 158 | 176 | CTAGCACAGGGCAACAACACCGGCGCT | B | 474 | 1278 | ++ |
| LAWIERSMA | B | 159 | 177 | CTAGCATGGATCGAGCGGAGCATGGCT | B | 475 | 1279 | ++ |
| LAYDNTAEA | B | 160 | 178 | CTAGCATACGACAACACCGCCGAGGCT | B | 476 | 1280 | ++ |
| LAGFHAIEA | B | 161 | 179 | CTAGCAGGCTTCCACGCCATCGAGGCT | B | 477 | 1281 | ++ |
| LAFGFSADA | B | 162 | 180 | CTAGCATTCGGCTTCAGCGCCGACGCT | B | 478 | 1282 | ++ |
| LAEGMTGIA | B | 163 | 181 | CTAGCAGAGGGCATGACCGGCATCGCT | B | 479 | 1283 | ++ |
| LAHDLHGGA | B | 164 | 182 | CTAGCACACGACCTGCACGGCGGCGCT | B | 480 | 1284 | ++ |
| LAQAGDTAA | B | 165 | 183 | CTAGCACAGGCCGGCGACACCGCCGCT | B | 481 | 1285 | ++ |
| LAGEVTKHA | B | 166 | 184 | CTAGCAGGCGAGGTGACCAAGCACGCT | B | 482 | 1286 | ++ |
| LALNAAATA | B | 167 | 185 | CTAGCACTGAACGCCGCCGCCACCGCT | B | 483 | 1287 | ++ |
| LADCMMINA | B | 168 | 186 | CTAGCAGACTGCATGATGATCAACGCT | B | 484 | 1288 | ++ |
| LAYFHSYAA | B | 169 | 187 | CTAGCATACTTCCACAGCTACGCCGCT | B | 485 | 1289 | ++ |
| LAISQPNPA | B | 170 | 188 | CTAGCAATCAGCCAGCCCAACCCCGCT | B | 486 | 1290 | ++ |
| LAVAVDCFA | B | 171 | 189 | CTAGCAGTGGCCGTGGACTGCTTCGCT | B | 487 | 1291 | ++ |
| LADHGNKPA | B | 172 | 190 | CTAGCAGACCACGGCAACAAGCCCGCT | B | 488 | 1292 | ++ |
| LAAEHIGRA | B | 173 | 191 | CTAGCAGCCGAGCACATCGGCCGGGCT | B | 489 | 1293 | ++ |
| LAVTPPWYA | B | 174 | 192 | CTAGCAGTGACCCCCCCCTGGTACGCT | B | 490 | 1294 | ++ |
| LAVPKADAA | B | 175 | 193 | CTAGCAGTGCCCAAGGCCGACGCCGCT | B | 491 | 1295 | ++ |
| LANMMAAGA | B | 176 | 194 | CTAGCAAACATGATGGCCGCCGGCGCT | B | 492 | 1296 | ++ |
| LANVTLQSA | B | 177 | 195 | CTAGCAAACGTGACCCTGCAGAGCGCT | B | 493 | 1297 | ++ |
| LAPIQLAQA | B | 178 | 196 | CTAGCACCCATCCAGCTGGCCCAGGCT | B | 494 | 1298 | ++ |
| LANQGFLTA | B | 179 | 197 | CTAGCAAACCAGGGCTTCCTGACCGCT | B | 495 | 1299 | ++ |
| LAPLQTQEA | B | 180 | 198 | CTAGCACCCCTGCAGACCCAGGAGGCT | B | 496 | 1300 | ++ |
| LAEQVLEKA | B | 181 | 199 | CTAGCAGAGCAGGTGCTGGAGAAGGCT | B | 497 | 1301 | ++ |
| LAVDIMRGA | B | 182 | 200 | CTAGCAGTGGACATCATGCGGGGCGCT | B | 498 | 1302 | ++ |
| LACTFKPNA | B | 183 | 201 | CTAGCATGCACCTTCAAGCCCAACGCT | B | 499 | 1303 | ++ |
| LATWCLVWA | B | 184 | 202 | CTAGCAACCTGGTGCCTGGTGTGGGCT | B | 500 | 1304 | + |
| LAKSAGILA | B | 185 | 203 | CTAGCAAAGAGCGCCGGCATCCTGGCT | B | 501 | 1305 | + |
| LAYGQQTTA | B | 186 | 204 | CTAGCATACGGCCAGCAGACCACCGCT | B | 502 | 1306 | + |
| LAHAGSSSA | B | 187 | 205 | CTAGCACACGCCGGCAGCAGCAGCGCT | B | 503 | 1307 | + |
| LAGKTISGA | B | 188 | 206 | CTAGCAGGCAAGACCATCAGCGGCGCT | B | 504 | 1308 | + |
| LASIGFSAA | B | 189 | 207 | CTAGCAAGCATCGGCTTCAGCGCCGCT | B | 505 | 1309 | + |
| LAFPCIKEA | B | 190 | 208 | CTAGCATTCCCCTGCATCAAGGAGGCT | B | 506 | 1310 | + |
| LAMLGGTSA | B | 191 | 209 | CTAGCAATGCTGGGCGGCACCAGCGCT | B | 507 | 1311 | + |
| LAGGSKLEA | B | 192 | 210 | CTAGCAGGCGGCAGCAAGCTGGAGGCT | B | 508 | 1312 | + |
| LAADPTFKA | B | 193 | 211 | CTAGCAGCCGACCCCACCTTCAAGGCT | B | 509 | 1313 | + |
| LAETTLTRA | B | 194 | 212 | CTAGCAGAGACCACCCTGACCCGGGCT | B | 510 | 1314 | + |
| LAHDWGAPA | B | 195 | 213 | CTAGCACACGACTGGGGCGCCCCCGCT | B | 511 | 1315 | + |
| LAMDREIKA | B | 196 | 214 | CTAGCAATGGACCGGGAGATCAAGGCT | B | 512 | 1316 | + |
| LAVFQETGA | B | 197 | 215 | CTAGCAGTGTTCCAGGAGACCGGCGCT | B | 513 | 1317 | + |
| LAQVEVSKA | B | 198 | 216 | CTAGCACAGGTGGAGGTGAGCAAGGCT | B | 514 | 1318 | + |
| LAPGNDLWA | B | 199 | 217 | CTAGCACCCGGCAACGACCTGTGGGCT | B | 515 | 1319 | + |
| LAIPVEANA | B | 200 | 218 | CTAGCAATCCCCGTGGAGGCCAACGCT | B | 516 | 1320 | + |
| LAKDGGKIA | B | 201 | 219 | CTAGCAAAGGACGGCGGCAAGATCGCT | B | 517 | 1321 | + |
| LAERNHGYA | B | 202 | 220 | CTAGCAGAGCGGAACCACGGCTACGCT | B | 518 | 1322 | + |
| LAMLACKGA | B | 203 | 221 | CTAGCAATGCTGGCCTGCAAGGGCGCT | B | 519 | 1323 | + |
| LAHHKESIA | B | 204 | 222 | CTAGCACACCACAAGGAGAGCATCGCT | B | 520 | 1324 | + |
| LAYHQWDDA | B | 205 | 223 | CTAGCATACCACCAGTGGGACGACGCT | B | 521 | 1325 | + |
| LAKVASMNA | B | 206 | 224 | CTAGCAAAGGTGGCCAGCATGAACGCT | B | 522 | 1326 | + |
| LANEGTIRA | B | 207 | 225 | CTAGCAAACGAGGGCACCATCCGGGCT | B | 523 | 1327 | + |
| LAGKIEENA | B | 208 | 226 | CTAGCAGGCAAGATCGAGGAGAACGCT | B | 524 | 1328 | + |
| LAPVKHTWA | B | 209 | 227 | CTAGCACCCGTGAAGCACACCTGGGCT | B | 525 | 1329 | + |
| LADEGREHA | B | 210 | 228 | CTAGCAGACGAGGGCCGGGAGCACGCT | B | 526 | 1330 | + |
| LALNNDQSA | B | 211 | 229 | CTAGCACTGAACAACGACCAGAGCGCT | B | 527 | 1331 | + |
| LADVVSVSA | B | 212 | 230 | CTAGCAGACGTGGTGAGCGTGAGCGCT | B | 528 | 1332 | + |
| LAPSKMGLA | B | 213 | 231 | CTAGCACCCAGCAAGATGGGCCTGGCT | B | 529 | 1333 | + |
| LASACMRAA | B | 214 | 232 | CTAGCAAGCGCCTGCATGCGGGCCGCT | B | 530 | 1334 | + |
| LAGDIVRNA | B | 215 | 233 | CTAGCAGGCGACATCGTGCGGAACGCT | B | 531 | 1335 | + |
| LADTYKPYA | B | 216 | 234 | CTAGCAGACACCTACAAGCCCTACGCT | B | 532 | 1336 | + |
| LAFPDKSIA | B | 217 | 235 | CTAGCATTCCCCGACAAGAGCATCGCT | B | 533 | 1337 | + |
| LANADFPLA | B | 218 | 236 | CTAGCAAACGCCGACTTCCCCCTGGCT | B | 534 | 1338 | + |
| LAKEGYRDA | B | 219 | 237 | CTAGCAAAGGAGGGCTACCGGGACGCT | B | 535 | 1339 | + |
| LADPMPTHA | B | 220 | 238 | CTAGCAGACCCCATGCCCACCCACGCT | B | 536 | 1340 | + |
| LAQNQKQIA | B | 221 | 239 | CTAGCACAGAACCAGAAGCAGATCGCT | B | 537 | 1341 | + |
| LASENLNRA | B | 222 | 240 | CTAGCAAGCGAGAACCTGAACCGGGCT | B | 538 | 1342 | + |
| LAFCTPRYA | B | 223 | 241 | CTAGCATTCTGCACCCCCCGGTACGCT | B | 539 | 1343 | + |
| LANLKNFSA | B | 224 | 242 | CTAGCAAACCTGAAGAACTTCAGCGCT | B | 540 | 1344 | + |
| LANAMEKHA | B | 225 | 243 | CTAGCAAACGCCATGGAGAAGCACGCT | B | 541 | 1345 | + |
| LAMHESYNA | B | 226 | 244 | CTAGCAATGCACGAGAGCTACAACGCT | B | 542 | 1346 | + |
| LAMKYVFDA | B | 227 | 245 | CTAGCAATGAAGTACGTGTTCGACGCT | B | 543 | 1347 | + |
| LAKGQAQNA | B | 228 | 246 | CTAGCAAAGGGCCAGGCCCAGAACGCT | B | 544 | 1348 | + |
| LAFRTMEYA | B | 229 | 247 | CTAGCATTCCGGACCATGGAGTACGCT | B | 545 | 1349 | + |
| LAKKVVDGA | B | 230 | 248 | CTAGCAAAGAAGGTGGTGGACGGCGCT | B | 546 | 1350 | + |
| LAKGMKEGA | B | 231 | 249 | CTAGCAAAGGGCATGAAGGAGGGCGCT | B | 547 | 1351 | + |
| LADDQAVNA | B | 232 | 250 | CTAGCAGACGACCAGGCCGTGAACGCT | B | 548 | 1352 | + |
| LANTWCKGA | B | 233 | 251 | CTAGCAAACACCTGGTGCAAGGGCGCT | B | 549 | 1353 | + |
| LANQTTREA | B | 234 | 252 | CTAGCAAACCAGACCACCCGGGAGGCT | B | 550 | 1354 | + |
| LADGIQKNA | B | 235 | 253 | CTAGCAGACGGCATCCAGAAGAACGCT | B | 551 | 1355 | + |
| LAHQGAIIA | B | 236 | 254 | CTAGCACACCAGGGCGCCATCATCGCT | B | 552 | 1356 | + |
| LADNCWCCA | B | 237 | 255 | CTAGCAGACAACTGCTGGTGCTGCGCT | B | 553 | 1357 | + |
| LADVGCENA | B | 238 | 256 | CTAGCAGACGTGGGCTGCGAGAACGCT | B | 554 | 1358 | + |
| LAGSRVNFA | B | 239 | 257 | CTAGCAGGCAGCCGGGTGAACTTCGCT | B | 555 | 1359 | + |
| LATKSTGSA | B | 240 | 258 | CTAGCAACCAAGAGCACCGGCAGCGCT | B | 556 | 1360 | + |
| LAAHQMASA | B | 241 | 259 | CTAGCAGCCCACCAGATGGCCAGCGCT | B | 557 | 1361 | + |
| LAREDHRQA | B | 242 | 260 | CTAGCACGGGAGGACCACCGGCAGGCT | B | 558 | 1362 | + |
| LAYLPLKYA | B | 243 | 261 | CTAGCATACCTGCCCCTGAAGTACGCT | B | 559 | 1363 | + |
| LALETTQRA | B | 244 | 262 | CTAGCACTGGAGACCACCCAGCGGGCT | B | 560 | 1364 | + |
| LAEGLSQFA | B | 245 | 263 | CTAGCAGAGGGCCTGAGCCAGTTCGCT | B | 561 | 1365 | + |
| LADWRTTPA | B | 246 | 264 | CTAGCAGACTGGCGGACCACCCCCGCT | B | 562 | 1366 | + |
| LAETVKYMA | B | 247 | 265 | CTAGCAGAGACCGTGAAGTACATGGCT | B | 563 | 1367 | + |
| LAAVKAVVA | B | 248 | 266 | CTAGCAGCCGTGAAGGCCGTGGTGGCT | B | 564 | 1368 | + |
| LAKGQSQGA | B | 249 | 267 | CTAGCAAAGGGCCAGAGCCAGGGCGCT | B | 565 | 1369 | + |
| LAVEPGPPA | B | 250 | 268 | CTAGCAGTGGAGCCCGGCCCCCCCGCT | B | 566 | 1370 | + |
| LAAEHKQTA | B | 251 | 269 | CTAGCAGCCGAGCACAAGCAGACCGCT | B | 567 | 1371 | + |
| LAHYPSAPA | B | 252 | 270 | CTAGCACACTACCCCAGCGCCCCCGCT | B | 568 | 1372 | + |
| LATRADIFA | B | 253 | 271 | CTAGCAACCCGGGCCGACATCTTCGCT | B | 569 | 1373 | + |
| LAYLRALKA | B | 254 | 272 | CTAGCATACCTGCGGGCCCTGAAGGCT | B | 570 | 1374 | + |
| LAQNQWTDA | B | 255 | 273 | CTAGCACAGAACCAGTGGACCGACGCT | B | 571 | 1375 | + |
| LAGGAWDWA | B | 256 | 274 | CTAGCAGGCGGCGCCTGGGACTGGGCT | B | 572 | 1376 | + |
| LADENLQAA | B | 257 | 275 | CTAGCAGACGAGAACCTGCAGGCCGCT | B | 573 | 1377 | + |
| LANRLSNVA | B | 258 | 276 | CTAGCAAACCGGCTGAGCAACGTGGCT | B | 574 | 1378 | + |
| LAEPPDPPA | B | 259 | 277 | CTAGCAGAGCCCCCCGACCCCCCCGCT | B | 575 | 1379 | + |
| LAQEAKLTA | B | 260 | 278 | CTAGCACAGGAGGCCAAGCTGACCGCT | B | 576 | 1380 | + |
| LAHTPDTFA | B | 261 | 279 | CTAGCACACACCCCCGACACCTTCGCT | B | 577 | 1381 | + |
| LAGESSYLA | B | 262 | 280 | CTAGCAGGCGAGAGCAGCTACCTGGCT | B | 578 | 1382 | + |
| LADLTLRAA | B | 263 | 281 | CTAGCAGACCTGACCCTGCGGGCCGCT | B | 579 | 1383 | + |
| LAKRDKPTA | B | 264 | 282 | CTAGCAAAGCGGGACAAGCCCACCGCT | B | 580 | 1384 | + |
| LANRWAIDA | B | 265 | 283 | CTAGCAAACCGGTGGGCCATCGACGCT | B | 581 | 1385 | + |
| LACRSDCVA | B | 266 | 284 | CTAGCATGCCGGAGCGACTGCGTGGCT | B | 582 | 1386 | + |
| LAKPTGKDA | B | 267 | 285 | CTAGCAAAGCCCACCGGCAAGGACGCT | B | 583 | 1387 | + |
| LAGHKHDEA | B | 268 | 286 | CTAGCAGGCCACAAGCACGACGAGGCT | B | 584 | 1388 | + |
| LAAQDVSWA | B | 269 | 287 | CTAGCAGCCCAGGACGTGAGCTGGGCT | B | 585 | 1389 | + |
| LADVGCENA | B | 270 | 288 | CTAGCAGACGTGGGCTGCGAGAACGCT | B | 586 | 1390 | + |
| LAKGPASIA | B | 271 | 289 | CTAGCAAAGGGCCCCGCCAGCATCGCT | B | 587 | 1391 | + |
| LAGPQRFQA | B | 272 | 290 | CTAGCAGGCCCCCAGCGGTTCCAGGCT | B | 588 | 1392 | + |
| LAATICTEA | B | 273 | 291 | CTAGCAGCCACCATCTGCACCGAGGCT | B | 589 | 1393 | + |
| LAMTNTFLA | B | 274 | 292 | CTAGCAATGACCAACACCTTCCTGGCT | B | 590 | 1394 | + |
| LAKGMKEGA | B | 275 | 293 | CTAGCAAAGGGCATGAAGGAGGGCGCT | B | 591 | 1395 | + |
| LAKGVTTAA | B | 276 | 294 | CTAGCAAAGGGCGTGACCACCGCCGCT | B | 592 | 1396 | + |
| LARFRGMCA | B | 277 | 295 | CTAGCACGGTTCCGGGGCATGTGCGCT | B | 593 | 1397 | + |
| TSHVSTSS | B | 278 | 296 | ACCAGCCACGTGAGCACCAGCAGC | B | 594 | 1398 | +++ |
| SGVLFTEG | B | 279 | 297 | AGCGGCGTGCTGTTCACCGAGGGC | B | 595 | 1399 | +++ |
| TSGMGAIG | B | 280 | 298 | ACCAGCGGCATGGGCGCCATCGGC | B | 596 | 1400 | +++ |
| TGDSEQQT | B | 281 | 299 | ACCGGCGACAGCGAGCAGCAGACC | B | 597 | 1401 | +++ |
| SGPGTSGT | B | 282 | 300 | AGCGGCCCCGGCACCAGCGGCACC | B | 598 | 1402 | +++ |
| SGSVGYVG | B | 283 | 301 | AGCGGCAGCGTGGGCTACGTGGGC | B | 599 | 1403 | +++ |
| TGEGPTKG | B | 284 | 302 | ACCGGCGAGGGCCCCACCAAGGGC | B | 600 | 1404 | +++ |
| TGDKWPQG | B | 285 | 303 | ACCGGCGACAAGTGGCCCCAGGGC | B | 601 | 1405 | +++ |
| SGEMTKPG | B | 286 | 304 | AGCGGCGAGATGACCAAGCCCGGC | B | 602 | 1406 | ++ |
| TTENHKPG | B | 287 | 305 | ACCACCGAGAACCACAAGCCCGGC | B | 603 | 1407 | ++ |
| SGREIHFG | B | 288 | 306 | AGCGGCCGGGAGATCCACTTCGGC | B | 604 | 1408 | ++ |
| SSENNKGG | B | 289 | 307 | AGCAGCGAGAACAACAAGGGCGGC | B | 605 | 1409 | ++ |
| TSQEKQNS | B | 290 | 308 | ACCAGCCAGGAGAAGCAGAACAGC | B | 606 | 1410 | ++ |
| GTMFNNST | B | 291 | 309 | GGCACCATGTTCAACAACAGCACC | B | 607 | 1411 | + |
| TTGMERPT | B | 292 | 310 | ACCACCGGCATGGAGCGGCCCACC | B | 608 | 1412 | + |
| SGPKETGG | B | 293 | 311 | AGCGGCCCCAAGGAGACCGGCGGC | B | 609 | 1413 | + |
| SGIDTRLG | B | 294 | 312 | AGCGGCATCGACACCCGGCTGGGC | B | 610 | 1414 | + |
| STVPLNMG | B | 295 | 313 | AGCACCGTGCCCCTGAACATGGGC | B | 611 | 1415 | + |
| TGIATSYS | B | 296 | 314 | ACCGGCATCGCCACCAGCTACAGC | B | 612 | 1416 | + |
| TGDERKPG | B | 297 | 315 | ACCGGCGACGAGCGGAAGCCCGGC | B | 613 | 1417 | + |
| TGPGTHNG | B | 298 | 316 | ACCGGCCCCGGCACCCACAACGGC | B | 614 | 1418 | + |
| STTKPM | B | 299 | 317 | AGCACCACCAAGCCCATG | B | 615 | 1419 | +++ |
| CKEITR | B | 300 | 318 | TGCAAGGAGATCACCCGG | B | 616 | 1420 | +++ |
| SKMRPA | B | 301 | 319 | AGCAAGATGCGGCCCGCC | B | 617 | 1421 | +++ |
| PPNQYI | B | 302 | 320 | CCCCCCAACCAGTACATC | B | 618 | 1422 | +++ |
| PDVSLL | B | 303 | 321 | CCCGACGTGAGCCTGCTG | B | 619 | 1423 | +++ |
| HSKDGA | B | 304 | 322 | CACAGCAAGGACGGCGCC | B | 620 | 1424 | +++ |
| KFRHTE | B | 305 | 323 | AAGTTCCGGCACACCGAG | B | 621 | 1425 | +++ |
| LWMTTV | B | 306 | 324 | CTGTGGATGACCACCGTG | B | 622 | 1426 | +++ |
| RPGRVS | B | 307 | 325 | CGGCCCGGCCGGGTGAGC | B | 623 | 1427 | +++ |
| NIKKAA | B | 308 | 326 | AACATCAAGAAGGCCGCC | B | 624 | 1428 | +++ |
| RLGRLS | B | 309 | 327 | CGGCTGGGCCGGCTGAGC | B | 625 | 1429 | ++ |
| FNKVLH | B | 310 | 328 | TTCAACAAGGTGCTGCAC | B | 626 | 1430 | ++ |
| RTGRNT | B | 311 | 329 | CGGACCGGCCGGAACACC | B | 627 | 1431 | ++ |
| RALKIS | B | 312 | 330 | CGGGCCCTGAAGATCAGC | B | 628 | 1432 | ++ |
| RLGKVP | B | 313 | 331 | CGGCTGGGCAAGGTGCCC | B | 629 | 1433 | + |
| KFTKAA | B | 314 | 332 | AAGTTCACCAAGGCCGCC | B | 630 | 1434 | + |
| HFPVFS | B | 315 | 333 | CACTTCCCCGTGTTCAGC | B | 631 | 1435 | + |
| NSKKLG | B | 316 | 334 | AACAGCAAGAAGCTGGGC | B | 632 | 1436 | + |
| KAMRTG | B | 317 | 335 | AAGGCCATGCGGACCGGC | B | 633 | 1437 | + |

*AAV2 vectors designed to include SEQ ID NO: 1 with the indicated sequence inclusion. The 8-mers and 9-mers were inserted between amino acid residues 587 and 588 of SEQ ID NO: 1, and the 6-mers were used as replacements for amino acid residues 585 to 590 of SEQ ID NO: 1.
+++ means that the modified AAV2 vectors had a performance that placed them within the top 1/3 of the rescued AAV vectors;
++ means the AAV2 vectors had a performance that placed them within the middle 1/3 of the rescued AAV vectors; and
+ means the AAV2 vectors had a performance that placed them within the bottom 1/3 of the rescued AAV vectors.
**SEQ ID NOs used for Sequence Listing.

TABLE 1C
Amino acid sequences that can be inserted into an AAV capsid polypeptide.

| | | | SEQ | | | | SEQ | |
|---|---|---|---|---|---|---|---|---|
| | SEQ | | ID | | | | ID | |
| Amino Acid | ID | | NO: | Nucleic Acid Sequence encoding the Amino | SEQ ID | | NO: | Activity |
| Sequence | NO: | | ** | Acid Sequence | NO: | | ** | Level* |
| LAEFRVGQA | C | 2 | 336 | CTAGCAGAGTTCCGGGTGGGCCAGGCT | C | 46 | 1438 | +++ |
| LAGIQQHVA | C | 3 | 337 | CTAGCAGGCATCCAGCAGCACGTGGCT | C | 47 | 1439 | +++ |
| LAQVTEMRA | C | 4 | 338 | CTAGCACAGGTGACCGAGATGCGGGCT | C | 48 | 1440 | +++ |
| LAFNGSTSA | C | 5 | 339 | CTAGCATTCAACGGCAGCACCAGCGCT | C | 49 | 1441 | +++ |
| LAKGFKTEA | C | 6 | 340 | CTAGCAAAGGGCTTCAAGACCGAGGCT | C | 50 | 1442 | +++ |
| LAAGQAQSA | C | 7 | 341 | CTAGCAGCCGGCCAGGCCCAGAGCGCT | C | 51 | 1443 | +++ |
| LAFLRGPEA | C | 8 | 342 | CTAGCATTCCTGCGGGGCCCCGAGGCT | C | 52 | 1444 | +++ |
| LAQVQVEGA | C | 9 | 343 | CTAGCACAGGTGCAGGTGGAGGGCGCT | C | 53 | 1445 | +++ |
| LAGSMAKLA | C | 10 | 344 | CTAGCAGGCAGCATGGCCAAGCTGGCT | C | 54 | 1446 | +++ |
| LAGHAACFA | C | 11 | 345 | CTAGCAGGCCACGCCGCCTGCTTCGCT | C | 55 | 1447 | +++ |
| LAVNRVDMA | C | 12 | 346 | CTAGCAGTGAACCGGGTGGACATGGCT | C | 56 | 1448 | +++ |
| LAGFHAIEA | C | 13 | 347 | CTAGCAGGCTTCCACGCCATCGAGGCT | C | 57 | 1449 | +++ |
| LAYFHSYAA | C | 14 | 348 | CTAGCATACTTCCACAGCTACGCCGCT | C | 58 | 1450 | +++ |
| LADHGNKPA | C | 15 | 349 | CTAGCAGACCACGGCAACAAGCCCGCT | C | 59 | 1451 | ++ |
| LAPIQLAQA | C | 16 | 350 | CTAGCACCCATCCAGCTGGCCCAGGCT | C | 60 | 1452 | ++ |
| LAPLQTQEA | C | 18 | 351 | CTAGCACCCCTGCAGACCCAGGAGGCT | C | 61 | 1453 | ++ |
| LAEQVLEKA | C | 19 | 352 | CTAGCAGAGCAGGTGCTGGAGAAGGCT | C | 62 | 1454 | ++ |
| LAFPCIKEA | C | 20 | 353 | CTAGCATTCCCCTGCATCAAGGAGGCT | C | 63 | 1455 | ++ |
| LAQVEVSKA | C | 21 | 354 | CTAGCACAGGTGGAGGTGAGCAAGGCT | C | 64 | 1456 | ++ |
| LAKDGGKIA | C | 22 | 355 | CTAGCAAAGGACGGCGGCAAGATCGCT | C | 65 | 1457 | ++ |
| LAKVASMNA | C | 23 | 356 | CTAGCAAAGGTGGCCAGCATGAACGCT | C | 66 | 1458 | ++ |
| LADEGREHA | C | 24 | 357 | CTAGCAGACGAGGGCCGGGAGCACGCT | C | 67 | 1459 | ++ |
| LASACMRAA | C | 25 | 358 | CTAGCAAGCGCCTGCATGCGGGCCGCT | C | 68 | 1460 | ++ |
| LAGDIVRNA | C | 26 | 359 | CTAGCAGGCGACATCGTGCGGAACGCT | C | 69 | 1461 | ++ |
| LADPMPTHA | C | 27 | 360 | CTAGCAGACCCCATGCCCACCCACGCT | C | 70 | 1462 | ++ |
| LAFCTPRYA | C | 28 | 361 | CTAGCATTCTGCACCCCCCGGTACGCT | C | 71 | 1463 | ++ |
| LANAMEKHA | C | 29 | 362 | CTAGCAAACGCCATGGAGAAGCACGCT | C | 72 | 1464 | + |
| LAMHESYNA | C | 30 | 363 | CTAGCAATGCACGAGAGCTACAACGCT | C | 73 | 1465 | + |
| LAKGQAQNA | C | 31 | 364 | CTAGCAAAGGGCCAGGCCCAGAACGCT | C | 74 | 1466 | + |
| LADDQAVNA | C | 32 | 365 | CTAGCAGACGACCAGGCCGTGAACGCT | C | 75 | 1467 | + |
| LADGIQKNA | C | 33 | 366 | CTAGCAGACGGCATCCAGAAGAACGCT | C | 76 | 1468 | 1 |
| LAAHQMASA | C | 34 | 367 | CTAGCAGCCCACCAGATGGCCAGCGCT | C | 77 | 1469 | + |
| LAETVKYMA | C | 35 | 368 | CTAGCAGAGACCGTGAAGTACATGGCT | C | 78 | 1470 | + |
| LAKGQSQGA | C | 36 | 369 | CTAGCAAAGGGCCAGAGCCAGGGCGCT | C | 79 | 1471 | + |
| LAVEPGPPA | C | 37 | 370 | CTAGCAGTGGAGCCCGGCCCCCCCGCT | C | 80 | 1472 | + |
| LAAEHKQTA | C | 38 | 371 | CTAGCAGCCGAGCACAAGCAGACCGCT | C | 81 | 1473 | + |
| LAEPPDPPA | C | 39 | 372 | CTAGCAGAGCCCCCCGACCCCCCCGCT | C | 82 | 1474 | + |
| LAHTPDTFA | C | 40 | 373 | CTAGCACACACCCCCGACACCTTCGCT | C | 83 | 1475 | + |
| LAKGPASIA | C | 41 | 374 | CTAGCAAAGGGCCCCGCCAGCATCGCT | C | 84 | 1476 | + |
| LAKGMKEGA | C | 42 | 375 | CTAGCAAAGGGCATGAAGGAGGGCGCT | C | 85 | 1477 | + |
| LAKGVTTAA | C | 43 | 376 | CTAGCAAAGGGCGTGACCACCGCCGCT | C | 86 | 1478 | + |
| NSKKLG | C | 44 | 377 | AACAGCAAGAAGCTGGGC | C | 87 | 1479 | + |
| KAMRTG | C | 45 | 378 | AAGGCCATGCGGACCGGC | C | 88 | 1480 | + |

*AAV2 vectors designed to include SEQ ID NO: 1 with the indicated sequence inclusion. The 9-mers were inserted between amino acid residues 587 and 588 of SEQ ID NO: 1, and the 6-mers were used as replacements for amino acid residues 585 to 590 of SEQ ID NO: 1. +++ means that the modified AAV2 vectors had a performance that placed them within the top 1/3 of the rescued AAV vectors; ++ means the AAV2 vectors had a performance that placed them within the middle 1/3 of the rescued AAV vectors; and + means the AAV2 vectors had a performance that placed them within the bottom 1/3 of the rescued AAV vectors.
**SEQ ID NOs used for Sequence Listing.

TABLE 1D
Amino acid sequences that can be inserted into an AAV capsid polypeptide.

| | | | SEQ | | | | SEQ | |
|---|---|---|---|---|---|---|---|---|
| | SEQ | | ID | | | | ID | |
| Amino Acid | ID | | NO: | Nucleic Acid Sequence encoding the Amino | SEQ ID | | NO: | Activity |
| Sequence | NO: | | ** | Acid Sequence | NO: | | ** | Level* |
| LAKINAQPA | D | 2 | 379 | CTAGCAAAGATCAACGCCCAGCCCGCT | D | 79 | 1481 | +++ |
| LAEEEKYGA | D | 3 | 380 | CTAGCAGAGGAGGAGAAGTACGGCGCT | D | 80 | 1482 | +++ |
| LALEVGDHA | D | 4 | 381 | CTAGCACTGGAGGTGGGCGACCACGCT | D | 81 | 1483 | +++ |
| LAIGSDTSA | D | 5 | 382 | CTAGCAATCGGCAGCGACACCAGCGCT | D | 82 | 1484 | +++ |
| LAAVHLMGA | D | 6 | 383 | CTAGCAGCCGTGCACCTGATGGGCGCT | D | 83 | 1485 | +++ |
| LAVRNEINA | D | 7 | 384 | CTAGCAGTGCGGAACGAGATCAACGCT | D | 84 | 1486 | +++ |
| LAQVKQGPA | D | 8 | 385 | CTAGCACAGGTGAAGCAGGGCCCCGCT | D | 85 | 1487 | +++ |
| LANKNTAIA | D | 9 | 386 | CTAGCAAACAAGAACACCGCCATCGCT | D | 86 | 1488 | +++ |
| LAEHKNMHA | D | 10 | 387 | CTAGCAGAGCACAAGAACATGCACGCT | D | 87 | 1489 | +++ |
| LAEVTYTGA | D | 11 | 388 | CTAGCAGAGGTGACCTACACCGGCGCT | D | 88 | 1490 | +++ |
| LASADHQPA | D | 12 | 389 | CTAGCAAGCGCCGACCACCAGCCCGCT | D | 89 | 1491 | +++ |
| LAEFDGDGA | D | 13 | 390 | CTAGCAGAGTTCGACGGCGACGGCGCT | D | 90 | 1492 | +++ |
| LADFPSEKA | D | 14 | 391 | CTAGCAGACTTCCCCAGCGAGAAGGCT | D | 91 | 1493 | +++ |
| LALDMWEPA | D | 15 | 392 | CTAGCACTGGACATGTGGGAGCCCGCT | D | 92 | 1494 | +++ |
| LAKFAAKDA | D | 16 | 393 | CTAGCAAAGTTCGCCGCCAAGGACGCT | D | 93 | 1495 | +++ |
| LANNAEPSA | D | 17 | 394 | CTAGCAAACAACGCCGAGCCCAGCGCT | D | 94 | 1496 | +++ |
| LAHDWGAPA | D | 18 | 395 | CTAGCACACGACTGGGGCGCCCCCGCT | D | 95 | 1497 | +++ |
| LAMDREIKA | D | 19 | 396 | CTAGCAATGGACCGGGAGATCAAGGCT | D | 96 | 1498 | +++ |
| LAQGNNTGA | D | 20 | 397 | CTAGCACAGGGCAACAACACCGGCGCT | D | 97 | 1499 | +++ |
| LADNLNTPA | D | 21 | 398 | CTAGCAGACAACCTGAACACCCCCGCT | D | 98 | 1500 | +++ |
| LAAANHDNA | D | 22 | 399 | CTAGCAGCCGCCAACCACGACAACGCT | D | 99 | 1501 | +++ |
| LAVPKADAA | D | 23 | 400 | CTAGCAGTGCCCAAGGCCGACGCCGCT | D | 100 | 1502 | +++ |
| LAVFQETGA | D | 24 | 401 | CTAGCAGTGTTCCAGGAGACCGGCGCT | D | 101 | 1503 | +++ |
| LAAVKAVVA | D | 25 | 402 | CTAGCAGCCGTGAAGGCCGTGGTGGCT | D | 102 | 1504 | +++ |
| LADNCWCCA | D | 26 | 403 | CTAGCAGACAACTGCTGGTGCTGCGCT | D | 103 | 1505 | +++ |
| LAHAGSSSA | D | 27 | 404 | CTAGCACACGCCGGCAGCAGCAGCGCT | D | 104 | 1506 | +++ |
| LAGIQQHVA | D | 28 | 405 | CTAGCAGGCATCCAGCAGCACGTGGCT | D | 105 | 1507 | ++ |
| LAHDLHGGA | D | 29 | 406 | CTAGCACACGACCTGCACGGCGGCGCT | D | 106 | 1508 | ++ |
| LAMHESYNA | D | 30 | 407 | CTAGCAATGCACGAGAGCTACAACGCT | D | 107 | 1509 | ++ |
| LATPNVNAA | D | 31 | 408 | CTAGCAACCCCCAACGTGAACGCCGCT | D | 108 | 1510 | ++ |
| LAFNGSTSA | D | 32 | 409 | CTAGCATTCAACGGCAGCACCAGCGCT | D | 109 | 1511 | ++ |
| LAEGLSQFA | D | 33 | 410 | CTAGCAGAGGGCCTGAGCCAGTTCGCT | D | 110 | 1512 | ++ |
| LAQVTEMRA | D | 34 | 411 | CTAGCACAGGTGACCGAGATGCGGGCT | D | 111 | 1513 | ++ |
| LAAHQMASA | D | 35 | 412 | CTAGCAGCCCACCAGATGGCCAGCGCT | D | 112 | 1514 | ++ |
| LASACMRAA | D | 36 | 413 | CTAGCAAGCGCCTGCATGCGGGCCGCT | D | 113 | 1515 | ++ |
| LADGIQKNA | D | 37 | 414 | CTAGCAGACGGCATCCAGAAGAACGCT | D | 114 | 1516 | ++ |
| LAHIQEPKA | D | 38 | 415 | CTAGCACACATCCAGGAGCCCAAGGCT | D | 115 | 1517 | ++ |
| LAAGQAQSA | D | 39 | 416 | CTAGCAGCCGGCCAGGCCCAGAGCGCT | D | 116 | 1518 | ++ |
| LADHGNKPA | D | 40 | 417 | CTAGCAGACCACGGCAACAAGCCCGCT | D | 117 | 1519 | ++ |
| LAFLRGPEA | D | 41 | 418 | CTAGCATTCCTGCGGGGCCCCGAGGCT | D | 118 | 1520 | ++ |
| LAETVKYMA | D | 42 | 419 | CTAGCAGAGACCGTGAAGTACATGGCT | D | 119 | 1521 | ++ |
| LADEGREHA | D | 43 | 420 | CTAGCAGACGAGGGCCGGGAGCACGCT | D | 120 | 1522 | ++ |
| LAGDIVRNA | D | 44 | 421 | CTAGCAGGCGACATCGTGCGGAACGCT | D | 121 | 1523 | ++ |
| LAGSMAKLA | D | 45 | 422 | CTAGCAGGCAGCATGGCCAAGCTGGCT | D | 122 | 1524 | ++ |
| LAKGMKEGA | D | 46 | 423 | CTAGCAAAGGGCATGAAGGAGGGCGCT | D | 123 | 1525 | ++ |
| LAEPPDPPA | D | 47 | 424 | CTAGCAGAGCCCCCCGACCCCCCCGCT | D | 124 | 1526 | ++ |
| LATWCLVWA | D | 48 | 425 | CTAGCAACCTGGTGCCTGGTGTGGGCT | D | 125 | 1527 | ++ |
| LAFPCIKEA | D | 49 | 426 | CTAGCATTCCCCTGCATCAAGGAGGCT | D | 126 | 1528 | ++ |
| LAKGFKTEA | D | 50 | 427 | CTAGCAAAGGGCTTCAAGACCGAGGCT | D | 127 | 1529 | + |
| LAKGQSQGA | D | 51 | 428 | CTAGCAAAGGGCCAGAGCCAGGGCGCT | D | 128 | 1530 | + |
| LAEQVLEKA | D | 52 | 429 | CTAGCAGAGCAGGTGCTGGAGAAGGCT | D | 129 | 1531 | + |
| LAKGPASIA | D | 53 | 430 | CTAGCAAAGGGCCCCGCCAGCATCGCT | D | 130 | 1532 | + |
| LAEFRVGQA | D | 54 | 431 | CTAGCAGAGTTCCGGGTGGGCCAGGCT | D | 131 | 1533 | + |
| LAVEPGPPA | D | 55 | 432 | CTAGCAGTGGAGCCCGGCCCCCCCGCT | D | 132 | 1534 | + |
| LAVNRVDMA | D | 56 | 433 | CTAGCAGTGAACCGGGTGGACATGGCT | D | 133 | 1535 | + |
| LAYFHSYAA | D | 57 | 434 | CTAGCATACTTCCACAGCTACGCCGCT | D | 134 | 1536 | + |
| LAHTPDTFA | D | 58 | 435 | CTAGCACACACCCCCGACACCTTCGCT | D | 135 | 1537 | + |
| LAFCTPRYA | D | 59 | 436 | CTAGCATTCTGCACCCCCCGGTACGCT | D | 136 | 1538 | + |
| LAAEHKQTA | D | 60 | 437 | CTAGCAGCCGAGCACAAGCAGACCGCT | D | 137 | 1539 | + |
| LAPIQLAQA | D | 61 | 438 | CTAGCACCCATCCAGCTGGCCCAGGCT | D | 138 | 1540 | + |
| LAQVEVSKA | D | 62 | 439 | CTAGCACAGGTGGAGGTGAGCAAGGCT | D | 139 | 1541 | + |
| LAPLQTQEA | D | 63 | 440 | CTAGCACCCCTGCAGACCCAGGAGGCT | D | 140 | 1542 | + |
| LARAITNEA | D | 64 | 441 | CTAGCACGGGCCATCACCAACGAGGCT | D | 141 | 1543 | + |
| LAGHAACFA | D | 65 | 442 | CTAGCAGGCCACGCCGCCTGCTTCGCT | D | 142 | 1544 | + |
| LAGFHAIEA | D | 66 | 443 | CTAGCAGGCTTCCACGCCATCGAGGCT | D | 143 | 1545 | + |
| LAQVQVEGA | D | 67 | 444 | CTAGCACAGGTGCAGGTGGAGGGCGCT | D | 144 | 1546 | + |
| LANAMEKHA | D | 68 | 445 | CTAGCAAACGCCATGGAGAAGCACGCT | D | 145 | 1547 | + |
| LADPMPTHA | D | 69 | 446 | CTAGCAGACCCCATGCCCACCCACGCT | D | 146 | 1548 | + |
| LAKVASMNA | D | 70 | 447 | CTAGCAAAGGTGGCCAGCATGAACGCT | D | 147 | 1549 | + |
| LAKGQAQNA | D | 71 | 448 | CTAGCAAAGGGCCAGGCCCAGAACGCT | D | 148 | 1550 | + |
| LAKDGGKIA | D | 72 | 449 | CTAGCAAAGGACGGCGGCAAGATCGCT | D | 149 | 1551 | + |
| LADDQAVNA | D | 73 | 450 | CTAGCAGACGACCAGGCCGTGAACGCT | D | 150 | 1552 | + |
| LAKGVTTAA | D | 74 | 451 | CTAGCAAAGGGCGTGACCACCGCCGCT | D | 151 | 1553 | + |
| SGPKETGG | D | 75 | 452 | AGCGGCCCCAAGGAGACCGGCGGC | D | 152 | 1554 | ++ |
| TGPGTHNG | D | 76 | 453 | ACCGGCCCCGGCACCCACAACGGC | D | 153 | 1555 | ++ |
| NSKKLG | D | 77 | 454 | AACAGCAAGAAGCTGGGC | D | 154 | 1556 | + |
| KAMRTG | D | 78 | 455 | AAGGCCATGCGGACCGGC | D | 155 | 1557 | + |

*AAV2 vectors designed to include SEQ ID NO: 1 with the indicated sequence inclusion. The 8-mers and 9-mers were inserted between amino acid residues 587 and 588 of SEQ ID NO: 1, and the 6-mers were used as replacements for amino acid residues 585 to 590 of SEQ ID NO: 1. +++ means that the modified AAV2 vectors had a performance that placed them within the top 1/3 of the rescued AAV vectors; ++ means the AAV2 vectors had a performance that placed them within the middle 1/3 of the rescued AAV vectors; and + means the AAV2 vectors had a performance that placed them within the bottom 1/3 of the rescued AAV vectors.
**SEQ ID NOs used for Sequence Listing.

TABLE 1E
Amino acid sequences that can be inserted into an AAV capsid polypeptide.

| | | | SEQ | | | | SEQ | |
|---|---|---|---|---|---|---|---|---|
| | SEQ | | ID | | SEQ | | ID | |
| Amino Acid | ID | | NO: | Nucleic Acid Sequence encoding the Amino | ID | | NO: | Activity |
| Sequence | NO: | | ** | Acid Sequence | NO: | | ** | Level* |
| LAELALIKA | E | 2 | 456 | CTAGCAGAGCTGGCCCTGATCAAGGCT | E | 84 | 1558 | +++ |
| LADLSDHHA | E | 3 | 457 | CTAGCAGACCTGAGCGACCACCACGCT | E | 85 | 1559 | +++ |
| LADPLYAVA | E | 4 | 458 | CTAGCAGACCCCCTGTACGCCGTGGCT | E | 86 | 1560 | +++ |
| LAPMRGVYA | E | 5 | 459 | CTAGCACCCATGCGGGGCGTGTACGCT | E | 87 | 1561 | +++ |
| LAITQSDIA | F | 6 | 460 | CTAGCAATCACCCAGAGCGACATCGCT | F | 88 | 1562 | +++ |
| LADDMIFVA | E | 7 | 461 | CTAGCAGACGACATGATCTTCGTGGCT | E | 89 | 1563 | +++ |
| LAEFRVGQA | E | 8 | 462 | CTAGCAGAGTTCCGGGTGGGCCAGGCT | E | 90 | 1564 | +++ |
| LAKALGMEA | E | 9 | 463 | CTAGCAAAGGCCCTGGGCATGGAGGCT | E | 91 | 1565 | +++ |
| LACPVAEWA | E | 10 | 464 | CTAGCATGCCCCGTGGCCGAGTGGGCT | E | 92 | 1566 | +++ |
| LANEAQGKA | E | 11 | 465 | CTAGCAAACGAGGCCCAGGGCAAGGCT | E | 93 | 1567 | +++ |
| LAQVTEMRA | E | 12 | 466 | CTAGCACAGGTGACCGAGATGCGGGCT | E | 94 | 1568 | +++ |
| LAIGERTTA | E | 13 | 467 | CTAGCAATCGGCGAGCGGACCACCGCT | E | 95 | 1569 | +++ |
| LANLKENEA | E | 14 | 468 | CTAGCAAACCTGAAGGAGAACGAGGCT | E | 96 | 1570 | +++ |
| LAFNGSTSA | E | 15 | 469 | CTAGCATTCAACGGCAGCACCAGCGCT | E | 97 | 1571 | +++ |
| LAQEQNAAA | E | 16 | 470 | CTAGCACAGGAGCAGAACGCCGCCGCT | E | 98 | 1572 | +++ |
| LAPTTIGGA | E | 17 | 471 | CTAGCACCCACCACCATCGGCGGCGCT | E | 99 | 1573 | +++ |
| LAPSGDRHA | E | 18 | 472 | CTAGCACCCAGCGGCGACCGGCACGCT | E | 100 | 1574 | +++ |
| LAKDHHVIA | E | 19 | 473 | CTAGCAAAGGACCACCACGTGATCGCT | E | 101 | 1575 | +++ |
| LAQKKLLEA | E | 20 | 474 | CTAGCACAGAAGAAGCTGCTGGAGGCT | E | 102 | 1576 | +++ |
| LAFLRGPEA | E | 21 | 475 | CTAGCATTCCTGCGGGGCCCCGAGGCT | E | 103 | 1577 | ++ |
| LARSSTLNA | E | 22 | 476 | CTAGCACGGAGCAGCACCCTGAACGCT | E | 104 | 1578 | ++ |
| LAHIQEPKA | E | 23 | 477 | CTAGCACACATCCAGGAGCCCAAGGCT | E | 105 | 1579 | ++ |
| LAQVQVEGA | E | 24 | 478 | CTAGCACAGGTGCAGGTGGAGGGCGCT | E | 106 | 1580 | ++ |
| LAPNTPKIA | E | 25 | 479 | CTAGCACCCAACACCCCCAAGATCGCT | F | 107 | 1581 | ++ |
| LAVNRVDMA | E | 26 | 480 | CTAGCAGTGAACCGGGTGGACATGGCT | E | 108 | 1582 | ++ |
| LAGFHAIEA | E | 27 | 481 | CTAGCAGGCTTCCACGCCATCGAGGCT | E | 109 | 1583 | ++ |
| LAEGMTGIA | E | 28 | 482 | CTAGCAGAGGGCATGACCGGCATCGCT | E | 110 | 1584 | ++ |
| LAGEVTKHA | E | 29 | 483 | CTAGCAGGCGAGGTGACCAAGCACGCT | E | 111 | 1585 | ++ |
| LAYFHSYAA | E | 30 | 484 | CTAGCATACTTCCACAGCTACGCCGCT | E | 112 | 1586 | ++ |
| LAPLQTQEA | E | 31 | 485 | CTAGCACCCCTGCAGACCCAGGAGGCT | E | 113 | 1587 | ++ |
| LAFPCIKEA | E | 32 | 486 | CTAGCATTCCCCTGCATCAAGGAGGCT | F | 114 | 1588 | ++ |
| LAQVEVSKA | E | 33 | 487 | CTAGCACAGGTGGAGGTGAGCAAGGCT | E | 115 | 1589 | ++ |
| LAPGNDLWA | E | 34 | 488 | CTAGCACCCGGCAACGACCTGTGGGCT | E | 116 | 1590 | ++ |
| LAKDGGKIA | E | 35 | 489 | CTAGCAAAGGACGGCGGCAAGATCGCT | E | 117 | 1591 | ++ |
| LAKVASMNA | E | 36 | 490 | CTAGCAAAGGTGGCCAGCATGAACGCT | E | 118 | 1592 | ++ |
| LAGKIEENA | E | 37 | 491 | CTAGCAGGCAAGATCGAGGAGAACGCT | E | 119 | 1593 | ++ |
| LAPVKHTWA | E | 38 | 492 | CTAGCACCCGTGAAGCACACCTGGGCT | E | 120 | 1594 | ++ |
| LALNNDQSA | E | 39 | 493 | CTAGCACTGAACAACGACCAGAGCGCT | E | 121 | 1595 | ++ |
| LAGDIVRNA | E | 40 | 494 | CTAGCAGGCGACATCGTGCGGAACGCT | E | 122 | 1596 | ++ |
| LANADFPLA | E | 41 | 495 | CTAGCAAACGCCGACTTCCCCCTGGCT | E | 123 | 1597 | ++ |
| LAKEGYRDA | E | 42 | 496 | CTAGCAAAGGAGGGCTACCGGGACGCT | E | 124 | 1598 | ++ |
| LADPMPTHA | E | 43 | 497 | CTAGCAGACCCCATGCCCACCCACGCT | E | 125 | 1599 | ++ |
| LAFCTPRYA | E | 44 | 498 | CTAGCATTCTGCACCCCCCGGTACGCT | E | 126 | 1600 | + |
| LANLKNFSA | F | 45 | 499 | CTAGCAAACCTGAAGAACTTCAGCGCT | E | 127 | 1601 | + |
| LANAMEKHA | F | 46 | 500 | CTAGCAAACGCCATGGAGAAGCACGCT | E | 128 | 1602 | + |
| LAKGQAQNA | E | 47 | 501 | CTAGCAAAGGGCCAGGCCCAGAACGCT | E | 129 | 1603 | + |
| LADDQAVNA | E | 48 | 502 | CTAGCAGACGACCAGGCCGTGAACGCT | E | 130 | 1604 | + |
| LANTWCKGA | E | 49 | 503 | CTAGCAAACACCTGGTGCAAGGGCGCT | E | 131 | 1605 | + |
| LADVGCENA | E | 50 | 504 | CTAGCAGACGTGGGCTGCGAGAACGCT | E | 132 | 1606 | + |
| LAGSRVNFA | E | 51 | 505 | CTAGCAGGCAGCCGGGTGAACTTCGCT | E | 133 | 1607 | + |
| LAAHQMASA | E | 52 | 506 | CTAGCAGCCCACCAGATGGCCAGCGCT | E | 134 | 1608 | + |
| LAREDHRQA | E | 53 | 507 | CTAGCACGGGAGGACCACCGGCAGGCT | E | 135 | 1609 | + |
| LAVEPGPPA | E | 54 | 508 | CTAGCAGTGGAGCCCGGCCCCCCCGCT | E | 136 | 1610 | + |
| LATRADIFA | E | 55 | 509 | CTAGCAACCCGGGCCGACATCTTCGCT | E | 137 | 1611 | + |
| LAQNQWTDA | E | 56 | 510 | CTAGCACAGAACCAGTGGACCGACGCT | E | 138 | 1612 | + |
| LADENLQAA | E | 57 | 511 | CTAGCAGACGAGAACCTGCAGGCCGCT | E | 139 | 1613 | + |
| LAEPPDPPA | E | 58 | 512 | CTAGCAGAGCCCCCCGACCCCCCCGCT | E | 140 | 1614 | + |
| LAQEAKLTA | E | 59 | 513 | CTAGCACAGGAGGCCAAGCTGACCGCT | E | 141 | 1615 | + |
| LAHTPDTFA | E | 60 | 514 | CTAGCACACACCCCCGACACCTTCGCT | E | 142 | 1616 | + |
| LADLTLRAA | E | 61 | 515 | CTAGCAGACCTGACCCTGCGGGCCGCT | E | 143 | 1617 | + |
| LANRWAIDA | E | 62 | 516 | CTAGCAAACCGGTGGGCCATCGACGCT | E | 144 | 1618 | + |
| LAKPTGKDA | E | 63 | 517 | CTAGCAAAGCCCACCGGCAAGGACGCT | E | 145 | 1619 | + |
| LAKGPASIA | E | 64 | 518 | CTAGCAAAGGGCCCCGCCAGCATCGCT | E | 146 | 1620 | + |
| LAKGVTTAA | E | 65 | 519 | CTAGCAAAGGGCGTGACCACCGCCGCT | E | 147 | 1621 | + |
| TSHVSTSS | E | 66 | 520 | ACCAGCCACGTGAGCACCAGCAGC | F | 148 | 1622 | +++ |
| SGVLFTEG | E | 67 | 521 | AGCGGCGTGCTGTTCACCGAGGGC | E | 149 | 1623 | +++ |
| SGPGTSGT | E | 68 | 522 | AGCGGCCCCGGCACCAGCGGCACC | E | 150 | 1624 | +++ |
| TGEGPTKG | E | 69 | 523 | ACCGGCGAGGGCCCCACCAAGGGC | E | 151 | 1625 | +++ |
| TGDKWPQG | E | 70 | 524 | ACCGGCGACAAGTGGCCCCAGGGC | E | 152 | 1626 | +++ |
| SGREIHFG | E | 71 | 525 | AGCGGCCGGGAGATCCACTTCGGC | E | 153 | 1627 | ++ |
| TSQEKQNS | E | 72 | 526 | ACCAGCCAGGAGAAGCAGAACAGC | E | 154 | 1628 | ++ |
| GTMFNNST | E | 73 | 527 | GGCACCATGTTCAACAACAGCACC | E | 155 | 1629 | ++ |
| TTGMERPT | E | 74 | 528 | ACCACCGGCATGGAGCGGCCCACC | E | 156 | 1630 | ++ |
| TGDERKPG | E | 75 | 529 | ACCGGCGACGAGCGGAAGCCCGGC | E | 157 | 1631 | + |
| CKEITR | E | 76 | 530 | TGCAAGGAGATCACCCGG | E | 158 | 1632 | +++ |
| PDVSLL | E | 77 | 531 | CCCGACGTGAGCCTGCTG | E | 159 | 1633 | +++ |
| RLGRLS | E | 78 | 532 | CGGCTGGGCCGGCTGAGC | E | 160 | 1634 | +++ |
| RLGKVP | E | 79 | 533 | CGGCTGGGCAAGGTGCCC | E | 161 | 1635 | + |
| KFTKAA | E | 80 | 534 | AAGTTCACCAAGGCCGCC | E | 162 | 1636 | + |
| HFPVFS | E | 81 | 535 | CACTTCCCCGTGTTCAGC | E | 163 | 1637 | + |
| NSKKLG | E | 82 | 536 | AACAGCAAGAAGCTGGGC | E | 164 | 1638 | + |
| KAMRTG | E | 83 | 537 | AAGGCCATGCGGACCGGC | E | 165 | 1639 | + |

*AAV2 vectors designed to include SEQ ID NO: 1 with the indicated sequence inclusion. The 8-mers and 9-mers were inserted between amino acid residues 587 and 588 of SEQ ID NO: 1, and the 6-mers were used as replacements for amino acid residues 585 to 590 of SEQ ID NO: 1. +++ means that the modified AAV2 vectors had a performance that placed them within the top 1/3 of the rescued AAV vectors; ++ means the AAV2 vectors had a performance that placed them within the middle 1/3 of the rescued AAV vectors; and + means the AAV2 vectors had a performance that placed them within the bottom 1/3 of the rescued AAV vectors.
**SEQ ID NOs used for Sequence Listing.

TABLE 1F
Amino acid sequences that can be inserted into an AAV capsid polypeptide.

| | | | SEQ | | | | SEQ | |
|---|---|---|---|---|---|---|---|---|
| | SEQ | | ID | | SEQ | | ID | |
| Amino Acid | ID | | NO: | Nucleic Acid Sequence encoding the Amino | ID | | NO: | Activity |
| Sequence | NO: | | ** | Acid Sequence | NO: | | ** | Level* |
| LAFNSCSYA | F | 2 | 538 | CTAGCATTCAACAGCTGCAGCTACGCT | F | 11 | 1640 | +++ |
| LAGGAWDWA | F | 3 | 539 | CTAGCAGGCGGCGCCTGGGACTGGGCT | F | 12 | 1641 | +++ |
| LANADFPLA | F | 4 | 540 | CTAGCAAACGCCGACTTCCCCCTGGCT | F | 13 | 1642 | +++ |
| LAAQDVSWA | F | 5 | 541 | CTAGCAGCCCAGGACGTGAGCTGGGCT | F | 14 | 1643 | ++ |
| LADVGCENA | F | 6 | 542 | CTAGCAGACGTGGGCTGCGAGAACGCT | F | 15 | 1644 | ++ |
| LAATICTEA | F | 7 | 543 | CTAGCAGCCACCATCTGCACCGAGGCT | F | 16 | 1645 | ++ |
| LAEGLSQFA | F | 8 | 544 | CTAGCAGAGGGCCTGAGCCAGTTCGCT | F | 17 | 1646 | + |
| LAKPTGKDA | F | 9 | 545 | CTAGCAAAGCCCACCGGCAAGGACGCT | F | 18 | 1647 | + |
| LARFRGMCA | F | 10 | 546 | CTAGCACGGTTCCGGGGCATGTGCGCT | F | 19 | 1648 | + |

*AAV2 vectors designed to include SEQ ID NO: 1 with the indicated sequence inclusion. The 9-mers were inserted between amino acid residues 587 and 588 of SEQ ID NO: 1. +++ means that the modified AAV2 vectors had a performance that placed them within the top 1/3 of the rescued AAV vectors; ++ means the AAV2 vectors had a performance that placed them within
the middle 1/3 of the rescued AAV vectors; and + means the AAV2 vectors had a performance that placed them within the bottom 1/3 of the rescued AAV vectors.
**SEQ ID NOs used for Sequence Listing.

TABLE 1G
Amino acid sequences that can be inserted into an AAV capsid polypeptide.

| | | | SEQ | | | | SEQ | |
|---|---|---|---|---|---|---|---|---|
| | | | ID | | | | ID | |
| Amino Acid | SEQ ID | | NO: | Nucleic Acid Sequence encoding the Amino Acid | SEQ ID | | NO: | Activity |
| Sequence | NO: | | ** | Sequence | NO: | | ** | Level* |
| LARRKSNEA | G | 2 | 547 | CTAGCACGGCGGAAGAGCAACGAGGCT | G | 78 | 1649 | +++ |
| LAPQNLDEA | G | 3 | 548 | CTAGCACCCCAGAACCTGGACGAGGCT | G | 79 | 1650 | +++ |
| LAMFVWVPA | G | 4 | 549 | CTAGCAATGTTCGTGTGGGTGCCCGCT | G | 80 | 1651 | +++ |
| LACTYTYAA | G | 5 | 550 | CTAGCATGCACCTACACCTACGCCGCT | G | 81 | 1652 | +++ |
| LAKINAQPA | G | 6 | 551 | CTAGCAAAGATCAACGCCCAGCCCGCT | G | 82 | 1653 | +++ |
| LADLSDHHA | G | 7 | 552 | CTAGCAGACCTGAGCGACCACCACGCT | G | 83 | 1654 | +++ |
| LAHEGRQAA | G | 8 | 553 | CTAGCACACGAGGGCCGGCAGGCCGCT | G | 84 | 1655 | +++ |
| LADAHSVTA | G | 9 | 554 | CTAGCAGACGCCCACAGCGTGACCGCT | G | 85 | 1656 | +++ |
| LADLGYPNA | G | 10 | 555 | CTAGCAGACCTGGGCTACCCCAACGCT | G | 86 | 1657 | +++ |
| LAQWFLWRA | G | 11 | 556 | CTAGCACAGTGGTTCCTGTGGCGGGCT | G | 87 | 1658 | +++ |
| LAYAEQSKA | G | 12 | 557 | CTAGCATACGCCGAGCAGAGCAAGGCT | G | 88 | 1659 | +++ |
| LAAKMYNGA | G | 13 | 558 | CTAGCAGCCAAGATGTACAACGGCGCT | G | 89 | 1660 | +++ |
| LAEADKWYA | G | 14 | 559 | CTAGCAGAGGCCGACAAGTGGTACGCT | G | 90 | 1661 | +++ |
| LATVYKSPA | G | 15 | 560 | CTAGCAACCGTGTACAAGAGCCCCGCT | G | 91 | 1662 | +++ |
| LASRTAVAA | G | 16 | 561 | CTAGCAAGCCGGACCGCCGTGGCCGCT | G | 92 | 1663 | +++ |
| LADDMIFVA | G | 17 | 562 | CTAGCAGACGACATGATCTTCGTGGCT | G | 93 | 1664 | +++ |
| LAEFRVGQA | G | 18 | 563 | CTAGCAGAGTTCCGGGTGGGCCAGGCT | G | 94 | 1665 | +++ |
| LAADPTFKA | G | 19 | 564 | CTAGCAGCCGACCCCACCTTCAAGGCT | G | 95 | 1666 | +++ |
| LAVSRHGEA | G | 20 | 565 | CTAGCAGTGAGCCGGCACGGCGAGGCT | G | 96 | 1667 | +++ |
| LAPMRGVYA | G | 21 | 566 | CTAGCACCCATGCGGGGCGTGTACGCT | G | 97 | 1668 | +++ |
| LAFPDKSIA | G | 22 | 567 | CTAGCATTCCCCGACAAGAGCATCGCT | G | 98 | 1669 | +++ |
| LAEFHIPTA | G | 23 | 568 | CTAGCAGAGTTCCACATCCCCACCGCT | G | 99 | 1670 | +++ |
| LALNNDQSA | G | 24 | 569 | CTAGCACTGAACAACGACCAGAGCGCT | G | 100 | 1671 | +++ |
| LAKALGMEA | G | 25 | 570 | CTAGCAAAGGCCCTGGGCATGGAGGCT | G | 101 | 1672 | ++ |
| LAYLPLKYA | G | 26 | 571 | CTAGCATACCTGCCCCTGAAGTACGCT | G | 102 | 1673 | ++ |
| LAHYPSAPA | G | 27 | 572 | CTAGCACACTACCCCAGCGCCCCCGCT | G | 103 | 1674 | ++ |
| LANLKENEA | G | 28 | 573 | CTAGCAAACCTGAAGGAGAACGAGGCT | G | 104 | 1675 | ++ |
| LARSSTLNA | G | 29 | 574 | CTAGCACGGAGCAGCACCCTGAACGCT | G | 105 | 1676 | ++ |
| LAIGERTTA | G | 30 | 575 | CTAGCAATCGGCGAGCGGACCACCGCT | G | 106 | 1677 | ++ |
| LAWIERSMA | G | 31 | 576 | CTAGCATGGATCGAGCGGAGCATGGCT | G | 107 | 1678 | ++ |
| LAYDNTAEA | G | 32 | 577 | CTAGCATACGACAACACCGCCGAGGCT | G | 108 | 1679 | ++ |
| LAERNHGYA | G | 33 | 578 | CTAGCAGAGCGGAACCACGGCTACGCT | G | 109 | 1680 | ++ |
| LADILVGQA | G | 34 | 579 | CTAGCAGACATCCTGGTGGGCCAGGCT | G | 110 | 1681 | ++ |
| LAFNGSTSA | G | 35 | 580 | CTAGCATTCAACGGCAGCACCAGCGCT | G | 111 | 1682 | ++ |
| LADWRTTPA | G | 36 | 581 | CTAGCAGACTGGCGGACCACCCCCGCT | G | 112 | 1683 | ++ |
| LAPSGDRHA | G | 37 | 582 | CTAGCACCCAGCGGCGACCGGCACGCT | G | 113 | 1684 | ++ |
| LAQEAKLTA | G | 38 | 583 | CTAGCACAGGAGGCCAAGCTGACCGCT | G | 114 | 1685 | ++ |
| LAAEHKQTA | G | 39 | 584 | CTAGCAGCCGAGCACAAGCAGACCGCT | G | 115 | 1686 | ++ |
| LAETTLTRA | G | 40 | 585 | CTAGCAGAGACCACCCTGACCCGGGCT | G | 116 | 1687 | ++ |
| LAGESSYLA | G | 41 | 586 | CTAGCAGGCGAGAGCAGCTACCTGGCT | G | 117 | 1688 | ++ |
| LAQVQVEGA | G | 42 | 587 | CTAGCACAGGTGCAGGTGGAGGGCGCT | G | 118 | 1689 | + |
| LAVNRVDMA | G | 43 | 588 | CTAGCAGTGAACCGGGTGGACATGGCT | G | 119 | 1690 | + |
| LAFCTPRYA | G | 44 | 589 | CTAGCATTCTGCACCCCCCGGTACGCT | G | 120 | 1691 | + |
| LADDQAVNA | G | 45 | 590 | CTAGCAGACGACCAGGCCGTGAACGCT | G | 121 | 1692 | + |
| LAREDHRQA | G | 46 | 591 | CTAGCACGGGAGGACCACCGGCAGGCT | G | 122 | 1693 | + |
| LAKVASMNA | G | 47 | 592 | CTAGCAAAGGTGGCCAGCATGAACGCT | G | 123 | 1694 | + |
| LAKDGGKIA | G | 48 | 593 | CTAGCAAAGGACGGCGGCAAGATCGCT | G | 124 | 1695 | + |
| LANRWAIDA | G | 49 | 594 | CTAGCAAACCGGTGGGCCATCGACGCT | G | 125 | 1696 | + |
| LADLTLRAA | G | 50 | 595 | CTAGCAGACCTGACCCTGCGGGCCGCT | G | 126 | 1697 | + |
| LAKEGYRDA | G | 51 | 596 | CTAGCAAAGGAGGGCTACCGGGACGCT | G | 127 | 1698 | + |
| LAGKIEENA | G | 52 | 597 | CTAGCAGGCAAGATCGAGGAGAACGCT | G | 128 | 1699 | + |
| LAPVKHTWA | G | 53 | 598 | CTAGCACCCGTGAAGCACACCTGGGCT | G | 129 | 1700 | + |
| LAQVEVSKA | G | 54 | 599 | CTAGCACAGGTGGAGGTGAGCAAGGCT | G | 130 | 1701 | + |
| LAPLQTQEA | G | 55 | 600 | CTAGCACCCCTGCAGACCCAGGAGGCT | G | 131 | 1702 | 1 |
| LAAHQMASA | G | 56 | 601 | CTAGCAGCCCACCAGATGGCCAGCGCT | G | 132 | 1703 | + |
| LAKKVVDGA | G | 57 | 602 | CTAGCAAAGAAGGTGGTGGACGGCGCT | G | 133 | 1704 | + |
| LAKGPASIA | G | 58 | 603 | CTAGCAAAGGGCCCCGCCAGCATCGCT | G | 134 | 1705 | + |
| LAKGQAQNA | G | 59 | 604 | CTAGCAAAGGGCCAGGCCCAGAACGCT | G | 135 | 1706 | + |
| SGVLFTEG | G | 60 | 605 | AGCGGCGTGCTGTTCACCGAGGGC | G | 136 | 1707 | +++ |
| SGPGTSGT | G | 61 | 606 | AGCGGCCCCGGCACCAGCGGCACC | G | 137 | 1708 | ++ |
| TGEGPTKG | G | 62 | 607 | ACCGGCGAGGGCCCCACCAAGGGC | G | 138 | 1709 | ++ |
| SGSVGYVG | G | 63 | 608 | AGCGGCAGCGTGGGCTACGTGGGC | G | 139 | 1710 | ++ |
| STVPLNMG | G | 64 | 609 | AGCACCGTGCCCCTGAACATGGGC | G | 140 | 1711 | ++ |
| SGREIHFG | G | 65 | 610 | AGCGGCCGGGAGATCCACTTCGGC | G | 141 | 1712 | ++ |
| GTMFNNST | G | 66 | 611 | GGCACCATGTTCAACAACAGCACC | G | 142 | 1713 | + |
| TGDKWPQG | G | 67 | 612 | ACCGGCGACAAGTGGCCCCAGGGC | G | 143 | 1714 | + |
| TSQEKQNS | G | 68 | 613 | ACCAGCCAGGAGAAGCAGAACAGC | G | 144 | 1715 | + |
| TTGMERPT | G | 69 | 614 | ACCACCGGCATGGAGCGGCCCACC | G | 145 | 1716 | + |
| TGDERKPG | G | 70 | 615 | ACCGGCGACGAGCGGAAGCCCGGC | G | 146 | 1717 | + |
| PDVSLL | G | 71 | 616 | CCCGACGTGAGCCTGCTG | G | 147 | 1718 | ++ |
| RTGRNT | G | 72 | 617 | CGGACCGGCCGGAACACC | G | 148 | 1719 | ++ |
| KFTKAA | G | 73 | 618 | AAGTTCACCAAGGCCGCC | G | 149 | 1720 | ++ |
| RLGRLS | G | 74 | 619 | CGGCTGGGCCGGCTGAGC | G | 150 | 1721 | + |
| KAMRTG | G | 75 | 620 | AAGGCCATGCGGACCGGC | G | 151 | 1722 | + |
| HFPVFS | G | 76 | 621 | CACTTCCCCGTGTTCAGC | G | 152 | 1723 | + |
| RLGKVP | G | 77 | 622 | CGGCTGGGCAAGGTGCCC | G | 153 | 1724 | + |

*AAV2 vectors designed to include SEQ ID NO: 1 with the indicated sequence inclusion. The 8-mers and 9-mers were inserted between amino acid residues 587 and 588 of SEQ ID NO: 1, and the 6-mers were used as replacements for amino acid residues 585 to 590 of SEQ ID NO: 1. +++ means that the modified AAV2 vectors had a performance that placed them within the top 1/3 of the rescued AAV vectors; ++ means the AAV2 vectors had a performance that placed them within the middle 1/3 of the rescued AAV vectors; and + means the AAV2 vectors had a performance that placed them within the bottom 1/3 of the rescued AAV vectors.
**SEQ ID NOs used for Sequence Listing.

TABLE 1H
Amino acid sequences that can be inserted into an AAV capsid polypeptide.

| | | | SEQ | | | | SEQ | |
|---|---|---|---|---|---|---|---|---|
| | | | ID | | | | ID | |
| Amino Acid | SEQ ID | | NO: | Nucleic Acid Sequence encoding the Amino | SEQ ID | | NO: | Activity |
| Sequence | NO: | | ** | Acid Sequence | NO: | | ** | Level* |
| LAHGGLREA | H | 2 | 623 | CTAGCACACGGCGGCCTGCGGGAGGCT | H | 259 | 1725 | +++ |
| LATDRTKTA | H | 3 | 624 | CTAGCAACCGACCGGACCAAGACCGCT | H | 260 | 1726 | +++ |
| LARRKSNEA | H | 4 | 625 | CTAGCACGGCGGAAGAGCAACGAGGCT | H | 261 | 1727 | +++ |
| LAVDNIWAA | H | 5 | 626 | CTAGCAGTGGACAACATCTGGGCCGCT | H | 262 | 1728 | +++ |
| LADPLYAVA | H | 6 | 627 | CTAGCAGACCCCCTGTACGCCGTGGCT | H | 263 | 1729 | +++ |
| LAGQAMMQA | H | 7 | 628 | CTAGCAGGCCAGGCCATGATGCAGGCT | H | 264 | 1730 | +++ |
| LAMTKITDA | H | 8 | 629 | CTAGCAATGACCAAGATCACCGACGCT | H | 265 | 1731 | +++ |
| LAFTHGTNA | H | 9 | 630 | CTAGCATTCACCCACGGCACCAACGCT | H | 266 | 1732 | +++ |
| LADPGWKYA | H | 10 | 631 | CTAGCAGACCCCGGCTGGAAGTACGCT | H | 267 | 1733 | +++ |
| LAGGEYETA | H | 11 | 632 | CTAGCAGGCGGCGAGTACGAGACCGCT | H | 268 | 1734 | +++ |
| LAALVDANA | H | 12 | 633 | CTAGCAGCCCTGGTGGACGCCAACGCT | H | 269 | 1735 | +++ |
| LADPTETPA | H | 13 | 634 | CTAGCAGACCCCACCGAGACCCCCGCT | H | 270 | 1736 | +++ |
| LALENIGQA | H | 14 | 635 | CTAGCACTGGAGAACATCGGCCAGGCT | H | 271 | 1737 | +++ |
| LALNDNVGA | H | 15 | 636 | CTAGCACTGAACGACAACGTGGGCGCT | H | 272 | 1738 | +++ |
| LAHRAPDSA | H | 16 | 637 | CTAGCACACCGGGCCCCCGACAGCGCT | H | 273 | 1739 | +++ |
| LAMFVWVPA | H | 17 | 638 | CTAGCAATGTTCGTGTGGGTGCCCGCT | H | 274 | 1740 | +++ |
| LAEGQEDSA | H | 18 | 639 | CTAGCAGAGGGCCAGGAGGACAGCGCT | H | 275 | 1741 | +++ |
| LAPDAKDWA | H | 19 | 640 | CTAGCACCCGACGCCAAGGACTGGGCT | H | 276 | 1742 | +++ |
| LAPHIAKDA | H | 20 | 641 | CTAGCACCCCACATCGCCAAGGACGCT | H | 277 | 1743 | +++ |
| LAAVTEYVA | H | 21 | 642 | CTAGCAGCCGTGACCGAGTACGTGGCT | H | 278 | 1744 | +++ |
| LAFCIKHCA | H | 22 | 643 | CTAGCATTCTGCATCAAGCACTGCGCT | H | 279 | 1745 | +++ |
| LAPPGNPLA | H | 23 | 644 | CTAGCACCCCCCGGCAACCCCCTGGCT | H | 280 | 1746 | +++ |
| LAHWQEEWA | H | 24 | 645 | CTAGCACACTGGCAGGAGGAGTGGGCT | H | 281 | 1747 | +++ |
| LAEFRVGQA | H | 25 | 646 | CTAGCAGAGTTCCGGGTGGGCCAGGCT | H | 282 | 1748 | +++ |
| LAYVIENDA | H | 26 | 647 | CTAGCATACGTGATCGAGAACGACGCT | H | 283 | 1749 | +++ |
| LASTTMNIA | H | 27 | 648 | CTAGCAAGCACCACCATGAACATCGCT | H | 284 | 1750 | +++ |
| LAEFHIPTA | H | 28 | 649 | CTAGCAGAGTTCCACATCCCCACCGCT | H | 285 | 1751 | +++ |
| LASRTAVAA | H | 29 | 650 | CTAGCAAGCCGGACCGCCGTGGCCGCT | H | 286 | 1752 | +++ |
| LAIGSDTSA | H | 30 | 651 | CTAGCAATCGGCAGCGACACCAGCGCT | H | 287 | 1753 | +++ |
| LADDMIFVA | H | 31 | 652 | CTAGCAGACGACATGATCTTCGTGGCT | H | 288 | 1754 | +++ |
| LAKPPPKDA | H | 32 | 653 | CTAGCAAAGCCCCCCCCCAAGGACGCT | H | 289 | 1755 | +++ |
| LAYVALFYA | H | 33 | 654 | CTAGCATACGTGGCCCTGTTCTACGCT | H | 290 | 1756 | +++ |
| LAFQQQRCA | H | 34 | 655 | CTAGCATTCCAGCAGCAGCGGTGCGCT | H | 291 | 1757 | +++ |
| LAAVHLMGA | H | 35 | 656 | CTAGCAGCCGTGCACCTGATGGGCGCT | H | 292 | 1758 | +++ |
| LACPVAEWA | H | 36 | 657 | CTAGCATGCCCCGTGGCCGAGTGGGCT | H | 293 | 1759 | +++ |
| LAVRNEINA | H | 37 | 658 | CTAGCAGTGCGGAACGAGATCAACGCT | H | 294 | 1760 | +++ |
| LADSSKWIA | H | 38 | 659 | CTAGCAGACAGCAGCAAGTGGATCGCT | H | 295 | 1761 | +++ |
| LAVMGNMLA | H | 39 | 660 | CTAGCAGTGATGGGCAACATGCTGGCT | H | 296 | 1762 | +++ |
| LANANTRPA | H | 40 | 661 | CTAGCAAACGCCAACACCCGGCCCGCT | H | 297 | 1763 | +++ |
| LAPNQGTPA | H | 41 | 662 | CTAGCACCCAACCAGGGCACCCCCGCT | H | 298 | 1764 | +++ |
| LAGENHISA | H | 42 | 663 | CTAGCAGGCGAGAACCACATCAGCGCT | H | 299 | 1765 | +++ |
| LALNSGKMA | H | 43 | 664 | CTAGCACTGAACAGCGGCAAGATGGCT | H | 300 | 1766 | +++ |
| LAYDAGGHA | H | 44 | 665 | CTAGCATACGACGCCGGCGGCCACGCT | H | 301 | 1767 | +++ |
| LARAITNEA | H | 45 | 666 | CTAGCACGGGCCATCACCAACGAGGCT | H | 302 | 1768 | +++ |
| LAWNKCPYA | H | 46 | 667 | CTAGCATGGAACAAGTGCCCCTACGCT | H | 303 | 1769 | +++ |
| LANLKENEA | H | 47 | 668 | CTAGCAAACCTGAAGGAGAACGAGGCT | H | 304 | 1770 | +++ |
| LAQVTEMRA | H | 48 | 669 | CTAGCACAGGTGACCGAGATGCGGGCT | H | 305 | 1771 | +++ |
| LARDQLKLA | H | 49 | 670 | CTAGCACGGGACCAGCTGAAGCTGGCT | H | 306 | 1772 | +++ |
| LAVSRHGEA | H | 50 | 671 | CTAGCAGTGAGCCGGCACGGCGAGGCT | H | 307 | 1773 | +++ |
| LAAVGMKMA | H | 51 | 672 | CTAGCAGCCGTGGGCATGAAGATGGCT | H | 308 | 1774 | +++ |
| LAEHKNMHA | H | 52 | 673 | CTAGCAGAGCACAAGAACATGCACGCT | H | 309 | 1775 | +++ |
| LAMNEPVTA | H | 53 | 674 | CTAGCAATGAACGAGCCCGTGACCGCT | H | 310 | 1776 | +++ |
| LAEFVHKGA | H | 54 | 675 | CTAGCAGAGTTCGTGCACAAGGGCGCT | H | 311 | 1777 | +++ |
| LAAYHDSAA | H | 55 | 676 | CTAGCAGCCTACCACGACAGCGCCGCT | H | 312 | 1778 | +++ |
| LAKALGMEA | H | 56 | 677 | CTAGCAAAGGCCCTGGGCATGGAGGCT | H | 313 | 1779 | +++ |
| LAVKGLVNA | H | 57 | 678 | CTAGCAGTGAAGGGCCTGGTGAACGCT | H | 314 | 1780 | +++ |
| LAGIQQHVA | H | 58 | 679 | CTAGCAGGCATCCAGCAGCACGTGGCT | H | 315 | 1781 | +++ |
| LADGGTRPA | H | 59 | 680 | CTAGCAGACGGCGGCACCCGGCCCGCT | H | 316 | 1782 | +++ |
| LAHEPMFNA | H | 60 | 681 | CTAGCACACGAGCCCATGTTCAACGCT | H | 317 | 1783 | +++ |
| LASVSTIQA | H | 61 | 682 | CTAGCAAGCGTGAGCACCATCCAGGCT | H | 318 | 1784 | +++ |
| LASAVMYWA | H | 62 | 683 | CTAGCAAGCGCCGTGATGTACTGGGCT | H | 319 | 1785 | +++ |
| LADKNRLQA | H | 63 | 684 | CTAGCAGACAAGAACCGGCTGCAGGCT | H | 320 | 1786 | +++ |
| LAPMGQNMA | H | 64 | 685 | CTAGCACCCATGGGCCAGAACATGGCT | H | 321 | 1787 | +++ |
| LAAVGVGYA | H | 65 | 686 | CTAGCAGCCGTGGGCGTGGGCTACGCT | H | 322 | 1788 | +++ |
| LAGEFTGKA | H | 66 | 687 | CTAGCAGGCGAGTTCACCGGCAAGGCT | H | 323 | 1789 | +++ |
| LAGEVTKHA | H | 67 | 688 | CTAGCAGGCGAGGTGACCAAGCACGCT | H | 324 | 1790 | +++ |
| LAITQSDIA | H | 68 | 689 | CTAGCAATCACCCAGAGCGACATCGCT | H | 325 | 1791 | +++ |
| LANEAQGKA | H | 69 | 690 | CTAGCAAACGAGGCCCAGGGCAAGGCT | H | 326 | 1792 | +++ |
| LAAKPKDGA | H | 70 | 691 | CTAGCAGCCAAGCCCAAGGACGGCGCT | H | 327 | 1793 | +++ |
| LAAGQAQSA | H | 71 | 692 | CTAGCAGCCGGCCAGGCCCAGAGCGCT | H | 328 | 1794 | +++ |
| LAPMRGVYA | H | 72 | 693 | CTAGCACCCATGCGGGGCGTGTACGCT | H | 329 | 1795 | +++ |
| LASRLNSPA | H | 73 | 694 | CTAGCAAGCCGGCTGAACAGCCCCGCT | H | 330 | 1796 | +++ |
| LACYPPTCA | H | 74 | 695 | CTAGCATGCTACCCCCCCACCTGCGCT | H | 331 | 1797 | +++ |
| LAKVSKGDA | H | 75 | 696 | CTAGCAAAGGTGAGCAAGGGCGACGCT | H | 332 | 1798 | +++ |
| LAGERETVA | H | 76 | 697 | CTAGCAGGCGAGCGGGAGACCGTGGCT | H | 333 | 1799 | ++ |
| LAYITEKSA | H | 77 | 698 | CTAGCATACATCACCGAGAAGAGCGCT | H | 334 | 1800 | ++ |
| LASADHQPA | H | 78 | 699 | CTAGCAAGCGCCGACCACCAGCCCGCT | H | 335 | 1801 | ++ |
| LAHIQEPKA | H | 79 | 700 | CTAGCACACATCCAGGAGCCCAAGGCT | H | 336 | 1802 | ++ |
| LAFNGSTSA | H | 80 | 701 | CTAGCATTCAACGGCAGCACCAGCGCT | H | 337 | 1803 | ++ |
| LAEFDGDGA | H | 81 | 702 | CTAGCAGAGTTCGACGGCGACGGCGCT | H | 338 | 1804 | ++ |
| LAWKCKGTA | H | 82 | 703 | CTAGCATGGAAGTGCAAGGGCACCGCT | H | 339 | 1805 | ++ |
| LAMGHNYVA | H | 83 | 704 | CTAGCAATGGGCCACAACTACGTGGCT | H | 340 | 1806 | ++ |
| LAVTPPWYA | H | 84 | 705 | CTAGCAGTGACCCCCCCCTGGTACGCT | H | 341 | 1807 | ++ |
| LAGHAACFA | H | 85 | 706 | CTAGCAGGCCACGCCGCCTGCTTCGCT | H | 342 | 1808 | ++ |
| LAAANHDNA | H | 86 | 707 | CTAGCAGCCGCCAACCACGACAACGCT | H | 343 | 1809 | ++ |
| LAKDHHVIA | H | 87 | 708 | CTAGCAAAGGACCACCACGTGATCGCT | H | 344 | 1810 | ++ |
| LALHQERQA | H | 88 | 709 | CTAGCACTGCACCAGGAGCGGCAGGCT | H | 345 | 1811 | ++ |
| LADMTRFHA | H | 89 | 710 | CTAGCAGACATGACCCGGTTCCACGCT | H | 346 | 1812 | ++ |
| LATMHNFEA | H | 90 | 711 | CTAGCAACCATGCACAACTTCGAGGCT | H | 347 | 1813 | ++ |
| LAEYHNNDA | H | 91 | 712 | CTAGCAGAGTACCACAACAACGACGCT | H | 348 | 1814 | ++ |
| LAGKTISGA | H | 92 | 713 | CTAGCAGGCAAGACCATCAGCGGCGCT | H | 349 | 1815 | ++ |
| LADFPSEKA | H | 93 | 714 | CTAGCAGACTTCCCCAGCGAGAAGGCT | H | 350 | 1816 | ++ |
| LALKLRTEA | H | 94 | 715 | CTAGCACTGAAGCTGCGGACCGAGGCT | H | 351 | 1817 | ++ |
| LAISQPNPA | H | 95 | 716 | CTAGCAATCAGCCAGCCCAACCCCGCT | H | 352 | 1818 | ++ |
| LAFGFSADA | H | 96 | 717 | CTAGCATTCGGCTTCAGCGCCGACGCT | H | 353 | 1819 | ++ |
| LAKDKGLIA | H | 97 | 718 | CTAGCAAAGGACAAGGGCCTGATCGCT | H | 354 | 1820 | ++ |
| LAIGERTTA | H | 98 | 719 | CTAGCAATCGGCGAGCGGACCACCGCT | H | 355 | 1821 | ++ |
| LAPQKQPVA | H | 99 | 720 | CTAGCACCCCAGAAGCAGCCCGTGGCT | H | 356 | 1822 | ++ |
| LAKFMWIHA | H | 100 | 721 | CTAGCAAAGTTCATGTGGATCCACGCT | H | 357 | 1823 | ++ |
| LAQEQNAAA | H | 101 | 722 | CTAGCACAGGAGCAGAACGCCGCCGCT | H | 358 | 1824 | ++ |
| LAQVQVEGA | H | 102 | 723 | CTAGCACAGGTGCAGGTGGAGGGCGCT | H | 359 | 1825 | ++ |
| LAALNSQTA | H | 103 | 724 | CTAGCAGCCCTGAACAGCCAGACCGCT | H | 360 | 1826 | ++ |
| LAHHKESIA | H | 104 | 725 | CTAGCACACCACAAGGAGAGCATCGCT | H | 361 | 1827 | ++ |
| LAPCYEPPA | H | 105 | 726 | CTAGCACCCTGCTACGAGCCCCCCGCT | H | 362 | 1828 | ++ |
| LANQTTREA | H | 106 | 727 | CTAGCAAACCAGACCACCCGGGAGGCT | H | 363 | 1829 | ++ |
| LADTHTTYA | H | 107 | 728 | CTAGCAGACACCCACACCACCTACGCT | H | 364 | 1830 | ++ |
| LAQKKLLEA | H | 108 | 729 | CTAGCACAGAAGAAGCTGCTGGAGGCT | H | 365 | 1831 | ++ |
| LAVDIMRGA | H | 109 | 730 | CTAGCAGTGGACATCATGCGGGGCGCT | H | 366 | 1832 | ++ |
| LAPIQLAQA | H | 110 | 731 | CTAGCACCCATCCAGCTGGCCCAGGCT | H | 367 | 1833 | ++ |
| LANIVQQDA | H | 111 | 732 | CTAGCAAACATCGTGCAGCAGGACGCT | H | 368 | 1834 | ++ |
| LAKSAGILA | H | 112 | 733 | CTAGCAAAGAGCGCCGGCATCCTGGCT | H | 369 | 1835 | ++ |
| LAHDLHGGA | H | 113 | 734 | CTAGCACACGACCTGCACGGCGGCGCT | H | 370 | 1836 | ++ |
| LAKGFKTEA | H | 114 | 735 | CTAGCAAAGGGCTTCAAGACCGAGGCT | H | 371 | 1837 | ++ |
| LAGSMAKLA | H | 115 | 736 | CTAGCAGGCAGCATGGCCAAGCTGGCT | H | 372 | 1838 | ++ |
| LAGFMEYYA | H | 116 | 737 | CTAGCAGGCTTCATGGAGTACTACGCT | H | 373 | 1839 | ++ |
| LAQGNNTGA | H | 117 | 738 | CTAGCACAGGGCAACAACACCGGCGCT | H | 374 | 1840 | ++ |
| LAVRTGSMA | H | 118 | 739 | CTAGCAGTGCGGACCGGCAGCATGGCT | H | 375 | 1841 | ++ |
| LAYGQQTTA | H | 119 | 740 | CTAGCATACGGCCAGCAGACCACCGCT | H | 376 | 1842 | ++ |
| LADCMMINA | H | 120 | 741 | CTAGCAGACTGCATGATGATCAACGCT | H | 377 | 1843 | ++ |
| LAGVVKTDA | H | 121 | 742 | CTAGCAGGCGTGGTGAAGACCGACGCT | H | 378 | 1844 | ++ |
| LASENLNRA | H | 122 | 743 | CTAGCAAGCGAGAACCTGAACCGGGCT | H | 379 | 1845 | ++ |
| LAQAGDTAA | H | 123 | 744 | CTAGCACAGGCCGGCGACACCGCCGCT | H | 380 | 1846 | ++ |
| LAMHESYNA | H | 124 | 745 | CTAGCAATGCACGAGAGCTACAACGCT | H | 381 | 1847 | ++ |
| LAVNRVDMA | H | 125 | 746 | CTAGCAGTGAACCGGGTGGACATGGCT | H | 382 | 1848 | ++ |
| LAVPKADAA | H | 126 | 747 | CTAGCAGTGCCCAAGGCCGACGCCGCT | H | 383 | 1849 | ++ |
| LAPSGDRHA | H | 127 | 748 | CTAGCACCCAGCGGCGACCGGCACGCT | H | 384 | 1850 | ++ |
| LAFRTMEYA | H | 128 | 749 | CTAGCATTCCGGACCATGGAGTACGCT | H | 385 | 1851 | ++ |
| LATWCLVWA | H | 129 | 750 | CTAGCAACCTGGTGCCTGGTGTGGGCT | H | 386 | 1852 | ++ |
| LAEGMTGIA | H | 130 | 751 | CTAGCAGAGGGCATGACCGGCATCGCT | H | 387 | 1853 | ++ |
| LAKEGYRDA | H | 131 | 752 | CTAGCAAAGGAGGGCTACCGGGACGCT | H | 388 | 1854 | ++ |
| LAAVTKDSA | H | 132 | 753 | CTAGCAGCCGTGACCAAGGACAGCGCT | H | 389 | 1855 | ++ |
| LAPLQTQEA | H | 133 | 754 | CTAGCACCCCTGCAGACCCAGGAGGCT | H | 390 | 1856 | ++ |
| LADHGNKPA | H | 134 | 755 | CTAGCAGACCACGGCAACAAGCCCGCT | H | 391 | 1857 | ++ |
| LADVVSVSA | H | 135 | 756 | CTAGCAGACGTGGTGAGCGTGAGCGCT | H | 392 | 1858 | ++ |
| LAQVEVSKA | H | 136 | 757 | CTAGCACAGGTGGAGGTGAGCAAGGCT | H | 393 | 1859 | ++ |
| LAPSKMGLA | H | 137 | 758 | CTAGCACCCAGCAAGATGGGCCTGGCT | H | 394 | 1860 | ++ |
| LAWIERSMA | H | 138 | 759 | CTAGCATGGATCGAGCGGAGCATGGCT | H | 395 | 1861 | ++ |
| LAYDNTAEA | H | 139 | 760 | CTAGCATACGACAACACCGCCGAGGCT | H | 396 | 1862 | ++ |
| LAHDWGAPA | H | 140 | 761 | CTAGCACACGACTGGGGCGCCCCCGCT | H | 397 | 1863 | ++ |
| LAMDREIKA | H | 141 | 762 | CTAGCAATGGACCGGGAGATCAAGGCT | H | 398 | 1864 | ++ |
| LAVFQETGA | H | 142 | 763 | CTAGCAGTGTTCCAGGAGACCGGCGCT | H | 399 | 1865 | ++ |
| LAKDGGKIA | H | 143 | 764 | CTAGCAAAGGACGGCGGCAAGATCGCT | H | 400 | 1866 | ++ |
| LALNAAATA | H | 144 | 765 | CTAGCACTGAACGCCGCCGCCACCGCT | H | 401 | 1867 | ++ |
| LAIPVEANA | H | 145 | 766 | CTAGCAATCCCCGTGGAGGCCAACGCT | H | 402 | 1868 | ++ |
| LARGTLMTA | H | 146 | 767 | CTAGCACGGGGCACCCTGATGACCGCT | H | 403 | 1869 | ++ |
| LAGDIVRNA | H | 147 | 768 | CTAGCAGGCGACATCGTGCGGAACGCT | H | 404 | 1870 | ++ |
| LAMLACKGA | H | 148 | 769 | CTAGCAATGCTGGCCTGCAAGGGCGCT | H | 405 | 1871 | ++ |
| LADLSDHHA | H | 149 | 770 | CTAGCAGACCTGAGCGACCACCACGCT | H | 406 | 1872 | ++ |
| LANMMAAGA | H | 150 | 771 | CTAGCAAACATGATGGCCGCCGGCGCT | H | 407 | 1873 | ++ |
| LALPEGRFA | H | 151 | 772 | CTAGCACTGCCCGAGGGCCGGTTCGCT | H | 408 | 1874 | ++ |
| LAKGQAQNA | H | 152 | 773 | CTAGCAAAGGGCCAGGCCCAGAACGCT | H | 409 | 1875 | + |
| LAAVKAVVA | H | 153 | 774 | CTAGCAGCCGTGAAGGCCGTGGTGGCT | H | 410 | 1876 | + |
| LANQGFLTA | H | 154 | 775 | CTAGCAAACCAGGGCTTCCTGACCGCT | H | 411 | 1877 | + |
| LAETTLTRA | H | 155 | 776 | CTAGCAGAGACCACCCTGACCCGGGCT | H | 412 | 1878 | + |
| LADGIQKNA | H | 156 | 777 | CTAGCAGACGGCATCCAGAAGAACGCT | H | 413 | 1879 | + |
| LANAMEKHA | H | 157 | 778 | CTAGCAAACGCCATGGAGAAGCACGCT | H | 414 | 1880 | + |
| LADIVGESA | H | 158 | 779 | CTAGCAGACATCGTGGGCGAGAGCGCT | H | 415 | 1881 | + |
| LANVTLQSA | H | 159 | 780 | CTAGCAAACGTGACCCTGCAGAGCGCT | H | 416 | 1882 | + |
| LAFPCIKEA | H | 160 | 781 | CTAGCATTCCCCTGCATCAAGGAGGCT | H | 417 | 1883 | + |
| LADEGREHA | H | 161 | 782 | CTAGCAGACGAGGGCCGGGAGCACGCT | H | 418 | 1884 | + |
| LAREDHRQA | H | 162 | 783 | CTAGCACGGGAGGACCACCGGCAGGCT | H | 419 | 1885 | + |
| LAQNQWTDA | H | 163 | 784 | CTAGCACAGAACCAGTGGACCGACGCT | H | 420 | 1886 | + |
| LAQNQKQIA | H | 164 | 785 | CTAGCACAGAACCAGAAGCAGATCGCT | H | 421 | 1887 | + |
| LATPNVNAA | H | 165 | 786 | CTAGCAACCCCCAACGTGAACGCCGCT | H | 422 | 1888 | + |
| LAPGNDLWA | H | 166 | 787 | CTAGCACCCGGCAACGACCTGTGGGCT | H | 423 | 1889 | + |
| LAMLGGTSA | H | 167 | 788 | CTAGCAATGCTGGGCGGCACCAGCGCT | H | 424 | 1890 | + |
| LAKKVVDGA | H | 168 | 789 | CTAGCAAAGAAGGTGGTGGACGGCGCT | H | 425 | 1891 | + |
| LAGGSKLEA | H | 169 | 790 | CTAGCAGGCGGCAGCAAGCTGGAGGCT | H | 426 | 1892 | + |
| LALYQCDTA | H | 170 | 791 | CTAGCACTGTACCAGTGCGACACCGCT | H | 427 | 1893 | + |
| LAHQGAIIA | H | 171 | 792 | CTAGCACACCAGGGCGCCATCATCGCT | H | 428 | 1894 | + |
| LAGFHAIEA | H | 172 | 793 | CTAGCAGGCTTCCACGCCATCGAGGCT | H | 429 | 1895 | + |
| LANEGTIRA | H | 173 | 794 | CTAGCAAACGAGGGCACCATCCGGGCT | H | 430 | 1896 | + |
| LADPMPTHA | H | 174 | 795 | CTAGCAGACCCCATGCCCACCCACGCT | H | 431 | 1897 | + |
| LAGKIEENA | H | 175 | 796 | CTAGCAGGCAAGATCGAGGAGAACGCT | H | 432 | 1898 | 1 |
| LAPVKHTWA | H | 176 | 797 | CTAGCACCCGTGAAGCACACCTGGGCT | H | 433 | 1899 | + |
| LARSSTLNA | H | 177 | 798 | CTAGCACGGAGCAGCACCCTGAACGCT | H | 434 | 1900 | + |
| LADDQAVNA | H | 178 | 799 | CTAGCAGACGACCAGGCCGTGAACGCT | H | 435 | 1901 | + |
| LATKSTGSA | H | 179 | 800 | CTAGCAACCAAGAGCACCGGCAGCGCT | H | 436 | 1902 | + |
| LAFLRGPEA | H | 180 | 801 | CTAGCATTCCTGCGGGGCCCCGAGGCT | H | 437 | 1903 | + |
| LADENLQAA | H | 181 | 802 | CTAGCAGACGAGAACCTGCAGGCCGCT | H | 438 | 1904 | + |
| LANADFPLA | H | 182 | 803 | CTAGCAAACGCCGACTTCCCCCTGGCT | H | 439 | 1905 | + |
| LAPTTIGGA | H | 183 | 804 | CTAGCACCCACCACCATCGGCGGCGCT | H | 440 | 1906 | + |
| LASACMRAA | H | 184 | 805 | CTAGCAAGCGCCTGCATGCGGGCCGCT | H | 441 | 1907 | + |
| LAKVASMNA | H | 185 | 806 | CTAGCAAAGGTGGCCAGCATGAACGCT | H | 442 | 1908 | + |
| LALNNDQSA | H | 186 | 807 | CTAGCACTGAACAACGACCAGAGCGCT | H | 443 | 1909 | + |
| LAGGKQALA | H | 187 | 808 | CTAGCAGGCGGCAAGCAGGCCCTGGCT | H | 444 | 1910 | + |
| LAVAVDCFA | H | 188 | 809 | CTAGCAGTGGCCGTGGACTGCTTCGCT | H | 445 | 1911 | + |
| LALETTQRA | H | 189 | 810 | CTAGCACTGGAGACCACCCAGCGGGCT | H | 446 | 1912 | + |
| LAPNTPKIA | H | 190 | 811 | CTAGCACCCAACACCCCCAAGATCGCT | H | 447 | 1913 | + |
| LASIGFSAA | H | 191 | 812 | CTAGCAAGCATCGGCTTCAGCGCCGCT | H | 448 | 1914 | + |
| LADVGCENA | H | 192 | 813 | CTAGCAGACGTGGGCTGCGAGAACGCT | H | 449 | 1915 | + |
| LAGSRVNFA | H | 193 | 814 | CTAGCAGGCAGCCGGGTGAACTTCGCT | H | 450 | 1916 | + |
| LADLTLRAA | H | 194 | 815 | CTAGCAGACCTGACCCTGCGGGCCGCT | H | 451 | 1917 | + |
| LAKGQSQGA | H | 195 | 816 | CTAGCAAAGGGCCAGAGCCAGGGCGCT | H | 452 | 1918 | + |
| LAHTPDTFA | H | 196 | 817 | CTAGCACACACCCCCGACACCTTCGCT | H | 453 | 1919 | + |
| LAAHQMASA | H | 197 | 818 | CTAGCAGCCCACCAGATGGCCAGCGCT | H | 454 | 1920 | + |
| LACRSDCVA | H | 198 | 819 | CTAGCATGCCGGAGCGACTGCGTGGCT | H | 455 | 1921 | + |
| LANTWCKGA | H | 199 | 820 | CTAGCAAACACCTGGTGCAAGGGCGCT | H | 456 | 1922 | + |
| LAAEHKQTA | H | 200 | 821 | CTAGCAGCCGAGCACAAGCAGACCGCT | H | 457 | 1923 | + |
| LAAEHIGRA | H | 201 | 822 | CTAGCAGCCGAGCACATCGGCCGGGCT | H | 458 | 1924 | + |
| LAEQVLEKA | H | 202 | 823 | CTAGCAGAGCAGGTGCTGGAGAAGGCT | H | 459 | 1925 | + |
| LAYFHSYAA | H | 203 | 824 | CTAGCATACTTCCACAGCTACGCCGCT | H | 460 | 1926 | + |
| LAEPPDPPA | H | 204 | 825 | CTAGCAGAGCCCCCCGACCCCCCCGCT | H | 461 | 1927 | + |
| LATRADIFA | H | 205 | 826 | CTAGCAACCCGGGCCGACATCTTCGCT | H | 462 | 1928 | + |
| LADTYKPYA | H | 206 | 827 | CTAGCAGACACCTACAAGCCCTACGCT | H | 463 | 1929 | + |
| LANRLSNVA | H | 207 | 828 | CTAGCAAACCGGCTGAGCAACGTGGCT | H | 464 | 1930 | + |
| LAMTNTFLA | H | 208 | 829 | CTAGCAATGACCAACACCTTCCTGGCT | H | 465 | 1931 | + |
| LAGESSYLA | H | 209 | 830 | CTAGCAGGCGAGAGCAGCTACCTGGCT | H | 466 | 1932 | + |
| LANRWAIDA | H | 210 | 831 | CTAGCAAACCGGTGGGCCATCGACGCT | H | 467 | 1933 | + |
| LAETVKYMA | H | 211 | 832 | CTAGCAGAGACCGTGAAGTACATGGCT | H | 468 | 1934 | + |
| LAMKYVFDA | H | 212 | 833 | CTAGCAATGAAGTACGTGTTCGACGCT | H | 469 | 1935 | + |
| LAKRDKPTA | H | 213 | 834 | CTAGCAAAGCGGGACAAGCCCACCGCT | H | 470 | 1936 | + |
| LADWRTTPA | H | 214 | 835 | CTAGCAGACTGGCGGACCACCCCCGCT | H | 471 | 1937 | + |
| LAKGVTTAA | H | 215 | 836 | CTAGCAAAGGGCGTGACCACCGCCGCT | H | 472 | 1938 | + |
| LAFCTPRYA | H | 216 | 837 | CTAGCATTCTGCACCCCCCGGTACGCT | H | 473 | 1939 | + |
| LANLKNFSA | H | 217 | 838 | CTAGCAAACCTGAAGAACTTCAGCGCT | H | 474 | 1940 | + |
| LAHYPSAPA | H | 218 | 839 | CTAGCACACTACCCCAGCGCCCCCGCT | H | 475 | 1941 | + |
| LAQEAKLTA | H | 219 | 840 | CTAGCACAGGAGGCCAAGCTGACCGCT | H | 476 | 1942 | + |
| LAVEPGPPA | H | 220 | 841 | CTAGCAGTGGAGCCCGGCCCCCCCGCT | H | 477 | 1943 | + |
| LAKGPASIA | H | 221 | 842 | CTAGCAAAGGGCCCCGCCAGCATCGCT | H | 478 | 1944 | + |
| LAEGLSQFA | H | 222 | 843 | CTAGCAGAGGGCCTGAGCCAGTTCGCT | H | 479 | 1945 | + |
| LAKPTGKDA | H | 223 | 844 | CTAGCAAAGCCCACCGGCAAGGACGCT | H | 480 | 1946 | + |
| LAKGMKEGA | H | 224 | 845 | CTAGCAAAGGGCATGAAGGAGGGCGCT | H | 481 | 1947 | + |
| TSHVSTSS | H | 225 | 846 | ACCAGCCACGTGAGCACCAGCAGC | H | 482 | 1948 | +++ |
| SGVLFTEG | H | 226 | 847 | AGCGGCGTGCTGTTCACCGAGGGC | H | 483 | 1949 | +++ |
| TSGMGAIG | H | 227 | 848 | ACCAGCGGCATGGGCGCCATCGGC | H | 484 | 1950 | +++ |
| TGDSEQQT | H | 228 | 849 | ACCGGCGACAGCGAGCAGCAGACC | H | 485 | 1951 | +++ |
| SGSVGYVG | H | 229 | 850 | AGCGGCAGCGTGGGCTACGTGGGC | H | 486 | 1952 | +++ |
| TGDKWPQG | H | 230 | 851 | ACCGGCGACAAGTGGCCCCAGGGC | H | 487 | 1953 | +++ |
| SGEMTKPG | H | 231 | 852 | AGCGGCGAGATGACCAAGCCCGGC | H | 488 | 1954 | +++ |
| SGPGTSGT | H | 232 | 853 | AGCGGCCCCGGCACCAGCGGCACC | H | 489 | 1955 | ++ |
| SSENNKGG | H | 233 | 854 | AGCAGCGAGAACAACAAGGGCGGC | H | 490 | 1956 | ++ |
| TTENHKPG | H | 234 | 855 | ACCACCGAGAACCACAAGCCCGGC | H | 491 | 1957 | ++ |
| TSQEKQNS | H | 235 | 856 | ACCAGCCAGGAGAAGCAGAACAGC | H | 492 | 1958 | ++ |
| GTMFNNST | H | 236 | 857 | GGCACCATGTTCAACAACAGCACC | H | 493 | 1959 | ++ |
| SGPKETGG | H | 237 | 858 | AGCGGCCCCAAGGAGACCGGCGGC | H | 494 | 1960 | + |
| SGIDTRLG | H | 238 | 859 | AGCGGCATCGACACCCGGCTGGGC | H | 495 | 1961 | 1 |
| TTGMERPT | H | 239 | 860 | ACCACCGGCATGGAGCGGCCCACC | H | 496 | 1962 | + |
| TGEGPTKG | H | 240 | 861 | ACCGGCGAGGGCCCCACCAAGGGC | H | 497 | 1963 | + |
| TGPGTHNG | H | 241 | 862 | ACCGGCCCCGGCACCCACAACGGC | H | 498 | 1964 | + |
| SGREIHFG | H | 242 | 863 | AGCGGCCGGGAGATCCACTTCGGC | H | 499 | 1965 | + |
| TGDERKPG | H | 243 | 864 | ACCGGCGACGAGCGGAAGCCCGGC | H | 500 | 1966 | + |
| STTKPM | H | 244 | 865 | AGCACCACCAAGCCCATG | H | 501 | 1967 | +++ |
| CKEITR | H | 245 | 866 | TGCAAGGAGATCACCCGG | H | 502 | 1968 | +++ |
| PDVSLL | H | 246 | 867 | CCCGACGTGAGCCTGCTG | H | 503 | 1969 | +++ |
| FNKVLH | H | 247 | 868 | TTCAACAAGGTGCTGCAC | H | 504 | 1970 | +++ |
| RLGRLS | H | 248 | 869 | CGGCTGGGCCGGCTGAGC | H | 505 | 1971 | +++ |
| RTGRNT | H | 249 | 870 | CGGACCGGCCGGAACACC | H | 506 | 1972 | ++ |
| RALKIS | H | 250 | 871 | CGGGCCCTGAAGATCAGC | H | 507 | 1973 | ++ |
| NIKKAA | H | 251 | 872 | AACATCAAGAAGGCCGCC | H | 508 | 1974 | ++ |
| RPGRVS | H | 252 | 873 | CGGCCCGGCCGGGTGAGC | H | 509 | 1975 | ++ |
| RLGKVP | H | 253 | 874 | CGGCTGGGCAAGGTGCCC | H | 510 | 1976 | + |
| NSKKLG | H | 254 | 875 | AACAGCAAGAAGCTGGGC | H | 511 | 1977 | + |
| KAMRTG | H | 255 | 876 | AAGGCCATGCGGACCGGC | H | 512 | 1978 | + |
| KFTKAA | H | 256 | 877 | AAGTTCACCAAGGCCGCC | H | 513 | 1979 | + |
| HSKDGA | H | 257 | 878 | CACAGCAAGGACGGCGCC | H | 514 | 1980 | + |
| HFPVFS | H | 258 | 879 | CACTTCCCCGTGTTCAGC | H | 515 | 1981 | + |

*AAV2 vectors designed to include SEQ ID NO: 1 with the indicated sequence inclusion. The 8-mers and 9-mers were inserted between amino acid residues 587 and 588 of SEQ ID NO: 1, and the 6-mers were used as replacements for amino acid residues 585 to 590 of SEQ ID NO: 1. +++ means that the modified AAV2 vectors had a performance that placed them within the top 1/3 of the rescued AAV vectors; ++ means the AAV2 vectors had a performance that placed them within the middle 1/3 of the rescued AAV vectors; and + means the AAV2 vectors had a performance that placed them within the bottom 1/3 of the rescued AAV vectors.
**SEQ ID NOs used for Sequence Listing.

TABLE 11
Amino acid sequences that can be inserted into an AAV capsid polypeptide.

| | | | SEQ | | | | SEQ | |
|---|---|---|---|---|---|---|---|---|
| | SEQ | | ID | | | | ID | |
| Amino Acid | ID | | NO: | Nucleic Acid Sequence encoding the Amino | SEQ ID | | NO: | Activity |
| Sequence | NO: | | ** | Acid Sequence | NO: | | ** | Level* |
| LAWRIMKHA | I | 2 | 880 | CTAGCATGGCGGATCATGAAGCACGCT | I | 75 | 1982 | +++ |
| LASNMERIA | I | 3 | 881 | CTAGCAAGCAACATGGAGCGGATCGCT | I | 76 | 1983 | +++ |
| LADYRNDKA | I | 4 | 882 | CTAGCAGACTACCGGAACGACAAGGCT | I | 77 | 1984 | +++ |
| LAPNQGTPA | I | 5 | 883 | CTAGCACCCAACCAGGGCACCCCCGCT | I | 78 | 1985 | +++ |
| LAKPPPKDA | I | 6 | 884 | CTAGCAAAGCCCCCCCCCAAGGACGCT | I | 79 | 1986 | +++ |
| LALPEGRFA | I | 7 | 885 | CTAGCACTGCCCGAGGGCCGGTTCGCT | I | 80 | 1987 | +++ |
| LAHPGTTFA | I | 8 | 886 | CTAGCACACCCCGGCACCACCTTCGCT | I | 81 | 1988 | +++ |
| LARGVAPIA | I | 9 | 887 | CTAGCACGGGGCGTGGCCCCCATCGCT | I | 82 | 1989 | +++ |
| LAHRAPDSA | I | 10 | 888 | CTAGCACACCGGGCCCCCGACAGCGCT | I | 83 | 1990 | +++ |
| LAELALIKA | I | 11 | 889 | CTAGCAGAGCTGGCCCTGATCAAGGCT | I | 84 | 1991 | +++ |
| LAVAVDCFA | I | 12 | 890 | CTAGCAGTGGCCGTGGACTGCTTCGCT | I | 85 | 1992 | +++ |
| LACSSTWYA | I | 13 | 891 | CTAGCATGCAGCAGCACCTGGTACGCT | I | 86 | 1993 | +++ |
| LAELDFKWA | I | 14 | 892 | CTAGCAGAGCTGGACTTCAAGTGGGCT | I | 87 | 1994 | +++ |
| LAHEPMFNA | I | 15 | 893 | CTAGCACACGAGCCCATGTTCAACGCT | I | 88 | 1995 | +++ |
| LAKDGMIGA | I | 16 | 894 | CTAGCAAAGGACGGCATGATCGGCGCT | I | 89 | 1996 | +++ |
| LAHEGRQAA | I | 17 | 895 | CTAGCACACGAGGGCCGGCAGGCCGCT | I | 90 | 1997 | +++ |
| LAEYHNNDA | I | 18 | 896 | CTAGCAGAGTACCACAACAACGACGCT | I | 91 | 1998 | +++ |
| LAAEHIGRA | I | 19 | 897 | CTAGCAGCCGAGCACATCGGCCGGGCT | I | 92 | 1999 | +++ |
| LAEPFPVAA | I | 20 | 898 | CTAGCAGAGCCCTTCCCCGTGGCCGCT | I | 93 | 2000 | +++ |
| LAHIQEPKA | I | 21 | 899 | CTAGCACACATCCAGGAGCCCAAGGCT | I | 94 | 2001 | +++ |
| LAFNGSTSA | I | 22 | 900 | CTAGCATTCAACGGCAGCACCAGCGCT | I | 95 | 2002 | +++ |
| LADNCWCCA | I | 23 | 901 | CTAGCAGACAACTGCTGGTGCTGCGCT | I | 96 | 2003 | +++ |
| LACPVAEWA | I | 24 | 902 | CTAGCATGCCCCGTGGCCGAGTGGGCT | I | 97 | 2004 | ++ |
| LAMGHNYVA | I | 25 | 903 | CTAGCAATGGGCCACAACTACGTGGCT | I | 98 | 2005 | ++ |
| LANQGFLTA | I | 26 | 904 | CTAGCAAACCAGGGCTTCCTGACCGCT | I | 99 | 2006 | ++ |
| LAPMRGVYA | I | 27 | 905 | CTAGCACCCATGCGGGGCGTGTACGCT | I | 100 | 2007 | ++ |
| LANEAQGKA | I | 28 | 906 | CTAGCAAACGAGGCCCAGGGCAAGGCT | I | 101 | 2008 | ++ |
| LAQKKLLEA | I | 29 | 907 | CTAGCACAGAAGAAGCTGCTGGAGGCT | I | 102 | 2009 | ++ |
| LAPCYEPPA | I | 30 | 908 | CTAGCACCCTGCTACGAGCCCCCCGCT | I | 103 | 2010 | ++ |
| LAISQPNPA | I | 31 | 909 | CTAGCAATCAGCCAGCCCAACCCCGCT | I | 104 | 2011 | ++ |
| LAQVQVEGA | I | 32 | 910 | CTAGCACAGGTGCAGGTGGAGGGCGCT | I | 105 | 2012 | ++ |
| LAQAGDTAA | I | 33 | 911 | CTAGCACAGGCCGGCGACACCGCCGCT | I | 106 | 2013 | ++ |
| LARSSTLNA | I | 34 | 912 | CTAGCACGGAGCAGCACCCTGAACGCT | I | 107 | 2014 | ++ |
| LAEFRVGQA | I | 35 | 913 | CTAGCAGAGTTCCGGGTGGGCCAGGCT | I | 108 | 2015 | ++ |
| LAGSMAKLA | I | 36 | 914 | CTAGCAGGCAGCATGGCCAAGCTGGCT | I | 109 | 2016 | ++ |
| LAPIQLAQA | I | 37 | 915 | CTAGCACCCATCCAGCTGGCCCAGGCT | I | 110 | 2017 | ++ |
| LAPSGDRHA | I | 38 | 916 | CTAGCACCCAGCGGCGACCGGCACGCT | I | 111 | 2018 | ++ |
| LANRLSNVA | I | 39 | 917 | CTAGCAAACCGGCTGAGCAACGTGGCT | I | 112 | 2019 | ++ |
| LADPMPTHA | I | 40 | 918 | CTAGCAGACCCCATGCCCACCCACGCT | I | 113 | 2020 | ++ |
| LAVEPGPPA | I | 41 | 919 | CTAGCAGTGGAGCCCGGCCCCCCCGCT | I | 114 | 2021 | ++ |
| LAYFHSYAA | I | 42 | 920 | CTAGCATACTTCCACAGCTACGCCGCT | I | 115 | 2022 | ++ |
| LAMTNTFLA | I | 43 | 921 | CTAGCAATGACCAACACCTTCCTGGCT | I | 116 | 2023 | ++ |
| LAFPCIKEA | I | 44 | 922 | CTAGCATTCCCCTGCATCAAGGAGGCT | I | 117 | 2024 | + |
| LAHTPDTFA | I | 45 | 923 | CTAGCACACACCCCCGACACCTTCGCT | I | 118 | 2025 | + |
| LAGEVTKHA | I | 46 | 924 | CTAGCAGGCGAGGTGACCAAGCACGCT | I | 119 | 2026 | + |
| LAGDIVRNA | I | 47 | 925 | CTAGCAGGCGACATCGTGCGGAACGCT | I | 120 | 2027 | + |
| LANRWAIDA | I | 48 | 926 | CTAGCAAACCGGTGGGCCATCGACGCT | I | 121 | 2028 | + |
| LANAMEKHA | I | 49 | 927 | CTAGCAAACGCCATGGAGAAGCACGCT | I | 122 | 2029 | + |
| LADENLQAA | I | 50 | 928 | CTAGCAGACGAGAACCTGCAGGCCGCT | I | 123 | 2030 | + |
| LATRADIFA | I | 51 | 929 | CTAGCAACCCGGGCCGACATCTTCGCT | I | 124 | 2031 | + |
| LAKDGGKIA | I | 52 | 930 | CTAGCAAAGGACGGCGGCAAGATCGCT | I | 125 | 2032 | + |
| LAGKIEENA | I | 53 | 931 | CTAGCAGGCAAGATCGAGGAGAACGCT | I | 126 | 2033 | + |
| LAPVKHTWA | I | 54 | 932 | CTAGCACCCGTGAAGCACACCTGGGCT | I | 127 | 2034 | + |
| LAKVASMNA | I | 55 | 933 | CTAGCAAAGGTGGCCAGCATGAACGCT | I | 128 | 2035 | + |
| LAQVEVSKA | I | 56 | 934 | CTAGCACAGGTGGAGGTGAGCAAGGCT | I | 129 | 2036 | 1 |
| LANLKNFSA | I | 57 | 935 | CTAGCAAACCTGAAGAACTTCAGCGCT | I | 130 | 2037 | + |
| LAKGQAQNA | I | 58 | 936 | CTAGCAAAGGGCCAGGCCCAGAACGCT | I | 131 | 2038 | + |
| LADDQAVNA | I | 59 | 937 | CTAGCAGACGACCAGGCCGTGAACGCT | I | 132 | 2039 | + |
| TSQEKQNS | I | 60 | 938 | ACCAGCCAGGAGAAGCAGAACAGC | I | 133 | 2040 | ++ |
| SGPGTSGT | I | 61 | 939 | AGCGGCCCCGGCACCAGCGGCACC | I | 134 | 2041 | ++ |
| TGIATSYS | I | 62 | 940 | ACCGGCATCGCCACCAGCTACAGC | I | 135 | 2042 | ++ |
| SGREIHFG | I | 63 | 941 | AGCGGCCGGGAGATCCACTTCGGC | I | 136 | 2043 | ++ |
| SGIDTRLG | I | 64 | 942 | AGCGGCATCGACACCCGGCTGGGC | I | 137 | 2044 | + |
| GTMFNNST | I | 65 | 943 | GGCACCATGTTCAACAACAGCACC | I | 138 | 2045 | + |
| TTGMERPT | I | 66 | 944 | ACCACCGGCATGGAGCGGCCCACC | I | 139 | 2046 | + |
| TGDERKPG | I | 67 | 945 | ACCGGCGACGAGCGGAAGCCCGGC | I | 140 | 2047 | + |
| CKEITR | I | 68 | 946 | TGCAAGGAGATCACCCGG | I | 141 | 2048 | +++ |
| PDVSLL | I | 69 | 947 | CCCGACGTGAGCCTGCTG | I | 142 | 2049 | +++ |
| NIKKAA | I | 70 | 948 | AACATCAAGAAGGCCGCC | I | 143 | 2050 | ++ |
| RLGRLS | I | 71 | 949 | CGGCTGGGCCGGCTGAGC | I | 144 | 2051 | + |
| KAMRTG | I | 72 | 950 | AAGGCCATGCGGACCGGC | I | 145 | 2052 | + |
| KFTKAA | I | 73 | 951 | AAGTTCACCAAGGCCGCC | I | 146 | 2053 | + |
| RLGKVP | I | 74 | 952 | CGGCTGGGCAAGGTGCCC | I | 147 | 2054 | + |

*AAV2 vectors designed to include SEQ ID NO: 1 with the indicated sequence inclusion. The 8-mers and 9-mers were inserted between amino acid residues 587 and 588 of SEQ ID NO: 1, and the 6-mers were used as replacements for amino acid residues 585 to 590 of SEQ ID NO: 1. +++ means that the modified AAV2 vectors had a performance that placed them within the top 1/3 of the rescued AAV vectors; ++ means the AAV2 vectors had a performance that placed them within the middle 1/3 of the rescued AAV vectors; and + means the AAV2 vectors had a performance that placed them within the bottom 1/3 of the rescued AAV vectors.
**SEQ ID NOs used for Sequence Listing.

TABLE 1J
Amino acid sequences that can be inserted into an AAV capsid polypeptide.

| | | | SEQ | | | | SEQ | |
|---|---|---|---|---|---|---|---|---|
| | SEQ | | ID | | | | ID | |
| Amino Acid | ID | | NO: | Nucleic Acid Sequence encoding the Amino | SEQ | | NO: | Activity |
| Sequence | NO: | | ** | Acid Sequence | ID NO: | | ** | Level* |
| LAHGGLREA | J | 2 | 953 | CTAGCACACGGCGGCCTGCGGGAGGCT | J | 65 | 2055 | +++ |
| LAFTHGTNA | J | 3 | 954 | CTAGCATTCACCCACGGCACCAACGCT | J | 66 | 2056 | +++ |
| LADPTETPA | J | 4 | 955 | CTAGCAGACCCCACCGAGACCCCCGCT | J | 67 | 2057 | +++ |
| LALNDNVGA | J | 5 | 956 | CTAGCACTGAACGACAACGTGGGCGCT | J | 68 | 2058 | +++ |
| LAFCIKHCA | J | 6 | 957 | CTAGCATTCTGCATCAAGCACTGCGCT | J | 69 | 2059 | +++ |
| LAHWQEEWA | J | 7 | 958 | CTAGCACACTGGCAGGAGGAGTGGGCT | J | 70 | 2060 | +++ |
| LAYVIENDA | J | 8 | 959 | CTAGCATACGTGATCGAGAACGACGCT | J | 71 | 2061 | +++ |
| LASTTMNIA | J | 9 | 960 | CTAGCAAGCACCACCATGAACATCGCT | J | 72 | 2062 | +++ |
| LAYVALFYA | J | 10 | 961 | CTAGCATACGTGGCCCTGTTCTACGCT | J | 73 | 2063 | +++ |
| LAFQQQRCA | J | 11 | 962 | CTAGCATTCCAGCAGCAGCGGTGCGCT | J | 74 | 2064 | +++ |
| LADSSKWIA | J | 12 | 963 | CTAGCAGACAGCAGCAAGTGGATCGCT | J | 75 | 2065 | +++ |
| LAVMGNMLA | J | 13 | 964 | CTAGCAGTGATGGGCAACATGCTGGCT | J | 76 | 2066 | +++ |
| LANANTRPA | J | 14 | 965 | CTAGCAAACGCCAACACCCGGCCCGCT | J | 77 | 2067 | +++ |
| LAGENHISA | J | 15 | 966 | CTAGCAGGCGAGAACCACATCAGCGCT | J | 78 | 2068 | +++ |
| LASVSTIQA | J | 16 | 967 | CTAGCAAGCGTGAGCACCATCCAGGCT | J | 79 | 2069 | +++ |
| LAAYHDSAA | J | 17 | 968 | CTAGCAGCCTACCACGACAGCGCCGCT | J | 80 | 2070 | +++ |
| LAVKGLVNA | J | 18 | 969 | CTAGCAGTGAAGGGCCTGGTGAACGCT | J | 81 | 2071 | +++ |
| LADGGTRPA | J | 19 | 970 | CTAGCAGACGGCGGCACCCGGCCCGCT | J | 82 | 2072 | +++ |
| LACYPPTCA | J | 20 | 971 | CTAGCATGCTACCCCCCCACCTGCGCT | J | 83 | 2073 | +++ |
| LAKVSKGDA | J | 21 | 972 | CTAGCAAAGGTGAGCAAGGGCGACGCT | J | 84 | 2074 | +++ |
| LAWKCKGTA | J | 22 | 973 | CTAGCATGGAAGTGCAAGGGCACCGCT | J | 85 | 2075 | ++ |
| LADMTRFHA | J | 23 | 974 | CTAGCAGACATGACCCGGTTCCACGCT | J | 86 | 2076 | ++ |
| LAGKTISGA | J | 24 | 975 | CTAGCAGGCAAGACCATCAGCGGCGCT | J | 87 | 2077 | ++ |
| LALKLRTEA | J | 25 | 976 | CTAGCACTGAAGCTGCGGACCGAGGCT | J | 88 | 2078 | ++ |
| LAAVTKDSA | J | 26 | 977 | CTAGCAGCCGTGACCAAGGACAGCGCT | J | 89 | 2079 | ++ |
| LAKDKGLIA | J | 27 | 978 | CTAGCAAAGGACAAGGGCCTGATCGCT | J | 90 | 2080 | ++ |
| LAHHKESIA | J | 28 | 979 | CTAGCACACCACAAGGAGAGCATCGCT | J | 91 | 2081 | ++ |
| LAVDIMRGA | J | 29 | 980 | CTAGCAGTGGACATCATGCGGGGCGCT | J | 92 | 2082 | ++ |
| LANIVQQDA | J | 30 | 981 | CTAGCAAACATCGTGCAGCAGGACGCT | J | 93 | 2083 | ++ |
| LAGERETVA | J | 31 | 982 | CTAGCAGGCGAGCGGGAGACCGTGGCT | J | 94 | 2084 | ++ |
| LADVVSVSA | J | 32 | 983 | CTAGCAGACGTGGTGAGCGTGAGCGCT | J | 95 | 2085 | ++ |
| LATPNVNAA | J | 33 | 984 | CTAGCAACCCCCAACGTGAACGCCGCT | J | 96 | 2086 | ++ |
| LALNAAATA | J | 34 | 985 | CTAGCACTGAACGCCGCCGCCACCGCT | J | 97 | 2087 | ++ |
| LADCMMINA | J | 35 | 986 | CTAGCAGACTGCATGATGATCAACGCT | J | 98 | 2088 | ++ |
| LAMLACKGA | J | 36 | 987 | CTAGCAATGCTGGCCTGCAAGGGCGCT | J | 99 | 2089 | ++ |
| LANMMAAGA | J | 37 | 988 | CTAGCAAACATGATGGCCGCCGGCGCT | J | 100 | 2090 | ++ |
| LADKNRLQA | J | 38 | 989 | CTAGCAGACAAGAACCGGCTGCAGGCT | J | 101 | 2091 | ++ |
| LAAVGMKMA | J | 39 | 990 | CTAGCAGCCGTGGGCATGAAGATGGCT | J | 102 | 2092 | ++ |
| LADDMIFVA | J | 40 | 991 | CTAGCAGACGACATGATCTTCGTGGCT | J | 103 | 2093 | ++ |
| LADIVGESA | J | 41 | 992 | CTAGCAGACATCGTGGGCGAGAGCGCT | J | 104 | 2094 | ++ |
| LAGGKQALA | J | 42 | 993 | CTAGCAGGCGGCAAGCAGGCCCTGGCT | J | 105 | 2095 | + |
| LAQNQKQIA | J | 43 | 994 | CTAGCACAGAACCAGAAGCAGATCGCT | J | 106 | 2096 | + |
| LAETTLTRA | J | 44 | 995 | CTAGCAGAGACCACCCTGACCCGGGCT | J | 107 | 2097 | + |
| LAMLGGTSA | J | 45 | 996 | CTAGCAATGCTGGGCGGCACCAGCGCT | J | 108 | 2098 | + |
| LAGFMEYYA | J | 46 | 997 | CTAGCAGGCTTCATGGAGTACTACGCT | J | 109 | 2099 | + |
| LAYGQQTTA | J | 47 | 998 | CTAGCATACGGCCAGCAGACCACCGCT | J | 110 | 2100 | + |
| LALNNDQSA | J | 48 | 999 | CTAGCACTGAACAACGACCAGAGCGCT | J | 111 | 2101 | + |
| LAFLRGPEA | J | 49 | 1000 | CTAGCATTCCTGCGGGGCCCCGAGGCT | J | 112 | 2102 | + |
| LACPVAEWA | J | 50 | 1001 | CTAGCATGCCCCGTGGCCGAGTGGGCT | J | 113 | 2103 | + |
| LALETTQRA | J | 51 | 1002 | CTAGCACTGGAGACCACCCAGCGGGCT | J | 114 | 2104 | + |
| LADTYKPYA | J | 52 | 1003 | CTAGCAGACACCTACAAGCCCTACGCT | J | 115 | 2105 | + |
| LAKRDKPTA | J | 53 | 1004 | CTAGCAAAGCGGGACAAGCCCACCGCT | J | 116 | 2106 | + |
| LAYFHSYAA | J | 54 | 1005 | CTAGCATACTTCCACAGCTACGCCGCT | J | 117 | 2107 | + |
| LAMTNTFLA | J | 55 | 1006 | CTAGCAATGACCAACACCTTCCTGGCT | J | 118 | 2108 | + |
| LAGESSYLA | J | 56 | 1007 | CTAGCAGGCGAGAGCAGCTACCTGGCT | J | 119 | 2109 | + |
| LAAEHIGRA | J | 57 | 1008 | CTAGCAGCCGAGCACATCGGCCGGGCT | J | 120 | 2110 | + |
| LADWRTTPA | J | 58 | 1009 | CTAGCAGACTGGCGGACCACCCCCGCT | J | 121 | 2111 | + |
| LAEGLSQFA | J | 59 | 1010 | CTAGCAGAGGGCCTGAGCCAGTTCGCT | J | 122 | 2112 | + |
| LAKPTGKDA | J | 60 | 1011 | CTAGCAAAGCCCACCGGCAAGGACGCT | J | 123 | 2113 | + |
| TGEGPTKG | J | 61 | 1012 | ACCGGCGAGGGCCCCACCAAGGGC | J | 124 | 2114 | + |
| RALKIS | J | 62 | 1013 | CGGGCCCTGAAGATCAGC | J | 125 | 2115 | ++ |
| NSKKLG | J | 63 | 1014 | AACAGCAAGAAGCTGGGC | J | 126 | 2116 | + |
| HFPVFS | J | 64 | 1015 | CACTTCCCCGTGTTCAGC | J | 127 | 2117 | + |

*AAV2 vectors designed to include SEQ ID NO: 1 with the indicated sequence inclusion. The 8-mers and 9-mers were inserted between amino acid residues 587 and 588 of SEQ ID NO: 1, and the 6-mers were used as replacements for amino acid residues 585 to 590 of SEQ ID NO: 1. +++ means that the modified AAV2 vectors had a performance that placed them within the top 1/3 of the rescued AAV vectors; ++ means the AAV2 vectors had a performance that placed them within the middle 1/3 of the rescued AAV vectors; and + means the AAV2 vectors had a performance that placed them within the bottom 1/3 of the rescued AAV vectors.
**SEQ ID NOs used for Sequence Listing.

TABLE 1K
Amino acid sequences that can be inserted into an AAV capsid polypeptide.

| | | | SEQ | | | | SEQ | |
|---|---|---|---|---|---|---|---|---|
| | SEQ | | ID | | | | ID | |
| Amino Acid | ID | | NO: | Nucleic Acid Sequence encoding the Amino | SEQ ID | | NO: | Activity |
| Sequence | NO: | | ** | Acid Sequence | NO: | | ** | Level* |
| LATDRTKTA | K | 2 | 1016 | CTAGCAACCGACCGGACCAAGACCGCT | K | 64 | 2118 | +++ |
| LAVDNIWAA | K | 3 | 1017 | CTAGCAGTGGACAACATCTGGGCCGCT | K | 65 | 2119 | +++ |
| LAGQAMMQA | K | 4 | 1018 | CTAGCAGGCCAGGCCATGATGCAGGCT | K | 66 | 2120 | +++ |
| LAMTKITDA | K | 5 | 1019 | CTAGCAATGACCAAGATCACCGACGCT | K | 67 | 2121 | +++ |
| LAGGEYETA | K | 6 | 1020 | CTAGCAGGCGGCGAGTACGAGACCGCT | K | 68 | 2122 | +++ |
| LAALVDANA | K | 7 | 1021 | CTAGCAGCCCTGGTGGACGCCAACGCT | K | 69 | 2123 | +++ |
| LALENIGQA | K | 8 | 1022 | CTAGCACTGGAGAACATCGGCCAGGCT | K | 70 | 2124 | +++ |
| LAHRAPDSA | K | 9 | 1023 | CTAGCACACCGGGCCCCCGACAGCGCT | K | 71 | 2125 | +++ |
| LAEGQEDSA | K | 10 | 1024 | CTAGCAGAGGGCCAGGAGGACAGCGCT | K | 72 | 2126 | +++ |
| LAPHIAKDA | K | 11 | 1025 | CTAGCACCCCACATCGCCAAGGACGCT | K | 73 | 2127 | +++ |
| LARDQLKLA | K | 12 | 1026 | CTAGCACGGGACCAGCTGAAGCTGGCT | K | 74 | 2128 | +++ |
| LALNSGKMA | K | 13 | 1027 | CTAGCACTGAACAGCGGCAAGATGGCT | K | 75 | 2129 | +++ |
| LAVSRHGEA | K | 14 | 1028 | CTAGCAGTGAGCCGGCACGGCGAGGCT | K | 76 | 2130 | +++ |
| LAMNEPVTA | K | 15 | 1029 | CTAGCAATGAACGAGCCCGTGACCGCT | K | 77 | 2131 | +++ |
| LAYITEKSA | K | 16 | 1030 | CTAGCATACATCACCGAGAAGAGCGCT | K | 78 | 2132 | ++ |
| LAYDAGGHA | K | 17 | 1031 | CTAGCATACGACGCCGGCGGCCACGCT | K | 79 | 2133 | ++ |
| LALHQERQA | K | 18 | 1032 | CTAGCACTGCACCAGGAGCGGCAGGCT | K | 80 | 2134 | ++ |
| LAAKPKDGA | K | 19 | 1033 | CTAGCAGCCAAGCCCAAGGACGGCGCT | K | 81 | 2135 | ++ |
| LAEYHNNDA | K | 20 | 1034 | CTAGCAGAGTACCACAACAACGACGCT | K | 82 | 2136 | ++ |
| LATWCLVWA | K | 21 | 1035 | CTAGCAACCTGGTGCCTGGTGTGGGCT | K | 83 | 2137 | ++ |
| LAFGFSADA | K | 22 | 1036 | CTAGCATTCGGCTTCAGCGCCGACGCT | K | 84 | 2138 | ++ |
| LAGVVKTDA | K | 23 | 1037 | CTAGCAGGCGTGGTGAAGACCGACGCT | K | 85 | 2139 | ++ |
| LAKFMWIHA | K | 24 | 1038 | CTAGCAAAGTTCATGTGGATCCACGCT | K | 86 | 2140 | ++ |
| LANQGFLTA | K | 25 | 1039 | CTAGCAAACCAGGGCTTCCTGACCGCT | K | 87 | 2141 | ++ |
| LAHDLHGGA | K | 26 | 1040 | CTAGCACACGACCTGCACGGCGGCGCT | K | 88 | 2142 | ++ |
| LASENLNRA | K | 27 | 1041 | CTAGCAAGCGAGAACCTGAACCGGGCT | K | 89 | 2143 | ++ |
| LAFRTMEYA | K | 28 | 1042 | CTAGCATTCCGGACCATGGAGTACGCT | K | 90 | 2144 | ++ |
| LAAANHDNA | K | 29 | 1043 | CTAGCAGCCGCCAACCACGACAACGCT | K | 91 | 2145 | ++ |
| LAPSKMGLA | K | 30 | 1044 | CTAGCACCCAGCAAGATGGGCCTGGCT | K | 92 | 2146 | ++ |
| LANEGTIRA | K | 31 | 1045 | CTAGCAAACGAGGGCACCATCCGGGCT | K | 93 | 2147 | ++ |
| LARGTLMTA | K | 32 | 1046 | CTAGCACGGGGCACCCTGATGACCGCT | K | 94 | 2148 | + |
| LATKSTGSA | K | 33 | 1047 | CTAGCAACCAAGAGCACCGGCAGCGCT | K | 95 | 2149 | + |
| LAALNSQTA | K | 34 | 1048 | CTAGCAGCCCTGAACAGCCAGACCGCT | K | 96 | 2150 | + |
| LANVTLQSA | K | 35 | 1049 | CTAGCAAACGTGACCCTGCAGAGCGCT | K | 97 | 2151 | + |
| LAAGQAQSA | K | 36 | 1050 | CTAGCAGCCGGCCAGGCCCAGAGCGCT | K | 98 | 2152 | + |
| LAHDWGAPA | K | 37 | 1051 | CTAGCACACGACTGGGGCGCCCCCGCT | K | 99 | 2153 | + |
| LAMDREIKA | K | 38 | 1052 | CTAGCAATGGACCGGGAGATCAAGGCT | K | 100 | 2154 | + |
| LANADFPLA | K | 39 | 1053 | CTAGCAAACGCCGACTTCCCCCTGGCT | K | 101 | 2155 | + |
| LAHOGAIIA | K | 40 | 1054 | CTAGCACACCAGGGCGCCATCATCGCT | K | 102 | 2156 | + |
| LAPNTPKIA | K | 41 | 1055 | CTAGCACCCAACACCCCCAAGATCGCT | K | 103 | 2157 | + |
| LAIPVEANA | K | 42 | 1056 | CTAGCAATCCCCGTGGAGGCCAACGCT | K | 104 | 2158 | + |
| LAIGERTTA | K | 43 | 1057 | CTAGCAATCGGCGAGCGGACCACCGCT | K | 105 | 2159 | + |
| LACRSDCVA | K | 44 | 1058 | CTAGCATGCCGGAGCGACTGCGTGGCT | K | 106 | 2160 | + |
| LANRLSNVA | K | 45 | 1059 | CTAGCAAACCGGCTGAGCAACGTGGCT | K | 107 | 2161 | + |
| LAQEQNAAA | K | 46 | 1060 | CTAGCACAGGAGCAGAACGCCGCCGCT | K | 108 | 2162 | + |
| LARSSTLNA | K | 47 | 1061 | CTAGCACGGAGCAGCACCCTGAACGCT | K | 109 | 2163 | + |
| LAMKYVFDA | K | 48 | 1062 | CTAGCAATGAAGTACGTGTTCGACGCT | K | 110 | 2164 | + |
| LAQEAKLTA | K | 49 | 1063 | CTAGCACAGGAGGCCAAGCTGACCGCT | K | 111 | 2165 | + |
| LAREDHRQA | K | 50 | 1064 | CTAGCACGGGAGGACCACCGGCAGGCT | K | 112 | 2166 | + |
| LAHYPSAPA | K | 51 | 1065 | CTAGCACACTACCCCAGCGCCCCCGCT | K | 113 | 2167 | + |
| LAKGMKEGA | K | 52 | 1066 | CTAGCAAAGGGCATGAAGGAGGGCGCT | K | 114 | 2168 | + |
| TSHVSTSS | K | 53 | 1067 | ACCAGCCACGTGAGCACCAGCAGC | K | 115 | 2169 | +++ |
| SGVLFTEG | K | 54 | 1068 | AGCGGCGTGCTGTTCACCGAGGGC | K | 116 | 2170 | +++ |
| TSGMGAIG | K | 55 | 1069 | ACCAGCGGCATGGGCGCCATCGGC | K | 117 | 2171 | +++ |
| TGDSEQQT | K | 56 | 1070 | ACCGGCGACAGCGAGCAGCAGACC | K | 118 | 2172 | +++ |
| SGSVGYVG | K | 57 | 1071 | AGCGGCAGCGTGGGCTACGTGGGC | K | 119 | 2173 | +++ |
| SSENNKGG | K | 58 | 1072 | AGCAGCGAGAACAACAAGGGCGGC | K | 120 | 2174 | ++ |
| SGPGTSGT | K | 59 | 1073 | AGCGGCCCCGGCACCAGCGGCACC | K | 121 | 2175 | ++ |
| TTENHKPG | K | 60 | 1074 | ACCACCGAGAACCACAAGCCCGGC | K | 122 | 2176 | + |
| FNKVLH | K | 61 | 1075 | TTCAACAAGGTGCTGCAC | K | 123 | 2177 | ++ |
| RTGRNT | K | 62 | 1076 | CGGACCGGCCGGAACACC | K | 124 | 2178 | ++ |
| RLGKVP | K | 63 | 1077 | CGGCTGGGCAAGGTGCCC | K | 125 | 2179 | + |

*AAV2 vectors designed to include SEQ ID NO: 1 with the indicated sequence inclusion. The 8-mers and 9-mers were inserted between amino acid residues 587 and 588 of SEQ ID NO: 1, and the 6-mers were used as replacements for amino acid residues 585 to 590 of SEQ ID NO: 1. +++ means that the modified AAV2 vectors had a performance that placed them within the top 1/3 of the rescued AAV vectors; ++ means the AAV2 vectors had a performance that placed them within the middle 1/3 of the rescued AAV vectors; and + means the AAV2 vectors had a performance that placed them within the bottom 1/3 of the rescued AAV vectors.
**SEQ ID NOs used for Sequence Listing.

TABLE 1L
Amino acid sequences that can be inserted into an AAV capsid polypeptide.

| | | | SEQ | | | | SEQ | |
|---|---|---|---|---|---|---|---|---|
| | SEQ | | ID | | SEQ | | ID | |
| Amino Acid | ID | | NO: | Nucleic Acid Sequence encoding the Amino | ID | | NO: | Activity |
| Sequence | NO: | | ** | Acid Sequence | NO: | | ** | Level* |
| LAWIERSMA | L | 2 | 1078 | CTAGCATGGATCGAGCGGAGCATGGCT | L | 28 | 2180 | +++ |
| LAYDNTAEA | L | 3 | 1079 | CTAGCATACGACAACACCGCCGAGGCT | L | 29 | 2181 | +++ |
| LAQVTEMRA | L | 4 | 1080 | CTAGCACAGGTGACCGAGATGCGGGCT | L | 30 | 2182 | ++ |
| LATRADIFA | L | 5 | 1081 | CTAGCAACCCGGGCCGACATCTTCGCT | L | 31 | 2183 | ++ |
| LAKDGGKIA | L | 6 | 1082 | CTAGCAAAGGACGGCGGCAAGATCGCT | L | 32 | 2184 | ++ |
| LAQVEVSKA | L | 7 | 1083 | CTAGCACAGGTGGAGGTGAGCAAGGCT | L | 33 | 2185 | ++ |
| LADTYKPYA | L | 8 | 1084 | CTAGCAGACACCTACAAGCCCTACGCT | L | 34 | 2186 | ++ |
| LAQEAKLTA | L | 9 | 1085 | CTAGCACAGGAGGCCAAGCTGACCGCT | L | 35 | 2187 | ++ |
| LACRSDCVA | L | 10 | 1086 | CTAGCATGCCGGAGCGACTGCGTGGCT | L | 36 | 2188 | ++ |
| LAKDHHVIA | L | 11 | 1087 | CTAGCAAAGGACCACCACGTGATCGCT | L | 37 | 2189 | + |
| LAEPPDPPA | L | 12 | 1088 | CTAGCAGAGCCCCCCGACCCCCCCGCT | L | 38 | 2190 | + |
| LACSSTWYA | L | 13 | 1089 | CTAGCATGCAGCAGCACCTGGTACGCT | L | 39 | 2191 | + |
| LADLTLRAA | L | 14 | 1090 | CTAGCAGACCTGACCCTGCGGGCCGCT | L | 40 | 2192 | + |
| LAKVASMNA | L | 15 | 1091 | CTAGCAAAGGTGGCCAGCATGAACGCT | L | 41 | 2193 | + |
| LAAQDVSWA | L | 16 | 1092 | CTAGCAGCCCAGGACGTGAGCTGGGCT | L | 42 | 2194 | + |
| LADVGCENA | L | 17 | 1093 | CTAGCAGACGTGGGCTGCGAGAACGCT | L | 43 | 2195 | + |
| TGPGTHNG | L | 18 | 1094 | ACCGGCCCCGGCACCCACAACGGC | L | 44 | 2196 | +++ |
| STVPLNMG | L | 19 | 1095 | AGCACCGTGCCCCTGAACATGGGC | L | 45 | 2197 | ++ |
| HSKDGA | L | 20 | 1096 | CACAGCAAGGACGGCGCC | L | 46 | 2198 | +++ |
| RTGRNT | L | 21 | 1097 | CGGACCGGCCGGAACACC | L | 47 | 2199 | +++ |
| NSKKLG | L | 22 | 1098 | AACAGCAAGAAGCTGGGC | L | 48 | 2200 | +++ |
| RPGRVS | L | 23 | 1099 | CGGCCCGGCCGGGTGAGC | L | 49 | 2201 | +++ |
| KFRHTE | L | 24 | 1100 | AAGTTCCGGCACACCGAG | L | 50 | 2202 | ++ |
| KAMRTG | L | 25 | 1101 | AAGGCCATGCGGACCGGC | L | 51 | 2203 | + |
| NIKKAA | L | 26 | 1102 | AACATCAAGAAGGCCGCC | L | 52 | 2204 | + |
| KFTKAA | L | 27 | 1103 | AAGTTCACCAAGGCCGCC | L | 53 | 2205 | + |

*AAV2 vectors designed to include SEQ ID NO: 1 with the indicated sequence inclusion. The 8-mers and 9-mers were inserted between amino acid residues 587 and 588 of SEQ ID NO: 1, and the 6-mers were used as replacements for amino acid residues 585 to 590 of SEQ ID NO: 1. +++ means that the modified AAV2 vectors had a performance that placed them within the top 1/3 of the rescued AAV vectors; ++ means the AAV2 vectors had a performance that placed them within the middle 1/3 of the rescued AAV vectors; and + means the AAV2 vectors had a performance that placed them within the bottom 1/3 of the rescued AAV vectors.
**SEQ ID NOs used for Sequence Listing.

Many of the amino acid sequences set forth in one or more of Tables 1A-1L start with “LA” and end with “A.” In such cases, that “LA” sequence can be a first linker sequence L1, and that “A” amino acid can be a second linker sequence L2, with the L1 and L2 linkers each independently being optional amino acid linkers having one, two, or three amino acids according to the following Formula A:

-L1-INSERT-L2-,
wherein L1 and L2 are each independently optional amino acid linkers having one, two, or three amino acids, and wherein INSERT represents the sequence of any of the sequence identifiers of Tables 1A-1L without the starting “LA” and the ending “A.” For example, in some cases, an AAV vector can be designed to have an AAV capsid polypeptide that includes an amino acid sequence set forth in SEQ ID NO:1093, which is LADVGCENA, or can be designed to have an AAV capsid polypeptide that includes an amino acid sequence based on SEQ ID NO:1093 according to Formula A, which is L1-DVGCEN-L2, with L1 and L2 being as defined herein.

As described herein, an AAV vector can be designed to have an AAV capsid polypeptide that includes an amino acid sequence of Formula A based on any of the amino acid sequences set forth in Tables 1A-1L. For example, an AAV vector can be designed to have an AAV capsid polypeptide of SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) that includes an amino acid sequence of Formula A based on any of the amino acid sequences set forth in Tables 1A-1L located between amino acid positions 587 and 588 of SEQ ID NO:1 (or the appropriate amino acid positions of the alternative sequence). In some cases, L1, L2, or both L1 and L2 can be absent. For example, an AAV capsid polypeptide that includes an amino acid sequence of Formula A based on SEQ ID NO:1093 can include the amino acid sequence of SEQ ID NO:1093 in the absence of the starting “LA” and the ending “A.” In some cases, L1 can be one amino acid X1, two amino acids X2-X1, or three amino acids X3-X2-X1. When X1 is present, it can be an amino acid residue selected from the group consisting of A, V, I, and L. When X2 is present, it can be an amino acid residue selected from the group consisting of A, V, I, and L. When X3 is present, it can be an amino acid residue selected from the group consisting of A, V, I, and L. In some cases, L2 can be one amino acid Z1, two amino acids Z1-Z2, or three amino acids Z1-Z2-Z3. When Z1 is present, it can be an amino acid residue selected from the group consisting of A, V, I, and L. When Z2 is present, it can be an amino acid residue selected from the group consisting of A, V, I, and L. When Z3 is present, it can be an amino acid residue selected from the group consisting of A, V, I, and L. Examples of an L1 linkers include, without limitation, A, V, I, L, AA, AV, AI, AL, VA, VV, VI, VL, IA, IV, II, IL, LA, LV, LI, LL, AAA, AAV, AAI, AAL, AVA, AVV, AVI, AVL, AIA, AIV, AII, AIL, ALA, ALV, ALI, ALL, VAA, VAV, VAI, VAL, VVA, VVV, VVI, VVL, VIA, VIV, VII, VIL, VLA, VLV, VLI, VLL, IAA, IAV, IAI, IAL, IVA, IVV, IVI, IVL, IIA, IIV, III, IIL, ILA, ILV, ILI, ILL, LAA, LAV, LAI, LAL, LVA, LVV, LVI, LVL, LIA, LIV, LII, LIL, LLA, LLV, LLI, and LLL. Examples of an L2 linkers include, without limitation, A, V, I, L, AA, AV, AI, AL, VA, VV, VI, VL, IA, IV, II, IL, LA, LV, LI, LL, AAA, AAV, AAI, AAL, AVA, AVV, AVI, AVL, AIA, AIV, All, AIL, ALA, ALV, ALI, ALL, VAA, VAV, VAI, VAL, VVA, VVV, VVI, VVL, VIA, VIV, VII, VIL, VLA, VLV, VLI, VLL, IAA, IAV, IAI, IAL, IVA, IVV, IVI, IVL, IIA, IIV, III, IIL, ILA, ILV, ILI, ILL, LAA, LAV, LAI, LAL, LVA, LVV, LVI, LVL, LIA, LIV, LII, LIL, LLA, LLV, LLI, and LLL.

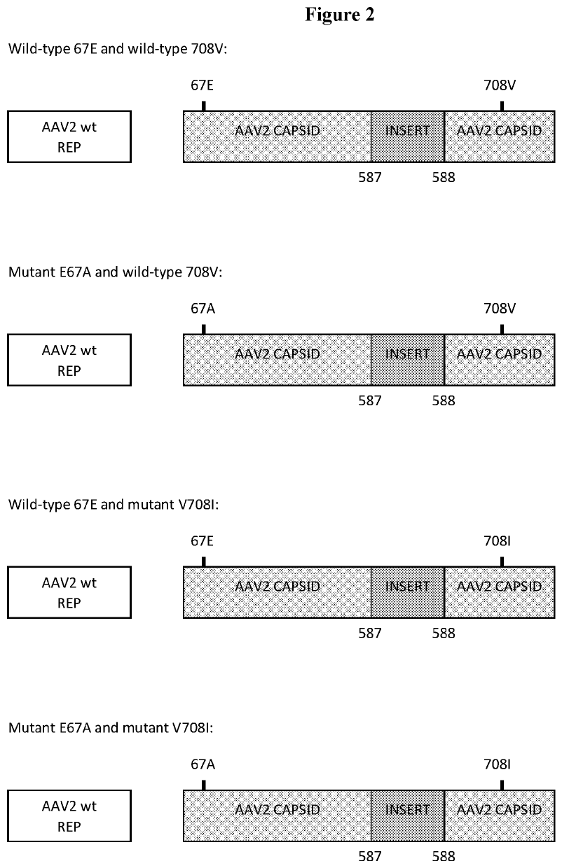

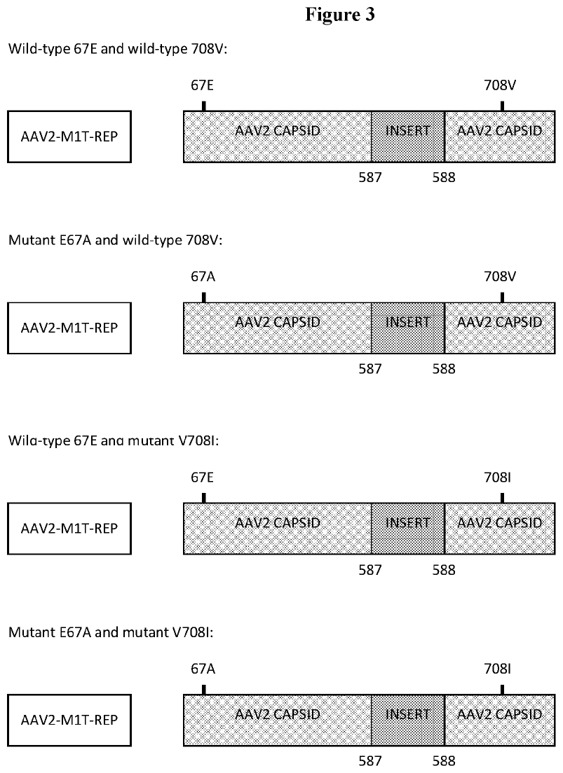

In some cases, an AAV2 capsid polypeptide provided herein can have the sequence set forth in SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO: 1) with an amino acid sequence set forth in any one or more of Tables 1A-1L (or a variant thereof) or according to Formula A based on such a set forth sequence inserted between asparagine-587 and arginine-588 (or the appropriate amino acid positions of the alternative sequence) (see, e.g., FIGS. 2-3). In some cases, an AAV2 capsid polypeptide provided herein can have the sequence set forth in SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) with an amino acid sequence set forth in any one of SEQ ID NOs:A2-A18, SEQ ID NOs:B2-B298, SEQ ID NOs:C2-C43, SEQ ID NOs:D2-D76, SEQ ID NOs:E2-E75, SEQ ID NOs:F2-F11, SEQ ID NOs:G2-G70, SEQ ID NOs:H2-H243, SEQ ID NOs:I2-I67, SEQ ID NOs:J2-J61, SEQ ID NOs:K2-K60, and SEQ ID NOs:L2-L19 (or a variant thereof) inserted between asparagine-587 and arginine-588 (or the appropriate amino acid positions of the alternative sequence).

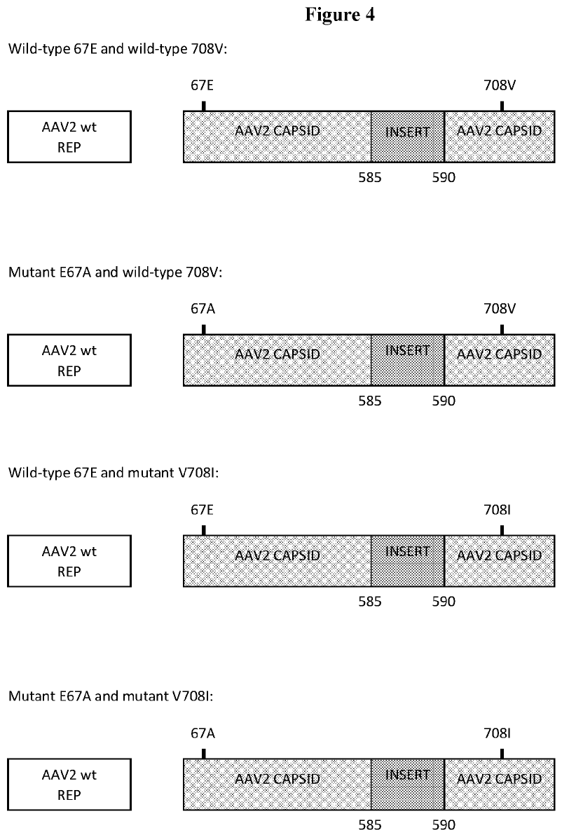

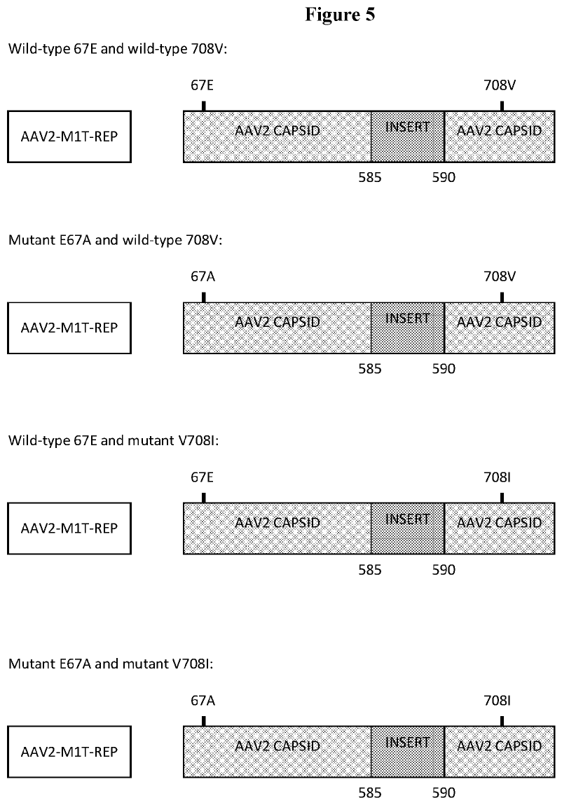

In some cases, when designing an AAV capsid polypeptide (e.g., an AAV2 capsid polypeptide) to include an amino acid sequence set forth in any one or more of Tables 1A-1L (or a variant thereof) or according to Formula A based on such a set forth sequence, that included amino acid sequence can be used to replace one or more naturally-occurring amino acid residues located at any appropriate location along the AAV capsid polypeptide (e.g., the AAV2 capsid polypeptide). For example, an amino acid sequence set forth in any one or more of Tables 1A-1L (or a variant thereof) such as any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-174, SEQ ID NOs:J2-J64, SEQ ID NOs:K2-K63, and SEQ ID NOs:L2-L27 can be used to replace the naturally-occurring amino acid residues at positions 585 to 590 of an AAV capsid polypeptide (e.g., an AAV2 capsid polypeptide) (see, e.g., FIGS. 4-5).

In some cases, an AAV2 capsid polypeptide provided herein can have the sequence set forth in SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) except that the amino acid residues at positions 585 to 590 (or the appropriate amino acid positions of the alternative sequence) are replaced with an amino acid sequence set forth in any one or more of Tables 1A-1L (or a variant thereof) or according to Formula A based on such a set forth sequence. In some cases, an AAV2 capsid polypeptide provided herein can have the sequence set forth in SEQ ID NO:1 (or an alternative sequence that is the amino acid sequence set forth in SEQ ID NO:2206 or that is an amino acid sequence at least 95%, at least 96%, at least 97%, at least 98%, or at least 99% identical to the amino acid sequence set forth in SEQ ID NO:1) with the exception that amino acid residues 585 to 590 (or the appropriate amino acid positions of the alternative sequence) are replaced with the amino acid sequence set forth in any one of SEQ ID NO:A19, SEQ ID NOs:B299-B317, SEQ ID NOs:C44-C45, SEQ ID NOs:D77-D78, SEQ ID NOs:E76-E83, SEQ ID NOs:F2-F10, SEQ ID NOs:G71-G77, SEQ ID NOs:H244-H258, SEQ ID NOs:I68-175, SEQ ID NOs:J62-J64, SEQ ID NOs:K61-K63, and SEQ ID NOs:L20-L27 (or a variant thereof).

In some cases, an AAV capsid polypeptide (e.g., an AAV2 capsid polypeptide) can be designed to include two or more amino acid sequences set forth in any one or more of Tables 1A-1L (or a variant thereof) or according to Formula A based on such a set forth sequence. For example, an AAV capsid polypeptide (e.g., an AAV2 capsid polypeptide) can be designed to include two, three, or four amino acid sequences set forth in any one or more of Tables 1A-1L (or a variant thereof) or according to Formula A based on such a set forth sequence.

As described herein, an AAV capsid polypeptide (e.g., an AAV2 capsid polypeptide) can be designed to include an amino acid sequence set forth in any one or more of Tables 1A-1L (or a variant thereof) or according to Formula A based on such a set forth sequence. A variant of an amino acid sequence set forth in any one or more of Tables 1A-1L refers to an amino acid sequence that is identical to that amino acid sequence set forth in any one or more of Tables 1A-1L except that it has one, two, or three amino acid additions, deletions, substitutions, or combinations thereof. For example, a variant of SEQ ID NO:A2 can be SEQ ID NO:A2 except that it has one, two, or three amino acid additions, deletions, substitutions, or combinations thereof. In some cases, a variant provided herein can be the amino acid sequence set forth in any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-174, SEQ ID NOs:J2-J64, SEQ ID NOs:K2-K63, and SEQ ID NOs:L2-L27 except that it contains one, two, or three amino acid additions. In some cases, a variant provided herein can be the amino acid sequence set forth in any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-174, SEQ ID NOs:J2-J64, SEQ ID NOs:K2-K63, and SEQ ID NOs:L2-L27 except that it contains one, two, or three amino acid deletions. In some cases, a variant provided herein can be the amino acid sequence set forth in any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-I74, SEQ ID NOs:J2-J64, SEQ ID NOs:K2-K63, and SEQ ID NOs:L2-L27 except that it contains one, two, or three amino acid substitutions. In some cases, a variant provided herein can be the amino acid sequence set forth in any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-I74, SEQ ID NOs:J2-J64, SEQ ID NOs:K2-K63, and SEQ ID NOs:L2-L27 except that it contains one amino acid addition, deletion, or substitution. In some cases, a variant provided herein can be the amino acid sequence set forth in any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-I74, SEQ ID NOs:J2-J64, SEQ ID NOs:K2-K63, and SEQ ID NOs:L2-L27 except that it contains two amino acid additions, deletions, substitutions, or a combination thereof. In some cases, a variant provided herein can be the amino acid sequence set forth in any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-174, SEQ ID NOs:J2-J64, SEQ ID NOs:K2-K63, and SEQ ID NOs:L2-L27 except that it contains three amino acid additions, deletions, substitutions, or a combination thereof.

In some cases, an amino acid substitution present in a variant can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution present in a variant can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

The percent sequence identity between a particular amino acid sequence and an amino acid sequence referenced by a particular sequence identification number is determined as follows. First, an amino acid sequence is compared to the sequence set forth in a particular sequence identification number using the BLAST 2 Sequences (Bl2seq) program from the stand-alone version of BLASTZ containing BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the B12seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq-i c:\seq1.txt-j c:\seq2.txt-p blastp-o c:\output.txt. If the two compared sequences share homology, then the designated output file will present those regions of homology as aligned sequences. If the two compared sequences do not share homology, then the designated output file will not present aligned sequences. Once aligned, the number of matches is determined by counting the number of positions where an identical amino acid residue is presented in both sequences. A matched position refers to a position in which an identical amino acid residue occurs at the same position in aligned sequences. The percent sequence identity is determined by dividing the number of matches by the length of the sequence set forth in the identified sequence (e.g., SEQ ID NO:1), followed by multiplying the resulting value by 100. For example, an amino acid sequence that has 725 matches when aligned with the sequence set forth in SEQ ID NO:1 is 98.6 percent identical to the sequence set forth in SEQ ID NO:1 (i.e., 725÷735×100=98.6). It is noted that the percent sequence identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

Methods for generating an amino acid sequence variant can include site-specific mutagenesis or random mutagenesis (e.g., by PCR) of a nucleic acid encoding an AAV capsid polypeptide. See, for example, Zoller, Curr Opin. Biotechnol. 3: 348-354 (1992).

The AAV vectors (e.g., AAV2 vectors) described herein can be designed to include one or more exogenous nucleic acid sequences. For example, an AAV vector (e.g., an AAV2 vector) described herein can be designed to include an exogenous nucleic acid sequence that encodes an RNA of interest and/or a polypeptide of interest. An exogenous nucleic acid sequence can be designed to encode any appropriate RNA of interest. Examples of RNAs of interest that can be encoded by an exogenous nucleic acid sequence designed to be included within an AAV vector provided herein include, without limitation, siRNAs, RNA components for gene editing, and microRNAs. In some cases, an RNA of interest that can be encoded by an exogenous nucleic acid sequence included within an AAV vector provided herein can be SIRNA-027 to treat, e.g., sub-foveal CNVM secondary to age-related macular degeneration (see, e.g., NCT00363714), Cand5/Bevasiranib to treat, e.g., diabetic macular edema (see, e.g., NCT00306904), PF-04523655 to treat, e.g., diabetic macular edema (see, e.g., NCT01445899), QPI-1007 to treat, e.g., optic nerve atrophy in NAION (see, e.g., NCT01064505), Aganirsen to treat, e.g., ischemic CRVO to prevent neovascular glaucoma (see, e.g., NCT02947867), QR-421a to treat, e.g., retinitis pigmentosa/Usher syndrome type 2 (see, e.g., NCT03780257), QR-1123 to treat, e.g., autosomal dominant retinitis pigmentosa (see, e.g., NCT04123626), IONIS-FB-LRx to treat, e.g., geographic atrophy secondary to age-related macular degeneration (see, e.g., NCT03815825), or Sepofarsen/QR-110 to treat, e.g., Leber's congenital amaurosis (see, e.g., NCT03913143).

An exogenous nucleic acid sequence can be designed to encode any appropriate polypeptide of interest. Examples of polypeptides of interest that can be encoded by an exogenous nucleic acid sequence designed to be included within an AAV vector provided herein include, without limitation, therapeutic polypeptides, trophic factor polypeptides, gene editing polypeptides (e.g., a Cas9 polypeptide, a TALEN polypeptide, or a zinc finger polypeptide), enzymes, optogenetic tool polypeptides (e.g., a ChR polypeptide, an NhpR polypeptide, or a ReachR polypeptide), antibodies, antibody domains (e.g., VH domains), cytokines, anti-angiogenic polypeptides, and neuroprotective polypeptides. Examples of polypeptides of interest that can be encoded by an exogenous nucleic acid sequence designed to be included within an AAV vector provided herein include, without limitation, an ABCA4 polypeptide, a CRB1 polypeptide, an NPHP5 polypeptide, an NR2E3 polypeptide, a PDE6A polypeptide, a PDE6B polypeptide, a PDE6C polypeptide, a PRPF31 polypeptide, a RPE65 polypeptide, a RPGR polypeptide, a RS1 polypeptide, a TYR polypeptide, a USH2A polypeptide, a MYO7A polypeptide, an REP1 polypeptide, an OPN1LW polypeptide, an OPN1MW polypeptide, a CNGA3 polypeptide, a CNGB3 polypeptide, a GUCY2D polypeptide, a GACA1A polypeptide, a GNAT2 polypeptide, a PDE6H polypeptide, a PROM1 polypeptide, a PRPH2 polypeptide, a CRX polypeptide, an NPHP5 polypeptide, an EYS polypeptide, an ND4 polypeptide, a CLN1-14 polypeptide (e.g., a CLN3 polypeptide, a CLN5 polypeptide, a CLN6 polypeptide, or a CLN8 polypeptide), an NYX polypeptide, a GRM6 polypeptide, a TRPM1 polypeptide, a GPR179 polypeptide, an LRIT3 polypeptide, a glial cell derived neurotrophic factor (GDNF) polypeptide, a brain-derived neurotrophic factor (BDNF) polypeptide, a fibroblast growth factor (FGF) polypeptide, a truncated rod-derived cone viability factor (RdCVF) polypeptide, a full-length rod-derived cone viability factor (RdCVFL) polypeptide, an X-linked inhibitor of apoptosis (XIAP) polypeptide, a soluble fms-related receptor tyrosine kinase 1 (sFLT) polypeptide, a CYP4V2 polypeptide, a palmitoyl protein thioesterase 1 polypeptide, a tripeptidyl peptidase 1 polypeptide, a DNAJC5 polypeptide, a MFSD8 polypeptide, a cathepsin D polypeptide, a granulin polypeptide, an ATP13A2 polypeptide, a cathepsin F polypeptide, a KCTD7 polypeptide, a “P” gene polypeptide, a TRP1 polypeptide, a MATP (SLC45A2) polypeptide, a SLC24A5 polypeptide, a LRMDA polypeptide, a GPR143 polypeptide, an RPGR-exon 1-ORF15 polypeptide, an USH2b polypeptide, an USHIC polypeptide, a CDH23 polypeptide, a PCDH15 polypeptide, a SANS polypeptide, an USH1H polypeptide, a CIB2 polypeptide, an USH1K polypeptide, an ADGRV1 polypeptide, a WHRN polypeptide, a PDZD7 polypeptide, a CLRN1 polypeptide, a HARS polypeptide, an RP2 polypeptide, a FAM161 polypeptide, a DLK polypeptide, a RHO polypeptide, a CHM polypeptide, a BEST1 polypeptide, a RP1 polypeptide, an OPA1 polypeptide, a CEP290 polypeptide, a RDH12 polypeptide, a CACNAIF polypeptide, a BBS1 polypeptide, a FAM161A polypeptide, a CERKL polypeptide, a PRPF8 polypeptide, a RP1L1 polypeptide, a SNRNP200 polypeptide, an IMPG2 polypeptide, a CDHR1 polypeptide, an IMPDH1 polypeptide, a CNGB1 polypeptide, a MERTK polypeptide, a KCNV2 polypeptide, an AIPL1 polypeptide, a RPGRIP1 polypeptide, a TULP1 polypeptide, a C2ORF71 (aka PCARE) polypeptide, a MAK polypeptide, a TIMP3 polypeptide, a GUCA1A polypeptide, an ALMS1 polypeptide, a BBS10 polypeptide, an IFT140 polypeptide, a CNGA1 polypeptide, a NMNAT1 polypeptide, a COL2A1 polypeptide, an EFEMPI polypeptide, a WFS1 polypeptide, a RDH5 polypeptide, a PRPF3 polypeptide, a LRP5 polypeptide, a TOPORS polypeptide, a DHDDS polypeptide, a LCA5 polypeptide, an IQCB1 polypeptide, a RP9 polypeptide, an ATXN7 polypeptide, a BBS2 polypeptide, a SAG RLBP1 polypeptide, a ND6 (MT-ND6) polypeptide, a CIQTNF5 polypeptide, a VPS13B polypeptide, a KIF11 polypeptide, a MT-TL1 polypeptide, a KLHL7 polypeptide, an ACO2 polypeptide, a C21orf2 (aka CFAP410) polypeptide, an AHI1 polypeptide, a KIZ polypeptide, a SPATA7 polypeptide, a TTLL5 polypeptide, an HGSNAT polypeptide, a NRL polypeptide, an OAT polypeptide, a FLVCR1 polypeptide, an ABCC6 polypeptide, a LRAT polypeptide, a CEP78 polypeptide, a CDH3 polypeptide, a FZD4 polypeptide, a BBS12 polypeptide, an HK1 polypeptide, a PRDM13 polypeptide, an ADAM9 polypeptide, a BBS7 polypeptide, a CABP4 polypeptide, an ABHD12 polypeptide, a COL18A1 polypeptide, a MFRP polypeptide, a RIMS1 polypeptide, a ROM1 polypeptide, a BBS4 polypeptide, an IMPG1 polypeptide, an INPP5E polypeptide, a VCAN polypeptide, a POC1B polypeptide, a RAX2 polypeptide, a TSPAN12 polypeptide, a CACNA2D4 polypeptide, a JAG1 polypeptide, a MKKS polypeptide, a NPHP4 polypeptide, a BBS9 polypeptide, a COL11A1 polypeptide, an ELOVL4 polypeptide, a NDP polypeptide, a NPHP1 polypeptide, a RGR polypeptide, a BBS5 polypeptide, a WDR19 polypeptide, a C8ORF37 polypeptide, a CTNNA1 polypeptide, a LAMP2 polypeptide, a PEX1 polypeptide, a PHYH polypeptide, an ATF6 polypeptide, a PRPS1 polypeptide, a SEMA4A polypeptide, an ARL6 polypeptide, a CNNM4 polypeptide, an OTX2 polypeptide, a PRPF6 polypeptide, a RBP3 polypeptide, a PNPLA6 polypeptide, a SLC24A1 polypeptide, an USHIG polypeptide, a PITPNM3 polypeptide, a TTC8 polypeptide, an ARSG polypeptide, a CWC27 polypeptide, a DRAM2 polypeptide, a PRCD polypeptide, a REEP6 polypeptide, a SSBP1 polypeptide, a LAMAI polypeptide, a RAB28 polypeptide, a ZNF408 polypeptide, a GNAT1 polypeptide, an IDH3A polypeptide, a PDE6G polypeptide, a PEX6 polypeptide, a TUB polypeptide, a CEP250 polypeptide, a FSCN2 polypeptide, a GRK1 polypeptide, a RBP4 polypeptide, a RD3 polypeptide, an AGBL5 polypeptide, a CAPN5 polypeptide, an IFT172 polypeptide, a KCNJ13 polypeptide, a PAX2 polypeptide, a CC2D2A polypeptide, a HMCN1 polypeptide, a MT-ATP6 polypeptide, a RCBTB1 polypeptide, an ARL2BP polypeptide, a CA4 polypeptide, a DFNB31 polypeptide, a GNB3 polypeptide, a MMACHC polypeptide, a PRPF4 polypeptide, a RGS9 polypeptide, an ARHGEF18 polypeptide, a KIAA1549 polypeptide, a MKS1 polypeptide, a MTTP (not MT-TP) polypeptide, a PLK4 polypeptide, a RPGRIP1L polypeptide, a SDCCAG8 polypeptide, a SRD5A3 polypeptide, a TUBB4B polypeptide, an ADAMTS18 polypeptide, an ARL3 polypeptide, a COL11A2 polypeptide, a MVK polypeptide, a NBAS polypeptide, an OFD1 polypeptide, a P3H2 polypeptide, a RGS9BP polypeptide, a CSPP1 polypeptide, an ITM2B polypeptide, a PANK2 polypeptide, a PEX7 polypeptide, a POMGNT1 polypeptide, a SLC4A7 polypeptide, a TMEM231 polypeptide, a TRNT1 polypeptide, a TUBGCP6 polypeptide, a ZNF513 polypeptide, an AFG3L2 polypeptide, an ARL13B polypeptide, a C5ORF42 (aka CPLANE1) polypeptide, a COL9AI polypeptide, a CTSD polypeptide, a DTHD1 polypeptide, a DYNC2H1 polypeptide, an IFT81 polypeptide, a KIAA0586 polypeptide, a MFN2 polypeptide, a NPHP3 polypeptide, a PCYT1A polypeptide, a PEX12 polypeptide, a PLA2G5 polypeptide, a POC5 polypeptide, a SCAPER polypeptide, a SLC25A46 polypeptide, a TMEM237 polypeptide, a TRAF3IP1 polypeptide, a TTC21B polypeptide, a TUBGCP4 polypeptide, an ADIPOR1 polypeptide, a CEP164 polypeptide, a CLCC1 polypeptide, a COL9A2 polypeptide, a CTNNB1 polypeptide, a DHX38 polypeptide, a GNPTG polypeptide, a GRN polypeptide, a GUCAIB polypeptide, an IFT27 polypeptide, an IFT74 polypeptide, a KIAA0556 polypeptide, a LRP2 polypeptide, a MAPKAPK3 polypeptide, a MIR204 polypeptide, a MT-ND3 polypeptide, a MT-RNR1 polypeptide, a MT-TS2 polypeptide, a ND5 (MT-ND5) polypeptide, a NEK2 polypeptide, an OPNlSW polypeptide, a PEX13 polypeptide, a PEX2 polypeptide, a RHBDD2 polypeptide, a SAMD11 polypeptide, a SCLT1 polypeptide, a SLC7A14 polypeptide, a TCTN1 polypeptide, a TCTN2 polypeptide, a TLCD3B polypeptide, a TREX1 polypeptide, a TTPA polypeptide, an UNC119 polypeptide, a WDPCP polypeptide, an ACBD5 polypeptide, an AHR polypeptide, an ARMC9 polypeptide, an ASRGL1 polypeptide, an ATOH7 polypeptide, a B9D1 polypeptide, a B9D2 polypeptide, a BBIP1 polypeptide, a C12ORF65 polypeptide, a C2CD3 polypeptide, a C5AR2 polypeptide, a CCDCl88 polypeptide, a CCT2 polypeptide, a CEP104 polypeptide, a CEP120 polypeptide, a CEP19 polypeptide, a CEP41 polypeptide, a CISD2 polypeptide, a CLUAP1 polypeptide, a COL9A3 polypeptide, a CRB2 polypeptide, a CTC1 polypeptide, a DACT2 polypeptide, a DDR1 polypeptide, an ENSA polypeptide, an ESPN polypeptide, an EXOSC2 polypeptide, a FBN3 polypeptide, a GDF6 polypeptide, a GPR125 polypeptide, a HKDC1 polypeptide, a HMX1 polypeptide, an IDH3B polypeptide, an IFT43 polypeptide, an IFT80 polypeptide, an INVS polypeptide, a KIAA0753 polypeptide, a KIF3B polypeptide, a KIF7 polypeptide, a LRRTM4 polypeptide, a LZTFL1 polypeptide, a MT-ATP8 polypeptide, a MT-CO1 polypeptide, a MT-C02 polypeptide, a MT-C03 polypeptide, a MT-CYB polypeptide, a MT-ND2 polypeptide, a MT-ND4L polypeptide, a MT-RNR2 polypeptide, a MT-TA polypeptide, a MT-TC polypeptide, a MT-TD polypeptide, a MT-TE polypeptide, a MT-TF polypeptide, a MT-TG polypeptide, a MT-TH polypeptide, a MT-TI polypeptide, a MT-TK polypeptide, a MT-TL2 polypeptide, a MT-TM polypeptide, a MT-TN polypeptide, a MT-TP (Not MTTP) polypeptide, a MT-TQ polypeptide, a MT-TR polypeptide, a MT-TS1 polypeptide, a MT-TT polypeptide, a MT-TV polypeptide, a MT-TW polypeptide, a MT-TY polypeptide, a NEURODI polypeptide, a PDE6D polypeptide, a PEX10 polypeptide, a PEX11B polypeptide, a PEX14 polypeptide, a PEX16 polypeptide, a PEX19 polypeptide, a PEX26 polypeptide, a PEX3 polypeptide, a PEX5 polypeptide, a PGK1 polypeptide, a PISD polypeptide, a PPP2R3C polypeptide, a PROS1 polypeptide, a PSEN1 polypeptide, a RDH11 polypeptide, a RRM2B polypeptide, a SMARCA4 polypeptide, a SPP2 polypeptide, a TCTN3 polypeptide, a TEAD1 polypeptide, a TMEM10⁷ polypeptide, a TMEM138 polypeptide, a TMEM216 polypeptide, a TMEM67 polypeptide, a TPP1 polypeptide, a TRIM32 polypeptide, a USP45 polypeptide, and a ZNF423 polypeptide. In some cases, a polypeptide of interest that can be encoded by an exogenous nucleic acid sequence designed to be included within an AAV vector provided herein can be a MYO7A polypeptide, an USH1C polypeptide, a PCDH15 polypeptide, an USH2A polypeptide, or CLRN1 polypeptide to treat, e.g., Usher Syndrome.

In some cases, one or more AAV vectors provided herein can be designed to carry out gene editing within one or more cells (e.g., retinal cells). Such gene editing can result in a genomic modification of one or more cells. Examples of such genomic modifications include, without limitation, a targeted insertion of a nucleic acid encoding an RNA and/or polypeptide of interest into one or more cells, a targeted modification (e.g., targeted inactivation or knock-out) of a genomic sequence of one or more cells, and a targeted replacement of nucleic acid (e.g., nucleic acid encoding an RNA, a regulatory nucleic acid sequence, and/or nucleic acid encoding a polypeptide of interest) within one or more cells.

Any appropriate gene editing components can be engineered into one or more AAV vectors provided herein such that those one or more AAV vectors can be used to deliver the gene editing components to target cells (e.g., one or more retinal cells) within a mammal (e.g., a human or a non-human primate) in a manner effective to edit the genome of those cells.

Typically, the gene editing components include, without limitation, a component that is capable of cleaving genomic nucleic acid at a desired location and an optional donor nucleic acid designed to be inserted into that desired location once it is cleaved. Any appropriate rare-cutting endonuclease can be used to cleave genomic nucleic acid at a desired location. Examples of such rare-cutting endonucleases include, without limitation, meganucleases, transcription activator-like effector (TALE) nucleases (TALENs™; Cellectis, Paris, France), zinc-finger-nucleases (ZFNs), and endonucleases of a clustered regularly interspaced short palindromic repeats (CRISPR)/Cas system (e.g., endonucleases of a CRISPR/Cas 9 system). See, e.g., Baker, Nature Methods, 9:23-26 (2012); International PCT Patent Application Publication No. WO 2004/067736; International PCT Patent Application Publication No. WO 2011/072246; U.S. Pat. No. 8,586,363; Porteus and Carroll, Nature Biotechnol., 23:967-973 (2005); Jinek et al., Science, 337:816-821 (2012); Mali et al., Science, 339:823-826 (2013); Li et al., Nature Biotechnology, 31(8):688-691 (2013); and Makarova et al., Nat. Rev. Microbiol., 9(6):467-477 (2011)).

In some cases, to facilitate gene replacement, two sequences in genomic nucleic acid of a cell (e.g., a retinal cell)—one on either side of a sequence to be removed—can be targeted for endonuclease cleavage. For example, a first target sequence adjacent to the 5′ end of a sequence to be removed and a second target sequence adjacent to the 3′ end of the sequence to be removed can be targeted by guide RNAs to enable Cas9 cleavage or can be targeted by TALENs designed to specifically recognize those targets. Delivery using one or more AAV vectors provided herein of (a) endonucleases targeted to the genomic DNA and (b) a donor nucleic acid construct can allow cleavage at both genomic targets, removal of the sequence between the genomic targets, and insertion of the donor sequence into the location of the deletion.

An AAV vector (e.g., an AAV2 vector) provided herein can include any appropriate promoter and/or other regulatory sequence (e.g., enhancers, transcription initiation sites, translation initiation sites, and termination signals) operably linked an exogenous nucleic acid sequence designed to be expressed. In some cases, a promoter used to drive expression can be a constitutive promotor, a regulatable promotor, a tissue-specific promoter, or a viral promotor. Examples of constitutive promotors that can be used as described herein include, without limitation, SV40 promotors, CMV promotors, and E1ALPHA promotors. Examples of regulatable promoters that can be used as described herein include, without limitation, inducible promotors and repressible promotors. Examples of tissue-specific promotors that can be used as described herein include, without limitation, rhodopsin promotors, cone arrestin promotors, and synapsin promotors. Examples of viral promotors that can be used as described herein include, without limitation, adenoviral promotors, vaccinia virus promotors, CMV promotors (e.g., immediate early CMV promotors), and AAV promoters.

In some cases, an AAV vector (e.g., an AAV2 vector) provided herein can include a total number of nucleotides up to about 5 kb. In some cases, an AAV vector (e.g., an AAV2 vector) provided herein can include a total number of nucleotides that is from about 1 kb to about 5 kb, from about 1 kb to about 4 kb, from about 1 kb to about 3 kb, from about 2 kb to about 5 kb, from about 2 kb to about 4 kb, from about 2 kb to about 3 kb, from about 3 kb to about 5 kb, from about 3 kb to about 4 kb, or from about 4 kb to about 5 kb.

An AAV vector (e.g., an AAV2 vector) described herein containing an AAV capsid polypeptide (e.g., an AAV2 capsid polypeptide) that includes an amino acid sequence set forth in Table 1A (or a variant thereof) or according to Formula A based on such a set forth sequence can have the ability to infect foveal cones in vivo and deliver exogenous nucleic acid sequence to the infected foveal cones such that the infected foveal cones express the exogenous nucleic acid sequence. In some cases, an AAV vector (e.g., an AAV2 vector) provided herein can have the ability to infect and drive RNA expression of an exogenous nucleic acid sequence in more cone cells present in the fovea of a mammal (e.g., a human or a non-human primate) when compared to wild-type AAV2. In some cases, an AAV vector (e.g., an AAV2 vector) provided herein can have the ability to drive a level of RNA expression of an exogenous nucleic acid sequence in foveal cones of a mammal (e.g., a human or a non-human primate) that is greater than the level of RNA expression of an exogenous nucleic acid sequence driven by a control AAV vector (e.g., wild-type AAV2) having an AAV capsid polypeptide that consists of the amino acid sequence set forth in SEQ ID NO:1 in foveal cones of a control mammal (e.g., a control human or a control non-human primate).

An AAV vector (e.g., an AAV2 vector) described herein containing an AAV capsid polypeptide (e.g., an AAV2 capsid polypeptide) that includes an amino acid sequence set forth in Table 1B (or a variant thereof) or according to Formula A based on such a set forth sequence can have the ability to infect retinal cells (e.g., retinal ganglion cells, photoreceptor cells, and bipolar cells) in vivo and deliver exogenous nucleic acid sequence to the infected retinal cells such that the infected retinal cells express the exogenous nucleic acid sequence (e.g., at high levels). In some cases, an AAV vector (e.g., an AAV2 vector) provided herein can have the ability to infect and drive RNA expression of an exogenous nucleic acid sequence in at least 2 percent (e.g., (e.g., at least 2.5 percent, at least 5 percent, at least 7.5 percent, at least 10 percent, or at least 25 percent) of retinal cells of an eye of a mammal (e.g., a human or a non-human primate). In some cases, an AAV vector (e.g., an AAV2 vector) provided herein can have the ability to drive a level of RNA expression of an exogenous nucleic acid sequence in retinal cells (e.g., retinal ganglion cells, photoreceptor cells, and bipolar cells) of a mammal (e.g., a human or a non-human primate) that is greater than (e.g., at least 2 percent greater than, at least 2.5 percent greater than, at least 5 percent greater than, at least 7.5 percent greater than, at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO:1 (e.g., a wild-type AAV2 vector) in retinal cells of a control mammal (e.g., a control human or a control non-human primate).

An AAV vector (e.g., an AAV2 vector) described herein containing an AAV capsid polypeptide (e.g., an AAV2 capsid polypeptide) that includes an amino acid sequence set forth in Table 1C (or a variant thereof) or according to Formula A based on such a set forth sequence can have the ability to infect retinal cells (e.g., retinal ganglion cells, photoreceptor cells, and bipolar cells) across retinal regions (e.g., across two, three, or four retinal regions) in vivo and deliver exogenous nucleic acid sequence to the infected retinal cells such that the infected retinal cells express the exogenous nucleic acid sequence (e.g., a high levels). In some cases, an AAV vector (e.g., an AAV2 vector) provided herein can have the ability to infect and drive RNA expression of an exogenous nucleic acid sequence in at least about 2 percent (e.g., at least about 2.5 percent, at least about 5 percent, at least about 7.5 percent, at least about 10 percent, or at least about 25 percent) of the retinal cells in the fovea region, at least about 2 percent (e.g., at least about 2.5 percent, at least about 5 percent, at least about 7.5 percent, at least about 10 percent, or at least about 25 percent) of the retinal cells in the parafovea region, at least about 2 percent (e.g., at least about 2.5 percent, at least about 5 percent, at least about 7.5 percent, at least about 10 percent, or at least about 25 percent) of the retinal cells in the vascular arcade region, and/or at least about 2 percent (e.g., at least about 2.5 percent, at least about 5 percent, at least about 7.5 percent, at least about 10 percent, or at least about 25 percent) of the retinal cells in the periphery region of an eye of a mammal (e.g., a human or a non-human primate). In some cases, an AAV vector (e.g., an AAV2 vector) provided herein can have the ability to drive a level of RNA expression of an exogenous nucleic acid sequence in retinal cells (e.g., retinal ganglion cells, photoreceptor cells, and bipolar cells) in at least two, three, or four different regions of an eye of a mammal (e.g., a human or a non-human primate) that is greater than the level of RNA expression of an exogenous nucleic acid sequence driven by a control AAV vector having an AAV capsid polypeptide that consists of the amino acid sequence set forth in SEQ ID NO:1 (e.g., a wild-type AAV2 vector) in retinal cells of those regions in a control mammal (e.g., a control human or a control non-human primate).

An AAV vector (e.g., an AAV2 vector) described herein containing an AAV capsid polypeptide (e.g., an AAV2 capsid polypeptide) that includes an amino acid sequence set forth in Table 1D (or a variant thereof) or according to Formula A based on such a set forth sequence can have the ability to infect retinal cells of the parafovea region of the eye in vivo and deliver exogenous nucleic acid sequence to the infected retinal cells such that the infected retinal cells express the exogenous nucleic acid sequence. In some cases, an AAV vector (e.g., an AAV2 vector) provided herein can have the ability to infect and drive RNA expression of an exogenous nucleic acid sequence in at least 2 percent (e.g., at least about 2.5 percent, at least about 5 percent, at least about 7.5 percent, at least about 10 percent, or at least about 25 percent) of retinal cells present in the parafovea region of the eye of a mammal (e.g., a human or a non-human primate). In some cases, an AAV vector (e.g., an AAV2 vector) provided herein can have the ability to drive a level of RNA expression of an exogenous nucleic acid sequence in retinal cells of the parafovea region of the eye of a mammal (e.g., a human or a non-human primate) that is greater than (e.g., at least 10 percent greater than, at least 25 percent greater than, at least 50 percent greater than, at least 75 percent greater than, or at least 100 percent greater than) the level of RNA expression of an exogenous nucleic acid sequence driven by a control AAV vector having an AAV capsid polypeptide that consists of the amino acid sequence set forth in SEQ ID NO:1 (e.g., a wild type AAV2 vector) in retinal cells of the parafovea region of the eye of a control mammal (e.g., a control human or a control non-human primate).

An AAV vector (e.g., an AAV2 vector) described herein containing an AAV capsid polypeptide (e.g., an AAV2 capsid polypeptide) that includes an amino acid sequence set forth in Table 1E (or a variant thereof) or according to Formula A based on such a set forth sequence can have the ability to infect two or more (e.g., two or more, three or more, four or more, five or more, six or more, or seven or more) different retinal cell types within an eye in vivo and deliver exogenous nucleic acid to the infected retinal cells such that the infected retinal cells express the exogenous nucleic acid. For example, an AAV vector (e.g., an AAV2 vector) described herein containing an AAV capsid polypeptide (e.g., an AAV2 capsid polypeptide) that includes an amino acid sequence set forth in Table 1E (or a variant thereof) or according to Formula A based on such a set forth sequence can have the ability to infect two, three, four, five, six, or seven of the following retinal cell types: retinal ganglion cells, amacrine cells, horizontal cells, bipolar cells, Muller glia cells, photoreceptor cells, and RPE cells. In some cases, an AAV vector (e.g., an AAV2 vector) described herein containing an AAV capsid polypeptide (e.g., an AAV2 capsid polypeptide) that includes an amino acid sequence set forth in Table 1E (or a variant thereof) or according to Formula A based on such a set forth sequence can have the ability to infect (a) retinal ganglion cells, amacrine cells, and horizontal cells, (b) retinal ganglion cells, amacrine cells, and bipolar cells, (c) retinal ganglion cells, amacrine cells, and Muller glia cells, (d) retinal ganglion cells, amacrine cells, and photoreceptor cells, (e) retinal ganglion cells, amacrine cells, and RPE cells, (f) amacrine cells, horizontal cells, and bipolar cells, (g) amacrine cells, horizontal cells, and Muller glia cells, (h) amacrine cells, horizontal cells, and photoreceptor cells, (i) amacrine cells, horizontal cells, and RPE cells, (j) horizontal cells, bipolar cells, and Muller glia cells, (k) horizontal cells, bipolar cells, and photoreceptor cells, (1) horizontal cells, bipolar cells, and RPE cells, (m) bipolar cells, Muller glia cells, and photoreceptor cells, (n) bipolar cells, Muller glia cells, and RPE cells, or (o) Muller glia cells, photoreceptor cells, and RPE cells. In some cases, an AAV vector (e.g., an AAV2 vector) provided herein can have the ability to infect and drive mRNA expression of an exogenous nucleic acid in at least 2 percent (e.g., at least about 2.5 percent, at least about 5 percent, at least about 7.5 percent, at least about percent, or at least about 25 percent) of the retinal ganglion cells, at least 2 percent (e.g., at least about 2.5 percent, at least about 5 percent, at least about 7.5 percent, at least about 10 percent, or at least about 25 percent) of the amacrine cells, at least 2 percent (e.g., at least about 2.5 percent, at least about 5 percent, at least about 7.5 percent, at least about 10 percent, or at least about 25 percent) of the horizontal cells, at least 2 percent (e.g., at least about 2.5 percent, at least about 5 percent, at least about 7.5 percent, at least about 10 percent, or at least about 25 percent) of the bipolar cells, at least 2 percent (e.g., at least about 2.5 percent, at least about 5 percent, at least about 7.5 percent, at least about 10 percent, or at least about 25 percent) of the Muller glia cells, at least 2 percent (e.g., at least about 2.5 percent, at least about 5 percent, at least about 7.5 percent, at least about 10 percent, or at least about 25 percent) of the photoreceptor cells, and/or at least 2 percent (e.g., at least about 2.5 percent, at least about 5 percent, at least about 7.5 percent, at least about 10 percent, or at least about 25 percent) of the RPE cells of an eye of a mammal (e.g., a human or a non-human primate) following, for example, an intravitreal administration.

An AAV vector (e.g., an AAV2 vector) described herein containing an AAV capsid polypeptide (e.g., an AAV2 capsid polypeptide) that includes an amino acid sequence set forth in Table 1F (or a variant thereof) or according to Formula A based on such a set forth sequence can have the ability to infect RPE cells in vivo and deliver exogenous nucleic acid sequence to the infected RPE cells such that the infected RPE cells express the exogenous nucleic acid sequence (e.g., at high levels). In some cases, an AAV vector (e.g., an AAV2 vector) provided herein can have the ability to infect and drive RNA expression of an exogenous nucleic acid sequence in at least 2 percent (e.g., at least about 2.5 percent, at least about 5 percent, at least about 7.5 percent, at least about 10 percent, or at least about 25 percent) of RPE cells of an eye of a mammal (e.g., a human or a non-human primate). In some cases, an AAV vector (e.g., an AAV2 vector) provided herein can have the ability to drive a level of RNA expression of an exogenous nucleic acid sequence in RPE cells of an eye of a mammal (e.g., a human or a non-human primate) that is greater than (e.g., at least 10 percent greater than, at least 25 percent greater than, at least 50 percent greater than, at least 75 percent greater than, or at least 100 percent greater than) the level of RNA expression of an exogenous nucleic acid sequence driven by a control AAV vector having an AAV capsid polypeptide that consists of the amino acid sequence set forth in SEQ ID NO:1 in RPE cells of an eye of a control mammal (e.g., a control human or a control non-human primate).

An AAV vector (e.g., an AAV2 vector) described herein containing an AAV capsid polypeptide (e.g., an AAV2 capsid polypeptide) that includes an amino acid sequence set forth in Table 1G (or a variant thereof) or according to Formula A based on such a set forth sequence can have the ability to infect photoreceptor cells in vivo and deliver exogenous nucleic acid sequence to the infected photoreceptor cells such that the infected photoreceptor cells express the exogenous nucleic acid sequence. For example, an AAV vector (e.g., an AAV2 vector) described herein containing an AAV capsid polypeptide (e.g., an AAV2 capsid polypeptide) that includes an amino acid sequence set forth in Table 1G (or a variant thereof) or according to Formula A based on such a set forth sequence can have the ability to infect photoreceptor cells in vivo to a greater extent than any other retinal cell type of an eye and deliver exogenous nucleic acid sequence to the infected photoreceptor cells such that the infected photoreceptor cells express the exogenous nucleic acid sequence. In some cases, an AAV vector (e.g., an AAV2 vector) provided herein can have the ability to infect and drive RNA expression of an exogenous nucleic acid sequence in at least 2 percent (e.g., at least about 2.5 percent, at least about 5 percent, at least about 7.5 percent, at least about 10 percent, or at least about 25 percent) of photoreceptor cells of an eye of a mammal (e.g., a human or a non-human primate). In some cases, an AAV vector (e.g., an AAV2 vector) provided herein can have the ability to drive a level of RNA expression of an exogenous nucleic acid sequence in photoreceptor cells of an eye of a mammal (e.g., a human or a non-human primate) that is greater than (e.g., at least 10 percent greater than, at least 25 percent greater than, at least 50 percent greater than, at least 75 percent greater than, or at least 100 percent greater than) the level of RNA expression of an exogenous nucleic acid sequence driven by a control AAV vector having an AAV capsid polypeptide that consists of the amino acid sequence set forth in SEQ ID NO:1 in photoreceptor cells of an eye of a control mammal (e.g., a control human or a control non-human primate).

An AAV vector (e.g., an AAV2 vector) described herein containing an AAV capsid polypeptide (e.g., an AAV2 capsid polypeptide) that includes an amino acid sequence set forth in Table 1H (or a variant thereof) or according to Formula A based on such a set forth sequence can have the ability to infect retinal ganglion cells in vivo and deliver exogenous nucleic acid sequence to the infected retinal ganglion cells such that the infected retinal ganglion cells express the exogenous nucleic acid sequence. For example, an AAV vector (e.g., an AAV2 vector) described herein containing an AAV capsid polypeptide (e.g., an AAV2 capsid polypeptide) that includes an amino acid sequence set forth in Table 1H (or a variant thereof) or according to Formula A based on such a set forth sequence can have the ability to infect retinal ganglion cells in vivo to a greater extent than any other retinal cell type of an eye and deliver exogenous nucleic acid sequence to the infected retinal ganglion cells such that the infected retinal ganglion cells express the exogenous nucleic acid sequence. In some cases, an AAV vector (e.g., an AAV2 vector) provided herein can have the ability to infect and drive RNA expression of an exogenous nucleic acid sequence in at least 2 percent (e.g., at least about 2.5 percent, at least about 5 percent, at least about 7.5 percent, at least about 10 percent, or at least about 25 percent) of retinal ganglion cells of an eye of a mammal (e.g., a human or a non-human primate). In some cases, an AAV vector (e.g., an AAV2 vector) provided herein can have the ability to drive a level of RNA expression of an exogenous nucleic acid sequence in retinal ganglion cells of an eye of a mammal (e.g., a human or a non-human primate) that is greater than (e.g., at least 10 percent greater than, at least 25 percent greater than, at least 50 percent greater than, at least 75 percent greater than, or at least 100 percent greater than) the level of RNA expression of an exogenous nucleic acid sequence driven by a control AAV vector having an AAV capsid polypeptide that consists of the amino acid sequence set forth in SEQ ID NO:1 in retinal ganglion cells of an eye of a control mammal (e.g., a control human or a control non-human primate).

An AAV vector (e.g., an AAV2 vector) described herein containing an AAV capsid polypeptide (e.g., an AAV2 capsid polypeptide) that includes an amino acid sequence set forth in Table 11 (or a variant thereof) or according to Formula A based on such a set forth sequence can have the ability to infect bipolar cells of the retina in vivo and deliver exogenous nucleic acid sequence to the infected bipolar cells such that the infected bipolar cells express the exogenous nucleic acid sequence. For example, an AAV vector (e.g., an AAV2 vector) described herein containing an AAV capsid polypeptide (e.g., an AAV2 capsid polypeptide) that includes an amino acid sequence set forth in Table 11 (or a variant thereof) or according to Formula A based on such a set forth sequence can have the ability to infect bipolar cells of the retina in vivo to a greater extent than any other retinal cell type of an eye and deliver exogenous nucleic acid sequence to the infected bipolar cells such that the infected bipolar cells express the exogenous nucleic acid sequence. In some cases, an AAV vector (e.g., an AAV2 vector) provided herein can have the ability to infect and drive RNA expression of an exogenous nucleic acid sequence in at least 2 percent (e.g., at least about 2.5 percent, at least about 5 percent, at least about 7.5 percent, at least about 10 percent, or at least about 25 percent) of bipolar cells of the retina of an eye of a mammal (e.g., a human or a non-human primate). In some cases, an AAV vector (e.g., an AAV2 vector) provided herein can have the ability to drive a level of RNA expression of an exogenous nucleic acid sequence in bipolar cells of the retina of an eye of a mammal (e.g., a human or a non-human primate) that is greater than (e.g., at least 10 percent greater than, at least percent greater than, at least 50 percent greater than, at least 75 percent greater than, or at least 100 percent greater than) the level of RNA expression of an exogenous nucleic acid sequence driven by a control AAV vector having an AAV capsid polypeptide that consists of the amino acid sequence set forth in SEQ ID NO:1 in bipolar cells of the retina of an eye of a control mammal (e.g., a control human or a control non-human primate).

An AAV vector (e.g., an AAV2 vector) described herein containing an AAV capsid polypeptide (e.g., an AAV2 capsid polypeptide) that includes an amino acid sequence set forth in Table 1J (or a variant thereof) or according to Formula A based on such a set forth sequence can have the ability to infect ON-retinal ganglion cells in vivo and deliver exogenous nucleic acid sequence to the infected ON-retinal ganglion cells such that the infected ON-retinal ganglion cells express the exogenous nucleic acid sequence. For example, an AAV vector (e.g., an AAV2 vector) described herein containing an AAV capsid polypeptide (e.g., an AAV2 capsid polypeptide) that includes an amino acid sequence set forth in Table 1J (or a variant thereof) or according to Formula A based on such a set forth sequence can have the ability to infect ON-retinal ganglion cells in vivo to a greater extent than any other retinal cell type of an eye (e.g., a greater extent than OFF-retinal ganglion cells) and deliver exogenous nucleic acid sequence to the infected ON-retinal ganglion cells such that the infected ON-retinal ganglion cells express the exogenous nucleic acid sequence. In some cases, an AAV vector (e.g., an AAV2 vector) provided herein can have the ability to infect and drive RNA expression of an exogenous nucleic acid sequence in at least 2 percent (e.g., at least about 2.5 percent, at least about 5 percent, at least about 7.5 percent, at least about 10 percent, or at least about 25 percent) of ON-retinal ganglion cells of an eye of a mammal (e.g., a human or a non-human primate). In some cases, an AAV vector (e.g., an AAV2 vector) provided herein can have the ability to drive a level of RNA expression of an exogenous nucleic acid sequence in ON-retinal ganglion cells of an eye of a mammal (e.g., a human or a non-human primate) that is greater than (e.g., at least 10 percent greater than, at least 25 percent greater than, at least 50 percent greater than, at least 75 percent greater than, or at least 100 percent greater than) the level of RNA expression of an exogenous nucleic acid sequence driven by a control AAV vector having an AAV capsid polypeptide that consists of the amino acid sequence set forth in SEQ ID NO:1 in ON-retinal ganglion cells of an eye of a control mammal (e.g., a control human or a control non-human primate).

An AAV vector (e.g., an AAV2 vector) described herein containing an AAV capsid polypeptide (e.g., an AAV2 capsid polypeptide) that includes an amino acid sequence set forth in Table 1K (or a variant thereof) or according to Formula A based on such a set forth sequence can have the ability to infect OFF-retinal ganglion cells in vivo and deliver exogenous nucleic acid sequence to the infected OFF-retinal ganglion cells such that the infected OFF-retinal ganglion cells express the exogenous nucleic acid sequence. For example, an AAV vector (e.g., an AAV2 vector) described herein containing an AAV capsid polypeptide (e.g., an AAV2 capsid polypeptide) that includes an amino acid sequence set forth in Table 1K (or a variant thereof) or according to Formula A based on such a set forth sequence can have the ability to infect OFF-retinal ganglion cells in vivo to a greater extent than any other retinal cell type of an eye (e.g., a greater extent than ON-retinal ganglion cells) and deliver exogenous nucleic acid sequence to the infected OFF-retinal ganglion cells such that the infected OFF-retinal ganglion cells express the exogenous nucleic acid sequence. In some cases, an AAV vector (e.g., an AAV2 vector) provided herein can have the ability to infect and drive RNA expression of an exogenous nucleic acid sequence in at least 2 percent (e.g., at least about 2.5 percent, at least about 5 percent, at least about 7.5 percent, at least about 10 percent, or at least about 25 percent) of OFF-retinal ganglion cells of an eye of a mammal (e.g., a human or a non-human primate). In some cases, an AAV vector (e.g., an AAV2 vector) provided herein can have the ability to drive a level of RNA expression of an exogenous nucleic acid sequence in OFF-retinal ganglion cells of an eye of a mammal (e.g., a human or a non-human primate) that is greater than (e.g., at least 10 percent greater than, at least 25 percent greater than, at least 50 percent greater than, at least 75 percent greater than, or at least 100 percent greater than) the level of RNA expression of an exogenous nucleic acid sequence driven by a control AAV vector having an AAV capsid polypeptide that consists of the amino acid sequence set forth in SEQ ID NO:1 in OFF-retinal ganglion cells of an eye of a control mammal (e.g., a control human or a control non-human primate).

An AAV vector (e.g., an AAV2 vector) described herein containing an AAV capsid polypeptide (e.g., an AAV2 capsid polypeptide) that includes an amino acid sequence set forth in Table 1L (or a variant thereof) or according to Formula A based on such a set forth sequence can have increased packaging efficiency, the ability to infect cells (e.g., retinal cells) in vivo or in vitro, and the ability to deliver exogenous nucleic acid sequence to the infected cells such that the infected cells express the exogenous nucleic acid sequence (e.g., at high levels). In some cases, an AAV vector (e.g., an AAV2 vector) provided herein can have a packaging efficiency that is greater than (e.g., at least 10 percent greater than, at least 25 percent greater than, at least 50 percent greater than, at least 75 percent greater than, or at least 100 percent greater than) the packaging efficiency of a comparable AAV vector having an AAV capsid polypeptide that consists of the amino acid sequence of SEQ ID NO:1 (e.g., a wild-type AAV2 vector).

Examples of retinal cells that can be infected by an AAV vector (e.g., an AAV2 vector) described herein containing an AAV capsid polypeptide (e.g., an AAV2 capsid polypeptide) that includes an amino acid sequence set forth in Table 1 (or a variant thereof) or Formula A include, without limitation, retinal ganglion cells, retinal pigment epithelium cells, photoreceptor cells, bipolar cells, amacrine cells, Muller glia, and horizontal cells.

This document also provides compositions containing one or more AAV vectors provided herein (e.g., one or more AAV2 vectors provided herein). For example, one or more AAV vectors provided herein (e.g., one or more AAV2 vectors provided herein) can be 19.5 formulated as a pharmaceutical composition for administration to a mammal (e.g., a human or a non-human primate) to treat that mammal. In some cases, one or more AAV vectors provided herein (e.g., one or more AAV2 vectors provided herein) can be formulated as a pharmaceutical composition for administration to a mammal (e.g., a human or a non-human primate) to deliver an exogenous nucleic acid sequence to foveal cones for expression within foveal cones. For example, an AAV vector (e.g., an AAV2 vector) provided herein can be formulated as a pharmaceutical composition for administration to a mammal (e.g., a human or a non-human primate). In some cases, a pharmaceutical composition provided herein can include a pharmaceutically acceptable carrier such as a buffer, a salt, a surfactant, a sugar, a tonicity modifier, or combinations thereof as, for example, described elsewhere (Gervasi et al., Eur. J. Pharmaceutics and Biopharmaceutics, 131:8-24 (2018)). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), or combinations thereof. For example, a pharmaceutical composition designed to include an AAV vector (e.g., an AAV2 vector) provided herein can be formulated to include a buffer (e.g., an acetate, citrate, histidine, succinate, phosphate, or hydroxymethyl-aminomethane (Tris) buffer), a surfactant (e.g., polysorbate 20, polysorbate 80, or poloxamer 188), and a sugar such as sucrose. Other ingredients that can be included within a pharmaceutical composition provided herein include, without limitation, amino acids such as glycine or arginine, antioxidants such as ascorbic acid, methionine, or ethylenediaminetetraacetic acid (EDTA), or combinations thereof.

In some cases, when a pharmaceutical composition is formulated to include one or more AAV vectors (e.g., one or more AAV2 vectors) provided herein, any appropriate titer of the AAV vectors can be used. For example, a pharmaceutical composition provided herein can be formulated to have AAV vectors (e.g., AAV2 vectors) provided herein at a titer that is greater than 1×10⁷ (e.g., greater than 1×10⁸, greater than 1×10⁹, greater than 1×10¹⁰, greater than 1×10¹¹, greater than 1×10¹², greater than 1×10¹³, or greater than 1×10¹⁴). In some cases, a pharmaceutical composition provided herein can be formulated to have AAV vectors (e.g., AAV2 vectors) provided herein at a titer that is from about 1×10⁷ to about 1×10¹⁴ (e.g., from about 1×10⁷ to about 1×10¹³, from about 1×10⁷ to about 1×10¹², from about 1×10⁷ to about 1×10¹¹, from about 1×10⁷ to about 1×10¹⁰, from about 1×10⁸ to about 1×10¹⁴, from about 1×10⁹ to about 1×10¹⁴, from about 1×10¹⁰ to about 1×10¹⁴, from about 1×10⁸ to about 1×10¹², or from about 1×10⁹ to about 1×10¹¹).

A pharmaceutical composition provided herein can be in any appropriate form. For example, a pharmaceutical composition provided herein can be designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized. In some cases, a pharmaceutical composition provided herein (e.g., a pharmaceutical composition that includes one or more AAV vectors provided herein such as one or more AAV2 vectors provided herein) can be formulated with a carrier or coating designed to protect against rapid release. For example, a pharmaceutical composition provided herein can be formulated as a controlled release formulation or as a regulated release formulation as described elsewhere (U.S. Patent Application Publication Nos. 2019/0241667; 2019/0233522; and 2019/0233498).

This document also provides nucleic acid molecules encoding an AAV capsid polypeptide that includes an amino acid sequence set forth in any one or more of Tables 1A-1L (or a variant thereof) or according to Formula A based on such a set forth sequence. In some cases, a nucleic acid molecule can be designed to encode an AAV capsid polypeptide that includes an amino acid sequence that is encoded by a DNA sequence set forth in any one or more of Tables 1A-1L (e.g., any one of SEQ ID NOs:A20-A37, SEQ ID NOs:B318-B649, SEQ ID NOs:C46-C88, SEQ ID NOs:D79-D155, SEQ ID NOs:E84-E165, SEQ ID NOs:F11-F19, SEQ ID NOs:G78-G153, SEQ ID NOs:H259-H515, SEQ ID NOs:I75-1147, SEQ ID NOs:J65-J127, SEQ ID NOs:K64-K125, and SEQ ID NOs:L28-L53).

This document also provides nucleic acid molecules encoding an AAV vector (e.g., an AAV2 vector) described herein. For example, an isolated nucleic acid molecule can be designed to encode one or more AAV vectors provided herein (e.g., an AAV having an AAV capsid polypeptide that includes an amino acid sequence set forth in any one or more of Tables 1A-1L (or a variant thereof) or according to Formula A based on such a set forth sequence). In some cases, a nucleic acid molecule can be designed to encode an AAV vector having an AAV capsid polypeptide that includes an amino acid sequence that is encoded by a DNA sequence set forth in any one or more of Tables 1A-1L (e.g., any one of SEQ ID NOs:A20-A37, SEQ ID NOs:B318-B649, SEQ ID NOs:C46-C88, SEQ ID NOs:D79-D155, SEQ ID NOs:E84-E165, SEQ ID NOs:F11-F19, SEQ ID NOs:G78-G153, SEQ ID NOs:H259-H515, SEQ ID NOs:I75-I147, SEQ ID NOs:J65-J127, SEQ ID NOs:K64-K125, and SEQ ID NOs:L28-L53).

This document also provides host cells containing a nucleic acid molecule provided herein. For example, a host cell can be designed to include a nucleic acid molecule encoding an AAV capsid polypeptide described herein and/or a nucleic acid molecule encoding an AAV vector described herein. In some cases, a host cell can be designed to include a nucleic acid molecule encoding an AAV capsid polypeptide that includes an amino acid sequence set forth in any one or more of Tables 1A-1L (or a variant thereof) or according to Formula A based on such a set forth sequence. In some cases, a host cell can be designed to include a nucleic acid molecule encoding an AAV vector having an AAV capsid polypeptide that includes an amino acid sequence set forth in any one or more of Tables 1A-1L (or a variant thereof) or according to Formula A based on such a set forth sequence. Examples of host cells that can be designed to include a nucleic acid molecule encoding an AAV capsid polypeptide described herein and/or a nucleic acid molecule encoding an AAV vector described herein include, without limitation, HEK293T cells (ATCC), 293AAV cells (Cell Biolabs), NEB 5-alpha cells, TakaraBio Stellar cells, and MegaX cells. Any appropriate method can be used to introduce a nucleic acid molecule provided herein (e.g., a nucleic acid molecule encoding an AAV capsid polypeptide described herein and/or an AAV vector described herein) into a cell. For example, viral transfection, electroporation, transient transfection, and gene gun techniques can be used to introduce a nucleic acid molecule provided herein into a cell.

This document also provides methods and materials for making an AAV vector (e.g., an AAV2 vector) provided herein. For example, this document provides methods and materials for making AAV vectors (e.g., AAV2 vectors) containing an AAV capsid polypeptide that includes an amino acid sequence set forth in any one or more of Tables 1A-1L (or a variant thereof) or according to Formula A based on such a set forth sequence. As described herein, an AAV vector can be constructed to include an AAV capsid polypeptide that includes an amino acid sequence set forth in any one or more of Tables 1A-1L (or a variant thereof) or according to Formula A based on such a set forth sequence. Any appropriate method can be used to construct an AAV vector having an AAV capsid polypeptide (e.g., an AAV2 capsid polypeptide) provided herein (e.g., a capsid polypeptide that includes an amino acid sequence set forth in any one or more of Tables 1A-1L (or a variant thereof) or according to Formula A based on such a set forth sequence). For example, molecular cloning and AAV vector production techniques such as those described elsewhere can be used to construct and produce an AAV vector having an AAV capsid polypeptide (e.g., an AAV2 capsid polypeptide) provided herein (see, e.g., Sambrook et al., Molecular Cloning: A Laboratory Manual, 2nd edition, Cold Spring Harbor Laboratory, NY (1989); Ausubel et al., Current Protocols in Molecular Biology, Green Publishing Associates and John Wiley & Sons, New York, N.Y. (1994); Grieger et al., Nat. Protoc., 1(3):1412-28 (2006); and Flannery et al., Methods Mol. Biol., 935:351-69 (2013)). In some cases, AAV vectors can be produced in HEK293T cells (ATCC) or 293AAV cells (Cell Biolabs) using a double or triple transfection method (see, e.g., Grieger et al., Nat. Protoc., 1(3):1412-28 (2006); and Flannery et al., Methods Mol. Biol., 935:351-69 (2013)).

This document also provides methods and materials for using an AAV vector (e.g., an AAV2 vector) provided herein. For example, this document provides methods and materials for using AAV vectors (e.g., AAV2 vectors) containing an AAV capsid polypeptide that includes an amino acid sequence set forth in any one or more of Tables 1A-1L (or a variant thereof) or according to Formula A based on such a set forth sequence. As described herein, an AAV vector provided herein (e.g., an AAV vector (e.g., an AAV2 vector) containing an AAV capsid polypeptide that includes an amino acid sequence set forth in Table 1A or according to Formula A based on such a set forth sequence) can be used to infect foveal cones in vivo and to deliver an exogenous nucleic acid sequence to the infected foveal cones such that the infected foveal cones express the exogenous nucleic acid sequence.

As described herein, an AAV vector provided herein (e.g., an AAV vector (e.g., an AAV2 vector) containing an AAV capsid polypeptide that includes an amino acid sequence set forth in Table 1B or according to Formula A based on such a set forth sequence) can be used to infect retinal cells (e.g., retinal ganglion cells, photoreceptor cells, and bipolar cells) in vivo and to deliver an exogenous nucleic acid sequence to the infected retinal cells such that the infected retinal cells express the exogenous nucleic acid sequence (e.g., at high levels).

As described herein, an AAV vector provided herein (e.g., an AAV vector (e.g., an AAV2 vector) containing an AAV capsid polypeptide that includes an amino acid sequence set forth in Table 1C or according to Formula A based on such a set forth sequence) can be used to infect retinal cells (e.g., retinal ganglion cells, photoreceptor cells, and bipolar cells) across retinal regions (e.g., across two, three, or four different retinal regions) in vivo and to deliver an exogenous nucleic acid sequence to the infected retinal cells such that the infected retinal cells express the exogenous nucleic acid sequence (e.g., at high levels). For example, an AAV vector provided herein (e.g., an AAV vector (e.g., an AAV2 vector) containing an AAV capsid polypeptide that includes an amino acid sequence set forth in Table 1C or according to Formula A based on such a set forth sequence) can be used to infect retinal cells (e.g., retinal ganglion cells, photoreceptor cells, and bipolar cells) across retinal regions such that the AAV vector infects at least about 2 percent (e.g., at least about 2.5 percent, at least about 5 percent, at least about 7.5 percent, at least about 10 percent, or at least about 25 percent) of the retinal cells in the fovea region, at least about 2 percent (e.g., at least about 2.5 percent, at least about 5 percent, at least about 7.5 percent, at least about 10 percent, or at least about 25 percent) of the retinal cells in the parafovea region, at least about 2 percent (e.g., at least about 2.5 percent, at least about 5 percent, at least about 7.5 percent, at least about 10 percent, or at least about 25 percent) of the retinal cells in the vascular arcade region, and/or at least about 2 percent (e.g., at least about 2.5 percent, at least about 5 percent, at least about 7.5 percent, at least about 10 percent, or at least about 25 percent) of the retinal cells in the periphery region of an eye of a mammal (e.g., a human or a non-human primate).

As described herein, an AAV vector provided herein (e.g., an AAV vector (e.g., an AAV2 vector) containing an AAV capsid polypeptide that includes an amino acid sequence set forth in Table 1D or according to Formula A based on such a set forth sequence) can be used to infect retinal cells of the parafovea region of the eye in vivo and to deliver an exogenous nucleic acid sequence to the infected retinal cells such that the infected retinal cells express the exogenous nucleic acid sequence.

As described herein, an AAV vector provided herein (e.g., an AAV vector (e.g., an AAV2 vector) containing an AAV capsid polypeptide that includes an amino acid sequence set forth in Table 1E or according to Formula A based on such a set forth sequence) can be used to infect two or more (e.g., two or more, three or more, four or more, five or more, six or more, or seven or more) different retinal cell types within an eye in vivo and to deliver an exogenous nucleic acid sequence to the infected retinal cells such that the infected retinal cells express the exogenous nucleic acid sequence (e.g., at high levels). For example, an AAV vector provided herein (e.g., an AAV vector (e.g., an AAV2 vector) containing an AAV capsid polypeptide that includes an amino acid sequence set forth in Table 1E or according to Formula A based on such a set forth sequence) can be used to infect (a) at least about 2 percent (e.g., at least about 2.5 percent, at least about 5 percent, at least about 7.5 percent, at least about 10 percent, or at least about 25 percent) of the retinal ganglion cells, (b) at least about 2 percent (e.g., at least about 2.5 percent, at least about 5 percent, at least about 7.5 percent, at least about 10 percent, or at least about 25 percent) of the amacrine cells, (c) at least about 2 percent (e.g., at least about 2.5 percent, at least about 5 percent, at least about 7.5 percent, at least about 10 percent, or at least about 25 percent) of the horizontal cells, (d) at least about 2 percent (e.g., at least about 2.5 percent, at least about 5 percent, at least about 7.5 percent, at least about 10 percent, or at least about 25 percent) of the bipolar cells, (e) at least about 2 percent (e.g., at least about 2.5 percent, at least about 5 percent, at least about 7.5 percent, at least about 10 percent, or at least about 25 percent) of the Muller glia cells, (f) at least about 2 percent (e.g., at least about 2.5 percent, at least about 5 percent, at least about 7.5 percent, at least about 10 percent, or at least about 25 percent) of the photoreceptor cells, (g) at least about 2 percent (e.g., at least about 2.5 percent, at least about 5 percent, at least about 7.5 percent, at least about 10 percent, or at least about 25 percent) of the RPE cells, all of (a)-(g), or any combination of two, three, four, five, or six of (a)-(g) of an eye of a mammal (e.g., a human or a non-human primate) following, for example, an intravitreal administration.

As described herein, an AAV vector provided herein (e.g., an AAV vector (e.g., an AAV2 vector) containing an AAV capsid polypeptide that includes an amino acid sequence set forth in Table 1F or according to Formula A based on such a set forth sequence) can be used to infect RPE cells in vivo and to deliver an exogenous nucleic acid sequence to the infected RPE cells such that the infected RPE cells express the exogenous nucleic acid sequence (e.g., at high levels). For example, an AAV vector provided herein (e.g., an AAV vector (e.g., an AAV2 vector) containing an AAV capsid polypeptide that includes an amino acid sequence set forth in Table 1F or according to Formula A based on such a set forth sequence) can be used to infect RPE cells such that the AAV vector infects at least about 2 percent (e.g., at least about 2.5 percent, at least about 5 percent, at least about 7.5 percent, at least about 10 percent, or at least about 25 percent) of RPE cells of an eye of a mammal (e.g., a human or a non-human primate). As described herein, an AAV vector provided herein (e.g., an AAV vector (e.g., an AAV2 vector) containing an AAV capsid polypeptide that includes an amino acid sequence set forth in Table 1G or according to Formula A based on such a set forth sequence) can be used to infect photoreceptor cells in vivo and to deliver an exogenous nucleic acid sequence to the infected photoreceptor cells such that the infected photoreceptor cells express the exogenous nucleic acid sequence. For example, an AAV vector provided herein (e.g., an AAV vector (e.g., an AAV2 vector) containing an AAV capsid polypeptide that includes an amino acid sequence set forth in Table 1G or according to Formula A based on such a set forth sequence) can be used to infect photoreceptor cells in vivo to a greater extent than any other retinal cell type of an eye and to deliver an exogenous nucleic acid sequence to the infected photoreceptor cells such that the infected photoreceptor cells express the exogenous nucleic acid sequence to a greater extent than any other retinal cell type of an eye.

As described herein, an AAV vector provided herein (e.g., an AAV vector (e.g., an AAV2 vector) containing an AAV capsid polypeptide that includes an amino acid sequence set forth in Table 1H or according to Formula A based on such a set forth sequence) can be used to infect retinal ganglion cells in vivo and to deliver an exogenous nucleic acid sequence to the infected retinal ganglion cells such that the infected retinal ganglion cells express the exogenous nucleic acid sequence. For example, an AAV vector provided herein (e.g., an AAV vector (e.g., an AAV2 vector) containing an AAV capsid polypeptide that includes an amino acid sequence set forth in Table 1H or according to Formula A based on such a set forth sequence) can be used to infect retinal ganglion cells in vivo to a greater extent than any other retinal cell type of an eye and to deliver an exogenous nucleic acid sequence to the infected retinal ganglion cells such that the infected retinal ganglion cells express the exogenous nucleic acid sequence to a greater extent than any other retinal cell type of an eye.

As described herein, an AAV vector provided herein (e.g., an AAV vector (e.g., an AAV2 vector) containing an AAV capsid polypeptide that includes an amino acid sequence set forth in Table 1I or according to Formula A based on such a set forth sequence) can be used to infect bipolar cells of the retina in vivo and to deliver an exogenous nucleic acid sequence to the infected bipolar cells such that the infected bipolar cells express the exogenous nucleic acid sequence. For example, an AAV vector provided herein (e.g., an AAV vector (e.g., an AAV2 vector) containing an AAV capsid polypeptide that includes an amino acid sequence set forth in Table 11 or according to Formula A based on such a set forth sequence) can be used to infect bipolar cells of the retina in vivo to a greater extent than any other retinal cell type of an eye and to deliver an exogenous nucleic acid sequence to the infected bipolar cells such that the infected bipolar cells express the exogenous nucleic acid sequence to a greater extent than any other retinal cell type of an eye.

As described herein, an AAV vector provided herein (e.g., an AAV vector (e.g., an AAV2 vector) containing an AAV capsid polypeptide that includes an amino acid sequence set forth in Table 1J or according to Formula A based on such a set forth sequence) can be used to infect ON-retinal ganglion cells in vivo and to deliver an exogenous nucleic acid sequence to the infected ON-retinal ganglion cells such that the infected ON-retinal ganglion cells express the exogenous nucleic acid sequence. For example, an AAV vector provided herein (e.g., an AAV vector (e.g., an AAV2 vector) containing an AAV capsid polypeptide that includes an amino acid sequence set forth in Table 1J or according to Formula A based on such a set forth sequence) can be used to infect ON-retinal ganglion cells in vivo to a greater extent than any other retinal cell type of an eye (e.g., a greater extent than that of OFF-retinal ganglion cells) and to deliver an exogenous nucleic acid sequence to the infected ON-retinal ganglion cells such that the infected ON-retinal ganglion cells express the exogenous nucleic acid sequence to a greater extent than any other retinal cell type of an eye (e.g., OFF-retinal ganglion cells).

As described herein, an AAV vector provided herein (e.g., an AAV vector (e.g., an AAV2 vector) containing an AAV capsid polypeptide that includes an amino acid sequence set forth in Table 1K or according to Formula A based on such a set forth sequence) can be used to infect OFF-retinal ganglion cells in vivo and to deliver an exogenous nucleic acid sequence to the infected OFF-retinal ganglion cells such that the infected OFF-retinal ganglion cells express the exogenous nucleic acid sequence. For example, an AAV vector provided herein (e.g., an AAV vector (e.g., an AAV2 vector) containing an AAV capsid polypeptide that includes an amino acid sequence set forth in Table 1K or according to Formula A based on such a set forth sequence) can be used to infect OFF-retinal ganglion cells in vivo to a greater extent than any other retinal cell type of an eye (e.g., a greater extent than that of ON-retinal ganglion cells) and to deliver an exogenous nucleic acid sequence to the infected OFF-retinal ganglion cells such that the infected OFF-retinal ganglion cells express the exogenous nucleic acid sequence to a greater extent than any other retinal cell type of an eye (e.g., ON-retinal ganglion cells).

As described herein, an AAV vector provided herein (e.g., an AAV vector (e.g., an AAV2 vector) containing an AAV capsid polypeptide that includes an amino acid sequence set forth in Table 1L or according to Formula A based on such a set forth sequence) can be used to improve packaging efficiency. In some cases, an AAV vector provided herein (e.g., an AAV vector (e.g., an AAV2 vector) containing an AAV capsid polypeptide that includes an amino acid sequence set forth in Table 1L or according to Formula A based on such a set forth sequence) can be used to infect cells (e.g., retinal cells) in vivo or in vitro and to deliver an exogenous nucleic acid sequence to the infected cells such that the infected cells express the exogenous nucleic acid sequence (e.g., at high levels).

In some cases, an AAV vector (e.g., an AAV2 vector) provided herein (e.g., an AAV vector containing an AAV capsid polypeptide that includes an amino acid sequence set forth in any one or more of Tables 1A-1L (or a variant thereof) or according to Formula A based on such a set forth sequence) can be used to treat a retinal condition (e.g., a retinal disease). For example, an AAV vector (e.g., an AAV2 vector) provided herein (e.g., an AAV vector (e.g., an AAV2 vector) containing an AAV capsid polypeptide that includes an amino acid sequence set forth in Table 1A or according to Formula A based on such a set forth sequence) that is designed to contain and drive expression of an exogenous nucleic acid sequence encoding an RNA and/or polypeptide capable of treating a retinal condition (e.g., a retinal disease) can be administered to a mammal (e.g., a human or a non-human primate) having a retinal condition in a manner such that the AAV vector (a) infects foveal cones and (b) drives expression of the delivered exogenous nucleic acid in the infected foveal cones, thereby reducing the severity of one or more symptoms of the retinal condition and/or slowing the progression of the retinal condition. For example, an AAV vector (e.g., an AAV2 vector) provided herein (e.g., an AAV vector (e.g., an AAV2 vector) containing an AAV capsid polypeptide that includes an amino acid sequence set forth in Table 1B or according to Formula A based on such a set forth sequence) that is designed to contain and drive expression of an exogenous nucleic acid sequence encoding an RNA and/or polypeptide capable of treating a retinal condition (e.g., a retinal disease) can be administered to a mammal (e.g., a human or a non-human primate) having a retinal condition in a manner such that the AAV vector (a) infects retinal cells (e.g., retinal ganglion cells, photoreceptor cells, and bipolar cells) and (b) drives expression of the delivered exogenous nucleic acid in the infected retinal cells (e.g., at high levels), thereby reducing the severity of one or more symptoms of the retinal condition and/or slowing the progression of the retinal condition. For example, an AAV vector (e.g., an AAV2 vector) provided herein (e.g., an AAV vector (e.g., an AAV2 vector) containing an AAV capsid polypeptide that includes an amino acid sequence set forth in Table 1C or according to Formula A based on such a set forth sequence) that is designed to contain and drive expression of an exogenous nucleic acid sequence encoding an RNA and/or polypeptide capable of treating a retinal condition (e.g., a retinal disease) can be administered to a mammal (e.g., a human or a non-human primate) having a retinal condition in a manner such that the AAV vector (a) infects retinal cells (e.g., retinal ganglion cells) across at least two, three, or four different retinal regions and (b) drives expression of the delivered exogenous nucleic acid in the infected retinal cells, thereby reducing the severity of one or more symptoms of the retinal condition and/or slowing the progression of the retinal condition. For example, an AAV vector (e.g., an AAV2 vector) provided herein (e.g., an AAV vector (e.g., an AAV2 vector) containing an AAV capsid polypeptide that includes an amino acid sequence set forth in Table 1D or according to Formula A based on such a set forth sequence) that is designed to contain and drive expression of an exogenous nucleic acid sequence encoding an RNA and/or polypeptide capable of treating a retinal condition (e.g., a retinal disease) can be administered to a mammal (e.g., a human or a non-human primate) having a retinal condition in a manner such that the AAV vector (a) infects retinal cells of the parafovea region of the eye and (b) drives expression of the delivered exogenous nucleic acid in the infected retinal cells of the parafovea region, thereby reducing the severity of one or more symptoms of the retinal condition and/or slowing the progression of the retinal condition. For example, an AAV vector (e.g., an AAV2 vector) provided herein (e.g., an AAV vector (e.g., an AAV2 vector) containing an AAV capsid polypeptide that includes an amino acid sequence set forth in Table 1E or according to Formula A based on such a set forth sequence) that is designed to contain and drive expression of an exogenous nucleic acid sequence encoding an RNA and/or polypeptide capable of treating a retinal condition (e.g., a retinal disease) can be administered to a mammal (e.g., a human or a non-human primate) having a retinal condition in a manner such that the AAV vector (a) infects two or more (e.g., two or more, three or more, four or more, five or more, six or more, or seven or more) different retinal cell types within an eye and (b) drives expression of the delivered exogenous nucleic acid in the infected retinal cells (e.g., at high levels), thereby reducing the severity of one or more symptoms of the retinal condition and/or slowing the progression of the retinal condition. For example, an AAV vector (e.g., an AAV2 vector) provided herein (e.g., an AAV vector (e.g., an AAV2 vector) containing an AAV capsid polypeptide that includes an amino acid sequence set forth in Table 1F or according to Formula A based on such a set forth sequence) that is designed to contain and drive expression of an exogenous nucleic acid sequence encoding an RNA and/or polypeptide capable of treating a retinal condition (e.g., a retinal disease) can be administered to a mammal (e.g., a human or a non-human primate) having a retinal condition in a manner such that the AAV vector (a) infects RPE cells and (b) drives expression of the delivered exogenous nucleic acid in the infected RPE cells (e.g., at high levels), thereby reducing the severity of one or more symptoms of the retinal condition and/or slowing the progression of the retinal condition. For example, an AAV vector (e.g., an AAV2 vector) provided herein (e.g., an AAV vector (e.g., an AAV2 vector) containing an AAV capsid polypeptide that includes an amino acid sequence set forth in Table 1G or according to Formula A based on such a set forth sequence) that is designed to contain and drive expression of an exogenous nucleic acid sequence encoding an RNA and/or polypeptide capable of treating a retinal condition (e.g., a retinal disease) can be administered to a mammal (e.g., a human or a non-human primate) having a retinal condition in a manner such that the AAV vector (a) infects photoreceptor cells and (b) drives expression of the delivered exogenous nucleic acid in the infected photoreceptor cells, thereby reducing the severity of one or more symptoms of the retinal condition and/or slowing the progression of the retinal condition. For example, an AAV vector (e.g., an AAV2 vector) provided herein (e.g., an AAV vector (e.g., an AAV2 vector) containing an AAV capsid polypeptide that includes an amino acid sequence set forth in Table 1H or according to Formula A based on such a set forth sequence) that is designed to contain and drive expression of an exogenous nucleic acid sequence encoding an RNA and/or polypeptide capable of treating a retinal condition (e.g., a retinal disease) can be administered to a mammal (e.g., a human or a non-human primate) having a retinal condition in a manner such that the AAV vector (a) infects retinal ganglion cells and (b) drives expression of the delivered exogenous nucleic acid in the infected retinal ganglion cells, thereby reducing the severity of one or more symptoms of the retinal condition and/or slowing the progression of the retinal condition. For example, an AAV vector (e.g., an AAV2 vector) provided herein (e.g., an AAV vector (e.g., an AAV2 vector) containing an AAV capsid polypeptide that includes an amino acid sequence set forth in Table 11 or according to Formula A based on such a set forth sequence) that is designed to contain and drive expression of an exogenous nucleic acid sequence encoding an RNA and/or polypeptide capable of treating a retinal condition (e.g., a retinal disease) can be administered to a mammal (e.g., a human or a non-human primate) having a retinal condition in a manner such that the AAV vector (a) infects bipolar cells of the retina and (b) drives expression of the delivered exogenous nucleic acid in the infected bipolar cells, thereby reducing the severity of one or more symptoms of the retinal condition and/or slowing the progression of the retinal condition. For example, an AAV vector (e.g., an AAV2 vector) provided herein (e.g., an AAV vector (e.g., an AAV2 vector) containing an AAV capsid polypeptide that includes an amino acid sequence set forth in Table 1J or according to Formula A based on such a set forth sequence) that is designed to contain and drive expression of an exogenous nucleic acid sequence encoding an RNA and/or polypeptide capable of treating a retinal condition (e.g., a retinal disease) can be administered to a mammal (e.g., a human or a non-human primate) having a retinal condition in a manner such that the AAV vector (a) infects ON-retinal ganglion cells and (b) drives expression of the delivered exogenous nucleic acid in the infected ON-retinal ganglion cells, thereby reducing the severity of one or more symptoms of the retinal condition and/or slowing the progression of the retinal condition. For example, an AAV vector (e.g., an AAV2 vector) provided herein (e.g., an AAV vector (e.g., an AAV2 vector) containing an AAV capsid polypeptide that includes an amino acid sequence set forth in Table 1K or according to Formula A based on such a set forth sequence) that is designed to contain and drive expression of an exogenous nucleic acid sequence encoding an RNA and/or polypeptide capable of treating a retinal condition (e.g., a retinal disease) can be administered to a mammal (e.g., a human or a non-human primate) having a retinal condition in a manner such that the AAV vector (a) infects OFF-retinal ganglion cells and (b) drives expression of the delivered exogenous nucleic acid in the infected OFF-retinal ganglion cells, thereby reducing the severity of one or more symptoms of the retinal condition and/or slowing the progression of the retinal condition. For example, an AAV vector (e.g., an AAV2 vector) provided herein (e.g., an AAV vector (e.g., an AAV2 vector) containing an AAV capsid polypeptide that includes an amino acid sequence set forth in Table 1L or according to Formula A based on such a set forth sequence) that is designed to contain and drive expression of an exogenous nucleic acid sequence encoding an RNA and/or polypeptide capable of treating a retinal condition (e.g., a retinal disease) can be administered to a mammal (e.g., a human or a non-human primate) having a retinal condition in a manner such that the AAV vector (a) infects retinal cells and (b) drives expression of the delivered exogenous nucleic acid in the infected retinal cells (e.g., at high levels), thereby reducing the severity of one or more symptoms of the retinal condition and/or slowing the progression of the retinal condition.

As described herein, an AAV vector (e.g., an AAV2 vector) provided herein can be designed to include and drive expression of an exogenous nucleic acid sequence encoding any appropriate RNA of interest and/or polypeptide of interest. When an AAV vector provided herein is designed to treat a retinal condition (e.g., a retinal disease), an exogenous nucleic acid sequence that encodes an RNA and/or polypeptide capable of treating the retinal condition can be included within the AAV vector. Examples of RNAs that can be encoded by an exogenous nucleic acid sequence designed to treat a retinal condition (e.g., a retinal disease) and designed to be included within an AAV vector provided herein include, without limitation, SIRNA-027 to treat, e.g., sub-foveal CNVM secondary to age-related macular degeneration (see, e.g., NCT00363714), Cand5/Bevasiranib to treat, e.g., diabetic macular edema (see, e.g., NCT00306904), PF-04523655 to treat, e.g., diabetic macular edema (see, e.g., NCT01445899), QPI-1007 to treat, e.g., optic nerve atrophy in NAION (see, e.g., NCT01064505), Aganirsen to treat, e.g., ischemic CRVO to prevent neovascular glaucoma (see, e.g., NCT02947867), QR-421a to treat, e.g., retinitis pigmentosa/Usher syndrome type 2 (see, e.g., NCT03780257), QR-1123 to treat, e.g., autosomal dominant retinitis pigmentosa (see, e.g., NCT04123626), IONIS-FB-LRx to treat, e.g., geographic atrophy secondary to age-related macular degeneration (see, e.g., NCT03815825), and Sepofarsen/QR-110 to treat, e.g., Leber's congenital amaurosis (see, e.g., NCT03913143). Examples of polypeptides that can be encoded by an exogenous nucleic acid sequence designed to treat a retinal condition (e.g., a retinal disease) and designed to be included within an AAV vector provided herein include, without limitation, an ABCA4 polypeptide, a CRB1 polypeptide, an NPHP5 polypeptide, an NR2E3 polypeptide, a PDE6A polypeptide, a PDE6B polypeptide, a PDE6C polypeptide, a PRPF31 polypeptide, a RPE65 polypeptide, a RPGR polypeptide, a RS1 polypeptide, a TYR polypeptide, a USH2A polypeptide, a MYO7A polypeptide, an REP1 polypeptide, an OPN1LW polypeptide, an OPN1MW polypeptide, a CNGA3 polypeptide, a CNGB3 polypeptide, a GUCY2D polypeptide, a GACA1A polypeptide, a GNAT2 polypeptide, a PDE6H polypeptide, a PROM1 polypeptide, a PRPH2 polypeptide, a CRX polypeptide, an NPHP5 polypeptide, an EYS polypeptide, an ND4 polypeptide, a CLN1-14 polypeptide (e.g., a CLN3 polypeptide, a CLN5 polypeptide, a CLN6 polypeptide, or a CLN8 polypeptide), an NYX polypeptide, a GRM6 polypeptide, a TRPM1 polypeptide, a GPR179 polypeptide, an LRIT3 polypeptide, a glial cell derived neurotrophic factor (GDNF) polypeptide, a brain-derived neurotrophic factor (BDNF) polypeptide, a fibroblast growth factor (FGF) polypeptide, a truncated rod-derived cone viability factor (RdCVF) polypeptide, a full-length rod-derived cone viability factor (RdCVFL) polypeptide, an X-linked inhibitor of apoptosis (XIAP) polypeptide, a soluble fms-related receptor tyrosine kinase 1 (sFLT) polypeptide, a CYP4V2 polypeptide, a palmitoyl protein thioesterase 1 polypeptide, a tripeptidyl peptidase 1 polypeptide, a DNAJC5 polypeptide, a MFSD8 polypeptide, a cathepsin D polypeptide, a granulin polypeptide, an ATP13A2 polypeptide, a cathepsin F polypeptide, a KCTD7 polypeptide, a “P” gene polypeptide, a TRP1 polypeptide, a MATP (SLC45A2) polypeptide, a SLC24A5 polypeptide, a LRMDA polypeptide, a GPR143 polypeptide, an RPGR-exon 1-ORF15 polypeptide, an USH2b polypeptide, an USH1C polypeptide, a CDH23 polypeptide, a PCDH15 polypeptide, a SANS polypeptide, an USH1H polypeptide, a CIB2 polypeptide, an USH1K polypeptide, an ADGRV1 polypeptide, a WHRN polypeptide, a PDZD7 polypeptide, a CLRN1 polypeptide, a HARS polypeptide, an RP2 polypeptide, a FAM161 polypeptide, a DLK polypeptide, a RHO polypeptide, a CHM polypeptide, a BEST1 polypeptide, a RP1 polypeptide, an OPA1 polypeptide, a CEP290 polypeptide, a RDH12 polypeptide, a CACNA1F polypeptide, a BBS1 polypeptide, a FAM161A polypeptide, a CERKL polypeptide, a PRPF8 polypeptide, a RP1L1 polypeptide, a SNRNP200 polypeptide, an IMPG2 polypeptide, a CDHR1 polypeptide, an IMPDH1 polypeptide, a CNGB1 polypeptide, a MERTK polypeptide, a KCNV2 polypeptide, an AIPL1 polypeptide, a RPGRIP1 polypeptide, a TULP1 polypeptide, a C2ORF71 (aka PCARE) polypeptide, a MAK polypeptide, a TIMP3 polypeptide, a GUCA1A polypeptide, an ALMS1 polypeptide, a BBS10 polypeptide, an IFT140 polypeptide, a CNGA1 polypeptide, a NMNAT1 polypeptide, a COL2A1 polypeptide, an EFEMPI polypeptide, a WFS1 polypeptide, a RDH5 polypeptide, a PRPF3 polypeptide, a LRP5 polypeptide, a TOPORS polypeptide, a DHDDS polypeptide, a LCA5 polypeptide, an IQCB1 polypeptide, a RP9 polypeptide, an ATXN7 polypeptide, a BBS2 polypeptide, a SAG RLBP1 polypeptide, a ND6 (MT-ND6) polypeptide, a C1QTNF5 polypeptide, a VPS13B polypeptide, a KIF11 polypeptide, a MT-TL1 polypeptide, a KLHL7 polypeptide, an ACO2 polypeptide, a C21orf2 (aka CFAP410) polypeptide, an AHI1 polypeptide, a KIZ polypeptide, a SPATA7 polypeptide, a TTLL5 polypeptide, an HGSNAT polypeptide, a NRL polypeptide, an OAT polypeptide, a FLVCR1 polypeptide, an ABCC6 polypeptide, a LRAT polypeptide, a CEP78 polypeptide, a CDH3 polypeptide, a FZD4 polypeptide, a BBS12 polypeptide, an HK1 polypeptide, a PRDM13 polypeptide, an ADAM9 polypeptide, a BBS7 polypeptide, a CABP4 polypeptide, an ABHD12 polypeptide, a COL18A1 polypeptide, a MFRP polypeptide, a RIMS1 polypeptide, a ROM1 polypeptide, a BBS4 polypeptide, an IMPG1 polypeptide, an INPP5E polypeptide, a VCAN polypeptide, a POClB polypeptide, a RAX2 polypeptide, a TSPAN12 polypeptide, a CACNA2D4 polypeptide, a JAG1 polypeptide, a MKKS polypeptide, a NPHP4 polypeptide, a BBS9 polypeptide, a COL11A1 polypeptide, an ELOVL4 polypeptide, a NDP polypeptide, a NPHP1 polypeptide, a RGR polypeptide, a BBS5 polypeptide, a WDR19 polypeptide, a C8ORF37 polypeptide, a CTNNA1 polypeptide, a LAMP2 polypeptide, a PEX1 polypeptide, a PHYH polypeptide, an ATF6 polypeptide, a PRPS1 polypeptide, a SEMA4A polypeptide, an ARL6 polypeptide, a CNNM4 polypeptide, an OTX2 polypeptide, a PRPF6 polypeptide, a RBP3 polypeptide, a PNPLA6 polypeptide, a SLC24A1 polypeptide, an USH1G polypeptide, a PITPNM3 polypeptide, a TTC8 polypeptide, an ARSG polypeptide, a CWC27 polypeptide, a DRAM2 polypeptide, a PRCD polypeptide, a REEP6 polypeptide, a SSBP1 polypeptide, a LAMA1 polypeptide, a RAB28 polypeptide, a ZNF408 polypeptide, a GNAT1 polypeptide, an IDH3A polypeptide, a PDE6G polypeptide, a PEX6 polypeptide, a TUB polypeptide, a CEP250 polypeptide, a FSCN2 polypeptide, a GRK1 polypeptide, a RBP4 polypeptide, a RD3 polypeptide, an AGBL5 polypeptide, a CAPN5 polypeptide, an IFT172 polypeptide, a KCNJ13 polypeptide, a PAX2 polypeptide, a CC2D2A polypeptide, a HMCN1 polypeptide, a MT-ATP6 polypeptide, a RCBTB1 polypeptide, an ARL2BP polypeptide, a CA4 polypeptide, a DFNB31 polypeptide, a GNB3 polypeptide, a MMACHC polypeptide, a PRPF4 polypeptide, a RGS9 polypeptide, an ARHGEF18 polypeptide, a KIAA1549 polypeptide, a MKS1 polypeptide, a MTTP (not MT-TP) polypeptide, a PLK4 polypeptide, a RPGRIP1L polypeptide, a SDCCAG8 polypeptide, a SRD5A3 polypeptide, a TUBB4B polypeptide, an ADAMTS18 polypeptide, an ARL3 polypeptide, a COL11A2 polypeptide, a MVK polypeptide, a NBAS polypeptide, an OFD1 polypeptide, a P3H2 polypeptide, a RGS9BP polypeptide, a CSPP1 polypeptide, an ITM2B polypeptide, a PANK2 polypeptide, a PEX7 polypeptide, a POMGNT1 polypeptide, a SLC4A7 polypeptide, a TMEM231 polypeptide, a TRNT1 polypeptide, a TUBGCP6 polypeptide, a ZNF513 polypeptide, an AFG3L2 polypeptide, an ARL13B polypeptide, a C5ORF42 (aka CPLANE1) polypeptide, a COL9A1 polypeptide, a CTSD polypeptide, a DTHD1 polypeptide, a DYNC2H1 polypeptide, an IFT81 polypeptide, a KIAA0586 polypeptide, a MFN2 polypeptide, a NPHP3 polypeptide, a PCYT1A polypeptide, a PEX12 polypeptide, a PLA2G5 polypeptide, a POC5 polypeptide, a SCAPER polypeptide, a SLC25A46 polypeptide, a TMEM237 polypeptide, a TRAF3IP1 polypeptide, a TTC21B polypeptide, a TUBGCP4 polypeptide, an ADIPOR1 polypeptide, a CEP164 polypeptide, a CLCC1 polypeptide, a COL9A2 polypeptide, a CTNNB1 polypeptide, a DHX38 polypeptide, a GNPTG polypeptide, a GRN polypeptide, a GUCAIB polypeptide, an IFT27 polypeptide, an IFT74 polypeptide, a KIAA0556 polypeptide, a LRP2 polypeptide, a MAPKAPK3 polypeptide, a MIR204 polypeptide, a MT-ND3 polypeptide, a MT-RNR1 polypeptide, a MT-TS2 polypeptide, a ND5 (MT-ND5) polypeptide, a NEK2 polypeptide, an OPN1 SW polypeptide, a PEX13 polypeptide, a PEX2 polypeptide, a RHBDD2 polypeptide, a SAMD11 polypeptide, a SCLT1 polypeptide, a SLC7A14 polypeptide, a TCTN1 polypeptide, a TCTN2 polypeptide, a TLCD3B polypeptide, a TREX1 polypeptide, a TTPA polypeptide, an UNC119 polypeptide, a WDPCP polypeptide, an ACBD5 polypeptide, an AHR polypeptide, an ARMC9 polypeptide, an ASRGL1 polypeptide, an ATOH7 polypeptide, a B9D1 polypeptide, a B9D2 polypeptide, a BBIP1 polypeptide, a C12ORF65 polypeptide, a C2CD3 polypeptide, a C5AR2 polypeptide, a CCDCl88 polypeptide, a CCT2 polypeptide, a CEP104 polypeptide, a CEP120 polypeptide, a CEP19 polypeptide, a CEP41 polypeptide, a CISD2 polypeptide, a CLUAP1 polypeptide, a COL9A3 polypeptide, a CRB2 polypeptide, a CTC1 polypeptide, a DACT2 polypeptide, a DDR1 polypeptide, an ENSA polypeptide, an ESPN polypeptide, an EXOSC2 polypeptide, a FBN3 polypeptide, a GDF6 polypeptide, a GPR125 polypeptide, a HKDC1 polypeptide, a HMX1 polypeptide, an IDH3B polypeptide, an IFT43 polypeptide, an IFT80 polypeptide, an INVS polypeptide, a KIAA0753 polypeptide, a KIF3B polypeptide, a KIF7 polypeptide, a LRRTM4 polypeptide, a LZTFL1 polypeptide, a MT-ATP8 polypeptide, a MT-CO1 polypeptide, a MT-C02 polypeptide, a MT-C03 polypeptide, a MT-CYB polypeptide, a MT-ND2 polypeptide, a MT-ND4L polypeptide, a MT-RNR2 polypeptide, a MT-TA polypeptide, a MT-TC polypeptide, a MT-TD polypeptide, a MT-TE polypeptide, a MT-TF polypeptide, a MT-TG polypeptide, a MT-TH polypeptide, a MT-TI polypeptide, a MT-TK polypeptide, a MT-TL2 polypeptide, a MT-TM polypeptide, a MT-TN polypeptide, a MT-TP (Not MTTP) polypeptide, a MT-TQ polypeptide, a MT-TR polypeptide, a MT-TS1 polypeptide, a MT-TT polypeptide, a MT-TV polypeptide, a MT-TW polypeptide, a MT-TY polypeptide, a NEURODI polypeptide, a PDE6D polypeptide, a PEX10 polypeptide, a PEX11B polypeptide, a PEX14 polypeptide, a PEX16 polypeptide, a PEX19 polypeptide, a PEX26 polypeptide, a PEX3 polypeptide, a PEX5 polypeptide, a PGK1 polypeptide, a PISD polypeptide, a PPP2R3C polypeptide, a PROS1 polypeptide, a PSEN1 polypeptide, a RDH11 polypeptide, a RRM2B polypeptide, a SMARCA4 polypeptide, a SPP2 polypeptide, a TCTN3 polypeptide, a TEAD1 polypeptide, a TMEM10⁷ polypeptide, a TMEM138 polypeptide, a TMEM216 polypeptide, a TMEM67 polypeptide, a TPP1 polypeptide, a TRIM32 polypeptide, a USP45 polypeptide, and a ZNF423 polypeptide. In some cases, a polypeptide of interest that can be encoded by an exogenous nucleic acid sequence designed to be included within an AAV vector provided herein can be a MYO7A polypeptide, an USH1C polypeptide, a PCDH15 polypeptide, an USH2A polypeptide, or CLRN1 polypeptide to treat, e.g., Usher Syndrome.

Any appropriate retinal condition (e.g., a retinal disease) can be treated using an AAV vector (e.g., an AAV2 vector) provided herein (e.g., an AAV vector containing an AAV capsid polypeptide that includes an amino acid sequence set forth in any one or more of Tables 1A-1L (or a variant thereof) or according to Formula A based on such a set forth sequence and an exogenous nucleic acid sequence encoding a therapeutic RNA and/or polypeptide). Examples of such retinal conditions include, without limitation, Leber congenital amaurosis (LCA), Leber hereditary optic neuropathy (LHON), oculocutaneous albinism type 1 (OCA1), retinitis pigmentosa, rod/cone dystrophy, cone dystrophy, rod dystrophy, Stargardt Disease, Usher syndrome, X-linked retinitis pigmentosa (XLRP), X-linked retinoschisis (XLRS), choroideremia, achromatopsia, blue cone monochromacy, color blindness, glaucoma, optic atrophy, Batten disease, congenital stationary night blindness (CSNB), macular degeneration, CRB1-related retinal dystrophy, and foveal cone dystrophy.

Examples of therapeutic RNAs and polypeptides that can be delivered using an AAV vector provided herein to treat particular retinal conditions are set forth in Tables 2 and 3. Examples of genomic nucleic acids that can be inactivated and/or knocked out to treat particular retinal conditions using one or more AAV vectors provided herein that are designed to deliver gene editing components are set forth in Table 3. Examples of genomic nucleic acids of disease causing alleles that can be replaced with healthy alleles to treat particular retinal conditions using one or more AAV vectors provided herein that are designed to deliver gene editing components are set forth in Table 3.

TABLE 2
Examples of therapeutic polypeptide for treating retinal conditions.

| Retinal Condition | Examples of therapeutic polypeptides |
|---|---|
| Stargardt Disease | ABCA4 |
| Leber congenital amaurosis 8 | CRB1 |
| Leber congenital amaurosis | NPHP5 |
| Retinitis pigmentosa 37 | NR2E3 |
| Rod/Cone dystrophy | PDE6A/PDE6B |
| Rod/Cone dystrophy | PDE6c |
| Retinitis pigmentosa 11 | PRPF31 |
| Leber congenital amaurosis 2 | RPE65 |
| X-linked retinitis pigmentosa | RPGR |
| X-linked retinoschisis | RS1 |
| Oculocutaneous albinism type 1 | TYR |
| Usher syndrome | PCDH15 |
| Usher syndrome | USH2a |
| Usher syndrome | USH2b |
| Usher syndrome, subtype IB caused by | MYO7A |
| mutations in the MYO7A gene | |
| Usher syndrome, subtype IC caused by | USH1C |
| mutations in the USH1C gene | |
| Usher syndrome, subtype ID caused by | CDH23 |
| mutations in the CDH23 gene | |
| Usher syndrome, subtype ID-F caused by | PCDH15 and/or CDH23 |
| mutations in the PCDH15 and/or CDH23 | |
| genes | |
| Usher syndrome, subtype IF caused by | PCDH15 |
| mutations in the PCDH15 gene | |
| Usher syndrome, subtype IG caused by | SANS |
| mutations in the SANS gene | |
| Usher syndrome, subtype IH caused by | USH1H |
| mutations in the USH1H gene | |
| Usher syndrome, subtype IJ caused by | CIB2 |
| mutations in the CIB2 gene | |
| Usher syndrome, subtype IK caused by | USH1K |
| mutations in the USH1K gene | |
| Usher syndrome, subtype IIA, caused by | USH2A |
| mutations in the USH2A | |
| Usher syndrome, subtype IIC caused by | ADGRV1 |
| mutations in the ADGRV1 gene | |
| Usher syndrome, subtype IID caused by | WHRN |
| mutations in the WHRN gene | |
| Usher syndrome, subtype IIC caused by | GPR98 and/or PDZD7 |
| mutations in the GPR98 and/or PDZD7 genes | |
| (ADGRV1 is also known as GPR98) | |
| Usher syndrome, subtype IIIA caused by | CLRN1 |
| mutations in the CLRN1 gene | |
| Usher syndrome, subtype IIIB caused by | IIIB, caused by mutations in the HARS gene |
| mutations in the HARS gene | |
| Retinitis pigmentosa | One or more trophic factors |
| Leber congenital amaurosis | One or more trophic factors |
| Achromatopsia | CNGA3, CNGB3, and/or PDE6H |
| Any blinding disease | Optogenetic tools: Chr, NhpR, ReachR, |
| | and/or others |
| Blue cone monochromacy | OPN1LW and/or OPN1MW |
| Cone dystrophy | GNAT2 |
| Cone-rod dystrophy | PDE6C and/or PROM1 |

TABLE 3
Examples of polypeptide that can be expressed to treat retinal conditions, examples
of polypeptides that can be knocked out to treat retinal conditions, and/or examples
of polypeptides that can be knocked out and replace with an alternative (e.g.,
wild-type or non-disease version) to treat retinal conditions.

| | | | | Gene |
|---|---|---|---|---|
| | | Gene | | editing- |
| Disease | Polypeptide | expression | Gene KO | replacement |
| Achromatopsia | CNGA3 | X | | |
| Achromatopsia | CNGB3 | X | | |
| Achromatopsia | PDE6H | X | | |
| Any blinding disease | Optogenetic | X | | |
| | tools: Chr, | | | |
| | NhpR, and/or | | | |
| | ReachR | | | |
| Batten disease | CTSD | X | | |
| Bietti crystalline dystrophy | CYP4V2 | X | | X |
| Blue cone monochromacy | OPN1LW | X | | |
| Blue cone monochromacy | OPN1MW | X | | |
| Choroideremia | REP1 | X | | |
| Cone dystrophy | PDE6c | X | | |
| Cone dystrophy | GNAT2 | X | | |
| Cone-rod dystrophy | PDE6C | X | | |
| Cone-rod dystrophy | PROM1 | X | | |
| Cone/rod dystrophy | PRPH2 | X | X | X |
| CSNB | NYX | X | | |
| CSNB | GRM6 | X | | |
| CSNB | TRPM1 | X | | |
| CSNB | GPR179 | X | | |
| CSNB | LRIT3 | X | | |
| Glaucoma | Trophic factors | X | | |
| Glaucoma | Complement | X | | |
| | inhibition factors | | | |
| Glaucoma | Apoptosis | X | | |
| | inhibition factors | | | |
| Glaucoma | Survival factors | X | | |
| Glaucoma | Neuroprotective | X | | |
| | factors | | | |
| LCA | NPHP5 | X | | |
| LCA | GUCY2D | X | | |
| Leber congenital amaurosis | RPE65 | X | | |
| Leber congenital amaurosis 8 | CRB1 | X | | X |
| LHON | ND4 | X | | |
| Macular dystrophy | CRX | X | X | X |
| Oculocutaneous albinism | TYR | X | | |
| type 1 | | | | |
| Optic atrophy | CLN1-14 | X | | |
| Retinitis pigmentosa | EYS | X | | X |
| Retinitis pigmentosa | RHO | X | X | X |
| Retinitis pigmentosa | PRPF31 | X | | |
| Retinitis pigmentosa | PDE6A | X | | |
| Bestrophinopathy | BEST1 | X | X | |
| Retinitis pigmentosa | PRPF3 | X | | |
| Retinitis pigmentosa | PRPF8 | X | | |
| Retinitis pigmentosa | TOPORS | X | X | X |
| Retinitis pigmentosa 37 | NR2E3 | X | X | X |
| Rod/Cone dystrophy | PDE6B | X | | |
| RP, LCA, Others | Trophic factors | X | | |
| Stargardt Disease | ABCA4 | X | | X |
| Usher syndrome | PCDH15 | X | | |
| Usher syndrome | USH2A | X | | X |
| Usher syndrome | MYO7A | X | | X |
| XLRP | RPGR | X | | |
| XLRS | RS1 | X | | |
| Wet AMD | Survival factors | X | | |
| Dry AMD | Survival factors | X | | |
| Diabetic retinopathy | Survival factors | X | | |
| Diabetic Macular Edema | Survival factors | X | | |
| Retinitis pigmentosa | Survival factors | X | | |
| Wet AMD | Apoptosis | X | | |
| | inhibition factors | | | |
| Dry AMD | Apoptosis | X | | |
| | inhibition factors | | | |
| Diabetic retinopathy | Apoptosis | X | | |
| | inhibition factors | | | |
| Diabetic Macular Edema | Apoptosis | X | | |
| | inhibition factors | | | |
| Retinitis pigmentosa | Apoptosis | X | | |
| | inhibition factors | | | |
| Wet AMD | Complement | X | | |
| | inhibition factors | | | |
| Dry AMD | Complement | X | | |
| | inhibition factors | | | |
| Diabetic retinopathy | Complement | X | | |
| | inhibition factors | | | |
| Diabetic Macular Edema | Complement | X | | |
| | inhibition factors | | | |
| Wet AMD | Neuroprotective | X | | |
| | factors | | | |
| Dry AMD | Neuroprotective | X | | |
| | factors | | | |
| Diabetic retinopathy | Neuroprotective | X | | |
| | factors | | | |
| Diabetic Macular Edema | Neuroprotective | X | | |
| | factors | | | |
| Retinitis pigmentosa | Neuroprotective | X | | |
| | factors | | | |
| Wet AMD | Anti-VEGF | X | | |
| | polypeptides | | | |
| Diabetic retinopathy | Anti-VEGF | X | | |
| | polypeptides | | | |
| Diabetic Macular Edema | Anti-VEGF | X | | |
| | polypeptides | | | |
| Wet AMD | Optogenetic tools: | X | | |
| | Chr, NhpR, and/or | | | |
| | ReachR | | | |
| Dry AMD | Optogenetic tools: | X | | |
| | Chr, NhpR, and/or | | | |
| | ReachR | | | |
| Diabetic retinopathy | Optogenetic tools: | X | | |
| | Chr, NhpR, and/or | | | |
| | ReachR | | | |
| Diabetic Macular Edema | Optogenetic tools: | X | | |
| | Chr, NhpR, and/or | | | |
| | ReachR | | | |
| Retinitis pigmentosa | Optogenetic tools: | X | | |
| | Chr, NhpR, and/or | | | |
| | ReachR | | | |

In some cases, a retinal condition can be treated using an AAV vector provided herein that is designed to express one or more polypeptides having the ability to inhibit vascular angiogenesis. Examples of polypeptides having the ability to inhibit vascular angiogenesis that can be used as described herein include, without limitation, monoclonal anti-VEGF antibody polypeptides, angiostatin polypeptides, siRNA polypeptides, and endostatin polypeptides. In some cases, wet AMD can be treated using an AAV vector provided herein that is designed to express a monoclonal anti-VEGF antibody polypeptide, an angiostatin polypeptide, an siRNA, and/or endostatin polypeptide. In some cases, diabetic retinopathy can be treated using an AAV vector provided herein that is designed to express a monoclonal anti-VEGF antibody polypeptide, an angiostatin polypeptide, an siRNA, and/or an endostatin polypeptide. In some cases, diabetic macular edema can be treated using an AAV vector provided herein that is designed to express a monoclonal anti-VEGF antibody polypeptide, an angiostatin polypeptide, an siRNA, and/or an endostatin polypeptide.

In some cases, a retinal condition can be treated using an AAV vector provided herein that is designed to express one or more polypeptides with neuroprotective capabilities. Examples of polypeptides having the ability to provide neuroprotective activity that can be used as described herein include, without limitation, GDNF polypeptides, CNTF polypeptides, IGF-1 polypeptides, VEGF polypeptides, and BDNF polypeptides. In some cases, wet AMD can be treated using an AAV vector provided herein that is designed to express a GDNF polypeptide, a CNTF polypeptide, an IGF-1 polypeptide, a VEGF polypeptide, and/or a BDNF polypeptide. In some cases, dry AMD can be treated using an AAV vector provided herein that is designed to express a GDNF polypeptide, a CNTF polypeptide, an IGF-1 polypeptide, a VEGF polypeptide, and/or a BDNF polypeptide. In some cases, diabetic retinopathy can be treated using an AAV vector provided herein that is designed to express a GDNF polypeptide, a CNTF polypeptide, an IGF-1 polypeptide, a VEGF polypeptide, and/or a BDNF polypeptide. In some cases, diabetic macular edema can be treated using an AAV vector provided herein that is designed to express a GDNF polypeptide, a CNTF polypeptide, an IGF-1 polypeptide, a VEGF polypeptide, and/or a BDNF polypeptide.

In some cases, a retinal condition can be treated using an AAV vector provided herein that is designed to express one or more polypeptides having the ability to provide optogenetic capabilities. Examples of polypeptides having the ability to provide optogenetic capabilities that can be used as described herein include, without limitation, ChR polypeptides, ChR2 polypeptides, ArchT polypeptides, NpHR polypeptides, and ChrimsonR polypeptides. In some cases, wet AMD can be treated using an AAV vector provided herein that is designed to express a ChR polypeptide, a ChR2 polypeptide, an ArchT polypeptide, a NpHR polypeptide, and/or a ChrimsonR polypeptide. In some cases, dry AMD can be treated using an AAV vector provided herein that is designed to express a ChR polypeptide, a ChR2 polypeptide, an ArchT polypeptide, a NpHR polypeptide, and/or a ChrimsonR polypeptide. In some cases, diabetic retinopathy can be treated using an AAV vector provided herein that is designed to express a ChR polypeptide, a ChR2 polypeptide, an ArchT polypeptide, a NpHR polypeptide, and/or a ChrimsonR polypeptide. In some cases, diabetic macular edema can be treated using an AAV vector provided herein that is designed to express a ChR polypeptide, a ChR2 polypeptide, an ArchT polypeptide, a NpHR polypeptide, and/or a ChrimsonR polypeptide.

In some cases, a retinal condition can be treated using an AAV vector provided herein that is designed to express one or more polypeptides having the ability to inhibit apoptosis. Examples of polypeptides having the ability to inhibit apoptosis that can be used as described herein include, without limitation, XIAP polypeptides, cIAP1 polypeptides, C-IAP2 polypeptides, Livin polypeptides, and Survivin polypeptides. In some cases, wet AMD can be treated using an AAV vector provided herein that is designed to express a XIAP polypeptide, a cIAP1 polypeptide, a C-IAP2 polypeptide, a Livin polypeptide, and/or a Survivin polypeptide. In some cases, diabetic retinopathy can be treated using an AAV vector provided herein that is designed to express a XIAP polypeptide, a cIAP1 polypeptide, a C-IAP2 polypeptide, a Livin polypeptide, and/or a Survivin polypeptide. In some cases, diabetic macular edema can be treated using an AAV vector provided herein that is designed to express a XIAP polypeptide, a cIAP1 polypeptide, a C-IAP2 polypeptide, a Livin polypeptide, and/or a Survivin polypeptide.

In some cases, a retinal condition can be treated using an AAV vector provided herein that is designed to express one or more polypeptides having the ability to inhibit complement. Examples of polypeptides having the ability to inhibit complement that can be used as described herein include, without limitation, Complement Factor I polypeptides, Complement factor H polypeptides, and sCD59 polypeptides. In some cases, wet AMD can be treated using an AAV vector provided herein that is designed to express a Complement Factor I polypeptide, a Complement factor H polypeptide, and/or a sCD59 polypeptide. In some cases, dry AMD can be treated using an AAV vector provided herein that is designed to express a Complement Factor I polypeptide, a Complement factor H polypeptide, and/or a sCD59 polypeptide. In some cases, diabetic retinopathy can be treated using an AAV vector provided herein that is designed to express a Complement Factor I polypeptide, a Complement factor H polypeptide, and/or a sCD59 polypeptide. In some cases, diabetic macular edema can be treated using an AAV vector provided herein that is designed to express a Complement Factor I polypeptide, a Complement factor H polypeptide, and/or a sCD59 polypeptide.

In some cases, a retinal condition can be treated using an AAV vector provided herein that is designed to express one or more polypeptides having the ability to induce survival factors. Examples of polypeptides having the ability to induce survival factors that can be used as described herein include, without limitation, RdCVF polypeptides, RdCVFL polypeptides, HIF-1 polypeptides, IAP family polypeptides, and BCL-2 family polypeptides. In some cases, wet AMD can be treated using an AAV vector provided herein that is designed to express a RdCVF polypeptide, a RdCVFL polypeptide, an HIF-1 polypeptide, an IAP family polypeptide, and/or a BCL-2 family polypeptide. In some cases, dry AMD can be treated using an AAV vector provided herein that is designed to express a RdCVF polypeptide, a RdCVFL polypeptide, an HIF-1 polypeptide, an IAP family polypeptide, and/or a BCL-2 family polypeptide. In some cases, diabetic retinopathy can be treated using an AAV vector provided herein that is designed to express a RdCVF polypeptide, a RdCVFL polypeptide, an HIF-1 polypeptide, an IAP family polypeptide, and/or a BCL-2 family polypeptide. In some cases, diabetic macular edema can be treated using an AAV vector provided herein that is designed to express a RdCVF polypeptide, a RdCVFL polypeptide, an HIF-1 polypeptide, an IAP family polypeptide, and/or a BCL-2 family polypeptide.

Any appropriate method can be used to administer an AAV vector provided herein or composition (e.g., a pharmaceutical composition) provided herein to a mammal (e.g., a human or a non-human primate). For example, a composition provided herein (e.g., a pharmaceutical composition containing one or more AAV vectors provided herein) can be administered to a mammal (e.g., a human or a non-human primate) intravitreally, intravenously (e.g., via an intravenous injection or infusion), subcutaneously (e.g., via a subcutaneous injection), intraperitoneally (e.g., via an intraperitoneal injection), orally, via inhalation, intramuscularly (e.g., via intramuscular injection), subretinally, intravitreally, systemically, or suprachoroidally. In some cases, the route and/or mode of administration of a composition (e.g., a pharmaceutical composition provided herein) can be adjusted for the mammal being treated.

In some cases, an effective amount of a composition containing an AAV vector provided herein (e.g., a pharmaceutical composition provided herein) to treat a retinal condition can be an amount that reduces the severity of one or more symptoms of the retinal condition and/or slows the progression of the retinal condition without producing significant toxicity to the mammal. For example, an effective amount of an AAV vector provided herein can be from about 1×10⁷ viral genomes to about 1×10¹⁴ viral genomes (e.g., from about 1×10¹⁴ viral genomes to about 1×10¹³ viral genomes, from about 1×10⁷ viral genomes to about 1×10¹² viral genomes, from about 1×10⁷ viral genomes to about 1×10¹¹ viral genomes, from about 1×10⁷ viral genomes to about 1×10¹⁰ viral genomes, from about 1×10⁸ viral genomes to about 1×10¹⁴ viral genomes, from about 1×10⁹ viral genomes to about 1×10¹⁴ viral genomes, from about 1×10¹⁰ viral genomes to about 1×10¹⁴ viral genomes, from about 1×10⁸ viral genomes to about 1×10¹² viral genomes, or from about 1×10⁹ viral genomes to about 1×10¹¹ viral genomes). In some cases, an effective amount of an AAV vector provided herein can be from about 1×10¹⁰ viral genomes/kg of body weight to about 1×10¹⁴ viral genomes/kg of body weight (e.g., from about 1×10¹⁰ viral genomes/kg of body weight to about 1×10¹³ viral genomes/kg of body weight, from about 1×10¹⁰ viral genomes/kg of body weight to about 1×10¹² viral genomes/kg of body weight, from about 1×10¹⁰ viral genomes/kg of body weight to about 1×10¹¹ viral genomes/kg of body weight). The effective amount can remain constant or can be adjusted as a sliding scale or variable dose depending on the mammal's response to treatment. Various factors can influence the actual effective amount used for a particular application. For example, the severity of a retinal condition, the route of administration, the age and general health condition of the mammal, excipient usage, the possibility of co-usage with other therapeutic or prophylactic treatments such as use of other retinal drugs, and the judgment of the treating physician may require an increase or decrease in the actual effective amount of a composition provided herein (e.g., a pharmaceutical composition containing an AAV vector provided herein) that is administered.

In some cases, an effective frequency of administration of a composition containing an AAV vector provided herein (e.g., a pharmaceutical composition provided herein) can be a frequency that reduces the severity of one or more symptoms of the retinal condition and/or slows the progression of the retinal condition without producing significant toxicity to the mammal. Various factors can influence the actual effective frequency used for a particular application. For example, the severity of a retinal condition, the route of administration, the age and general health condition of the mammal, excipient usage, the possibility of co-usage with other therapeutic or prophylactic treatments such as use of other retinal drugs, and the judgment of the treating physician may require an increase or decrease in the actual effective frequency of administration of a composition provided herein.

In some cases, an effective duration of administration of a composition containing an AAV vector provided herein (e.g., a pharmaceutical composition provided herein) can be a duration that reduces the severity of one or more symptoms of the retinal condition and/or slows the progression of the retinal condition without producing significant toxicity to the mammal. For example, an effective duration of administration of a pharmaceutical composition provided herein can vary from a single time point of administration to several weeks to several months (e.g., 4 to 12 weeks). In some cases, the duration can be for as long as the mammal is alive. Multiple factors can influence the actual effective duration used for a particular application. For example, the severity of a retinal condition, the route of administration, the age and general health condition of the mammal, excipient usage, the possibility of co-usage with other therapeutic or prophylactic treatments such as use of other retinal drugs, and the judgment of the treating physician may require an increase or decrease in the actual effective duration of administration of a composition provided herein (e.g., a pharmaceutical composition containing an AAV vector provided herein).

In some cases, an effective amount of a composition containing an AAV vector provided herein (e.g., a pharmaceutical composition provided herein) to treat a retinal condition can be administered once or twice to a mammal (e.g., a human or a non-human primate) to treat that mammal.

The invention will be further described in the following examples, which do not limit the scope of the invention described in the claims.

### EXAMPLES

### Example 1—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting foveal cones in the retina. See, e.g., Öztürk et al., bioRxiv, 2020.10.01.323196 (2020) and Öztürk et al., eLife, 10:e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10X Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence of any one of SEQ ID NOs:A2-A18 located between amino acid residues 587 and 588 of SEQ ID NO:1 or an amino acid sequence of SEQ ID NO:A1 as a replacement of amino acid residues 585 to 590 of SEQ ID NO:1 mediated expression in foveal cells (Table 1A). The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression in foveal cells. No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1A or according to Formula A based on such a set forth sequence can have the ability to mediate transgene expression in foveal cones following intravitreal injection.

### Example 2—Treating a Retinal Condition Using an AAV Vector

An AAV vector is constructed to include an AAV2 capsid polypeptide having an amino acid sequence set forth in Table 1A (e.g., SEQ ID NO:A2) or according to Formula A based on such a set forth sequence and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 3—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10:e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10X Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence of any one of SEQ ID NOs:B2-B298 located between amino acid residues 587 and 588 of SEQ ID NO:1 or an amino acid sequence of any one of SEQ ID NOs:B299-B317 as a replacement of amino acid residues 585 to 590 of SEQ ID NO:1 mediated expression in retinal cells (Table 1). The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression in retinal cells. No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1B or according to Formula A based on such a set forth sequence can have the ability to mediate transgene expression (e.g., high expression) in retinal cells following intravitreal injection.

### Example 4—Treating a Retinal Condition Using an AAV Vector

An AAV vector is constructed to include an AAV2 capsid polypeptide having an amino acid sequence set forth in Table 1B (e.g., SEQ ID NO:B2) or according to Formula A based on such a set forth sequence and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 5—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10:e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10X Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence of any one of SEQ ID NOs:C2-C43 located between amino acid residues 587 and 588 of SEQ ID NO:1 or an amino acid sequence of any one of SEQ ID NOs:C44-C45 as a replacement of amino acid residues 585 to 590 of SEQ ID NO:1 mediated expression in retinal cells across two or more retinal regions. The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression across retinal regions. No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1C or according to Formula A based on such a set forth sequence can have the ability to deliver nucleic acid to and express nucleic acid in retinal cells in at least two different retinal regions.

### Example 6—Treating a Retinal Condition Using an AAV Vector

An AAV vector is constructed to include an AAV2 capsid polypeptide having an amino acid sequence set forth in Table 1C (e.g., SEQ ID NO:C2) or according to Formula A based on such a set forth sequence and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 7—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells of the parafovea region of the eye. See, e.g., Öztürk et al., bioRxiv, 2020.10.01.323196 (2020). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10X Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence of any one of SEQ ID NOs:D2-D76 located between amino acid residues 587 and 588 of SEQ ID NO:1 or an amino acid sequence of any one of SEQ ID NOs:D77D78 as a replacement of amino acid residues 585 to 590 of SEQ ID NO:1 mediated expression in retinal cells of the parafovea region (Table 1D). The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression in the parafoveal region of the retina. No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1D or according to Formula A based on such a set forth sequence can have the ability to mediate transgene expression in retinal cells of the parafovea region following intravitreal injection.

### Example 8—Treating a Retinal Condition Using an AAV Vector

An AAV vector is constructed to include an AAV2 capsid polypeptide having an amino acid sequence set forth in Table 1D (e.g., SEQ ID NO:D2) or according to Formula A based on such a set forth sequence and an exogenous nucleic acid sequence encoding a therapeutic polypeptide. The constructed AAV vector is administered intravitreally to a human identified as having a retinal condition in an amount that is from about 1×10¹⁴ to about 1×10¹⁴ AAV vectors. After the administration, the severity of one or more symptoms of the retinal condition is reduced and/or the progression of the retinal condition is slowed.

### Example 9—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10:e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10X Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence of any one of SEQ ID NOs:E2-E75 located between amino acid residues 587 and 588 of SEQ ID NO:1 or an amino acid sequence of any one of SEQ ID NOs:E76-E83 as a replacement of amino acid residues 585 to 590 of SEQ ID NO:1 mediated expression the ability to deliver nucleic acid to and express nucleic acid in multiple different retinal cells types within an eye, thereby providing an efficient way to obtain nucleic acid delivery to many different retinal cell types (Table 1E). The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression across cell types. No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1E or according to Formula A based on such a set forth sequence can have the ability to mediate transgene expression in multiple different retinal cells types within an eye following intravitreal injection.

### Example 10—Treating a Retinal Condition Using an AAV Vector

An AAV vector is constructed to include an AAV2 capsid polypeptide having an amino acid sequence set forth in Table 1E (e.g., SEQ ID NO:E2) or according to Formula A based on such a set forth sequence and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 11—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10:e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10X Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence of any one of SEQ ID NOs:F2-F10 located between amino acid residues 587 and 588 of SEQ ID NO:1 mediated expression in RPE cells (Table 1F). The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression in RPE cells. No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1F or according to Formula A based on such a set forth sequence can have the ability to mediate transgene expression in RPE cells following intravitreal injection.

### Example 12—Treating a Retinal Condition Using an AAV Vector

An AAV vector is constructed to include an AAV2 capsid polypeptide having an amino acid sequence set forth in Table 1F (e.g., SEQ ID NO:F2) or according to Formula A based on such a set forth sequence and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 13—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10:e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10X Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence of any one of SEQ ID NOs:G2-G70 located between amino acid residues 587 and 588 of SEQ ID NO:1 or an amino acid sequence of any one of SEQ ID NOs:G71-G77 as a replacement of amino acid residues 585 to 590 of SEQ ID NO:1 mediated expression preferentially in photoreceptor cells (Table 1G). The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression in retinal cells. No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1G or according to Formula A based on such a set forth sequence can have the ability to mediate transgene expression preferentially in photoreceptor cells following intravitreal injection.

### Example 14—Treating a Retinal Condition Using an AAV Vector

An AAV vector is constructed to include an AAV2 capsid polypeptide having an amino acid sequence set forth in Table 1G (e.g., SEQ ID NO:G2) or according to Formula Abased on such a set forth sequence and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 15—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10:e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10X Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence of any one of SEQ ID NOs:H2-H243 located between amino acid residues 587 and 588 of SEQ ID NO:1 or an amino acid sequence of any one of SEQ ID NOs:H244-H258 as a replacement of amino acid residues 585 to 590 of SEQ ID NO:1 mediated expression preferentially in retinal ganglion cells (Table 1H). The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression in retinal cells. No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1H or according to Formula A based on such a set forth sequence can have the ability to mediate transgene expression preferentially in retinal ganglion cells following intravitreal injection.

### Example 16—Treating a Retinal Condition Using an AAV Vector

An AAV vector is constructed to include an AAV2 capsid polypeptide having an amino acid sequence set forth in Table 1H (e.g., SEQ ID NO:H2) or according to Formula Abased on such a set forth sequence and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 17—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10:e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10X Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence of any one of SEQ ID NOs:I2-I67 located between amino acid residues 587 and 588 of SEQ ID NO:1 or an amino acid sequence of any one of SEQ ID NOs:I68-I74 as a replacement of amino acid residues 585 to 590 of SEQ ID NO:1 mediated expression preferentially in bipolar cells (Table 1I). The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression in retinal cells. No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1I or according to Formula A based on such a set forth sequence can have the ability to mediate transgene expression preferentially in bipolar cells following intravitreal injection.

### Example 18—Treating a Retinal Condition Using an AAV Vector

An AAV vector is constructed to include an AAV2 capsid polypeptide having an amino acid sequence set forth in Table 1I (e.g., SEQ ID NO:I2) or according to Formula A based on such a set forth sequence and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 19—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10:e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10X Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence of any one of SEQ ID NOs:J2-J61 located between amino acid residues 587 and 588 of SEQ ID NO:1 or an amino acid sequence of any one of SEQ ID NOs:J62-J64 as a replacement of amino acid residues 585 to 590 of SEQ ID NO:1 mediated expression preferentially in ON-retinal ganglion cells (Table 1J). The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression in retinal cells. No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1J or according to Formula A based on such a set forth sequence can have the ability to mediate transgene expression preferentially in ON-retinal ganglion cells following intravitreal injection.

### Example 20—Treating a Retinal Condition Using an AAV Vector

An AAV vector is constructed to include an AAV2 capsid polypeptide having an amino acid sequence set forth in Table 1J (e.g., SEQ ID NO:J2) or according to Formula A based on such a set forth sequence and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 21—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10:e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10X Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence of any one of SEQ ID NOs:K2-K60 located between amino acid residues 587 and 588 of SEQ ID NO:1 or an amino acid sequence of any one of SEQ ID NOs:K61-K63 as a replacement of amino acid residues 585 to 590 of SEQ ID NO:1 mediated expression preferentially in OFF-retinal ganglion cells (Table 1K). The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression in retinal cells. No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1K or according to Formula A based on such a set forth sequence can have the ability to mediate transgene expression preferentially in OFF-retinal ganglion cells following intravitreal injection.

### Example 22—Treating a Retinal Condition Using an AAV Vector

An AAV vector is constructed to include an AAV2 capsid polypeptide having an amino acid sequence set forth in Table 1K (e.g., SEQ ID NO:K2) or according to Formula A based on such a set forth sequence and an exogenous nucleic acid sequence encoding a therapeutic polypeptide. The constructed AAV vector is administered intravitreally to a human identified as having a retinal condition in an amount that is from about 1×10¹⁴ to about 1×10¹⁴ AAV vectors. After the administration, the severity of one or more symptoms of the retinal condition is reduced and/or the progression of the retinal condition is slowed.

### Example 23—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10:e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library.

The performance of AAV capsid polypeptides for packaging was evaluated on the basis of successful packaging in the original and the “repack” library, reflecting the ability of the AAV vectors to package.

AAV vectors having capsid polypeptides that included an amino acid sequence of any one of SEQ ID NOs:L2-L19 located between amino acid residues 587 and 588 of SEQ ID NO:1 or an amino acid sequence of any one of SEQ ID NOs:L20-L27 as a replacement of amino acid residues 585 to 590 of SEQ ID NO:1 mediated efficient packaging (Table 1L). The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested.

Taken together, these results demonstrate that AAV vectors that include an AAV capsid polypeptide (e.g., an AAV2 capsid polypeptide) having an amino acid sequence set forth in Table 1L or according to Formula A based on such a set forth sequence can have the ability to mediate efficient packaging.

### Example 24—Treating a Retinal Condition Using an AAV Vector

An AAV vector is constructed to include an AAV2 capsid polypeptide having an amino acid sequence set forth in Table 1L (e.g., SEQ ID NO:L2) or according to Formula A based on such a set forth sequence and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 25—Additional Embodiments

Embodiment 1A. An adeno-associated virus (AAV) vector comprising an AAV capsid polypeptide, wherein said capsid polypeptide comprises the amino acid sequence of any one of SEQ ID NOs:A2-A19.

Embodiment 2A. The vector of Embodiment 1A, wherein said capsid polypeptide comprises the amino acid sequence of SEQ ID NO:1 except that said amino acid sequence of any one of SEQ ID NOs:A2-A19 is located between amino acid positions 587 and 588 of SEQ ID NO:1.

Embodiment 3A. The vector of Embodiment 1A, wherein said capsid polypeptide comprises the amino acid sequence of SEQ ID NO:1 except that said amino acid sequence of any one of SEQ ID NOs:A2-A18 is located between amino acid positions 587 and 588 of SEQ ID NO:1.

Embodiment 4A. The vector of Embodiment 1A, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:A2-A19.

Embodiment 5A. The vector of Embodiment 1A, wherein said capsid polypeptide comprises the amino acid sequence of SEQ ID NO:1 except that the amino acids from position 585 to 590 of SEQ ID NO:1 are replaced with said amino acid sequence of SEQ ID NO:A19.

Embodiment 6A. The vector of any one of Embodiments 1A-5A, wherein said vector is an AAV2 vector.

Embodiment 7A. The vector of any one of Embodiments 1A-6A, wherein said vector infects greater than 50 percent of foveal cones when a titer of at least 1×10⁷ of said vector is administered intravitreally to an eye of a human (or a non-human primate).

Embodiment 8A. The vector of any one of Embodiments 1A-7A, wherein said vector comprises an exogenous nucleic acid encoding an RNA or a polypeptide.

Embodiment 9A. The vector of Embodiment 8A, wherein said exogenous nucleic acid encodes an RNA.

Embodiment 10A. The vector of Embodiment 9A, wherein said RNA is an siRNA or microRNA.

Embodiment 11A. The vector of Embodiment 8A, wherein said exogenous nucleic acid encodes a polypeptide.

Embodiment 12A. The vector of Embodiment 11A, wherein said polypeptide is an ABCA4 polypeptide, a CRB1 polypeptide, an NPHP5 polypeptide, or an NR2E3 polypeptide.

Embodiment 13A. The vector of any one of Embodiments 1A-12A, wherein said vector expresses more nucleic acid in foveal cones than the level of expression from a comparable AAV vector comprising a capsid polypeptide consisting of the amino acid sequence set forth in SEQ ID NO:1.

Embodiment 14A. An AAV capsid polypeptide comprising the amino acid sequence of any one of SEQ ID NOs:A2-A19.

Embodiment 15A. The polypeptide of Embodiment 14A, wherein said polypeptide comprises the amino acid sequence of SEQ ID NO:1 except that said amino acid sequence of any one of SEQ ID NOs:A2-A19 is located between amino acid positions 587 and 588 of SEQ ID NO:1.

Embodiment 16A. The polypeptide of Embodiment 14A, wherein said polypeptide comprises the amino acid sequence of SEQ ID NO:1 except that said amino acid sequence of any one of SEQ ID NOs:A2-A18 is located between amino acid positions 587 and 588 of SEQ ID NO:1.

Embodiment 17A. The polypeptide of Embodiment 14A, wherein said polypeptide comprises the amino acid sequence of SEQ ID NO:1 except that the amino acids from position 585 to 590 of SEQ ID NO:1 are replaced with said amino acid sequence of any one of SEQ ID NOs:A2-A19.

Embodiment 18A. The polypeptide of Embodiment 14A, wherein said polypeptide comprises the amino acid sequence of SEQ ID NO:1 except that the amino acids from position 585 to 590 of SEQ ID NO:1 are replaced with said amino acid sequence of SEQ ID NO:A19.

Embodiment 19A. The polypeptide of any one of Embodiments 14A-18A, wherein an AAV vector comprising said polypeptide infects greater than 50 percent of foveal cones when a titer of at least 1×10⁷ of said vector is administered intravitreally to an eye of a human (or a non-human primate).

Embodiment 20A. The polypeptide of any one of Embodiments 14A-19A, wherein an AAV vector comprising said polypeptide expresses more nucleic acid in foveal cones than the level of expression from a comparable AAV vector comprising a capsid polypeptide consisting of the amino acid sequence set forth in SEQ ID NO:1.

Embodiment 21A. A nucleic acid molecule encoding a vector of any one of Embodiments 1A-13A or a polypeptide of any one of Embodiments 14A-20A.

Embodiment 22A. The nucleic acid molecule of Embodiment 21A, wherein said nucleic acid molecule is DNA.

Embodiment 23A. A host cell comprising a nucleic acid molecule of any one of Embodiments 21A-22A.

Embodiment 24A. The host cell of Embodiment 23A, wherein said host cell expresses said vector.

Embodiment 25A. The host cell of Embodiment 23A, wherein said host cell expresses said polypeptide.

Embodiment 26A. A host cell comprising a vector of any one of Embodiments 1A-13A or a polypeptide of any one of Embodiments 14A-20A.

Embodiment 27A. The host cell of any one of Embodiments 23A-26A, wherein said host cell is a retinal cell.

Embodiment 28A. A composition comprising a vector of any one of Embodiments 1A-13A.

Embodiment 29A. The composition of Embodiment 28A, wherein said composition comprises from about 1×10⁷ to about 1×10¹⁴ of said vector.

Embodiment 30A. The composition of any one of Embodiments 28A-29A, wherein said composition comprises phosphate buffered saline, Hank's Balanced Salt Solution, or Pluronic F68.

Embodiment 31A. A method for delivering an exogenous nucleic acid sequence to a foveal cone within a mammal, wherein said method comprises contacting said foveal cone with an AAV vector comprising an AAV capsid polypeptide and said exogenous nucleic acid sequence, wherein said capsid polypeptide comprises the amino acid sequence of any one of SEQ ID NOs:A2-A19, wherein said AAV vector infects said foveal cone, thereby delivering said exogenous nucleic acid sequence to said foveal cone.

Embodiment 32A. The method of Embodiment 31A, wherein said capsid polypeptide comprises the amino acid sequence of SEQ ID NO:1 except that said amino acid sequence of any one of SEQ ID NOs:A2-A19 is located between amino acid positions 587 and 588 of SEQ ID NO:1.

Embodiment 33A. The method of Embodiment 31A, wherein said capsid polypeptide comprises the amino acid sequence of SEQ ID NO:1 except that said amino acid sequence of any one of SEQ ID NOs:A2-A18 is located between amino acid positions 587 and 588 of SEQ ID NO:1.

Embodiment 34A. The method of Embodiment A31, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:A2-A19.

Embodiment 35A. The method of Embodiment 31A, wherein said capsid polypeptide comprises the amino acid sequence of SEQ ID NO:1 except that the amino acids from position 585 to 590 of SEQ ID NO:1 are replaced with said amino acid sequence of SEQ ID NO:A19.

Embodiment 36A. The method of any one of Embodiments 31A-35A, wherein said mammal is a human.

Embodiment 37A. The method of any one of Embodiments 31A-36A, wherein said vector is an AAV2 vector.

Embodiment 38A. The method of any one of Embodiments 31A-37A, wherein said vector infects greater than 50 percent of foveal cones when a titer of at least 1×10⁷ of said vector is administered intravitreally to an eye of a human (or a non-human primate).

Embodiment 39A. The method of any one of Embodiments 31A-38A, wherein said exogenous nucleic acid sequence encodes an RNA or a polypeptide.

Embodiment 40A. The method of Embodiment 39A, wherein said exogenous nucleic acid encodes an RNA.

Embodiment 41A. The method of Embodiment 40A, wherein said RNA is an siRNA or microRNA.

Embodiment 42A. The method of Embodiment 39A, wherein said exogenous nucleic acid encodes a polypeptide.

Embodiment 43A. The method of Embodiment 42A, wherein said polypeptide is an ABCA4 polypeptide, a CRB1 polypeptide, an NPHP5 polypeptide, or an NR2E3 polypeptide.

Embodiment 44A. The method of any one of Embodiments 31A-43A, wherein said vector expresses more of said exogenous nucleic acid sequence in said foveal cone than the level of expression in a foveal cone from a comparable AAV vector comprising a capsid polypeptide consisting of the amino acid sequence set forth in SEQ ID NO:1.

Embodiment 45A. The method of any one of Embodiments 31A-44A, wherein said method comprises intravitreally administering a composition comprising said vector to said mammal, thereby contacting said foveal cone with said vector.

Embodiment 46A. The method of Embodiment 45A, wherein said composition comprises from about 1×10⁷ to about 1×10⁴ of said vector.

Embodiment 47A. A method for treating a retinal condition, wherein said method comprises contacting foveal cones of a mammal having said retinal condition with AAV vectors comprising an AAV capsid polypeptide and an exogenous nucleic acid sequence, wherein said capsid polypeptide comprises the amino acid sequence of any one of SEQ ID NOs:A2-A19, wherein said AAV vectors infect said foveal cones and drive expression of said exogenous nucleic acid sequence within said foveal cones, thereby treating said retinal condition.

Embodiment 48A. The method of Embodiment 47A, wherein said mammal is a human.

Embodiment 49A. The method of any one of Embodiments 47A-48A, wherein said retinal condition is selected from the group consisting of cone dystrophy, cone/rod dystrophy, retinitis pigmentosa, macular degeneration, achromatopsia, blue cone monochromcy, and color blindness.

Embodiment 50A. The method of any one of Embodiments 47A-49A, wherein said capsid polypeptide comprises the amino acid sequence of SEQ ID NO:1 except that said amino acid sequence of any one of SEQ ID NOs:A2-A19 is located between amino acid positions 587 and 588 of SEQ ID NO:1.

Embodiment 51A. The method of any one of Embodiments 47A-49A, wherein said capsid polypeptide comprises the amino acid sequence of SEQ ID NO:1 except that said amino acid sequence of any one of SEQ ID NOs:A2-A18 is located between amino acid positions 587 and 588 of SEQ ID NO:1.

Embodiment 52A. The method of any one of Embodiments 47A-49A, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:A2-A19.

Embodiment 53A. The method of any one of Embodiments 47A-49A, wherein said capsid polypeptide comprises the amino acid sequence of SEQ ID NO:1 except that the amino acids from position 585 to 590 of SEQ ID NO:1 are replaced with said amino acid sequence of SEQ ID NO:A19.

Embodiment 54A. The method of any one of Embodiments 47A-53A, wherein said vectors are AAV2 vectors.

Embodiment 55A. The method of any one of Embodiments 47A-54A, wherein said vectors infect greater than 50 percent of foveal cones when a titer of at least 1×10⁷ of said vectors is administered intravitreally to an eye of said mammal.

Embodiment 56A. The method of any one of Embodiments 47A-55A, wherein said exogenous nucleic acid sequence encodes an RNA.

Embodiment 57A. The method of Embodiment 56A, wherein said RNA is an siRNA or a microRNA.

Embodiment 58A. The method of any one of Embodiments 47A-55A, wherein said exogenous nucleic acid encodes a polypeptide.

Embodiment 59A. The method of Embodiment 58A, wherein said polypeptide is an ABCA4 polypeptide, a CRB1 polypeptide, an NPHP5 polypeptide, and an NR2E3 polypeptide.

Embodiment 60A. The method of any one of Embodiments 47A-59A, wherein said vectors express more of said exogenous nucleic acid sequence in said foveal cones than the level of expression in foveal cones from a comparable AAV vector comprising a capsid polypeptide consisting of the amino acid sequence set forth in SEQ ID NO:1.

Embodiment 61A. The method of any one of Embodiments 47A-60A, wherein said method comprises intravitreally administering a composition comprising said vectors to said mammal, thereby contacting said foveal cones with said vectors.

Embodiment 62A. The method of Embodiment 61A, wherein said composition comprises from about 1×10⁷ to about 1×10¹⁴ of said vectors.

Embodiment 1B. An adeno-associated virus (AAV) vector comprising an AAV capsid polypeptide, wherein said capsid polypeptide comprises the amino acid sequence of any one of SEQ ID NOs:B2-B317.

Embodiment 2B. The vector of Embodiment 1B, wherein said capsid polypeptide comprises the amino acid sequence of SEQ ID NO:1 except that said amino acid sequence of any one of SEQ ID NOs:B2-B317 is located between amino acid positions 587 and 588 of SEQ ID NO:1.

Embodiment 3B. The vector of Embodiment 1B, wherein said capsid polypeptide comprises the amino acid sequence of SEQ ID NO:1 except that said amino acid sequence of any one of SEQ ID NOs:B2-B298 is located between amino acid positions 587 and 588 of SEQ ID NO:1.

Embodiment 4B. The vector of Embodiment 1B, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:B2-B317.

Embodiment 5B. The vector of Embodiment 1B, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:B299-B317.

Embodiment 6B. The vector of any one of Embodiments 1B-5B, wherein said vector is an AAV2 vector.

Embodiment 7B. The vector of any one of Embodiments 1B-6B, wherein said vector infects greater than 2.5 percent of retinal cells when a titer of at least 1×10⁷ of said vector is administered intravitreally to an eye of a human (or a non-human primate).

Embodiment 8B. The vector of any one of Embodiments 1B-7B, wherein said vector comprises an exogenous nucleic acid encoding an RNA or a polypeptide.

Embodiment 9B. The vector of Embodiment 8B, wherein said exogenous nucleic acid encodes an RNA.

Embodiment 10B. The vector of Embodiment 9B, wherein said RNA is an siRNA or microRNA.

Embodiment 11B. The vector of Embodiment 8B, wherein said exogenous nucleic acid encodes a polypeptide.

Embodiment 12B. The vector of Embodiment 11B, wherein said polypeptide is an ABCA4 polypeptide, a CRB1 polypeptide, an NPHP5 polypeptide, or an NR2E3 polypeptide.

Embodiment 13B. The vector of any one of Embodiments 1B-12B, wherein said vector expresses more nucleic acid in retinal cells than the level of expression from a comparable AAV vector comprising a capsid polypeptide consisting of the amino acid sequence set forth in SEQ ID NO:1.

Embodiment 14B. An AAV capsid polypeptide comprising the amino acid sequence of any one of SEQ ID NOs:B2-B317.

Embodiment 15B. The polypeptide of Embodiment 14B, wherein said polypeptide comprises the amino acid sequence of SEQ ID NO:1 except that said amino acid sequence of any one of SEQ ID NOs:B2-B317 is located between amino acid positions 587 and 588 of SEQ ID NO: 1.

Embodiment 16B. The polypeptide of Embodiment 14B, wherein said polypeptide comprises the amino acid sequence of SEQ ID NO:1 except that said amino acid sequence of any one of SEQ ID NOs:B2-B298 is located between amino acid positions 587 and 588 of SEQ ID NO:1.

Embodiment 17B. The polypeptide of Embodiment 14B, wherein said polypeptide comprises the amino acid sequence of SEQ ID NO:1 except that the amino acids from position 585 to 590 of SEQ ID NO:1 are replaced with said amino acid sequence of any one of SEQ ID NOs:B2-B317.

Embodiment 18B. The polypeptide of Embodiment 14B, wherein said polypeptide comprises the amino acid sequence of SEQ ID NO:1 except that the amino acids from position 585 to 590 of SEQ ID NO:1 are replaced with said amino acid sequence of any one of SEQ ID NOs:B299-B317.

Embodiment 19B. The polypeptide of any one of Embodiments 14B-18B, wherein an AAV vector comprising said polypeptide infects greater than 2.5 percent of retinal cells when a titer of at least 1×10⁷ of said vector is administered intravitreally to an eye of a human (or a non-human primate).

Embodiment 20B. The polypeptide of any one of Embodiments 14B-19B, wherein an AAV vector comprising said polypeptide expresses more nucleic acid in retinal cells than the level of expression from a comparable AAV vector comprising a capsid polypeptide consisting of the amino acid sequence set forth in SEQ ID NO:1.

Embodiment 21B. A nucleic acid molecule encoding a vector of any one of Embodiments 1B-13B or a polypeptide of any one of Embodiments 14B-20B.

Embodiment 22B. The nucleic acid molecule of Embodiment 21B, wherein said nucleic acid molecule is DNA.

Embodiment 23B. A host cell comprising a nucleic acid molecule of any one of Embodiments 21B-22B.

Embodiment 24B. The host cell of Embodiment 23B, wherein said host cell expresses said vector.

Embodiment 25B. The host cell of Embodiment 23B, wherein said host cell expresses said polypeptide.

Embodiment 26B. A host cell comprising a vector of any one of Embodiments 1B-13B or a polypeptide of any one of Embodiments 14B-20B.

Embodiment 27B. The host cell of any one of Embodiments 23B-26B, wherein said host cell is a retinal cell.

Embodiment 28B. A composition comprising a vector of any one of Embodiments 1B-13B.

Embodiment 29B. The composition of Embodiment 28B, wherein said composition comprises from about 1×10⁷ to about 1×10¹⁴ of said vector.

Embodiment 30B. The composition of any one of Embodiments 28B-29B, wherein said composition comprises phosphate buffered saline, Hank's Balanced Salt Solution, or Pluronic F68.

Embodiment 31B. A method for delivering an exogenous nucleic acid sequence to a retinal cell within a mammal, wherein said method comprises contacting said retinal cell with an AAV vector comprising an AAV capsid polypeptide and said exogenous nucleic acid sequence, wherein said capsid polypeptide comprises the amino acid sequence of any one of SEQ ID NOs:B2-B317, wherein said AAV vector infects said retinal cell, thereby delivering said exogenous nucleic acid sequence to said retinal cell.

Embodiment 32B. The method of Embodiment 31B, wherein said capsid polypeptide comprises the amino acid sequence of SEQ ID NO:1 except that said amino acid sequence of any one of SEQ ID NOs:B2-B317 is located between amino acid positions 587 and 588 of SEQ ID NO:1.

Embodiment 33B. The method of Embodiment 31B, wherein said capsid polypeptide comprises the amino acid sequence of SEQ ID NO:1 except that said amino acid sequence of any one of SEQ ID NOs:B2-B298 is located between amino acid positions 587 and 588 of SEQ ID NO:1.

Embodiment 34B. The method of Embodiment 31B, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:B2-B317.

Embodiment 35B. The method of Embodiment 31B, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:B299-B317.

Embodiment 36B. The method of any one of Embodiments 31B-35B, wherein said mammal is a human.

Embodiment 37B. The method of any one of Embodiments 31B-36B, wherein said vector is an AAV2 vector.

Embodiment 38B. The method of any one of Embodiments 31B-37B, wherein said vector infects greater than 2.5 percent of retinal cells when a titer of at least 1×10⁷ of said vector is administered intravitreally to an eye of a human (or a non-human primate).

Embodiment 39B. The method of any one of Embodiments 31B-38B, wherein said exogenous nucleic acid sequence encodes an RNA or a polypeptide.

Embodiment 40B. The method of Embodiment 39B, wherein said exogenous nucleic acid encodes an RNA.

Embodiment 41B. The method of Embodiment 40B, wherein said RNA is an siRNA or microRNA.

Embodiment 42B. The method of Embodiment 39B, wherein said exogenous nucleic acid encodes a polypeptide.

Embodiment 43B. The method of Embodiment 42B, wherein said polypeptide is an ABCA4 polypeptide, a CRB1 polypeptide, an NPHP5 polypeptide, or an NR2E3 polypeptide.

Embodiment 44B. The method of any one of Embodiments 31B-43B, wherein said vector expresses more of said exogenous nucleic acid sequence in said retinal cell than the level of expression in a retinal cell from a comparable AAV vector comprising a capsid polypeptide consisting of the amino acid sequence set forth in SEQ ID NO:1.

Embodiment 45B. The method of any one of Embodiments 31B-44B, wherein said method comprises intravitreally administering a composition comprising said vector to said mammal, thereby contacting said retinal cell with said vector.

Embodiment 46B. The method of Embodiment 45B, wherein said composition comprises from about 1×10⁷ to about 1×10¹⁴ of said vector.

Embodiment 47B. A method for treating a retinal condition, wherein said method comprises contacting retinal cells of a mammal having said retinal condition with AAV vectors comprising an AAV capsid polypeptide and an exogenous nucleic acid sequence, wherein said capsid polypeptide comprises the amino acid sequence of any one of SEQ ID NOs:B2-B317, wherein said AAV vectors infect said retinal cells and drive expression of said exogenous nucleic acid sequence within said retinal cells, thereby treating said retinal condition.

Embodiment 48B. The method of Embodiment 47B, wherein said mammal is a human.

Embodiment 49B. The method of any one of Embodiments 47B-48B, wherein said retinal condition is selected from the group consisting of LCA, OCA1, retinitis pigmentosa, rod/cone dystrophy, cone dystrophy, Stargardt Disease, Usher syndrome, XLRP, and XLRS.

Embodiment 50B. The method of any one of Embodiments 47B-49B, wherein said capsid polypeptide comprises the amino acid sequence of SEQ ID NO:1 except that said amino acid sequence of any one of SEQ ID NOs:B2-B317 is located between amino acid positions 587 and 588 of SEQ ID NO:1.

Embodiment 51B. The method of any one of Embodiments 47B-49B, wherein said capsid polypeptide comprises the amino acid sequence of SEQ ID NO:1 except that said amino acid sequence of any one of SEQ ID NOs:B2-B298 is located between amino acid positions 587 and 588 of SEQ ID NO:1.

Embodiment 52B. The method of any one of Embodiments 47B-49B, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:B2-B317.

Embodiment 53B. The method of any one of Embodiments 47B-49B, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:B299-B317.

Embodiment 54B. The method of any one of Embodiments 47B-53B, wherein said vectors are AAV2 vectors.

Embodiment 55B. The method of any one of Embodiments 47B-54B, wherein said vectors infect greater than 2.5 percent of retinal cells when a titer of at least 1×10⁷ of said vectors is administered intravitreally to an eye of said mammal.

Embodiment 56B. The method of any one of Embodiments 47B-55B, wherein said exogenous nucleic acid sequence encodes an RNA.

Embodiment 57B. The method of Embodiment 56B, wherein said RNA is an siRNA or a microRNA.

Embodiment 58B. The method of any one of Embodiments 47B-55B, wherein said exogenous nucleic acid encodes a polypeptide.

Embodiment 59B. The method of Embodiment 58B, wherein said polypeptide is an ABCA4 polypeptide, a CRB1 polypeptide, an NPHP5 polypeptide, and an NR2E3 polypeptide.

Embodiment 60B. The method of any one of Embodiments 47B-59B, wherein said vectors express more of said exogenous nucleic acid sequence in said retinal cells than the level of expression in retinal cells from a comparable AAV vector comprising a capsid polypeptide consisting of the amino acid sequence set forth in SEQ ID NO:1.

Embodiment 61B. The method of any one of Embodiments 47B-60B, wherein said method comprises intravitreally administering a composition comprising said vectors to said mammal, thereby contacting said retinal cells with said vectors.

Embodiment 62B. The method of Embodiment 61B, wherein said composition comprises from about 1×10⁷ to about 1×10⁴ of said vectors.

Embodiment 1C. An adeno-associated virus (AAV) vector comprising an AAV capsid polypeptide, wherein said capsid polypeptide comprises the amino acid sequence of any one of SEQ ID NOs:C2-C45.

Embodiment 2C. The vector of Embodiment 1C, wherein said capsid polypeptide comprises the amino acid sequence of SEQ ID NO:1 except that said amino acid sequence of any one of SEQ ID NOs:C2-C45 is located between amino acid positions 587 and 588 of SEQ ID NO:1.

Embodiment 3C. The vector of Embodiment 1C, wherein said capsid polypeptide comprises the amino acid sequence of SEQ ID NO:1 except that said amino acid sequence of any one of SEQ ID NOs:C2-C43 is located between amino acid positions 587 and 588 of SEQ ID NO:1.

Embodiment 4C. The vector of Embodiment 1C, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:C2-C45.

Embodiment 5C. The vector of Embodiment 1C, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:C44-C45.

Embodiment 6C. The vector of any one of Embodiments 1C-5C, wherein said vector is an AAV2 vector.

Embodiment 7C. The vector of any one of Embodiments 1C-6C, wherein said vector infects greater than 2 percent of retinal cells within two or more retinal regions when a titer of at least 1×10⁷ of said vector is administered intravitreally to an eye of a human (or a non-human primate).

Embodiment 8C. The vector of any one of Embodiments 1C-7C, wherein said vector comprises an exogenous nucleic acid encoding an RNA or a polypeptide.

Embodiment 9C. The vector of Embodiment 8C, wherein said exogenous nucleic acid encodes an RNA.

Embodiment 10C. The vector of Embodiment 9C, wherein said RNA is an siRNA or microRNA.

Embodiment 11C. The vector of Embodiment 8C, wherein said exogenous nucleic acid encodes a polypeptide.

Embodiment 12C. The vector of Embodiment 11C, wherein said polypeptide is an ABCA4 polypeptide, a CRB1 polypeptide, an NPHP5 polypeptide, or an NR2E3 polypeptide.

Embodiment 13C. The vector of any one of Embodiments 1C-12C, wherein said vector expresses more nucleic acid in retinal cells in at least two retinal regions than the level of expression from a comparable AAV vector comprising a capsid polypeptide consisting of the amino acid sequence set forth in SEQ ID NO:1, wherein said at least two retinal regions are selected from the group consisting of a fovea region, a parafovea region, a vascular arcade region, and a periphery region.

Embodiment 14C. An AAV capsid polypeptide comprising the amino acid sequence of any one of SEQ ID NOs:C2-C45.

Embodiment 15C. The polypeptide of Embodiment 14C, wherein said polypeptide comprises the amino acid sequence of SEQ ID NO:1 except that said amino acid sequence of any one of SEQ ID NOs:C2-C45 is located between amino acid positions 587 and 588 of SEQ ID NO:1.

Embodiment 16C. The polypeptide of Embodiment 14C, wherein said polypeptide comprises the amino acid sequence of SEQ ID NO:1 except that said amino acid sequence of any one of SEQ ID NOs:C2-C43 is located between amino acid positions 587 and 588 of SEQ ID NO:1.

Embodiment 17C. The polypeptide of Embodiment 14C, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:C2-C45.

Embodiment 18C. The polypeptide of Embodiment 14C, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:C44-C45.

Embodiment 19C. The polypeptide of any one of Embodiments 14C-18C, wherein an AAV vector comprising said polypeptide infects greater than 2 percent of retinal cells within two or more retinal regions when a titer of at least 1×10⁷ of said vector is administered intravitreally to an eye of a human (or a non-human primate).

Embodiment 20C. The polypeptide of any one of Embodiments 14C-19C, wherein an AAV vector comprising said polypeptide expresses more nucleic acid in retinal cells in at least two retinal regions than the level of expression from a comparable AAV vector comprising a capsid polypeptide consisting of the amino acid sequence set forth in SEQ ID NO:1, wherein said at least two retinal regions are selected from the group consisting of a fovea region, a parafovea region, a vascular arcade region, and a periphery region.

Embodiment 21C. A nucleic acid molecule encoding a vector of any one of Embodiments 1C-13C or a polypeptide of any one of Embodiments 14C-20C.

Embodiment 22C. The nucleic acid molecule of Embodiment 21C, wherein said nucleic acid molecule is DNA.

Embodiment 23C. A host cell comprising a nucleic acid molecule of any one of Embodiments 21C-22C.

Embodiment 24C. The host cell of Embodiment 23C, wherein said host cell expresses said vector.

Embodiment 25C. The host cell of Embodiment 23C, wherein said host cell expresses said polypeptide.

Embodiment 26C. A host cell comprising a vector of any one of Embodiments 1C-13C or a polypeptide of any one of Embodiments 14C-20C.

Embodiment 27C. The host cell of any one of Embodiments 23C-26C, wherein said host cell is a retinal cell.

Embodiment 28C. A composition comprising a vector of any one of Embodiments 1C-13C.

Embodiment 29C. The composition of Embodiment 28C, wherein said composition comprises from about 1×10⁷ to about 1×10¹⁴ of said vector.

Embodiment 30C. The composition of any one of Embodiments 28C-29C, wherein said composition comprises phosphate buffered saline, Hank's Balanced Salt Solution, or Pluronic F68.

Embodiment 31C. A method for delivering an exogenous nucleic acid sequence to retinal cells within at least two different retinal regions of an eye of a mammal, wherein said method comprises contacting said retinal cells with an AAV vector comprising an AAV capsid polypeptide and said exogenous nucleic acid sequence, wherein said capsid polypeptide comprises the amino acid sequence of any one of SEQ ID NOs:C2-C45, wherein said AAV vector infects retinal cells within said at least two different retinal regions, thereby delivering said exogenous nucleic acid sequence to said retinal cells, wherein said at least two retinal regions are selected from the group consisting of a fovea region, a parafovea region, a vascular arcade region, and a periphery region.

Embodiment 32C. The method of Embodiment 31C, wherein said capsid polypeptide comprises the amino acid sequence of SEQ ID NO:1 except that said amino acid sequence of any one of SEQ ID NOs:C2-C45 is located between amino acid positions 587 and 588 of SEQ ID NO:1.

Embodiment 33C. The method of Embodiment 31C, wherein said capsid polypeptide comprises the amino acid sequence of SEQ ID NO:1 except that said amino acid sequence of any one of SEQ ID NOs:C2-C43 is located between amino acid positions 587 and 588 of SEQ ID NO:1.

Embodiment 34C. The method of Embodiment 31C, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:C2-C45.

Embodiment 35C. The method of Embodiment 31C, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:C44-C45.

Embodiment 36C. The method of any one of Embodiments 31C-35C, wherein said mammal is a human.

Embodiment 37C. The method of any one of Embodiments 31C-36C, wherein said vector is an AAV2 vector.

Embodiment 38C. The method of any one of Embodiments 31C-37C, wherein said vector infects greater than 2 percent of retinal cells within said at least two retinal regions when a titer of at least 1×10⁷ of said vector is administered intravitreally to an eye of a human (or a non-human primate).

Embodiment 39C. The method of any one of Embodiments 31C-38C, wherein said exogenous nucleic acid sequence encodes an RNA or a polypeptide.

Embodiment 40C. The method of Embodiment 39C, wherein said exogenous nucleic acid encodes an RNA.

Embodiment 41C. The method of Embodiment 40C, wherein said RNA is an siRNA or microRNA.

Embodiment 42C. The method of Embodiment 39C, wherein said exogenous nucleic acid encodes a polypeptide.

Embodiment 43C. The method of Embodiment 42C, wherein said polypeptide is an ABCA4 polypeptide, a CRB1 polypeptide, an NPHP5 polypeptide, or an NR2E3 polypeptide.

Embodiment 44C. The method of any one of Embodiments 31C-43C, wherein said vector expresses more of said exogenous nucleic acid sequence in said retinal cells of said at least two retinal regions than the level of expression in a retinal cell from a comparable AAV vector comprising a capsid polypeptide consisting of the amino acid sequence set forth in SEQ ID NO:1.

Embodiment 45C. The method of any one of Embodiments 31C-44C, wherein said method comprises intravitreally administering a composition comprising said vector to said mammal, thereby contacting said retinal cells with said vector.

Embodiment 46C. The method of Embodiment 45C, wherein said composition comprises from about 1×10⁷ to about 1×10¹⁴ of said vector.

Embodiment 47C. A method for treating a retinal condition, wherein said method comprises contacting retinal cells of at least two retinal regions of an eye of a mammal having said retinal condition with AAV vectors comprising an AAV capsid polypeptide and an exogenous nucleic acid sequence, wherein said capsid polypeptide comprises the amino acid sequence of any one of SEQ ID NOs:C2-C45, wherein said AAV vectors infect said retinal cells of said at least two retinal regions and drive expression of said exogenous nucleic acid sequence within said retinal cells of said at least two retinal regions, thereby treating said retinal condition.

Embodiment 48C. The method of Embodiment 47C, wherein said mammal is a human.

Embodiment 49C. The method of any one of Embodiments 47C-48C, wherein said retinal condition is selected from the group consisting of LCA, OCA1, retinitis pigmentosa, rod/cone dystrophy, cone dystrophy, Stargardt Disease, Usher syndrome, XLRP, and XLRS.

Embodiment 50C. The method of any one of Embodiments 47C-49C, wherein said capsid polypeptide comprises the amino acid sequence of SEQ ID NO:1 except that said amino acid sequence of any one of SEQ ID NOs:C2-C45 is located between amino acid positions 587 and 588 of SEQ ID NO:1.

Embodiment 51C. The method of any one of Embodiments 47C-49C, wherein said capsid polypeptide comprises the amino acid sequence of SEQ ID NO:1 except that said amino acid sequence of any one of SEQ ID NOs:C2-C43 is located between amino acid positions 587 and 588 of SEQ ID NO:1.

Embodiment 52C. The method of any one of Embodiments 47C-49C, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:C2-C45.

Embodiment 53C. The method of any one of Embodiments 47C-49C, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:C44-C45.

Embodiment 54C. The method of any one of Embodiments 47C-53C, wherein said vectors are AAV2 vectors.

Embodiment 55C. The method of any one of Embodiments 47C-54C, wherein said vectors infect greater than 2 percent of retinal cells in said at least two retinal regions when a titer of at least 1×10¹⁴ of said vectors is administered intravitreally to an eye of said mammal.

Embodiment 56C. The method of any one of Embodiments 47C-55C, wherein said exogenous nucleic acid sequence encodes an RNA.

Embodiment 57C. The method of Embodiment 56C, wherein said RNA is an siRNA or a microRNA.

Embodiment 58C. The method of any one of Embodiments 47C-55C, wherein said exogenous nucleic acid encodes a polypeptide.

Embodiment 59C. The method of Embodiment 58C, wherein said polypeptide is an ABCA4 polypeptide, a CRB1 polypeptide, an NPHP5 polypeptide, and an NR2E3 polypeptide.

Embodiment 60C. The method of any one of Embodiments 47C-59C, wherein said vectors express more of said exogenous nucleic acid sequence in said retinal cells of said at least two retinal regions than the level of expression in retinal cells of said at least two retinal regions from a comparable AAV vector comprising a capsid polypeptide consisting of the amino acid sequence set forth in SEQ ID NO:1.

Embodiment 61C. The method of any one of Embodiments 47C-60C, wherein said method comprises intravitreally administering a composition comprising said vectors to said mammal, thereby contacting said retinal cells of said at least two retinal regions with said vectors.

Embodiment 62C. The method of Embodiment 61C, wherein said composition comprises from about 1×10⁷ to about 1×10¹⁴ of said vectors.

Embodiment 63C. The method of any one of Embodiments C47-C62, wherein said at least two retinal regions are selected from the group consisting of a fovea region, a parafovea region, a vascular arcade region, and a periphery region.

Embodiment 1D. An adeno-associated virus (AAV) vector comprising an AAV capsid polypeptide, wherein said capsid polypeptide comprises the amino acid sequence of any one of SEQ ID NOs:D2-D78.

Embodiment 2D. The vector of Embodiment 1D, wherein said capsid polypeptide comprises the amino acid sequence of SEQ ID NO:1 except that said amino acid sequence of any one of SEQ ID NOs:D2-D78 is located between amino acid positions 587 and 588 of SEQ ID NO:1.

Embodiment 3D. The vector of Embodiment 1D, wherein said capsid polypeptide comprises the amino acid sequence of SEQ ID NO:1 except that said amino acid sequence of any one of SEQ ID NOs:D2-D76 is located between amino acid positions 587 and 588 of SEQ ID NO:1.

Embodiment 4D. The vector of Embodiment 1D, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:D2-D78.

Embodiment 5D. The vector of Embodiment 1D, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:D77-D78.

Embodiment 6D. The vector of any one of Embodiments 1D-5D, wherein said vector is an AAV2 vector.

Embodiment 7D. The vector of any one of Embodiments 1D-6D, wherein said vector infects greater than 2 percent of retinal cells of a parafovea region of an eye when a titer of at least 1×10⁷ of said vector is administered intravitreally to an eye of a human (or a non-human primate).

Embodiment 8D. The vector of any one of Embodiments 1D-7D, wherein said vector comprises an exogenous nucleic acid encoding an RNA or a polypeptide.

Embodiment 9D. The vector of Embodiment 8D, wherein said exogenous nucleic acid encodes an RNA.

Embodiment 10D. The vector of Embodiment 9D, wherein said RNA is an siRNA or microRNA.

Embodiment 11D. The vector of Embodiment 8D, wherein said exogenous nucleic acid encodes a polypeptide.

Embodiment 12D. The vector of Embodiment 11D, wherein said polypeptide is an ABCA4 polypeptide, a CRB1 polypeptide, an NPHP5 polypeptide, or an NR2E3 polypeptide.

Embodiment 13D. The vector of any one of Embodiments 1D-12D, wherein said vector expresses more nucleic acid in retinal cells of a parafovea region of an eye than the level of expression from a comparable AAV vector comprising a capsid polypeptide consisting of the amino acid sequence set forth in SEQ ID NO:1.

Embodiment 14D. An AAV capsid polypeptide comprising the amino acid sequence of any one of SEQ ID NOs:D2-D78.

Embodiment 15D. The polypeptide of Embodiment 14D, wherein said polypeptide comprises the amino acid sequence of SEQ ID NO:1 except that said amino acid sequence of any one of SEQ ID NOs:D2-D78 is located between amino acid positions 587 and 588 of SEQ ID NO:1.

Embodiment 16D. The polypeptide of Embodiment 14D, wherein said polypeptide comprises the amino acid sequence of SEQ ID NO:1 except that said amino acid sequence of any one of SEQ ID NOs:D2-D76 is located between amino acid positions 587 and 588 of SEQ ID NO:1.

Embodiment 17D. The polypeptide of Embodiment 14D, wherein said polypeptide comprises the amino acid sequence of SEQ ID NO:1 except that the amino acids from position 585 to 590 of SEQ ID NO:1 are replaced with said amino acid sequence of any one of SEQ ID NOs:D2-D78.

Embodiment 18D. The polypeptide of Embodiment 14D, wherein said polypeptide comprises the amino acid sequence of SEQ ID NO:1 except that the amino acids from position 585 to 590 of SEQ ID NO:1 are replaced with said amino acid sequence of any one of SEQ ID NOs:D77-D78.

Embodiment 19D. The polypeptide of any one of Embodiments 14D-18D, wherein an AAV vector comprising said polypeptide infects greater than 2 percent of retinal cells of a parafovea region of an eye when a titer of at least 1×10⁷ of said vector is administered intravitreally to an eye of a human (or a non-human primate).

Embodiment 20D. The polypeptide of any one of Embodiments 14D-19D, wherein an AAV vector comprising said polypeptide expresses more nucleic acid in retinal cells of a parafovea region of an eye than the level of expression from a comparable AAV vector comprising a capsid polypeptide consisting of the amino acid sequence set forth in SEQ ID NO:1.

Embodiment 21D. A nucleic acid molecule encoding a vector of any one of Embodiments 1D-13D or a polypeptide of any one of Embodiments 14D-20D.

Embodiment 22D. The nucleic acid molecule of Embodiment 21D, wherein said nucleic acid molecule is DNA.

Embodiment 23D. A host cell comprising a nucleic acid molecule of any one of Embodiments 21D-22D.

Embodiment 24D. The host cell of Embodiment 23D, wherein said host cell expresses said vector.

Embodiment 25D. The host cell of Embodiment 23D, wherein said host cell expresses said polypeptide.

Embodiment 26D. A host cell comprising a vector of any one of Embodiments 1D-13D or a polypeptide of any one of Embodiments 14D-20D.

Embodiment 27D. The host cell of any one of Embodiments 23D-26D, wherein said host cell is a retinal cell.

Embodiment 28D. A composition comprising a vector of any one of Embodiments 1D-13D.

Embodiment 29D. The composition of Embodiment 28D, wherein said composition comprises from about 1×10⁷ to about 1×10¹⁴ of said vector.

Embodiment 30D. The composition of any one of Embodiments 28D-29D, wherein said composition comprises phosphate buffered saline, Hank's Balanced Salt Solution, or Pluronic F68.

Embodiment 31D. A method for delivering an exogenous nucleic acid sequence to retinal cells of a parafovea region of an eye within a mammal, wherein said method comprises contacting said retinal cells with an AAV vector comprising an AAV capsid polypeptide and said exogenous nucleic acid sequence, wherein said capsid polypeptide comprises the amino acid sequence of any one of SEQ ID NOs:D2-D78, wherein said AAV vector infects said retinal cells, thereby delivering said exogenous nucleic acid sequence to said retinal cells.

Embodiment 32D. The method of Embodiment 31D, wherein said capsid polypeptide comprises the amino acid sequence of SEQ ID NO:1 except that said amino acid sequence of any one of SEQ ID NOs:D2-D78 is located between amino acid positions 587 and 588 of SEQ ID NO:1.

Embodiment 33D. The method of Embodiment 31D, wherein said capsid polypeptide comprises the amino acid sequence of SEQ ID NO:1 except that said amino acid sequence of any one of SEQ ID NOs:D2-D76 is located between amino acid positions 587 and 588 of SEQ ID NO:1.

Embodiment 34D. The method of Embodiment 31D, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:D2-D78.

Embodiment 35D. The method of Embodiment 31D, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:D77-D78.

Embodiment 36D. The method of any one of Embodiments 31D-35D, wherein said mammal is a human.

Embodiment 37D. The method of any one of Embodiments 31D-36D, wherein said vector is an AAV2 vector.

Embodiment 38D. The method of any one of Embodiments 31D-37D, wherein said vector infects greater than 2 percent of retinal cells of said parafovea region of said eye when a titer of at least 1×10¹⁴ of said vector is administered intravitreally.

Embodiment 39D. The method of any one of Embodiments 31D-38D, wherein said exogenous nucleic acid sequence encodes an RNA or a polypeptide.

Embodiment 40D. The method of Embodiment 39D, wherein said exogenous nucleic acid encodes an RNA.

Embodiment 41D. The method of Embodiment 40D, wherein said RNA is an siRNA or microRNA.

Embodiment 42D. The method of Embodiment 39D, wherein said exogenous nucleic acid encodes a polypeptide.

Embodiment 43D. The method of Embodiment 42D, wherein said polypeptide is an ABCA4 polypeptide, a CRB1 polypeptide, an NPHP5 polypeptide, or an NR2E3 polypeptide.

Embodiment 44D. The method of any one of Embodiments 31D-43D, wherein said vector expresses more of said exogenous nucleic acid sequence in said retinal cells than the level of expression in retinal cells of a parafovea region of an eye from a comparable AAV vector comprising a capsid polypeptide consisting of the amino acid sequence set forth in SEQ ID NO:1.

Embodiment 45D. The method of any one of Embodiments 31D-44D, wherein said method comprises intravitreally administering a composition comprising said vector to said mammal, thereby contacting said retinal cells of said parafovea region with said vector.

Embodiment 46D. The method of Embodiment 45D, wherein said composition comprises from about 1×10⁷ to about 1×10⁴ of said vector.

Embodiment 47D. A method for treating a retinal condition, wherein said method comprises contacting retinal cells of a parafovea region of an eye of a mammal having said retinal condition with AAV vectors comprising an AAV capsid polypeptide and an exogenous nucleic acid sequence, wherein said capsid polypeptide comprises the amino acid sequence of any one of SEQ ID NOs:D2-D78, wherein said AAV vectors infect said retinal cells and drive expression of said exogenous nucleic acid sequence within said retinal cells, thereby treating said retinal condition.

Embodiment 48D. The method of Embodiment 47D, wherein said mammal is a human.

Embodiment 49D. The method of any one of Embodiments 47D-48D, wherein said retinal condition is selected from the group consisting of cone dystrophy, cone/rod dystrophy, retinitis pigmentosa, macular degeneration, achromatopsia, blue cone monochromcy, and color blindness.

Embodiment 50D. The method of any one of Embodiments 47D-49D, wherein said capsid polypeptide comprises the amino acid sequence of SEQ ID NO:1 except that said amino acid sequence of any one of SEQ ID NOs:D2-D78 is located between amino acid positions 587 and 588 of SEQ ID NO:1.

Embodiment 51D. The method of any one of Embodiments 47D-49D, wherein said capsid polypeptide comprises the amino acid sequence of SEQ ID NO:1 except that said amino acid sequence of any one of SEQ ID NOs:D2-D76 is located between amino acid positions 587 and 588 of SEQ ID NO:1.

Embodiment 52D. The method of any one of Embodiments 47D-49D, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:D2-D78.

Embodiment 53D. The method of any one of Embodiments 47D-49D, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:D77-D78.

Embodiment 54D. The method of any one of Embodiments 47D-53D, wherein said vectors are AAV2 vectors.

Embodiment 55D. The method of any one of Embodiments 47D-54D, wherein said vectors infect greater than 2 percent of retinal cells of said parafovea region when a titer of at least 1×10⁷ of said vectors is administered intravitreally.

Embodiment 56D. The method of any one of Embodiments 47D-55D, wherein said exogenous nucleic acid sequence encodes an RNA.

Embodiment 57D. The method of Embodiment 56D, wherein said RNA is an siRNA or a microRNA.

Embodiment 58D. The method of any one of Embodiments 47D-55D, wherein said exogenous nucleic acid encodes a polypeptide.

Embodiment 59D. The method of Embodiment 58D, wherein said polypeptide is an ABCA4 polypeptide, a CRB1 polypeptide, an NPHP5 polypeptide, and an NR2E3 polypeptide.

Embodiment 60D. The method of any one of Embodiments 47D-59D, wherein said vectors express more of said exogenous nucleic acid sequence in said retinal cells of said parafovea region than the level of expression in retinal cells of a parafovea region from a comparable AAV vector comprising a capsid polypeptide consisting of the amino acid sequence set forth in SEQ ID NO:1.

Embodiment 61D. The method of any one of Embodiments 47D-60D, wherein said method comprises intravitreally administering a composition comprising said vectors to said mammal, thereby contacting said retinal cells of said parafovea region with said vectors.

Embodiment 62D. The method of Embodiment 61D, wherein said composition comprises from about 1×10⁷ to about 1×10¹⁴ of said vectors.

Embodiment 1E. An adeno-associated virus (AAV) vector comprising an AAV capsid polypeptide, wherein said capsid polypeptide comprises the amino acid sequence of any one of SEQ ID NOs:E2-E83.

Embodiment 2E. The vector of Embodiment 1E, wherein said capsid polypeptide comprises the amino acid sequence of SEQ ID NO:1 except that said amino acid sequence of any one of SEQ ID NOs:E2-E83 is located between amino acid positions 587 and 588 of SEQ ID NO:1.

Embodiment 3E. The vector of Embodiment 1E, wherein said capsid polypeptide comprises the amino acid sequence of SEQ ID NO:1 except that said amino acid sequence of any one of SEQ ID NOs:E2-E75 is located between amino acid positions 587 and 588 of SEQ ID NO:1.

Embodiment 4E. The vector of Embodiment 1E, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:E2-E83.

Embodiment 5E. The vector of Embodiment 1E, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:E76-E83.

Embodiment 6E. The vector of any one of Embodiments 1E-5E, wherein said vector is an AAV2 vector.

Embodiment 7E. The vector of any one of Embodiments 1E-6E, wherein said vector infects at least three different retinal cell types when a titer of at least 1×10⁷ of said vector is administered intravitreally to an eye of a human (or a non-human primate), wherein said at least three different retinal cell types are selected from the group consisting of retinal ganglion cells, amacrine cells, horizontal cells, bipolar cells, Muller glia cells, photoreceptor cells, and RPE cells.

Embodiment 8E. The vector of any one of Embodiments 1E-7E, wherein said vector comprises an exogenous nucleic acid encoding an RNA or a polypeptide.

Embodiment 9E. The vector of Embodiment 8E, wherein said exogenous nucleic acid encodes an RNA.

Embodiment 10E. The vector of Embodiment 9E, wherein said RNA is an siRNA or microRNA.

Embodiment 11E. The vector of Embodiment 8E, wherein said exogenous nucleic acid encodes a polypeptide.

Embodiment 12E. The vector of Embodiment 11E, wherein said polypeptide is an ABCA4 polypeptide, a CRB1 polypeptide, an NPHP5 polypeptide, or an NR2E3 polypeptide.

Embodiment 13E. The vector of any one of Embodiments 1E-12E, wherein said vector expresses more nucleic acid in at least three different retinal cell types than the level of expression from a comparable AAV vector comprising a capsid polypeptide consisting of the amino acid sequence set forth in SEQ ID NO:1, wherein said at least three different retinal cell types are selected from the group consisting of retinal ganglion cells, amacrine cells, horizontal cells, bipolar cells, Muller glia cells, photoreceptor cells, and RPE cells.

Embodiment 14E. An AAV capsid polypeptide comprising the amino acid sequence of any one of SEQ ID NOs:E2-E83.

Embodiment 15E. The polypeptide of Embodiment 14E, wherein said polypeptide comprises the amino acid sequence of SEQ ID NO:1 except that said amino acid sequence of any one of SEQ ID NOs:E2-E83 is located between amino acid positions 587 and 588 of SEQ ID NO:1.

Embodiment 16E. The polypeptide of Embodiment 14E, wherein said polypeptide comprises the amino acid sequence of SEQ ID NO:1 except that said amino acid sequence of any one of SEQ ID NOs:E2-E75 is located between amino acid positions 587 and 588 of SEQ ID NO:1.

Embodiment 17E. The polypeptide of Embodiment 14E, wherein said polypeptide comprises the amino acid sequence of SEQ ID NO:1 except that the amino acids from position 585 to 590 of SEQ ID NO:1 are replaced with said amino acid sequence of any one of SEQ ID NOs:E2-E83.

Embodiment 18E. The polypeptide of Embodiment 14E, wherein said polypeptide comprises the amino acid sequence of SEQ ID NO:1 except that the amino acids from position 585 to 590 of SEQ ID NO:1 are replaced with said amino acid sequence of any one of SEQ ID NOs:E76-E83.

Embodiment 19E. The polypeptide of any one of Embodiments 14E-18E, wherein an AAV vector comprising said polypeptide infects at least three different retinal cell types when a titer of at least 1×10⁷ of said vector is administered intravitreally to an eye of a human (or a non-human primate), wherein said at least three different retinal cell types are selected from the group consisting of retinal ganglion cells, amacrine cells, horizontal cells, bipolar cells, Muller glia cells, photoreceptor cells, and RPE cells.

Embodiment 20E. The polypeptide of any one of Embodiments 14E-19E, wherein an AAV vector comprising said polypeptide expresses more nucleic acid in at least three different retinal cell types than the level of expression from a comparable AAV vector comprising a capsid polypeptide consisting of the amino acid sequence set forth in SEQ ID NO:1, wherein said at least three different retinal cell types are selected from the group consisting of retinal ganglion cells, amacrine cells, horizontal cells, bipolar cells, Muller glia cells, photoreceptor cells, and RPE cells.

Embodiment 21E. A nucleic acid molecule encoding a vector of any one of Embodiments 1E-13E or a polypeptide of any one of Embodiments 14E-20E.

Embodiment 22E. The nucleic acid molecule of Embodiment 21E, wherein said nucleic acid molecule is DNA.

Embodiment 23E. A host cell comprising a nucleic acid molecule of any one of Embodiments 21E-22E.

Embodiment 24E. The host cell of Embodiment 23E, wherein said host cell expresses said vector.

Embodiment 25E. The host cell of Embodiment 23E, wherein said host cell expresses said polypeptide.

Embodiment 26E. A host cell comprising a vector of any one of Embodiments 1E-13E or a polypeptide of any one of Embodiments 14E-20E.

Embodiment 27E. The host cell of any one of Embodiments 23E-26E, wherein said host cell is a retinal cell.

Embodiment 28E. A composition comprising a vector of any one of Embodiments 1E-13E.

Embodiment 29E. The composition of Embodiment 28E, wherein said composition comprises from about 1×10⁷ to about 1×10¹⁴ of said vector.

Embodiment 30E. The composition of any one of Embodiments 28E-29E, wherein said composition comprises phosphate buffered saline, Hank's Balanced Salt Solution, or Pluronic E68.

Embodiment 31E. A method for delivering an exogenous nucleic acid sequence to at least three different retinal cell types of an eye of a mammal, wherein said method comprises contacting said at least three different retinal cell types with an AAV vector comprising an AAV capsid polypeptide and said exogenous nucleic acid sequence, wherein said capsid polypeptide comprises the amino acid sequence of any one of SEQ ID NOs:E2-E83, wherein said AAV vector infects said at least three different retinal cell types, thereby delivering said exogenous nucleic acid sequence to said at least three different retinal cell types, wherein said at least three different retinal cell types are selected from the group consisting of retinal ganglion cells, amacrine cells, horizontal cells, bipolar cells, Muller glia cells, photoreceptor cells, and RPE cells.

Embodiment 32E. The method of Embodiment 31E, wherein said capsid polypeptide comprises the amino acid sequence of SEQ ID NO:1 except that said amino acid sequence of any one of SEQ ID NOs:E2-E83 is located between amino acid positions 587 and 588 of SEQ ID NO:1.

Embodiment 33E. The method of Embodiment 31E, wherein said capsid polypeptide comprises the amino acid sequence of SEQ ID NO:1 except that said amino acid sequence of any one of SEQ ID NOs:E2-E75 is located between amino acid positions 587 and 588 of SEQ ID NO:1.

Embodiment 34E. The method of Embodiment 31E, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:E2-E83.

Embodiment 35E. The method of Embodiment 31E, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:E76-E83.

Embodiment 36E. The method of any one of Embodiments 31E-35E, wherein said mammal is a human.

Embodiment 37E. The method of any one of Embodiments 31E-36E, wherein said vector is an AAV2 vector.

Embodiment 38E. The method of any one of Embodiments 31E-37E, wherein said vector infects greater than 2 percent of said at least three different retinal cell types within an eye when a titer of at least 1×10⁷ of said vector is administered intravitreally to said eye.

Embodiment 39E. The method of any one of Embodiments 31E-38E, wherein said exogenous nucleic acid sequence encodes an RNA or a polypeptide.

Embodiment 40E. The method of Embodiment 39E, wherein said exogenous nucleic acid encodes an RNA.

Embodiment 41E. The method of Embodiment 40E, wherein said RNA is an siRNA or microRNA.

Embodiment 42E. The method of Embodiment 39E, wherein said exogenous nucleic acid encodes a polypeptide.

Embodiment 43E. The method of Embodiment 42E, wherein said polypeptide is an ABCA4 polypeptide, a CRB1 polypeptide, an NPHP5 polypeptide, or an NR2E3 polypeptide.

Embodiment 44E. The method of any one of Embodiments 31E-43E, wherein said vector expresses more of said exogenous nucleic acid sequence in said at least three different retinal cell types than the level of expression in said at least three different retinal cell types from a comparable AAV vector comprising a capsid polypeptide consisting of the amino acid sequence set forth in SEQ ID NO:1.

Embodiment 45E. The method of any one of Embodiments 31E-44E, wherein said method comprises intravitreally administering a composition comprising said vector to said mammal, thereby contacting said at least three different retinal cell types with said vector.

Embodiment 46E. The method of Embodiment 45E, wherein said composition comprises from about 1×10⁷ to about 1×10¹⁴ of said vector.

Embodiment 47E. A method for treating a retinal condition, wherein said method comprises contacting at least three different retinal cell types of an eye of a mammal having said retinal condition with AAV vectors comprising an AAV capsid polypeptide and an exogenous nucleic acid sequence, wherein said capsid polypeptide comprises the amino acid sequence of any one of SEQ ID NOs:E2-E83, wherein said AAV vectors infect said at least three different retinal cell types and drive expression of said exogenous nucleic acid sequence within said at least three different retinal cell types, thereby treating said retinal condition.

Embodiment 48E. The method of Embodiment 47E, wherein said mammal is a human.

Embodiment 49E. The method of any one of Embodiments 47E-48E, wherein said retinal condition is selected from the group consisting of cone dystrophy, cone/rod dystrophy, retinitis pigmentosa, macular degeneration, achromatopsia, blue cone monochromcy, and color blindness.

Embodiment 50E. The method of any one of Embodiments 47E-49E, wherein said capsid polypeptide comprises the amino acid sequence of SEQ ID NO:1 except that said amino acid sequence of any one of SEQ ID NOs:E2-E83 is located between amino acid positions 587 and 588 of SEQ ID NO:1.

Embodiment 51E. The method of any one of Embodiments 47E-49E, wherein said capsid polypeptide comprises the amino acid sequence of SEQ ID NO:1 except that said amino acid sequence of any one of SEQ ID NOs:E2-E75 is located between amino acid positions 587 and 588 of SEQ ID NO:1.

Embodiment 52E. The method of any one of Embodiments 47E-49E, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:E2-E83.

Embodiment 53E. The method of any one of Embodiments 47E-49E, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:E76-E83.

Embodiment 54E. The method of any one of Embodiments 47E-53E, wherein said vectors are AAV2 vectors.

Embodiment 55E. The method of any one of Embodiments 47E-53E, wherein said vectors infect greater than 2 percent of said at least three different retinal cell types within said eye when a titer of at least 1×10⁷ of said vectors is administered intravitreally.

Embodiment 56E. The method of any one of Embodiments 47E-55E, wherein said exogenous nucleic acid sequence encodes an RNA.

Embodiment 57E. The method of Embodiment 56E, wherein said RNA is an siRNA or microRNA.

Embodiment 58E. The method of any one of Embodiments 47E-55E, wherein said exogenous nucleic acid encodes a polypeptide.

Embodiment 59E. The method of Embodiment 58E, wherein said polypeptide is an ABCA4 polypeptide, a CRB1 polypeptide, an NPHIP5 polypeptide, or an NR2E3 polypeptide.

Embodiment 60E. The method of any one of Embodiments 47E-59E, wherein said vectors express more of said exogenous nucleic acid sequence in said at least three different retinal cell types than the level of expression in said at least three different retinal cell types from a comparable AAV vector comprising a capsid polypeptide consisting of the amino acid sequence set forth in SEQ ID NO:1.

Embodiment 61E. The method of any one of Embodiments 47E-60E, wherein said method comprises intravitreally administering a composition comprising said vectors to said mammal, thereby contacting said at least three different retinal cell types with said vectors.

Embodiment 62E. The method of Embodiment 61E, wherein said composition comprises from about 1×10⁷ to about 1×10¹⁴ of said vector.

Embodiment 63E. The method of any one of Embodiments 44E-62E, wherein said at least three different retinal cell types are selected from the group consisting of retinal ganglion cells, amacrine cells, horizontal cells, bipolar cells, Muller glia cells, photoreceptor cells, and RPE cells.

Embodiment 1F. An adeno-associated virus (AAV) vector comprising an AAV capsid polypeptide, wherein said capsid polypeptide comprises the amino acid sequence of any one of SEQ ID NOs:F2-F10.

Embodiment 2F. The vector of Embodiment 1F, wherein said capsid polypeptide comprises the amino acid sequence of SEQ ID NO:1 except that said amino acid sequence of any one of SEQ ID NOs:F2-F10 is located between amino acid positions 587 and 588 of SEQ ID NO: 1.

Embodiment 3F. The vector of Embodiment 1F, wherein said capsid polypeptide comprises the amino acid sequence of SEQ ID NO:1 except that said amino acid sequence of any one of SEQ ID NOs:F2-F5 is located between amino acid positions 587 and 588 of SEQ ID NO:1.

Embodiment 4F. The vector of Embodiment 1F, wherein said capsid polypeptide comprises the amino acid sequence of SEQ ID NO:1 except that said amino acid sequence of any one of SEQ ID NOs:F6-F10 is located between amino acid positions 587 and 588 of SEQ ID NO: 1.

Embodiment 5F. The vector of Embodiment 1F, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:F2-F10.

Embodiment 6F. The vector of any one of Embodiments 1F-5F, wherein said vector is an AAV2 vector.

Embodiment 7F. The vector of any one of Embodiments 1F-6F, wherein said vector infects greater than 2 percent of RPE cells when a titer of at least 1×10⁷ of said vector is administered intravitreally to an eye of a human (or a non-human primate).

Embodiment 8F. The vector of any one of Embodiments 1F-7F, wherein said vector comprises an exogenous nucleic acid encoding an RNA or a polypeptide.

Embodiment 9F. The vector of Embodiment 8F, wherein said exogenous nucleic acid encodes an RNA.

Embodiment 10F. The vector of Embodiment 9F, wherein said RNA is an siRNA or microRNA.

Embodiment 11F. The vector of Embodiment 8F, wherein said exogenous nucleic acid encodes a polypeptide.

Embodiment 12F. The vector of Embodiment 11F, wherein said polypeptide is an ABCA4 polypeptide, a CRB1 polypeptide, an NPHP5 polypeptide, or an NR2E3 polypeptide.

Embodiment 13F. The vector of any one of Embodiments 1F-12F, wherein said vector expresses more nucleic acid in RPE cells than the level of expression from a comparable AAV vector comprising a capsid polypeptide consisting of the amino acid sequence set forth in SEQ ID NO:1.

Embodiment 14F. An AAV capsid polypeptide comprising the amino acid sequence of any one of SEQ ID NOs:F2-F10.

Embodiment 15F. The polypeptide of Embodiment 14F, wherein said polypeptide comprises the amino acid sequence of SEQ ID NO:1 except that said amino acid sequence of any one of SEQ ID NOs:F2-F10 is located between amino acid positions 587 and 588 of SEQ ID NO: 1.

Embodiment 16F. The polypeptide of Embodiment 14F, wherein said polypeptide comprises the amino acid sequence of SEQ ID NO:1 except that said amino acid sequence of any one of SEQ ID NOs:F2-F5 is located between amino acid positions 587 and 588 of SEQ ID NO:1.

Embodiment 17F. The polypeptide of Embodiment 14F, wherein said capsid polypeptide comprises the amino acid sequence of SEQ ID NO:1 except that said amino acid sequence of any one of SEQ ID NOs:F6-F10 is located between amino acid positions 587 and 588 of SEQ ID NO:1.

Embodiment 18F. The polypeptide of Embodiment 14F, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:F2-F10.

Embodiment 19F. The polypeptide of any one of Embodiments 14F-18F, wherein an AAV vector comprising said polypeptide infects greater than 2 percent of RPE cells when a titer of at least 1×10⁷ of said vector is administered intravitreally to an eye of a human (or a non-human primate).

Embodiment 20F. The polypeptide of any one of Embodiments 14F-19F, wherein an AAV vector comprising said polypeptide expresses more nucleic acid in RPE cells than the level of expression from a comparable AAV vector comprising a capsid polypeptide consisting of the amino acid sequence set forth in SEQ ID NO:1.

Embodiment 21F. A nucleic acid molecule encoding a vector of any one of Embodiments 1F-13F or a polypeptide of any one of Embodiments 14F-20F.

Embodiment 22F. The nucleic acid molecule of Embodiment 21F, wherein said nucleic acid molecule is DNA.

Embodiment 23F. A host cell comprising a nucleic acid molecule of any one of Embodiments 21F-22F.

Embodiment 24F. The host cell of Embodiment 23F, wherein said host cell expresses said vector.

Embodiment 25F. The host cell of Embodiment 23F, wherein said host cell expresses said polypeptide.

Embodiment 26F. A host cell comprising a vector of any one of Embodiments 1F-13F or a polypeptide of any one of Embodiments 14F-20F.

Embodiment 27F. The host cell of any one of Embodiments 23F-26F, wherein said host cell is a retinal cell.

Embodiment 28F. A composition comprising a vector of any one of Embodiments 1F-13F.

Embodiment 29F. The composition of Embodiment 28F, wherein said composition comprises from about 1×10⁷ to about 1×10¹⁴ of said vector.

Embodiment 30F. The composition of any one of Embodiments 28F-29F, wherein said composition comprises phosphate buffered saline, Hank's Balanced Salt Solution, or Pluronic F68.

Embodiment 31F. A method for delivering an exogenous nucleic acid sequence to RPE cells of an eye of a mammal, wherein said method comprises contacting said RPE cells with an AAV vector comprising an AAV capsid polypeptide and said exogenous nucleic acid sequence, wherein said capsid polypeptide comprises the amino acid sequence of any one of SEQ ID NOs:F2-F10, wherein said AAV vector infects RPE cells, thereby delivering said exogenous nucleic acid sequence to said RPE cells.

Embodiment 32F. The method of Embodiment 31F, wherein said capsid polypeptide comprises the amino acid sequence of SEQ ID NO:1 except that said amino acid sequence of any one of SEQ ID NOs:F2-F10 is located between amino acid positions 587 and 588 of SEQ ID NO:1.

Embodiment 33F. The method of Embodiment 31F, wherein said capsid polypeptide comprises the amino acid sequence of SEQ ID NO:1 except that said amino acid sequence of any one of SEQ ID NOs:F2-F5 is located between amino acid positions 587 and 588 of SEQ ID NO:1.

Embodiment 34F. The method of Embodiment 31F, wherein said capsid polypeptide comprises the amino acid sequence of SEQ ID NO:1 except that said amino acid sequence of any one of SEQ ID NOs:F6-F10 is located between amino acid positions 587 and 588 of SEQ ID NO:1.

Embodiment 35F. The method of Embodiment 31F, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:F2-F10.

Embodiment 36F. The method of any one of Embodiments 31F-35F, wherein said mammal is a human.

Embodiment 37F. The method of any one of Embodiments 31F-36F, wherein said vector is an AAV2 vector.

Embodiment 38F. The method of any one of Embodiments 31F-37F, wherein said vector infects greater than 2 percent of RPE cells of an eye when a titer of at least 1×10⁷ of said vector is administered intravitreally to said eye.

Embodiment 39F. The method of any one of Embodiments 31F-38F, wherein said exogenous nucleic acid sequence encodes an RNA or a polypeptide.

Embodiment 40F. The method of Embodiment 39F, wherein said exogenous nucleic acid encodes an RNA.

Embodiment 41F. The method of Embodiment 40F, wherein said RNA is an siRNA or microRNA.

Embodiment 42F. The method of Embodiment 39F, wherein said exogenous nucleic acid encodes a polypeptide.

Embodiment 43F. The method of Embodiment 42F, wherein said polypeptide is an ABCA4 polypeptide, a CRB1 polypeptide, an NPHP5 polypeptide, or an NR2E3 polypeptide.

Embodiment 44F. The method of any one of Embodiments 31F-43F, wherein said vector expresses more of said exogenous nucleic acid sequence in said RPE cells than the level of expression in a RPE cell from a comparable AAV vector comprising a capsid polypeptide consisting of the amino acid sequence set forth in SEQ ID NO:1.

Embodiment 45F. The method of any one of Embodiments 31F-44F, wherein said method comprises intravitreally administering a composition comprising said vector to said mammal, thereby contacting said RPE cells with said vector.

Embodiment 46F. The method of Embodiment 45F, wherein said composition comprises from about 1×10⁷ to about 1×10¹⁴ of said vector.

Embodiment 47F. A method for treating a retinal condition, wherein said method comprises contacting RPE cells of an eye of a mammal having said retinal condition with AAV vectors comprising an AAV capsid polypeptide and an exogenous nucleic acid sequence, wherein said capsid polypeptide comprises the amino acid sequence of any one of SEQ ID NOs:F2-F10, wherein said AAV vectors infect said RPE cells and drive expression of said exogenous nucleic acid sequence within said RPE cells, thereby treating said retinal condition.

Embodiment 48F. The method of Embodiment 47F, wherein said mammal is a human.

Embodiment 49F. The method of any one of Embodiments 47F-48F, wherein said retinal condition is selected from the group consisting of cone dystrophy, cone/rod dystrophy, retinitis pigmentosa, macular degeneration, achromatopsia, blue cone monochromcy, and color blindness.

Embodiment 50F. The method of any one of Embodiments 47F-49F, wherein said capsid polypeptide comprises the amino acid sequence of SEQ ID NO:1 except that said amino acid sequence of any one of SEQ ID NOs:F2-F10 is located between amino acid positions 587 and 588 of SEQ ID NO:1.

Embodiment 51F. The method of any one of Embodiments 47F-49F, wherein said capsid polypeptide comprises the amino acid sequence of SEQ ID NO:1 except that said amino acid sequence of any one of SEQ ID NOs:F2-F5 is located between amino acid positions 587 and 588 of SEQ ID NO:1.

Embodiment 52F. The method of any one of Embodiments 47F-49F, wherein said capsid polypeptide comprises the amino acid sequence of SEQ ID NO:1 except that said amino acid sequence of any one of SEQ ID NOs:F6-F10 is located between amino acid positions 587 and 588 of SEQ ID NO:1.

Embodiment 53F. The method of any one of Embodiments 47F-49F, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:F2-F10.

Embodiment 54F. The method of any one of Embodiments 47F-53F, wherein said vectors are AAV2 vectors.

Embodiment 55F. The method of any one of Embodiments 47F-54F, wherein said vectors infect greater than 2 percent of RPE cells when a titer of at least 1×10⁷ of said vectors is administered intravitreally to an eye of said mammal.

Embodiment 56F. The method of any one of Embodiments 47F-55F, wherein said exogenous nucleic acid sequence encodes an RNA.

Embodiment 57F. The method of Embodiment 56F, wherein said RNA is an siRNA or microRNA.

Embodiment 58F. The method of any one of Embodiments 47F-55F, wherein said exogenous nucleic acid encodes a polypeptide.

Embodiment 59F. The method of Embodiment 58F, wherein said polypeptide is an ABCA4 polypeptide, a CRB1 polypeptide, an NPHP5 polypeptide, or an NR2E3 polypeptide.

Embodiment 60F. The method of any one of Embodiments 47F-59F, wherein said vectors express more of said exogenous nucleic acid sequence in said RPE cells than the level of expression in RPE cells from a comparable AAV vector comprising a capsid polypeptide consisting of the amino acid sequence set forth in SEQ ID NO:1.

Embodiment 61F. The method of any one of Embodiments 47F-60F, wherein said method comprises intravitreally administering a composition comprising said vectors to said mammal, thereby contacting said RPE cells with said vectors.

Embodiment 62F. The method of Embodiment 61F, wherein said composition comprises from about 1×10⁷ to about 1×10¹⁴ of said vector.

Embodiment 1G. An adeno-associated virus (AAV) vector comprising an AAV capsid polypeptide, wherein said capsid polypeptide comprises the amino acid sequence of any one of SEQ ID NOs:G2-G77.

Embodiment 2G. The vector of Embodiment 1G, wherein said capsid polypeptide comprises the amino acid sequence of SEQ ID NO:1 except that said amino acid sequence of any one of SEQ ID NOs:G2-G77 is located between amino acid positions 587 and 588 of SEQ ID NO:1.

Embodiment 3G. The vector of Embodiment 1G, wherein said capsid polypeptide comprises the amino acid sequence of SEQ ID NO:1 except that said amino acid sequence of any one of SEQ ID NOs:G2-G70 is located between amino acid positions 587 and 588 of SEQ ID NO:1.

Embodiment 4G. The vector of Embodiment 1G, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:G2-G77.

Embodiment 5G. The vector of Embodiment 1G, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:G71-G77.

Embodiment 6G. The vector of any one of Embodiments 1G-5G, wherein said vector is an AAV2 vector.

Embodiment 7G. The vector of any one of Embodiments 1G-6G, wherein said vector infects greater than 2 percent of photoreceptor cells of an eye when a titer of at least 1×10⁷ of said vector is administered intravitreally to said eye.

Embodiment 8G. The vector of any one of Embodiments 1G-7G, wherein said vector comprises an exogenous nucleic acid encoding an RNA or a polypeptide.

Embodiment 9G. The vector of Embodiment 8G, wherein said exogenous nucleic acid encodes an RNA.

Embodiment 10G. The vector of Embodiment 9G, wherein said RNA is an siRNA or microRNA.

Embodiment 11G. The vector of Embodiment 8G, wherein said exogenous nucleic acid encodes a polypeptide.

Embodiment 12G. The vector of Embodiment 11G, wherein said polypeptide is an ABCA4 polypeptide, a CRB1 polypeptide, an NPHP5 polypeptide, or an NR2E3 polypeptide.

Embodiment 13G. The vector of any one of Embodiments 1G-12G, wherein said vector expresses more nucleic acid in photoreceptor cells than the level of expression from a comparable AAV vector comprising a capsid polypeptide consisting of the amino acid sequence set forth in SEQ ID NO:1.

Embodiment 14G. An AAV capsid polypeptide comprising the amino acid sequence of any one of SEQ ID NOs:G2-G77.

Embodiment 15G. The polypeptide of Embodiment 14G, wherein said polypeptide comprises the amino acid sequence of SEQ ID NO:1 except that said amino acid sequence of any one of SEQ ID NOs:G2-G77 is located between amino acid positions 587 and 588 of SEQ ID NO:1.

Embodiment 16G. The polypeptide of Embodiment 14G, wherein said polypeptide comprises the amino acid sequence of SEQ ID NO:1 except that said amino acid sequence of any one of SEQ ID NOs:G2-G70 is located between amino acid positions 587 and 588 of SEQ ID NO:1.

Embodiment 17G. The polypeptide of Embodiment 14G, wherein said polypeptide comprises the amino acid sequence of SEQ ID NO.1 except that the amino acids from position 585 to 590 of SEQ ID NO:1 are replaced with said amino acid sequence of any one of SEQ ID NOs:G2-G77.

Embodiment 18G. The polypeptide of Embodiment 14G, wherein said polypeptide comprises the amino acid sequence of SEQ ID NO:1 except that the amino acids from position 585 to 590 of SEQ ID NO:1 are replaced with said amino acid sequence of any one of SEQ ID NOs:G71-G77.

Embodiment 19G. The polypeptide of any one of Embodiments 14G-18G, wherein an AAV vector comprising said polypeptide infects greater than 2 percent of photoreceptor cells of an eye when a titer of at least 1×10¹⁴ of said vector is administered intravitreally to said eye.

Embodiment 20G. The polypeptide of any one of Embodiments 14G-19G, wherein an AAV vector comprising said polypeptide expresses more nucleic acid in photoreceptor cells than the level of expression from a comparable AAV vector comprising a capsid polypeptide consisting of the amino acid sequence set forth in SEQ ID NO:1.

Embodiment 21G. A nucleic acid molecule encoding a vector of any one of Embodiments 1G-13G or a polypeptide of any one of Embodiments 14G-20G.

Embodiment 22G. The nucleic acid molecule of Embodiment 21G, wherein said nucleic acid molecule is DNA.

Embodiment 23G. A host cell comprising a nucleic acid molecule of any one of Embodiments 21G-22G.

Embodiment 24G. The host cell of Embodiment 23G, wherein said host cell expresses said vector.

Embodiment 25G. The host cell of Embodiment 23G, wherein said host cell expresses said polypeptide.

Embodiment 26G. A host cell comprising a vector of any one of Embodiments 1G-13G or a polypeptide of any one of Embodiments 14G-20G.

Embodiment 27G. The host cell of any one of Embodiments 23G-26G, wherein said host cell is a retinal cell.

Embodiment 28G. A composition comprising a vector of any one of Embodiments 1G-13G.

Embodiment 29G. The composition of Embodiment 28G, wherein said composition comprises from about 1×10⁷ to about 1×10¹⁴ of said vector.

Embodiment 30G. The composition of any one of Embodiments 28G-29G, wherein said composition comprises phosphate buffered saline, Hank's Balanced Salt Solution, or Pluronic F68.

Embodiment 31G. A method for delivering an exogenous nucleic acid sequence to a photoreceptor cell within a mammal, wherein said method comprises contacting said photoreceptor cell with an AAV vector comprising an AAV capsid polypeptide and said exogenous nucleic acid sequence, wherein said capsid polypeptide comprises the amino acid sequence of any one of SEQ ID NOs:G2-G77, wherein said AAV vector infects said photoreceptor cell, thereby delivering said exogenous nucleic acid sequence to said photoreceptor cell.

Embodiment 32G. The method of Embodiment 31G, wherein said capsid polypeptide comprises the amino acid sequence of SEQ ID NO:1 except that said amino acid sequence of any one of SEQ ID NOs:G2-G77 is located between amino acid positions 587 and 588 of SEQ ID NO:1.

Embodiment 33G. The method of Embodiment 31G, wherein said capsid polypeptide comprises the amino acid sequence of SEQ ID NO:1 except that said amino acid sequence of any one of SEQ ID NOs:G2-G70 is located between amino acid positions 587 and 588 of SEQ ID NO:1.

Embodiment 34G. The method of Embodiment 31G, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:G2-G77.

Embodiment 35G. The method of Embodiment 31G, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:G71-G77.

Embodiment 36G. The method of any one of Embodiments 31G-35G, wherein said mammal is a human.

Embodiment 37G. The method of any one of Embodiments 31G-36G, wherein said vector is an AAV2 vector.

Embodiment 38G. The method of any one of Embodiments 31G-37G, wherein said vector infects greater than 2 percent of photoreceptor cells of an eye when a titer of at least 1×10⁷ of said vector is administered intravitreally to said eye.

Embodiment 39G. The method of any one of Embodiments 31G-38G, wherein said exogenous nucleic acid sequence encodes an RNA or a polypeptide.

Embodiment 40G. The method of Embodiment 39G, wherein said exogenous nucleic acid encodes an RNA.

Embodiment 41G. The method of Embodiment 40G, wherein said RNA is an siRNA or microRNA.

Embodiment 42G. The method of Embodiment 39G, wherein said exogenous nucleic acid encodes a polypeptide.

Embodiment 43G. The method of Embodiment 42G, wherein said polypeptide is an ABCA4 polypeptide, a CRB1 polypeptide, an NPHP5 polypeptide, or an NR2E3 polypeptide.

Embodiment 44G. The method of any one of Embodiments 31G-43G, wherein said vector expresses more of said exogenous nucleic acid sequence in said photoreceptor cell than the level of expression in a photoreceptor cell from a comparable AAV vector comprising a capsid polypeptide consisting of the amino acid sequence set forth in SEQ ID NO:1.

Embodiment 45G. The method of any one of Embodiments 31G-44G, wherein said method comprises intravitreally administering a composition comprising said vector to said mammal, thereby contacting said photoreceptor cell with said vector.

Embodiment 46G. The method of Embodiment 45G, wherein said composition comprises from about 1×10⁷ to about 1×10⁴ of said vector.

Embodiment 47G. A method for treating a retinal condition, wherein said method comprises contacting photoreceptor cells of a mammal having said retinal condition with AAV vectors comprising an AAV capsid polypeptide and an exogenous nucleic acid sequence, wherein said capsid polypeptide comprises the amino acid sequence of any one of SEQ ID NOs:G2-G77, wherein said AAV vectors infect said photoreceptor cells and drive expression of said exogenous nucleic acid sequence within said photoreceptor cells, thereby treating said retinal condition.

Embodiment 48G. The method of Embodiment 47G, wherein said mammal is a human.

Embodiment 49G. The method of any one of Embodiments 47G-48G, wherein said retinal condition is selected from the group consisting of cone dystrophy, cone/rod dystrophy, retinitis pigmentosa, macular degeneration, achromatopsia, blue cone monochromcy, and color blindness.

Embodiment 50G. The method of any one of Embodiments 47G-49G, wherein said capsid polypeptide comprises the amino acid sequence of SEQ ID NO:1 except that said amino acid sequence of any one of SEQ ID NOs:G2-G77 is located between amino acid positions 587 and 588 of SEQ ID NO:1.

Embodiment 51G. The method of any one of Embodiments 47G-49G, wherein said capsid polypeptide comprises the amino acid sequence of SEQ ID NO:1 except that said amino acid sequence of any one of SEQ ID NOs:G2-G70 is located between amino acid positions 587 and 588 of SEQ ID NO:1.

Embodiment 52G. The method of any one of Embodiments 47G-49G, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:G2-G77.

Embodiment 53G. The method of any one of Embodiments 47G-49G, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:G71-G77.

Embodiment 54G. The method of any one of Embodiments 47G-53G, wherein said vectors are AAV2 vectors.

Embodiment 55G. The method of any one of Embodiments 47G-54G, wherein said vectors infect greater than 2 percent of photoreceptor cells when a titer of at least 1×10⁷ of said vectors is administered intravitreally to an eye of said mammal.

Embodiment 56G. The method of any one of Embodiments 47G-55G, wherein said exogenous nucleic acid sequence encodes an RNA.

Embodiment 57G. The method of Embodiment 56G, wherein said RNA is an siRNA or microRNA.

Embodiment 58G. The method of any one of Embodiments 47G-55G, wherein said exogenous nucleic acid encodes a polypeptide.

Embodiment 59G. The method of Embodiment 58G, wherein said polypeptide is an ABCA4 polypeptide, a CRB1 polypeptide, an NPHP5 polypeptide, or an NR2E3 polypeptide.

Embodiment 60G. The method of any one of Embodiments 47G-59G, wherein said vectors express more of said exogenous nucleic acid sequence in said photoreceptor cells than the level of expression in photoreceptor cells from a comparable AAV vector comprising a capsid polypeptide consisting of the amino acid sequence set forth in SEQ ID NO:1.

Embodiment 61G. The method of any one of Embodiments 47G-60G, wherein said method comprises intravitreally administering a composition comprising said vectors to said mammal, thereby contacting said photoreceptor cells with said vectors.

Embodiment 62G. The method of Embodiment 61G, wherein said composition comprises from about 1×10⁷ to about 1×10¹⁴ of said vector.

Embodiment 1H. An adeno-associated virus (AAV) vector comprising an AAV capsid polypeptide, wherein said capsid polypeptide comprises the amino acid sequence of any one of SEQ ID NOs:H2-H258.

Embodiment 2H. The vector of Embodiment 1H, wherein said capsid polypeptide comprises the amino acid sequence of SEQ ID NO:1 except that said amino acid sequence of any one of SEQ ID NOs:H2-H258 is located between amino acid positions 587 and 588 of SEQ ID NO:1.

Embodiment 3H. The vector of Embodiment 1H, wherein said capsid polypeptide comprises the amino acid sequence of SEQ ID NO:1 except that said amino acid sequence of any one of SEQ ID NOs:H2-H243 is located between amino acid positions 587 and 588 of SEQ ID NO:1.

Embodiment 4H. The vector of Embodiment 1H, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:H2-H258.

Embodiment 5H. The vector of Embodiment 1H, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:H244-H258.

Embodiment 6H. The vector of any one of Embodiments 1H-5H, wherein said vector is an AAV2 vector.

Embodiment 7H. The vector of any one of Embodiments 1H-6H, wherein said vector infects greater than 2 percent of retinal ganglion cells of an eye when a titer of at least 1×10⁷ of said vector is administered intravitreally to said eye.

Embodiment 8H. The vector of any one of Embodiments 1H-7H, wherein said vector comprises an exogenous nucleic acid encoding an RNA or a polypeptide.

Embodiment 9H. The vector of Embodiment 8H, wherein said exogenous nucleic acid encodes an RNA.

Embodiment 10H. The vector of Embodiment 9H, wherein said RNA is an siRNA or microRNA.

Embodiment 11H. The vector of Embodiment 8H, wherein said exogenous nucleic acid encodes a polypeptide.

Embodiment 12H. The vector of Embodiment 11H, wherein said polypeptide is an ABCA4 polypeptide, a CRB1 polypeptide, an NPHP5 polypeptide, or an NR2E3 polypeptide.

Embodiment 13H. The vector of any one of Embodiments 1H-12H, wherein said vector expresses more nucleic acid in retinal ganglion cells than the level of expression from a comparable AAV vector comprising a capsid polypeptide consisting of the amino acid sequence set forth in SEQ ID NO:1.

Embodiment 14H. An AAV capsid polypeptide comprising the amino acid sequence of any one of SEQ ID NOs:H2-H258.

Embodiment 15H. The polypeptide of Embodiment 14H, wherein said polypeptide comprises the amino acid sequence of SEQ ID NO:1 except that said amino acid sequence of any one of SEQ ID NOs:H2-H258 is located between amino acid positions 587 and 588 of SEQ ID NO:1.

Embodiment 16H. The polypeptide of Embodiment 14H, wherein said polypeptide comprises the amino acid sequence of SEQ ID NO:1 except that said amino acid sequence of any one of SEQ ID NOs:H2-H243 is located between amino acid positions 587 and 588 of SEQ ID NO:1.

Embodiment 17H. The polypeptide of Embodiment 14H, wherein said polypeptide comprises the amino acid sequence of SEQ ID NO:1 except that the amino acids from position 585 to 590 of SEQ ID NO:1 are replaced with said amino acid sequence of any one of SEQ ID NOs:H2-H258.

Embodiment 18H. The polypeptide of Embodiment 14H, wherein said polypeptide comprises the amino acid sequence of SEQ ID NO:1 except that the amino acids from position 585 to 590 of SEQ ID NO:1 are replaced with said amino acid sequence of any one of SEQ ID NOs:H244-H258.

Embodiment 19H. The polypeptide of any one of Embodiments 14H-18H, wherein an AAV vector comprising said polypeptide infects greater than 2 percent of retinal ganglion cells of an eye when a titer of at least 1×10⁷ of said vector is administered intravitreally to said eye.

Embodiment 20H. The polypeptide of any one of Embodiments 14H-19H, wherein an AAV vector comprising said polypeptide expresses more nucleic acid in retinal ganglion cells than the level of expression from a comparable AAV vector comprising a capsid polypeptide consisting of the amino acid sequence set forth in SEQ ID NO:1.

Embodiment 21H. A nucleic acid molecule encoding a vector of any one of Embodiments 1H-13H or a polypeptide of any one of Embodiments 14H-20H.

Embodiment 22H. The nucleic acid molecule of Embodiment 21H, wherein said nucleic acid molecule is DNA.

Embodiment 23H. A host cell comprising a nucleic acid molecule of any one of Embodiments 21H-22H.

Embodiment 24H. The host cell of Embodiment 23H, wherein said host cell expresses said vector.

Embodiment 25H. The host cell of Embodiment 23H, wherein said host cell expresses said polypeptide.

Embodiment 26H. A host cell comprising a vector of any one of Embodiments 1H-13H or a polypeptide of any one of Embodiments 14H-20H.

Embodiment 27H. The host cell of any one of Embodiments 23H-26H, wherein said host cell is a retinal cell.

Embodiment 28H. A composition comprising a vector of any one of Embodiments 1H-13H.

Embodiment 29H. The composition of Embodiment 28H, wherein said composition comprises from about 1×10⁷ to about 1×10¹⁴ of said vector.

Embodiment 30H. The composition of any one of Embodiments 28H-29H, wherein said composition comprises phosphate buffered saline, Hank's Balanced Salt Solution, or Pluronic F68.

Embodiment 31H. A method for delivering an exogenous nucleic acid sequence to a retinal ganglion cell within a mammal, wherein said method comprises contacting said retinal ganglion cell with an AAV vector comprising an AAV capsid polypeptide and said exogenous nucleic acid sequence, wherein said capsid polypeptide comprises the amino acid sequence of any one of SEQ ID NOs:H2-H258, wherein said AAV vector infects said photoreceptor cell, thereby delivering said exogenous nucleic acid sequence to said photoreceptor cell.

Embodiment 32H. The method of Embodiment 31H, wherein said capsid polypeptide comprises the amino acid sequence of SEQ ID NO:1 except that said amino acid sequence of any one of SEQ ID NOs:H2-H258 is located between amino acid positions 587 and 588 of SEQ ID NO:1.

Embodiment 33H. The method of Embodiment 31H, wherein said capsid polypeptide comprises the amino acid sequence of SEQ ID NO:1 except that said amino acid sequence of any one of SEQ ID NOs:H2-H243 is located between amino acid positions 587 and 588 of SEQ ID NO:1.

Embodiment 34H. The method of Embodiment 31H, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:H2-H258.

Embodiment 35H. The method of Embodiment 31H, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:H244-H258.

Embodiment 36H. The method of any one of Embodiments 31H-35H, wherein said mammal is a human.

Embodiment 37H. The method of any one of Embodiments 31H-36H, wherein said vector is an AAV2 vector.

Embodiment 38H. The method of any one of Embodiments 31H-37H, wherein said vector infects greater than 2 percent of retinal ganglion cells of an eye when a titer of at least 1×10⁷ of said vector is administered intravitreally to said eye.

Embodiment 39H. The method of any one of Embodiments 31H-38H, wherein said exogenous nucleic acid sequence encodes an RNA or a polypeptide.

Embodiment 40H. The method of Embodiment 39H, wherein said exogenous nucleic acid encodes an RNA.

Embodiment 41H. The method of Embodiment 40H, wherein said RNA is an siRNA or microRNA.

Embodiment 42H. The method of Embodiment 39H, wherein said exogenous nucleic acid encodes a polypeptide.

Embodiment 43H. The method of Embodiment 42H, wherein said polypeptide is an ABCA4 polypeptide, a CRB1 polypeptide, an NPHP5 polypeptide, or an NR2E3 polypeptide.

Embodiment 44H. The method of any one of Embodiments 31H-43H, wherein said vector expresses more of said exogenous nucleic acid sequence in said retinal ganglion cell than the level of expression in a retinal ganglion cell from a comparable AAV vector comprising a capsid polypeptide consisting of the amino acid sequence set forth in SEQ ID NO:1.

Embodiment 45H. The method of any one of Embodiments 31H-44H, wherein said method comprises intravitreally administering a composition comprising said vector to said mammal, thereby contacting said retinal ganglion cell with said vector.

Embodiment 46H. The method of Embodiment 45H, wherein said composition comprises from about 1×10⁷ to about 1×10¹⁴ of said vector.

Embodiment 47H. A method for treating a retinal condition, wherein said method comprises contacting retinal ganglion cells of a mammal having said retinal condition with AAV vectors comprising an AAV capsid polypeptide and an exogenous nucleic acid sequence, wherein said capsid polypeptide comprises the amino acid sequence of any one of SEQ ID NOs:H2-H258, wherein said AAV vectors infect said retinal ganglion cells and drive expression of said exogenous nucleic acid sequence within said retinal ganglion cells, thereby treating said retinal condition.

Embodiment 48H. The method of Embodiment 47H, wherein said mammal is a human.

Embodiment 49H. The method of any one of Embodiments 47H-48H, wherein said retinal condition is selected from the group consisting of cone dystrophy, cone/rod dystrophy, retinitis pigmentosa, macular degeneration, achromatopsia, blue cone monochromcy, and color blindness.

Embodiment 50H. The method of any one of Embodiments 47H-49H, wherein said capsid polypeptide comprises the amino acid sequence of SEQ ID NO:1 except that said amino acid sequence of any one of SEQ ID NOs:H2-H258 is located between amino acid positions 587 and 588 of SEQ ID NO:1.

Embodiment 51H. The method of any one of Embodiments 47H-49H, wherein said capsid polypeptide comprises the amino acid sequence of SEQ ID NO:1 except that said amino acid sequence of any one of SEQ ID NOs:H2-H243 is located between amino acid positions 587 and 588 of SEQ ID NO:1.

Embodiment 52H. The method of any one of Embodiments 47H-49H, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:H2-H258.

Embodiment 53H. The method of any one of Embodiments 47H-49H, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:H244-H258.

Embodiment 54H. The method of any one of Embodiments 47H-53H, wherein said vectors are AAV2 vectors.

Embodiment 55H. The method of any one of Embodiments 47H-54H, wherein said vectors infect greater than 2 percent of retinal ganglion cells when a titer of at least 1×10⁷ of said vectors is administered intravitreally to an eye of said mammal.

Embodiment 56H. The method of any one of Embodiments 47H-55H, wherein said exogenous nucleic acid sequence encodes an RNA.

Embodiment 57H. The method of Embodiment 56H, wherein said RNA is an siRNA or microRNA.

Embodiment 58H. The method of any one of Embodiments 47H-55H, wherein said exogenous nucleic acid encodes a polypeptide.

Embodiment 59H. The method of Embodiment 58H, wherein said polypeptide is an ABCA4 polypeptide, a CRB1 polypeptide, an NPHP5 polypeptide, or an NR2E3 polypeptide.

Embodiment 60H. The method of any one of Embodiments 47H-59H, wherein said vectors express more of said exogenous nucleic acid sequence in said retinal ganglion cells than the level of expression in retinal ganglion cells from a comparable AAV vector comprising a capsid polypeptide consisting of the amino acid sequence set forth in SEQ ID NO:1.

Embodiment 61H. The method of any one of Embodiments 47H-60H, wherein said method comprises intravitreally administering a composition comprising said vectors to said mammal, thereby contacting said retinal ganglion cells with said vectors.

Embodiment 62H. The method of Embodiment 61H, wherein said composition comprises from about 1×10⁷ to about 1×10¹⁴ of said vector.

Embodiment 1I. An adeno-associated virus (AAV) vector comprising an AAV capsid polypeptide, wherein said capsid polypeptide comprises the amino acid sequence of any one of SEQ ID NOs:I2-I74.

Embodiment 2I. The vector of Embodiment 1I, wherein said capsid polypeptide comprises the amino acid sequence of SEQ ID NO:1 except that said amino acid sequence of any one of SEQ ID NOs:I2-174 is located between amino acid positions 587 and 588 of SEQ ID NO:1.

Embodiment 3I. The vector of Embodiment 1I, wherein said capsid polypeptide comprises the amino acid sequence of SEQ ID NO:1 except that said amino acid sequence of any one of SEQ ID NOs:I2-167 is located between amino acid positions 587 and 588 of SEQ ID NO:1.

Embodiment 4I. The vector of Embodiment 1I, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:I2-174.

Embodiment 5I. The vector of Embodiment 1I, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:I68-174.

Embodiment 6I. The vector of any one of Embodiments 1I-5I, wherein said vector is an AAV2 vector.

Embodiment 7I. The vector of any one of Embodiments 1I-6I, wherein said vector infects greater than 2 percent of bipolar cells of the retina of an eye when a titer of at least 1×10⁷ of said vector is administered intravitreally to said eye.

Embodiment 8I. The vector of any one of Embodiments 1I-7I, wherein said vector comprises an exogenous nucleic acid encoding an RNA or a polypeptide.

Embodiment 9I. The vector of Embodiment 8I, wherein said exogenous nucleic acid encodes an RNA.

Embodiment 10I. The vector of Embodiment 9I, wherein said RNA is an siRNA or microRNA.

Embodiment 11I. The vector of Embodiment 8I, wherein said exogenous nucleic acid encodes a polypeptide.

Embodiment 12I. The vector of Embodiment 11I, wherein said polypeptide is an ABCA4 polypeptide, a CRB1 polypeptide, an NPHP5 polypeptide, or an NR2E3 polypeptide.

Embodiment 13I. The vector of any one of Embodiments 1I-12I, wherein said vector expresses more nucleic acid in bipolar cells of the retina than the level of expression from a comparable AAV vector comprising a capsid polypeptide consisting of the amino acid sequence set forth in SEQ ID NO:1.

Embodiment 14I. An AAV capsid polypeptide comprising the amino acid sequence of any one of SEQ ID NOs:I2-174.

Embodiment 15I. The polypeptide of Embodiment 14I, wherein said polypeptide comprises the amino acid sequence of SEQ ID NO:1 except that said amino acid sequence of any one of SEQ ID NOs:I2-I74 is located between amino acid positions 587 and 588 of SEQ ID NO:1.

Embodiment 16I. The polypeptide of Embodiment 14I, wherein said polypeptide comprises the amino acid sequence of SEQ ID NO:1 except that said amino acid sequence of any one of SEQ ID NOs:I2-167 is located between amino acid positions 587 and 588 of SEQ ID NO:1.

Embodiment 17I. The polypeptide of Embodiment 14I, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:I2-I74.

Embodiment 18I. The polypeptide of Embodiment 14I, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:I68-I74.

Embodiment 19I. The polypeptide of any one of Embodiments 14I-18I, wherein an AAV vector comprising said polypeptide infects greater than 2 percent of bipolar cells of the retina of an eye when a titer of at least 1×10⁷ of said vector is administered intravitreally to said eye.

Embodiment 20I. The polypeptide of any one of Embodiments 14I-19I, wherein an AAV vector comprising said polypeptide expresses more nucleic acid in bipolar cells of the retina than the level of expression from a comparable AAV vector comprising a capsid polypeptide consisting of the amino acid sequence set forth in SEQ ID NO:1.

Embodiment 21I. A nucleic acid molecule encoding a vector of any one of Embodiments 1I-13I or a polypeptide of any one of Embodiments 14I-20I.

Embodiment 22I. The nucleic acid molecule of Embodiment 21I, wherein said nucleic acid molecule is DNA.

Embodiment 23I. A host cell comprising a nucleic acid molecule of any one of Embodiments 21I-22I.

Embodiment 24I. The host cell of Embodiment 23I, wherein said host cell expresses said vector.

Embodiment 25I. The host cell of Embodiment 23I, wherein said host cell expresses said polypeptide.

Embodiment 26I. A host cell comprising a vector of any one of Embodiments 1I-13I or a polypeptide of any one of Embodiments 14I-201.

Embodiment 27I. The host cell of any one of Embodiments 23I-26I, wherein said host cell is a retinal cell.

Embodiment 28I. A composition comprising a vector of any one of Embodiments 1I-13I.

Embodiment 29I. The composition of Embodiment 28I, wherein said composition comprises from about 1×10⁷ to about 1×10¹⁴ of said vector.

Embodiment 30I. The composition of any one of Embodiments 28I-29I, wherein said composition comprises phosphate buffered saline, Hank's Balanced Salt Solution, or Pluronic F68.

Embodiment 31I. A method for delivering an exogenous nucleic acid sequence to a bipolar cell of the retina within a mammal, wherein said method comprises contacting said bipolar cell with an AAV vector comprising an AAV capsid polypeptide and said exogenous nucleic acid sequence, wherein said capsid polypeptide comprises the amino acid sequence of any one of SEQ ID NOs:I2-I74, wherein said AAV vector infects said bipolar cell, thereby delivering said exogenous nucleic acid sequence to said bipolar cell.

Embodiment 32I. The method of Embodiment 31I, wherein said capsid polypeptide comprises the amino acid sequence of SEQ ID NO:1 except that said amino acid sequence of any one of SEQ ID NOs:I2-174 is located between amino acid positions 587 and 588 of SEQ ID NO:1.

Embodiment 33I. The method of Embodiment 31I, wherein said capsid polypeptide comprises the amino acid sequence of SEQ ID NO:1 except that said amino acid sequence of any one of SEQ ID NOs:I2-167 is located between amino acid positions 587 and 588 of SEQ ID NO:1.

Embodiment 34I. The method of Embodiment 31I, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:I2-I74.

Embodiment 35I. The method of Embodiment 31I, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:I68-I74.

Embodiment 36I. The method of any one of Embodiments 31I-35I, wherein said mammal is a human.

Embodiment 37I. The method of any one of Embodiments 31I-36I, wherein said vector is an AAV2 vector.

Embodiment 38I. The method of any one of Embodiments 31I-37I, wherein said vector infects greater than 2 percent of bipolar cells of the retina of an eye when a titer of at least 1×10⁷ of said vector is administered intravitreally to said eye.

Embodiment 39I. The method of any one of Embodiments 31I-38I, wherein said exogenous nucleic acid sequence encodes an RNA or a polypeptide.

Embodiment 40I. The method of Embodiment 39I, wherein said exogenous nucleic acid encodes an RNA.

Embodiment 41I. The method of Embodiment 40I, wherein said RNA is an siRNA or microRNA.

Embodiment 42I. The method of Embodiment 39I, wherein said exogenous nucleic acid encodes a polypeptide.

Embodiment 43I. The method of Embodiment 42I, wherein said polypeptide is an ABCA4 polypeptide, a CRB1 polypeptide, an NPHP5 polypeptide, or an NR2E3 polypeptide.

Embodiment 44I. The method of any one of Embodiments 31I-43I, wherein said vector expresses more of said exogenous nucleic acid sequence in said bipolar cell than the level of expression in a bipolar cell of the retina from a comparable AAV vector comprising a capsid polypeptide consisting of the amino acid sequence set forth in SEQ ID NO:1.

Embodiment 45I. The method of any one of Embodiments 31I-44I, wherein said method comprises intravitreally administering a composition comprising said vector to said mammal, thereby contacting said bipolar cell with said vector.

Embodiment 46I. The method of Embodiment 45I, wherein said composition comprises from about 1×10⁷ to about 1×10¹⁴ of said vector.

Embodiment 47I. A method for treating a retinal condition, wherein said method comprises contacting bipolar cells of the retina of a mammal having said retinal condition with AAV vectors comprising an AAV capsid polypeptide and an exogenous nucleic acid sequence, wherein said capsid polypeptide comprises the amino acid sequence of any one of SEQ ID NOs:I2-I74, wherein said AAV vectors infect said bipolar cells and drive expression of said exogenous nucleic acid sequence within said bipolar cells, thereby treating said retinal condition.

Embodiment 48I. The method of Embodiment 47I, wherein said mammal is a human.

Embodiment 49I. The method of any one of Embodiments 47I-48I, wherein said retinal condition is selected from the group consisting of cone dystrophy, cone/rod dystrophy, retinitis pigmentosa, macular degeneration, achromatopsia, blue cone monochromcy, and color blindness.

Embodiment 50I. The method of any one of Embodiments 47I-49I, wherein said capsid polypeptide comprises the amino acid sequence of SEQ ID NO:1 except that said amino acid sequence of any one of SEQ ID NOs:I2-174 is located between amino acid positions 587 and 588 of SEQ ID NO:1.

Embodiment 51I. The method of any one of Embodiments 47I-49I, wherein said capsid polypeptide comprises the amino acid sequence of SEQ ID NO:1 except that said amino acid sequence of any one of SEQ ID NOs:I2-T67 is located between amino acid positions 587 and 588 of SEQ ID NO:1.

Embodiment 52I. The method of any one of Embodiments 47I-49I, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:I2-I74.

Embodiment 53I. The method of any one of Embodiments 47I-49I, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:I68-I74.

Embodiment 54I. The method of any one of Embodiments 47I-53I, wherein said vectors are AAV2 vectors.

Embodiment 55I. The method of any one of Embodiments 47I-54I, wherein said vectors infect greater than 2 percent of bipolar cells of the retina when a titer of at least 1×10¹⁴ of said vectors is administered intravitreally to an eye of said mammal.

Embodiment 56I. The method of any one of Embodiments 47I-55I, wherein said exogenous nucleic acid sequence encodes an RNA.

Embodiment 57I. The method of Embodiment 56I, wherein said RNA is an siRNA or microRNA.

Embodiment 58I. The method of any one of Embodiments 47I-55I, wherein said exogenous nucleic acid encodes a polypeptide.

Embodiment 59I. The method of Embodiment 58I, wherein said polypeptide is an ABCA4 polypeptide, a CRB1 polypeptide, an NPHP5 polypeptide, or an NR2E3 polypeptide.

Embodiment 60I. The method of any one of Embodiments 47I-59I, wherein said vectors express more of said exogenous nucleic acid sequence in said bipolar cells than the level of expression in bipolar cells of the retina from a comparable AAV vector comprising a capsid polypeptide consisting of the amino acid sequence set forth in SEQ ID NO:1.

Embodiment 61I. The method of any one of Embodiments 47I-60I, wherein said method comprises intravitreally administering a composition comprising said vectors to said mammal, thereby contacting said bipolar cells with said vectors.

Embodiment 62I. The method of Embodiment 61I, wherein said composition comprises from about 1×10⁷ to about 1×10¹⁴ of said vector.

Embodiment 1J. An adeno-associated virus (AAV) vector comprising an AAV capsid polypeptide, wherein said capsid polypeptide comprises the amino acid sequence of any one of SEQ ID NOs:J2-J64.

Embodiment 2J. The vector of Embodiment 1J, wherein said capsid polypeptide comprises the amino acid sequence of SEQ ID NO:1 except that said amino acid sequence of any one of SEQ ID NOs:J2-J64 is located between amino acid positions 587 and 588 of SEQ ID NO: 1.

Embodiment 3J. The vector of Embodiment 1J, wherein said capsid polypeptide comprises the amino acid sequence of SEQ ID NO:1 except that said amino acid sequence of any one of SEQ ID NOs:J2-J61 is located between amino acid positions 587 and 588 of SEQ ID NO: 1.

Embodiment 4J. The vector of Embodiment 1J, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:J2-J64.

Embodiment 5J. The vector of Embodiment 1J, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:J62-J64.

Embodiment 6J. The vector of any one of Embodiments 1J-5J, wherein said vector is an AAV2 vector.

Embodiment 7J. The vector of any one of Embodiments 1J-6J, wherein said vector infects greater than 2 percent of ON-retinal ganglion cells of an eye when a titer of at least 1×10⁷ of said vector is administered intravitreally to said eye.

Embodiment 8J. The vector of any one of Embodiments 1J-7J, wherein said vector comprises an exogenous nucleic acid encoding an RNA or a polypeptide.

Embodiment 9J. The vector of Embodiment 8J, wherein said exogenous nucleic acid encodes an RNA.

Embodiment 10J. The vector of Embodiment 9J, wherein said RNA is an siRNA or microRNA.

Embodiment 11J. The vector of Embodiment 8J, wherein said exogenous nucleic acid encodes a polypeptide.

Embodiment 12J. The vector of Embodiment 11J, wherein said polypeptide is an ABCA4 polypeptide, a CRB1 polypeptide, an NPHP5 polypeptide, or an NR2E3 polypeptide.

Embodiment 13J. The vector of any one of Embodiments 1J-12J, wherein said vector expresses more nucleic acid in ON-retinal ganglion cells than the level of expression from a comparable AAV vector comprising a capsid polypeptide consisting of the amino acid sequence set forth in SEQ ID NO:1.

Embodiment 14J. An AAV capsid polypeptide comprising the amino acid sequence of any one of SEQ ID NOs:J2-J64.

Embodiment 15J. The polypeptide of Embodiment 14J, wherein said polypeptide comprises the amino acid sequence of SEQ ID NO:1 except that said amino acid sequence of any one of SEQ ID NOs:J2-J64 is located between amino acid positions 587 and 588 of SEQ ID NO: 1.

Embodiment 16J. The polypeptide of Embodiment 14J, wherein said polypeptide comprises the amino acid sequence of SEQ ID NO:1 except that said amino acid sequence of any one of SEQ ID NOs:J2-J61 is located between amino acid positions 587 and 588 of SEQ ID NO: 1.

Embodiment 17J. The polypeptide of Embodiment 14J, wherein said polypeptide comprises the amino acid sequence of SEQ ID NO:1 except that the amino acids from position 585 to 590 of SEQ ID NO:1 are replaced with said amino acid sequence of any one of SEQ ID NOs:J2-J64.

Embodiment 18J. The polypeptide of Embodiment 14J, wherein said polypeptide comprises the amino acid sequence of SEQ ID NO:1 except that the amino acids from position 585 to 590 of SEQ ID NO:1 are replaced with said amino acid sequence of any one of SEQ ID NOs:J62-J64.

Embodiment 19J. The polypeptide of any one of Embodiments 14J-18J, wherein an AAV vector comprising said polypeptide infects greater than 2 percent of ON-retinal ganglion cells of an eye when a titer of at least 1×10⁷ of said vector is administered intravitreally to said eye.

Embodiment 20J. The polypeptide of any one of Embodiments 14J-19J, wherein an AAV vector comprising said polypeptide expresses more nucleic acid in ON-retinal ganglion cells than the level of expression from a comparable AAV vector comprising a capsid polypeptide consisting of the amino acid sequence set forth in SEQ ID NO:1.

Embodiment 21J. A nucleic acid molecule encoding a vector of any one of Embodiments 1J-13J or a polypeptide of any one of Embodiments 14J-20J.

Embodiment 22J. The nucleic acid molecule of Embodiment 21J, wherein said nucleic acid molecule is DNA.

Embodiment 23J. A host cell comprising a nucleic acid molecule of any one of Embodiments 21J-22J.

Embodiment 24J. The host cell of Embodiment 23J, wherein said host cell expresses said vector.

Embodiment 25J. The host cell of Embodiment 23J, wherein said host cell expresses said polypeptide.

Embodiment 26J. A host cell comprising a vector of any one of Embodiments 1J-13J or a polypeptide of any one of Embodiments 14J-20J.

Embodiment 27J. The host cell of any one of Embodiments 23J-26J, wherein said host cell is a retinal cell.

Embodiment 28J. A composition comprising a vector of any one of Embodiments 1J-13J.

Embodiment 29J. The composition of Embodiment 28J, wherein said composition comprises from about 1×10⁷ to about 1×10¹⁴ of said vector.

Embodiment 30J. The composition of any one of Embodiments 28J-29J, wherein said composition comprises phosphate buffered saline, Hank's Balanced Salt Solution, or Pluronic F68.

Embodiment 31J. A method for delivering an exogenous nucleic acid sequence to an ON-retinal ganglion cell within a mammal, wherein said method comprises contacting said ON-retinal ganglion cell with an AAV vector comprising an AAV capsid polypeptide and said exogenous nucleic acid sequence, wherein said capsid polypeptide comprises the amino acid sequence of any one of SEQ ID NOs:2J-64J, wherein said AAV vector infects said ON-retinal ganglion cell, thereby delivering said exogenous nucleic acid sequence to said ON-retinal ganglion cell.

Embodiment 32J. The method of Embodiment 31J, wherein said capsid polypeptide comprises the amino acid sequence of SEQ ID NO:1 except that said amino acid sequence of any one of SEQ ID NOs:J2-J64 is located between amino acid positions 587 and 588 of SEQ ID NO:1.

Embodiment 33J. The method of Embodiment 31J, wherein said capsid polypeptide comprises the amino acid sequence of SEQ ID NO:1 except that said amino acid sequence of any one of SEQ ID NOs:J2-J61 is located between amino acid positions 587 and 588 of SEQ ID NO:1.

Embodiment 34J. The method of Embodiment 31J, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:J2-J64.

Embodiment 35J. The method of Embodiment J31, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:J62-J64.

Embodiment 36J. The method of any one of Embodiments 31J-35J, wherein said mammal is a human.

Embodiment 37J. The method of any one of Embodiments 31J-36J, wherein said vector is an AAV2 vector.

Embodiment 38J. The method of any one of Embodiments 31J-37J, wherein said vector infects greater than 2 percent of ON-retinal ganglion cells of an eye when a titer of at least 1×10⁷ of said vector is administered intravitreally to said eye.

Embodiment 39J. The method of any one of Embodiments 31J-38J, wherein said exogenous nucleic acid sequence encodes an RNA or a polypeptide.

Embodiment 40J. The method of Embodiment 39J, wherein said exogenous nucleic acid encodes an RNA.

Embodiment 41J. The method of Embodiment 40J, wherein said RNA is an siRNA or microRNA.

Embodiment 42J. The method of Embodiment 39J, wherein said exogenous nucleic acid encodes a polypeptide.

Embodiment 43J. The method of Embodiment 42J, wherein said polypeptide is an ABCA4 polypeptide, a CRB1 polypeptide, an NPHP5 polypeptide, or an NR2E3 polypeptide.

Embodiment 44J. The method of any one of Embodiments 31J-43J, wherein said vector expresses more of said exogenous nucleic acid sequence in said ON-retinal ganglion cell than the level of expression in an ON-retinal ganglion cell from a comparable AAV vector comprising a capsid polypeptide consisting of the amino acid sequence set forth in SEQ ID NO:1.

Embodiment 45J. The method of any one of Embodiments 31J-44J, wherein said method comprises intravitreally administering a composition comprising said vector to said mammal, thereby contacting said ON-retinal ganglion cell with said vector.

Embodiment 46J. The method of Embodiment 45J, wherein said composition comprises from about 1×10⁷ to about 1×10¹⁴ of said vector.

Embodiment 47J. A method for treating a retinal condition, wherein said method comprises contacting ON-retinal ganglion cells of a mammal having said retinal condition with AAV vectors comprising an AAV capsid polypeptide and an exogenous nucleic acid sequence, wherein said capsid polypeptide comprises the amino acid sequence of any one of SEQ ID NOs:J2-J64, wherein said AAV vectors infect said ON-retinal ganglion cells and drive expression of said exogenous nucleic acid sequence within said ON-retinal ganglion cells, thereby treating said retinal condition.

Embodiment 48J. The method of Embodiment 47J, wherein said mammal is a human.

Embodiment 49J. The method of any one of Embodiments 47J-48J, wherein said retinal condition is selected from the group consisting of cone dystrophy, cone/rod dystrophy, retinitis pigmentosa, macular degeneration, achromatopsia, blue cone monochromcy, and color blindness.

Embodiment 50J. The method of any one of Embodiments 47J-49J, wherein said capsid polypeptide comprises the amino acid sequence of SEQ ID NO:1 except that said amino acid sequence of any one of SEQ ID NOs:J2-J64 is located between amino acid positions 587 and 588 of SEQ ID NO:1.

Embodiment 51J. The method of any one of Embodiments 47J-49J, wherein said capsid polypeptide comprises the amino acid sequence of SEQ ID NO:1 except that said amino acid sequence of any one of SEQ ID NOs:J2-J61 is located between amino acid positions 587 and 588 of SEQ ID NO:1.

Embodiment 52J. The method of any one of Embodiments 47J-49J, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:J2-J64.

Embodiment 53J. The method of any one of Embodiments 47J-49J, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:J62-J64.

Embodiment 54J. The method of any one of Embodiments 47J-53J, wherein said vectors are AAV2 vectors.

Embodiment 55J. The method of any one of Embodiments 47J-54J, wherein said vectors infect greater than 2 percent of ON-retinal ganglion cells when a titer of at least 1×10⁷ of said vectors is administered intravitreally to an eye of said mammal.

Embodiment 56J. The method of any one of Embodiments 47J-55J, wherein said exogenous nucleic acid sequence encodes an RNA.

Embodiment 57J. The method of Embodiment 56J, wherein said RNA is an siRNA or microRNA.

Embodiment 58J. The method of any one of Embodiments 47J-55J, wherein said exogenous nucleic acid encodes a polypeptide.

Embodiment 59J. The method of Embodiment 58J, wherein said polypeptide is an ABCA4 polypeptide, a CRB1 polypeptide, an NPHP5 polypeptide, or an NR2E3 polypeptide.

Embodiment 60J. The method of any one of Embodiments 47J-59J, wherein said vectors express more of said exogenous nucleic acid sequence in said ON-retinal ganglion cells than the level of expression in ON-retinal ganglion cells from a comparable AAV vector comprising a capsid polypeptide consisting of the amino acid sequence set forth in SEQ ID NO:1.

Embodiment 61J. The method of any one of Embodiments 47J-60J, wherein said method comprises intravitreally administering a composition comprising said vectors to said mammal, thereby contacting said ON-retinal ganglion cells with said vectors.

Embodiment 62J. The method of Embodiment 61J, wherein said composition comprises from about 1×10⁷ to about 1×10¹⁴ of said vector.

Embodiment 1K. An adeno-associated virus (AAV) vector comprising an AAV capsid polypeptide, wherein said capsid polypeptide comprises the amino acid sequence of any one of SEQ ID NOs:K2-K63.

Embodiment 2K. The vector of Embodiment 1K, wherein said capsid polypeptide comprises the amino acid sequence of SEQ ID NO:1 except that said amino acid sequence of any one of SEQ ID NOs:K2-K63 is located between amino acid positions 587 and 588 of SEQ ID NO:1.

Embodiment 3K. The vector of Embodiment 1K, wherein said capsid polypeptide comprises the amino acid sequence of SEQ ID NO:1 except that said amino acid sequence of any one of SEQ ID NOs:K2-K60 is located between amino acid positions 587 and 588 of SEQ ID NO:1.

Embodiment 4K. The vector of Embodiment 1K, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:K2-K63.

Embodiment 5K. The vector of Embodiment 1K, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:K61-K63.

Embodiment 6K. The vector of any one of Embodiments 1K-5K, wherein said vector is an AAV2 vector.

Embodiment 7K. The vector of any one of Embodiments 1K-6K, wherein said vector infects greater than 2 percent of OFF-retinal ganglion cells of an eye when a titer of at least 1×10⁷ of said vector is administered intravitreally to said eye.

Embodiment 8K. The vector of any one of Embodiments 1K-7K, wherein said vector comprises an exogenous nucleic acid encoding an RNA or a polypeptide.

Embodiment 9K. The vector of Embodiment 8K, wherein said exogenous nucleic acid encodes an RNA.

Embodiment 10K. The vector of Embodiment 9K, wherein said RNA is an siRNA or microRNA.

Embodiment 11K. The vector of Embodiment 8K, wherein said exogenous nucleic acid encodes a polypeptide.

Embodiment 12K. The vector of Embodiment 11K, wherein said polypeptide is an ABCA4 polypeptide, a CRB1 polypeptide, an NPHP5 polypeptide, or an NR2E3 polypeptide.

Embodiment 13K. The vector of any one of Embodiments 1K-12K, wherein said vector expresses more nucleic acid in OFF-retinal ganglion cells than the level of expression from a comparable AAV vector comprising a capsid polypeptide consisting of the amino acid sequence set forth in SEQ ID NO:1.

Embodiment 14K. An AAV capsid polypeptide comprising the amino acid sequence of any one of SEQ ID NOs:K2-K63.

Embodiment 15K. The polypeptide of Embodiment 14K, wherein said polypeptide comprises the amino acid sequence of SEQ ID NO:1 except that said amino acid sequence of any one of SEQ ID NOs:K2-K63 is located between amino acid positions 587 and 588 of SEQ ID NO:1.

Embodiment 16K. The polypeptide of Embodiment 14K, wherein said polypeptide comprises the amino acid sequence of SEQ ID NO:1 except that said amino acid sequence of any one of SEQ ID NOs:K2-K60 is located between amino acid positions 587 and 588 of SEQ ID NO:1.

Embodiment 17K. The polypeptide of Embodiment 14K, wherein said polypeptide comprises the amino acid sequence of SEQ ID NO:1 except that the amino acids from position 585 to 590 of SEQ ID NO:1 are replaced with said amino acid sequence of any one of SEQ ID NOs:K2-K63.

Embodiment 18K. The polypeptide of Embodiment 14K, wherein said polypeptide comprises the amino acid sequence of SEQ ID NO:1 except that the amino acids from position 585 to 590 of SEQ ID NO:1 are replaced with said amino acid sequence of any one of SEQ ID NOs:K61-K63.

Embodiment 19K. The polypeptide of any one of Embodiments 14K-18K, wherein an AAV vector comprising said polypeptide infects greater than 2 percent of OFF-retinal ganglion cells of an eye when a titer of at least 1×10⁷ of said vector is administered intravitreally to said eye.

Embodiment 20K. The polypeptide of any one of Embodiments 14K-19K, wherein an AAV vector comprising said polypeptide expresses more nucleic acid in OFF-retinal ganglion cells than the level of expression from a comparable AAV vector comprising a capsid polypeptide consisting of the amino acid sequence set forth in SEQ ID NO:1.

Embodiment 21K. A nucleic acid molecule encoding a vector of any one of Embodiments 1K-13K or a polypeptide of any one of Embodiments 14K-20K.

Embodiment 22K. The nucleic acid molecule of Embodiment 21K, wherein said nucleic acid molecule is DNA.

Embodiment 23K. A host cell comprising a nucleic acid molecule of any one of Embodiments 21K-22K.

Embodiment 24K. The host cell of Embodiment 23K, wherein said host cell expresses said vector.

Embodiment 25K. The host cell of Embodiment 23K, wherein said host cell expresses said polypeptide.

Embodiment 26K. A host cell comprising a vector of any one of Embodiments 1K-13K or a polypeptide of any one of Embodiments 14K-20K.

Embodiment 27K. The host cell of any one of Embodiments 23K-26K, wherein said host cell is a retinal cell.

Embodiment 28K. A composition comprising a vector of any one of Embodiments 1K-13K.

Embodiment 29K. The composition of Embodiment 28K, wherein said composition comprises from about 1×10⁷ to about 1×10¹⁴ of said vector.

Embodiment 30K. The composition of any one of Embodiments 28K-29K, wherein said composition comprises phosphate buffered saline, Hank's Balanced Salt Solution, or Pluronic F68.

Embodiment 31K. A method for delivering an exogenous nucleic acid sequence to an OFF-retinal ganglion cell within a mammal, wherein said method comprises contacting said OFF-retinal ganglion cell with an AAV vector comprising an AAV capsid polypeptide and said exogenous nucleic acid sequence, wherein said capsid polypeptide comprises the amino acid sequence of any one of SEQ ID NOs:K2-K63, wherein said AAV vector infects said OFF-retinal ganglion cell, thereby delivering said exogenous nucleic acid sequence to said OFF-retinal ganglion cell.

Embodiment 32K. The method of Embodiment 31K, wherein said capsid polypeptide comprises the amino acid sequence of SEQ ID NO:1 except that said amino acid sequence of any one of SEQ ID NOs:K2-K63 is located between amino acid positions 587 and 588 of SEQ ID NO:1.

Embodiment 33K. The method of Embodiment 31K, wherein said capsid polypeptide comprises the amino acid sequence of SEQ ID NO:1 except that said amino acid sequence of any one of SEQ ID NOs:K2-K60 is located between amino acid positions 587 and 588 of SEQ ID NO:1.

Embodiment 34K. The method of Embodiment 31K, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:K2-K63.

Embodiment 35K. The method of Embodiment 31K, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:K61-K63.

Embodiment 36K. The method of any one of Embodiments 31K-35K, wherein said mammal is a human.

Embodiment 37K. The method of any one of Embodiments 31K-36K, wherein said vector is an AAV2 vector.

Embodiment 38K. The method of any one of Embodiments 31K-37K, wherein said vector infects greater than 2 percent of OFF-retinal ganglion cells of an eye when a titer of at least 1×10⁷ of said vector is administered intravitreally to said eye.

Embodiment 39K. The method of any one of Embodiments 31K-38K, wherein said exogenous nucleic acid sequence encodes an RNA or a polypeptide.

Embodiment 40K. The method of Embodiment 39K, wherein said exogenous nucleic acid encodes an RNA.

Embodiment 41K. The method of Embodiment 40K, wherein said RNA is an siRNA or microRNA.

Embodiment 42K. The method of Embodiment 39K, wherein said exogenous nucleic acid encodes a polypeptide.

Embodiment 43K. The method of Embodiment 42K, wherein said polypeptide is an ABCA4 polypeptide, a CRB1 polypeptide, an NPHP5 polypeptide, or an NR2E3 polypeptide.

Embodiment 44K. The method of any one of Embodiments 31K-43K, wherein said vector expresses more of said exogenous nucleic acid sequence in said OFF-retinal ganglion cell than the level of expression in an OFF-retinal ganglion cell from a comparable AAV vector comprising a capsid polypeptide consisting of the amino acid sequence set forth in SEQ ID NO:1.

Embodiment 45K. The method of any one of Embodiments 31K-44K, wherein said method comprises intravitreally administering a composition comprising said vector to said mammal, thereby contacting said OFF-retinal ganglion cell with said vector.

Embodiment 46K. The method of Embodiment 45K, wherein said composition comprises from about 1×10⁷ to about 1×10¹⁴ of said vector.

Embodiment 47K. A method for treating a retinal condition, wherein said method comprises contacting OFF-retinal ganglion cells of a mammal having said retinal condition with AAV vectors comprising an AAV capsid polypeptide and an exogenous nucleic acid sequence, wherein said capsid polypeptide comprises the amino acid sequence of any one of SEQ ID NOs:K2-K63, wherein said AAV vectors infect said OFF-retinal ganglion cells and drive expression of said exogenous nucleic acid sequence within said OFF-retinal ganglion cells, thereby treating said retinal condition.

Embodiment 48K. The method of Embodiment 47K, wherein said mammal is a human.

Embodiment 49K. The method of any one of Embodiments 47K-48K, wherein said retinal condition is selected from the group consisting of cone dystrophy, cone/rod dystrophy, retinitis pigmentosa, macular degeneration, achromatopsia, blue cone monochromcy, and color blindness.

Embodiment 50K. The method of any one of Embodiments 47K-49K, wherein said capsid polypeptide comprises the amino acid sequence of SEQ ID NO:1 except that said amino acid sequence of any one of SEQ ID NOs:K2-K63 is located between amino acid positions 587 and 588 of SEQ ID NO:1.

Embodiment 51K. The method of any one of Embodiments 47K-49K, wherein said capsid polypeptide comprises the amino acid sequence of SEQ ID NO:1 except that said amino acid sequence of any one of SEQ ID NOs:K2-K60 is located between amino acid positions 587 and 588 of SEQ ID NO:1.

Embodiment 52K. The method of any one of Embodiments 47K-49K, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:K2-K63.

Embodiment 53K. The method of any one of Embodiments 47K-49K, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:K61-K63.

Embodiment 54K. The method of any one of Embodiments 47K-53K, wherein said vectors are AAV2 vectors.

Embodiment 55K. The method of any one of Embodiments 47K-54K, wherein said vectors infect greater than 2 percent of OFF-retinal ganglion cells when a titer of at least 1×10⁷ of said vectors is administered intravitreally to an eye of said mammal.

Embodiment 56K. The method of any one of Embodiments 47K-55K, wherein said exogenous nucleic acid sequence encodes an RNA.

Embodiment 57K. The method of Embodiment 56K, wherein said RNA is an siRNA or microRNA.

Embodiment 58K. The method of any one of Embodiments 47K-55K, wherein said exogenous nucleic acid encodes a polypeptide.

Embodiment 59K. The method of Embodiment 58K, wherein said polypeptide is an ABCA4 polypeptide, a CRB1 polypeptide, an NPHP5 polypeptide, or an NR2E3 polypeptide.

Embodiment 60K. The method of any one of Embodiments 47K-59K, wherein said vectors express more of said exogenous nucleic acid sequence in said OFF-retinal ganglion cells than the level of expression in OFF-retinal ganglion cells from a comparable AAV vector comprising a capsid polypeptide consisting of the amino acid sequence set forth in SEQ ID NO:1.

Embodiment 61K. The method of any one of Embodiments 47K-60K, wherein said method comprises intravitreally administering a composition comprising said vectors to said mammal, thereby contacting said OFF-retinal ganglion cells with said vectors.

Embodiment 62K. The method of Embodiment 61K, wherein said composition comprises from about 1×10⁷ to about 1×10¹⁴ of said vector.

Embodiment 1L. An adeno-associated virus (AAV) vector comprising an AAV capsid polypeptide, wherein said capsid polypeptide comprises the amino acid sequence of any one of SEQ ID NOs:L2-L27.

Embodiment 2L. The vector of Embodiment 1L, wherein said capsid polypeptide comprises the amino acid sequence of SEQ ID NO:1 except that said amino acid sequence of any one of SEQ ID NOs:L2-L27 is located between amino acid positions 587 and 588 of SEQ ID NO: 1.

Embodiment 3L. The vector of Embodiment 1L, wherein said capsid polypeptide comprises the amino acid sequence of SEQ ID NO:1 except that said amino acid sequence of any one of SEQ ID NOs:L2-L19 is located between amino acid positions 587 and 588 of SEQ ID NO: 1.

Embodiment 4L. The vector of Embodiment 1L, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:L2-L27.

Embodiment 5L. The vector of Embodiment 1L, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:L20-L27.

Embodiment 6L. The vector of any one of Embodiments 1L-5L, wherein said vector is an AAV2 vector.

Embodiment 7L. The vector of any one of Embodiments 1L-6L, wherein said vector has a packaging efficiency greater than that of a comparable AAV vector comprising a capsid polypeptide consisting of the amino acid sequence set forth in SEQ ID NO:1.

Embodiment 8L. The vector of any one of Embodiments 1L-7L, wherein said vector comprises an exogenous nucleic acid encoding an RNA or a polypeptide.

Embodiment 9L. The vector of Embodiment 8L, wherein said exogenous nucleic acid encodes an RNA.

Embodiment 10L. The vector of Embodiment 9L, wherein said RNA is an siRNA or microRNA.

Embodiment 11L. The vector of Embodiment 8L, wherein said exogenous nucleic acid encodes a polypeptide.

Embodiment 12L. The vector of Embodiment 11L, wherein said polypeptide is an ABCA4 polypeptide, a CRB1 polypeptide, an NPHP5 polypeptide, or an NR2E3 polypeptide.

Embodiment 13L. The vector of any one of Embodiments 1L-12L, wherein said vector has a packaging efficiency at least 10 percent greater than that of a comparable AAV vector comprising a capsid polypeptide consisting of the amino acid sequence set forth in SEQ ID NO:1.

Embodiment 14L. An AAV capsid polypeptide comprising the amino acid sequence of any one of SEQ ID NOs:L2-L27.

Embodiment 15L. The polypeptide of Embodiment 14L, wherein said polypeptide comprises the amino acid sequence of SEQ ID NO:1 except that said amino acid sequence of any one of SEQ ID NOs:L2-L27 is located between amino acid positions 587 and 588 of SEQ ID NO: 1.

Embodiment 16L. The polypeptide of Embodiment 14L, wherein said polypeptide comprises the amino acid sequence of SEQ ID NO:1 except that said amino acid sequence of any one of SEQ ID NOs:L2-L19 is located between amino acid positions 587 and 588 of SEQ ID NO: 1.

Embodiment 17L. The polypeptide of Embodiment 14L, wherein said polypeptide comprises the amino acid sequence of SEQ ID NO:1 except that the amino acids from position 585 to 590 of SEQ ID NO:1 are replaced with said amino acid sequence of any one of SEQ ID NOs:L2-L27.

Embodiment 18L. The polypeptide of Embodiment 14L, wherein said polypeptide comprises the amino acid sequence of SEQ ID NO:1 except that the amino acids from position 585 to 590 of SEQ ID NO:1 are replaced with said amino acid sequence of any one of SEQ ID NOs:L20-L27.

Embodiment 19L. The polypeptide of any one of Embodiments 14L-18L, wherein said vector has a packaging efficiency greater than that of a comparable AAV vector comprising a capsid polypeptide consisting of the amino acid sequence set forth in SEQ ID NO:1.

Embodiment 20L. The polypeptide of any one of Embodiments 14L-19L, wherein said vector has a packaging efficiency at least 10 percent greater than that of a comparable AAV vector comprising a capsid polypeptide consisting of the amino acid sequence set forth in SEQ ID NO:1.

Embodiment 21L. A nucleic acid molecule encoding a vector of any one of Embodiments 1L-13L or a polypeptide of any one of Embodiments 14L-20L.

Embodiment 22L. The nucleic acid molecule of Embodiment 21L, wherein said nucleic acid molecule is DNA.

Embodiment 23L. A host cell comprising a nucleic acid molecule of any one of Embodiments 21L-22L.

Embodiment 24L. The host cell of Embodiment 23L, wherein said host cell expresses said vector.

Embodiment 25L. The host cell of Embodiment 23L, wherein said host cell expresses said polypeptide.

Embodiment 26L. A host cell comprising a vector of any one of Embodiments 1L-13L or a polypeptide of any one of Embodiments 14L-20L.

Embodiment 27L. The host cell of any one of Embodiments 23L-26L, wherein said host cell is a retinal cell.

Embodiment 28L. A composition comprising a vector of any one of Embodiments 1L-13L.

Embodiment 29L. The composition of Embodiment 28L, wherein said composition comprises from about 1×10⁷ to about 1×10¹⁴ of said vector.

Embodiment 30L. The composition of any one of Embodiments 28L-29L, wherein said composition comprises phosphate buffered saline, Hank's Balanced Salt Solution, or Pluronic F68.

Embodiment 31L. A method for delivering an exogenous nucleic acid sequence to a cell within a mammal, wherein said method comprises contacting said cell with an AAV vector comprising an AAV capsid polypeptide and said exogenous nucleic acid sequence, wherein said capsid polypeptide comprises the amino acid sequence of any one of SEQ ID NOs:L2-L27, wherein said AAV vector infects said cell, thereby delivering said exogenous nucleic acid sequence to said cell.

Embodiment 32L. The method of Embodiment 31L, wherein said capsid polypeptide comprises the amino acid sequence of SEQ ID NO:1 except that said amino acid sequence of any one of SEQ ID NOs:L2-L27 is located between amino acid positions 587 and 588 of SEQ ID NO:1.

Embodiment 33L. The method of Embodiment 31L, wherein said capsid polypeptide comprises the amino acid sequence of SEQ ID NO:1 except that said amino acid sequence of any one of SEQ ID NOs:L2-L19 is located between amino acid positions 587 and 588 of SEQ ID NO:1.

Embodiment 34L. The method of Embodiment 31L, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:L2-L27.

Embodiment 35L. The method of Embodiment 31L, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:L20-L27.

Embodiment 36L. The method of any one of Embodiments 31L-35L, wherein said mammal is a human.

Embodiment 37L. The method of any one of Embodiments 31L-36L, wherein said vector is an AAV2 vector.

Embodiment 38L. The method of any one of Embodiments 31L-37L, wherein said vector has a packaging efficiency greater than that of a comparable AAV vector comprising a capsid polypeptide consisting of the amino acid sequence set forth in SEQ ID NO:1.

Embodiment 39L. The method of any one of Embodiments 31L-38L, wherein said exogenous nucleic acid sequence encodes an RNA or a polypeptide.

Embodiment 40L. The method of Embodiment 39L, wherein said exogenous nucleic acid encodes an RNA.

Embodiment 41L. The method of Embodiment 40L, wherein said RNA is an siRNA or microRNA.

Embodiment 42L. The method of Embodiment 39L, wherein said exogenous nucleic acid encodes a polypeptide.

Embodiment 43L. The method of Embodiment 42L, wherein said polypeptide is an ABCA4 polypeptide, a CRB1 polypeptide, an NPHP5 polypeptide, or an NR2E3 polypeptide.

Embodiment 44L. The method of any one of Embodiments 31L-43L, wherein said vector has a packaging efficiency at least 10 percent greater than that of a comparable AAV vector comprising a capsid polypeptide consisting of the amino acid sequence set forth in SEQ ID NO:1.

Embodiment 45L. The method of any one of Embodiments 31L-44L, wherein said method comprises intravitreally administering a composition comprising said vector to said mammal, thereby contacting said cell with said vector.

Embodiment 46L. The method of Embodiment 45L, wherein said composition comprises from about 1×10⁷ to about 1×10¹⁴ of said vector.

Embodiment 47L. A method for treating a retinal condition, wherein said method comprises contacting retinal cells of a mammal having said retinal condition with AAV vectors comprising an AAV capsid polypeptide and an exogenous nucleic acid sequence, wherein said capsid polypeptide comprises the amino acid sequence of any one of SEQ ID NOs:L2-L27, wherein said AAV vectors infect said retinal cells and drive expression of said exogenous nucleic acid sequence within said retinal cells, thereby treating said retinal condition.

Embodiment 48L. The method of Embodiment 47L, wherein said mammal is a human.

Embodiment 49L. The method of any one of Embodiments 47L-48L, wherein said retinal condition is selected from the group consisting of cone dystrophy, cone/rod dystrophy, retinitis pigmentosa, macular degeneration, achromatopsia, blue cone monochromcy, and color blindness.

Embodiment 50L. The method of any one of Embodiments 47L-49L, wherein said capsid polypeptide comprises the amino acid sequence of SEQ ID NO:1 except that said amino acid sequence of any one of SEQ ID NOs:L2-L27 is located between amino acid positions 587 and 588 of SEQ ID NO:1.

Embodiment 51L. The method of any one of Embodiments 47L-49L, wherein said capsid polypeptide comprises the amino acid sequence of SEQ ID NO:1 except that said amino acid sequence of any one of SEQ ID NOs:L2-L19 is located between amino acid positions 587 and 588 of SEQ ID NO:1.

Embodiment 52L. The method of any one of Embodiments 47L-49L, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:L2-L27.

Embodiment 53L. The method of any one of Embodiments 47L-49L, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:L19-L27.

Embodiment 54L. The method of any one of Embodiments 47L-53L, wherein said vectors are AAV2 vectors.

Embodiment 55L. The method of any one of Embodiments 47L-54L, wherein said vector has a packaging efficiency greater than that of a comparable AAV vector comprising a capsid polypeptide consisting of the amino acid sequence set forth in SEQ ID NO:1.

Embodiment 56L. The method of any one of Embodiments 47L-55L, wherein said exogenous nucleic acid sequence encodes an RNA.

Embodiment 57L. The method of Embodiment 56L, wherein said RNA is an siRNA or microRNA.

Embodiment 58L. The method of any one of Embodiments 47L-55L, wherein said exogenous nucleic acid encodes a polypeptide.

Embodiment 59L. The method of Embodiment 58L, wherein said polypeptide is an ABCA4 polypeptide, a CRB1 polypeptide, an NPHP5 polypeptide, or an NR2E3 polypeptide.

Embodiment 60L. The method of any one of Embodiments 47L-59L, wherein said vector has a packaging efficiency at least 10 percent greater than that of a comparable AAV vector comprising a capsid polypeptide consisting of the amino acid sequence set forth in SEQ ID NO:1.

Embodiment 61L. The method of any one of Embodiments 47L-60L, wherein said method comprises intravitreally administering a composition comprising said vectors to said mammal, thereby contacting said retinal cells with said vectors.

Embodiment 62L. The method of Embodiment 61L, wherein said composition comprises from about 1×10⁷ to about 1×10¹⁴ of said vector.

Embodiment 1M. An adeno-associated virus (AAV) vector comprising an AAV capsid polypeptide, wherein said capsid polypeptide comprises the amino acid sequence of any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-I74, SEQ ID NOs:J2-J64, SEQ ID NOs:K2-K63, and SEQ ID NOs:L2-L27.

Embodiment 2M. The vector of Embodiment 1M, wherein said capsid polypeptide comprises the amino acid sequence of SEQ ID NO:1 except that said amino acid sequence of any one of SEQ ID NOs: A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-174, SEQ ID NOs:J2-J64, SEQ ID NOs:K2-K63, and SEQ ID NOs:L2-L27 is located between amino acid positions 587 and 588 of SEQ ID NO: 1.

Embodiment 3M. The vector of Embodiment 1M, wherein said capsid polypeptide comprises the amino acid sequence of SEQ ID NO:1 except that said amino acid sequence of any one of SEQ ID NOs:A2-A18, SEQ ID NOs:B2-B298, SEQ ID NOs:C2-C43, SEQ ID NOs:D2-D76, SEQ ID NOs:E2-E75, SEQ ID NOs:F2-F10, SEQ ID NOs:G2-G70, SEQ ID NOs:H2-H243, SEQ ID NOs:I2-167, SEQ ID NOs:J2-J61, SEQ ID NOs:K2-K60, and SEQ ID NOs:L2-L19 is located between amino acid positions 587 and 588 of SEQ ID NO: 1.

Embodiment 4M. The vector of Embodiment 1M, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-I74, SEQ ID NOs:J2-J64, SEQ ID NOs:K2-K63, and SEQ ID NOs:L2-L27.

Embodiment 5M. The vector of Embodiment 1M, wherein said capsid polypeptide comprises the amino acid sequence of SEQ ID NO:1 except that the amino acids from position 585 to 590 of SEQ ID NO:1 are replaced with said amino acid sequence set forth in any one of SEQ ID NO:A19, SEQ ID NOs:B299-B317, SEQ ID NOs:C44-C45, SEQ ID NOs:D77-D78, SEQ ID NOs:E76-E83, SEQ ID NOs:F2-F10, SEQ ID NOs:G71-G77, SEQ ID NOs:H243-H258, SEQ ID NOs:I68-I74, SEQ ID NOs:J62-J64, SEQ ID NOs:K61-K63, and SEQ ID NOs:L20-L27.

Embodiment 6M. The vector of any one of Embodiments 1M-5M, wherein said vector is an AAV2 vector.

Embodiment 7M. The vector of any one of Embodiments 1M-6M, wherein said vector comprises an exogenous nucleic acid encoding an RNA or a polypeptide.

Embodiment 8N. The vector of Embodiment 7N, wherein said exogenous nucleic acid encodes an RNA.

Embodiment 9M. The vector of Embodiment 8M, wherein said RNA is an siRNA or microRNA.

Embodiment 10M. The vector of Embodiment 7M, wherein said exogenous nucleic acid encodes a polypeptide.

Embodiment 11M. The vector of Embodiment 10M, wherein said polypeptide is an ABCA4 polypeptide, a CRB1 polypeptide, an NPHP5 polypeptide, or an NR2E3 polypeptide.

Embodiment 12M. An AAV capsid polypeptide comprising the amino acid sequence of any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-174, SEQ ID NOs:J2-J64, SEQ ID NOs:K2-K63, and SEQ ID NOs:L2-L27.

Embodiment 13M. The polypeptide of Embodiment 12M, wherein said polypeptide comprises the amino acid sequence of SEQ ID NO:1 except that said amino acid sequence of any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-I74, SEQ ID NOs:J2-J64, SEQ ID NOs:K2-K63, and SEQ ID NOs:L2-L27 is located between amino acid positions 587 and 588 of SEQ ID NO: 1.

Embodiment 14M. The polypeptide of Embodiment 12M, wherein said polypeptide comprises the amino acid sequence of SEQ ID NO:1 except that said amino acid sequence of any one of SEQ ID NOs:A2-A18, SEQ ID NOs:B2-B298, SEQ ID NOs:C2-C43, SEQ ID NOs:D2-D76, SEQ ID NOs:E2-E75, SEQ ID NOs:F2-F10, SEQ ID NOs:G2-G70, SEQ ID NOs:H2-H243, SEQ ID NOs:I2-167, SEQ ID NOs:J2-J61, SEQ ID NOs:K2-K60, and SEQ ID NOs:L2-L19 is located between amino acid positions 587 and 588 of SEQ ID NO:1.

Embodiment 15M. The polypeptide of Embodiment 12M, wherein said polypeptide comprises the amino acid sequence of SEQ ID NO:1 except that the amino acids from position 585 to 590 of SEQ ID NO:1 are replaced with said amino acid sequence of any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-I74, SEQ ID NOs:J2-J64, SEQ ID NOs:K2-K63, and SEQ ID NOs:L2-L27.

Embodiment 16M. The polypeptide of Embodiment 12M, wherein said polypeptide comprises the amino acid sequence of SEQ ID NO:1 except that the amino acids from position 585 to 590 of SEQ ID NO:1 are replaced with said amino acid sequence set forth in any one of SEQ ID NO:A19, SEQ ID NOs:B299-B317, SEQ ID NOs:C44-C45, SEQ ID NOs:D77-D78, SEQ ID NOs:E76-E83, SEQ ID NOs:F2-F10, SEQ ID NOs:G71-G77, SEQ ID NOs:H243-H258, SEQ ID NOs:I68-I74, SEQ ID NOs:J62-J64, SEQ ID NOs:K61-K63, and SEQ ID NOs:L20-L27.

Embodiment 17M. A nucleic acid molecule encoding a vector of any one of Embodiments 1M-11M or a polypeptide of any one of Embodiments 12M-16M.

Embodiment 18M. The nucleic acid molecule of Embodiment 17M, wherein said nucleic acid molecule is DNA.

Embodiment 19M. A host cell comprising a nucleic acid molecule of any one of Embodiments 17M-18M.

Embodiment 20M. The host cell of Embodiment 19M, wherein said host cell expresses said vector.

Embodiment 21M. The host cell of Embodiment 20M, wherein said host cell expresses said polypeptide.

Embodiment 22M. A host cell comprising a vector of any one of Embodiments 1M-11M or a polypeptide of any one of Embodiments 12M-16M.

Embodiment 23M. The host cell of any one of Embodiments 19M-22M, wherein said host cell is a retinal cell.

Embodiment 24M. A composition comprising a vector of any one of Embodiments 1M-11M.

Embodiment 25M. The composition of Embodiment 24M, wherein said composition comprises from about 1×10⁷ to about 1×10¹⁴ of said vector.

Embodiment 26M. The composition of any one of Embodiments 24M-25M, wherein said composition comprises phosphate buffered saline, Hank's Balanced Salt Solution, or Pluronic F68.

Embodiment 27M. A method for delivering an exogenous nucleic acid sequence to a retinal cell within a mammal, wherein said method comprises contacting said retinal cell with an AAV vector comprising an AAV capsid polypeptide and said exogenous nucleic acid sequence, wherein said capsid polypeptide comprises the amino acid sequence of any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-I74, SEQ ID NOs:J2-J64, SEQ ID NOs:K2-K63, and SEQ ID NOs:L2-L27, wherein said AAV vector infects said retinal cell, thereby delivering said exogenous nucleic acid sequence to said retinal cell.

Embodiment 28M. The method of Embodiment 27M, wherein said capsid polypeptide comprises the amino acid sequence of SEQ ID NO:1 except that said amino acid sequence of any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-174, SEQ ID NOs:J2-J64, SEQ ID NOs:K2-K63, and SEQ ID NOs:L2-L27 is located between amino acid positions 587 and 588 of SEQ ID NO: 1.

Embodiment 29M. The method of Embodiment 27M, wherein said capsid polypeptide comprises the amino acid sequence of SEQ ID NO:1 except that said amino acid sequence of any one of SEQ ID NOs:A2-A18, SEQ ID NOs:B2-B298, SEQ ID NOs:C2-C43, SEQ ID NOs:D2-D76, SEQ ID NOs:E2-E75, SEQ ID NOs:F2-F10, SEQ ID NOs:G2-G70, SEQ ID NOs:H2-H243, SEQ ID NOs:I2-167, SEQ ID NOs:J2-J61, SEQ ID NOs:K2-K60, and SEQ ID NOs:L2-L19 is located between amino acid positions 587 and 588 of SEQ ID NO: 1.

Embodiment 30M. The method of Embodiment 27M, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-I74, SEQ ID NOs:J2-J64, SEQ ID NOs:K2-K63, and SEQ ID NOs:L2-L27.

Embodiment 31M. The method of Embodiment 27M, wherein said capsid polypeptide comprises the amino acid sequence of SEQ ID NO:1 except that the amino acids from position 585 to 590 of SEQ ID NO:1 are replaced with said amino acid sequence set forth in any one of SEQ ID NO:A19, SEQ ID NOs:B299-B317, SEQ ID NOs:C44-C45, SEQ ID NOs:D77-D78, SEQ ID NOs:E76-E83, SEQ ID NOs:F2-F10, SEQ ID NOs:G71-G77, SEQ ID NOs:H243-H258, SEQ ID NOs:I68-174, SEQ ID NOs:J62-J64, SEQ ID NOs:K61-K63, and SEQ ID NOs:L20-L27.

Embodiment 32M. The method of any one of Embodiments 27M-31M, wherein said mammal is a human.

Embodiment 33M. The method of any one of Embodiments 27M-32M, wherein said vector is an AAV2 vector.

Embodiment 34M. The method of any one of Embodiments 27M-33M, wherein said exogenous nucleic acid sequence encodes an RNA or a polypeptide.

Embodiment 35M. The method of Embodiment 34M, wherein said exogenous nucleic acid encodes an RNA.

Embodiment 36M. The method of Embodiment 35M, wherein said RNA is an siRNA or microRNA.

Embodiment 37M. The method of Embodiment 34M, wherein said exogenous nucleic acid encodes a polypeptide.

Embodiment 38M. The method of Embodiment 37M, wherein said polypeptide is an ABCA4 polypeptide, a CRB1 polypeptide, an NPHP5 polypeptide, or an NR2E3 polypeptide.

Embodiment 39M. The method of any one of Embodiments 27M-38M, wherein said method comprises intravitreally administering a composition comprising said vector to said mammal, thereby contacting said retinal cell with said vector.

Embodiment 40M. The method of Embodiment 39M, wherein said composition comprises from about 1×10⁷ to about 1×10¹⁴ of said vector.

Embodiment 41M. A method for treating a retinal condition, wherein said method comprises contacting retinal cells of a mammal having said retinal condition with AAV vectors comprising an AAV capsid polypeptide and an exogenous nucleic acid sequence, wherein said capsid polypeptide comprises the amino acid sequence of any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-I74, SEQ ID NOs:J2-J64, SEQ ID NOs:K2-K63, and SEQ ID NOs:L2-L27, wherein said AAV vectors infect said retinal cells and drive expression of said exogenous nucleic acid sequence within said retinal cells, thereby treating said retinal condition.

Embodiment 42M. The method of Embodiment 41M, wherein said mammal is a human.

Embodiment 43M. The method of any one of Embodiments 41M-42M, wherein said retinal condition is selected from the group consisting of cone dystrophy, cone/rod dystrophy, retinitis pigmentosa, macular degeneration, achromatopsia, blue cone monochromcy, and color blindness.

Embodiment 44M. The method of any one of Embodiments 41M-43M, wherein said capsid polypeptide comprises the amino acid sequence of SEQ ID NO:1 except that said amino acid sequence of any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-I74, SEQ ID NOs:J2-J64, SEQ ID NOs:K2-K63, and SEQ ID NOs:L2-L27 is located between amino acid positions 587 and 588 of SEQ ID NO:1.

Embodiment 45M. The method of any one of Embodiments 41M-43M, wherein said capsid polypeptide comprises the amino acid sequence of SEQ ID NO:1 except that said amino acid sequence of any one of SEQ ID NOs:A2-A18, SEQ ID NOs:B2-B298, SEQ ID NOs:C2-C43, SEQ ID NOs:D2-D76, SEQ ID NOs:E2-E75, SEQ ID NOs:F2-F10, SEQ ID NOs:G2-G70, SEQ ID NOs:H2-H243, SEQ ID NOs:I2-I67, SEQ ID NOs:J2-J61, SEQ ID NOs:K2-K60, and SEQ ID NOs:L2-L19 is located between amino acid positions 587 and 588 of SEQ ID NO:1.

Embodiment 46M. The method of any one of Embodiments 41M-43M, wherein said capsid polypeptide comprises the amino acid sequence of SEQ ID NO:1 except that the amino acids from position 585 to 590 of SEQ ID NO:1 are replaced with said amino acid sequence of any one of SEQ ID NOs:A2-A19, SEQ ID NOs:B2-B317, SEQ ID NOs:C2-C45, SEQ ID NOs:D2-D78, SEQ ID NOs:E2-E83, SEQ ID NOs:F2-F10, SEQ ID NOs:G2-G77, SEQ ID NOs:H2-H258, SEQ ID NOs:I2-I74, SEQ ID NOs:J2-J64, SEQ ID NOs:K2-K63, and SEQ ID NOs:L2-L27.

Embodiment 47M. The method of any one of Embodiments 41M-43M, wherein said capsid polypeptide comprises the amino acid sequence of SEQ ID NO:1 except that the amino acids from position 585 to 590 of SEQ ID NO:1 are replaced with said amino acid sequence set forth in any one of SEQ ID NO:A19, SEQ ID NOs:B299-B317, SEQ ID NOs:C44-C45, SEQ ID NOs:D77-D78, SEQ ID NOs:E76-E83, SEQ ID NOs:F2-F10, SEQ ID NOs:G71-G77, SEQ ID NOs:H243-H258, SEQ ID NOs:I68-174, SEQ ID NOs:J62-J64, SEQ ID NOs:K61-K63, and SEQ ID NOs:L20-L27.

Embodiment 48M. The method of any one of Embodiments 41M-47M, wherein said vectors are AAV2 vectors.

Embodiment 49M. The method of any one of Embodiments 41M-48M, wherein said exogenous nucleic acid sequence encodes an RNA.

Embodiment 50M. The method of Embodiment 49M, wherein said RNA is an siRNA or a microRNA.

Embodiment 51M. The method of any one of Embodiments 41M-50M, wherein said exogenous nucleic acid encodes a polypeptide.

Embodiment 52M. The method of Embodiment 51M, wherein said polypeptide is an ABCA4 polypeptide, a CRB1 polypeptide, an NPHP5 polypeptide, and an NR2E3 polypeptide.

Embodiment 53M. The method of any one of Embodiments 41M-52M, wherein said method comprises intravitreally administering a composition comprising said vectors to said mammal, thereby contacting said retinal cells with said vectors.

Embodiment 54M. The method of Embodiment 53M, wherein said composition comprises from about 1×10⁷ to about 1×10¹⁴ of said vectors.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An adeno-associated virus (AAV) vector comprising an AAV capsid polypeptide, wherein said capsid polypeptide comprises the amino acid sequence of any one of SEQ ID NOs:2-1103.

2. The vector of claim 1, wherein said capsid polypeptide comprises the amino acid sequence of SEQ ID NO:1 or SEQ ID NO:2206 except that said amino acid sequence of any one of SEQ ID NOs:2-1103 is located between amino acid positions 587 and 588 of SEQ ID NO:1 or SEQ ID NO:2206.

3. The vector of claim 1, wherein said capsid polypeptide comprises the amino acid sequence of SEQ ID NO:1 or SEQ ID NO:2206 except that the amino acids from position 585 to 590 of SEQ ID NO:1 or SEQ ID NO:2206 are replaced with said amino acid sequence of any one of SEQ ID NOs:2-1103.

4. The vector of any one of claims 1-3, wherein said vector is an AAV2 vector.

5. The vector of any one of claims 1-4, wherein said vector comprises an exogenous nucleic acid encoding an RNA or a polypeptide.

6. The vector of claim 5, wherein said exogenous nucleic acid encodes an RNA.

7. The vector of claim 6, wherein said RNA is an siRNA or microRNA.

8. The vector of claim 5, wherein said exogenous nucleic acid encodes a polypeptide.

9. The vector of claim 8, wherein said polypeptide is an ABCA4 polypeptide, a CRB1 polypeptide, an NPHP5 polypeptide, or an NR2E3 polypeptide.

10. An AAV capsid polypeptide comprising the amino acid sequence of any one of SEQ ID NOs:2-1103.

11. The polypeptide of claim 10, wherein said polypeptide comprises the amino acid sequence of SEQ ID NO:1 or SEQ ID NO:2206 except that said amino acid sequence of any one of SEQ ID NOs:2-1103 is located between amino acid positions 587 and 588 of SEQ ID NO:1 or SEQ ID NO:2206.

12. The polypeptide of claim 10, wherein said polypeptide comprises the amino acid sequence of SEQ ID NO:1 or SEQ ID NO:2206 except that the amino acids from position 585 to 590 of SEQ ID NO:1 or SEQ ID NO:2206 are replaced with said amino acid sequence of any one of SEQ ID NOs:2-1103.

13. A nucleic acid molecule encoding a vector of any one of claims 1-9 or a polypeptide of any one of claim 10-12.

14. The nucleic acid molecule of claim 13, wherein said nucleic acid molecule is DNA.

15. A host cell comprising a nucleic acid molecule of any one of claims 13-14.

16. The host cell of claim 15, wherein said host cell expresses said vector.

17. The host cell of claim 15, wherein said host cell expresses said polypeptide.

18. A host cell comprising a vector of any one of claims 1-9 or a polypeptide of any one of claims 10-12.

19. The host cell of any one of claims 15-18, wherein said host cell is a retinal cell.

20. A composition comprising a vector of any one of claims 1-9, and a pharmaceutically acceptable excipient.

21. The composition of claim 20, wherein said composition comprises from about 1×10⁷ to about 1×10¹⁴ of said vector.

22. The composition of any one of claim 20-21, wherein said pharmaceutically acceptable excipient comprises one or more of phosphate buffered saline, Hank's Balanced Salt Solution, and Pluronic F68.

23. A method for delivering an exogenous nucleic acid sequence to a retinal cell within a mammal, wherein said method comprises contacting said retinal cell with an AAV vector comprising an AAV capsid polypeptide and said exogenous nucleic acid sequence, wherein said capsid polypeptide comprises the amino acid sequence of any one of SEQ ID NOs:2-1103, wherein said AAV vector infects said retinal cell, thereby delivering said exogenous nucleic acid sequence to said retinal cell.

24. The method of claim 23, wherein said capsid polypeptide comprises the amino acid sequence of SEQ ID NO:1 or SEQ ID NO:2206 except that said amino acid sequence of any one of SEQ ID NOs:2-1103 is located between amino acid positions 587 and 588 of SEQ ID NO:1 or SEQ ID NO:2206.

25. The method of 23, wherein said capsid polypeptide comprises the amino acid sequence of SEQ ID NO:1 or SEQ ID NO:2206 except that the amino acids from position 585 to 590 of SEQ ID NO:1 or SEQ ID NO:2206 are replaced with said amino acid sequence of any one of SEQ ID NOs:2-1103.

26. The method of any one of claims 23-25, wherein said mammal is a human.

27. The method of any one of claims 23-26, wherein said vector is an AAV2 vector.

28. The method of any one of claims 23-27, wherein said exogenous nucleic acid sequence encodes an RNA or a polypeptide.

29. The method of claim 28, wherein said exogenous nucleic acid encodes an RNA.

30. The method of claim 29, wherein said RNA is an siRNA or microRNA.

31. The method of claim 28, wherein said exogenous nucleic acid encodes a polypeptide.

32. The method of claim 31, wherein said polypeptide is an ABCA4 polypeptide, a CRB1 polypeptide, an NPHP5 polypeptide, or an NR2E3 polypeptide.

33. The method of any one of claims 23-32, wherein said method comprises intravitreally administering a composition comprising said vector to said mammal, thereby contacting said retinal cell with said vector.

34. The method of claim 33, wherein said composition comprises from about 1×10⁷ to about 1×10¹⁴ of said vector.

35. A method for treating a retinal condition in a mammal in need thereof, wherein said method comprises contacting retinal cells of a mammal having said retinal condition with AAV vectors comprising an AAV capsid polypeptide and an exogenous nucleic acid sequence, wherein said capsid polypeptide comprises the amino acid sequence of any one of SEQ ID NOs:2-1103, wherein said AAV vectors infect said retinal cells and drive expression of said exogenous nucleic acid sequence within said retinal cells, thereby treating said retinal condition.

36. The method of claim 35, wherein said mammal is a human.

37. The method of any one of claims 35-36, wherein said retinal condition is selected from the group consisting of cone dystrophy, cone/rod dystrophy, retinitis pigmentosa, macular degeneration, achromatopsia, blue cone monochromcy, and color blindness.

38. The method of any one of claims 35-37, wherein said capsid polypeptide comprises the amino acid sequence of SEQ ID NO:1 or SEQ ID NO:2206 except that said amino acid sequence of any one of SEQ ID NOs:2-1103 is located between amino acid positions 587 and 588 of SEQ ID NO:1 or SEQ ID NO:2206.

39. The method of any one of claims 35-37, wherein said capsid polypeptide comprises the amino acid sequence of SEQ ID NO:1 or SEQ ID NO:2206 except that the amino acids from position 585 to 590 of SEQ ID NO:1 are replaced with said amino acid sequence of any one of SEQ ID NOs:2-1103 or SEQ ID NO:2206.

40. The method of any one of claims 35-39, wherein said vectors are AAV2 vectors.

41. The method of any one of claims 35-40, wherein said exogenous nucleic acid sequence encodes an RNA.

42. The method of claim 41, wherein said RNA is an siRNA or a microRNA.

43. The method of any one of claims 35-40, wherein said exogenous nucleic acid encodes a polypeptide.

44. The method of claim 43, wherein said polypeptide is an ABCA4 polypeptide, a CRB1 polypeptide, an NPHP5 polypeptide, and an NR2E3 polypeptide.

45. The method of any one of claims 35-44, wherein said method comprises intravitreally administering a composition comprising said vectors to said mammal, thereby contacting said retinal cells with said vectors.

46. The method of claim 45, wherein said composition comprises from about 1×10⁷ to about 1×10¹⁴ of said vectors.

47. A non-naturally occurring AAV capsid polypeptide, wherein said capsid polypeptide comprises the amino acid sequence of SEQ ID NO:1 or SEQ ID NO:2206 comprising an amino acid sequence insert of Formula A located between amino acid positions 587 and 588 of SEQ ID NO:1 or SEQ ID NO:2206, wherein said Formula A is:
-L1-INSERT-L2-,
wherein said L1 and said L2 are each independently optional amino acid linkers having one, two, or three amino acids, and wherein INSERT represents the amino acid sequence of any of the sequence identifiers of Tables 1A-1L without the starting “LA” and the ending “A.”

48. The capsid polypeptide of claim 47, wherein said L1 is one amino acid X1.

49. The capsid polypeptide of claim 48, wherein said X1 is selected from the group of amino acid residues consisting of A, V, I, and L.

50. The capsid polypeptide of claim 48, wherein said X1 is A.

51. The capsid polypeptide of claim 47, wherein said L1 is two amino acids X2-X1.

52. The capsid polypeptide of claim 51, wherein said X1 is selected from the group of amino acid residues consisting of A, V, I, and L.

53. The capsid polypeptide of claim 51, wherein said X1 is A.

54. The capsid polypeptide of any one of claims 51-53, wherein said X2 is selected from the group of amino acid residues consisting of A, V, I, and L.

55. The capsid polypeptide of claim 54, wherein said X2 is L.

56. The capsid polypeptide of claim 51, wherein said X2-X1 is LA.

57. The capsid polypeptide of claim 47, wherein said L1 is three amino acids X3-X2-X1.

58. The capsid polypeptide of claim 57, wherein said X1 is selected from the group of amino acid residues consisting of A, V, I, and L.

59. The capsid polypeptide of claim 58, wherein said X1 is A.

60. The capsid polypeptide of any one of claims 57-59-111, wherein said X2 is selected from the group of amino acid residues consisting of A, V, I, and L.

61. The capsid polypeptide of claim 60, wherein said X2 is L.

62. The capsid polypeptide of claim 57, wherein said X2-X1 is LA.

63. The capsid polypeptide of any one of claims 57-62, wherein said X3 is selected from the group of amino acid residues consisting of A, V, I, and L.

64. The capsid polypeptide of claim 47, wherein said L1 is absent.

65. The capsid polypeptide of any one of claims 47-64, wherein said L2 is one amino acid Z1.

66. The capsid polypeptide of claim 65, wherein said Z1 is selected from the group of amino acid residues consisting of A, V, I, and L.

67. The capsid polypeptide of claim 66, wherein said Z1 is A.

68. The capsid polypeptide of any one of claims 47-64, wherein said L2 is two amino acids Z1-Z2.

69. The capsid polypeptide of claim 68, wherein said Z1 is selected from the group of amino acid residues consisting of A, V, I, and L.

70. The capsid polypeptide of claim 69, wherein said Z1 is A.

71. The capsid polypeptide of any one of claims 68-70, wherein said Z2 is selected from the group of amino acid residues consisting of A, V, I, and L.

72. The capsid polypeptide of claim 71, wherein said Z2 is L.

73. The capsid polypeptide of claim 68, wherein said Z1-Z2 is AL.

74. The capsid polypeptide of any one of claims 47-64, wherein said L2 is three amino acids Z1-Z2-Z3.

75. The capsid polypeptide of claim 74, wherein said Z1 is selected from the group of amino acid residues consisting of A, V, I, and L.

76. The capsid polypeptide of claim 75, wherein said Z1 is A.

77. The capsid polypeptide of any one of claims 74-76, wherein said Z2 is selected from the group of amino acid residues consisting of A, V, I, and L.

78. The capsid polypeptide of claim 77, wherein said Z2 is L.

79. The capsid polypeptide of claim 74, wherein said Z1-Z2 is AL.

80. The capsid polypeptide of any one of claims 74-79, wherein said Z3 is selected from the group of amino acid residues consisting of A, V, I, and L.

81. The capsid polypeptide of any one of claims 47-64, wherein said L2 is absent.

82. The capsid polypeptide of claim 47, wherein said amino acid sequence insert comprises any one of the amino acid sequences of a sequence identifier of Tables 1A-1L that starts with “LA” and ends with “A.”

83. A non-naturally occurring adeno-associated virus (AAV) vector comprising an AAV capsid polypeptide according to any one of claims 47-82.

84. A viral particle comprising a capsid polypeptide of any one of claims 47-82.

85. A method for administering an exogenous nucleic acid sequence to a mammal in need thereof, wherein said method comprises administering an effective amount of a vector of claim 83 to said mammal, wherein said vector comprising said exogenous nucleic acid sequence.

86. The method of claim 85, wherein said mammal is a human.

87. The method of any one of claims 85-86, wherein said administering comprises administering said effective amount to an eye of said mammal.

88. The method of any one of claims 85-87, wherein said administering is sufficient to allow for expression of said exogenous nucleic acid sequence in a cell of said mammal.

89. The method of any one of claims 85-88, wherein said exogenous nucleic acid sequence encodes a therapeutic polypeptide.

90. A method of treating a retinal disorder in a patient in need thereof, comprising administering to the patient's eye an effective amount of an AAV vector, wherein the AAV vector comprises an AAV capsid polypeptide and an exogenous nucleic acid sequence, wherein the AAV capsid polypeptide is represented by Formula A.
